(19) United States
(12) Patent Application Publication
(10) Pub. No.: US 2010/0333234 A1
(43) Pub. Date: Dec. 30, 2010

(54) Transgenic Plants with Increased Stress Tolerance and Yield

(75) Inventors: Amber Shirley, Durham, NC (US); Damian Allen, Champaign, IL (US); Bryan D. McKersie, Research Triangle, NC (US); Nanfei Xu, Cary, NC (US); Piotr Puzio, Mariakerke (Gent) (BE); Richard Trethewey, Berlin (DE); Rodrigo Sarria-Millan, Durham, NC (US); Amy McCaskill, Apex, NC (US); Larissa Wilson, Cary, NC (US); Lalitree Darnielle, Durham, NC (US); Resham Kulkarni, Cary, NC (US)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 12/744,728

(22) PCT Filed: Nov. 27, 2008
(86) PCT No.: PCT/EP2008/066278

§ 371 Date: May 26, 2010

Related U.S. Application Data
(60) Provisional application No. 60/990,326, filed on Nov. 27, 2007.
(60) Provisional application No. 61/018,732, filed on Jan. 3, 2008.
(60) Provisional application No. 61/018,711, filed on Jan. 3, 2008.
(60) Provisional application No. 61/043,422, filed on Apr. 9, 2008.
(60) Provisional application No. 61/044,069, filed on Apr. 11, 2008.
(60) Provisional application No. 61/059,984, filed on Jun. 9, 2008.
(60) Provisional application No. 61/074,291, filed on Jun. 20, 2008.

(51) Int. Cl. A01H 1/00; A01H 5/00; C07H 21/04; C07K 14/00

(52) U.S. Cl. 800/290; 800/295; 536/23.6; 530/300; 800/278

(57) ABSTRACT

Polynucleotides are disclosed which are capable of enhancing a growth, yield under water-limited conditions, and/or increased tolerance to an environmental stress of a plant transformed to contain such polynucleotides. Also provided are methods of using such polynucleotides and transgenic plants and agricultural products, including seeds, containing such polynucleotides as transgenes.

This application claims priority benefit of the following U.S. provisional applications: U.S. Ser. No. 60/990,326, filed Nov. 27, 2007; U.S. Ser. No. 61/018,711, filed Jan. 3, 2008; U.S. Ser. No. 61/018,732, filed Jan. 3, 2008; U.S. Ser. No. 61/043,422, filed Apr. 9, 2008; U.S. Ser. No. 61/044,069, filed Apr. 11, 2008; U.S. Ser. No. 61/059,984, filed Jun. 9, 2008 and U.S. Ser. No. 61/074,291, filed Jun. 20, 2008, the entire contents of each of which being hereby incorporated by reference.

### FIELD OF THE INVENTION

This invention relates generally to transgenic plants which overexpress nucleic acid sequences encoding polypeptides capable of conferring increased stress tolerance and consequently, increased plant growth and crop yield, under normal or abiotic stress conditions. Additionally, the invention relates to novel isolated nucleic acid sequences encoding polypeptides that confer upon a plant increased tolerance under abiotic stress conditions, and/or increased plant growth and/or increased yield under normal or abiotic stress conditions.

In another embodiment, this invention relates to transgenic plants which overexpress isolated polynucleotides that encode polypeptides active in fatty acid and sterol metabolism, in specific plant tissues and organelles, thereby improving yield of said plants.

### BACKGROUND OF THE INVENTION

Abiotic environmental stresses, such as drought, salinity, heat, and cold, are major limiting factors of plant growth and crop yield. Crop yield is defined herein as the number of bushels of relevant agricultural product (such as grain, forage, or seed) harvested per acre. Crop losses and crop yield losses of major crops such as soybean, rice, maize (corn), cotton, and wheat caused by these stresses represent a significant economic and political factor and contribute to food shortages in many underdeveloped countries.

Water availability is an important aspect of the abiotic stresses and their effects on plant growth. Continuous exposure to drought conditions causes major alterations in the plant metabolism which ultimately lead to cell death and consequently to yield losses. Because high salt content in some soils results in less water being available for cell intake, high salt concentration has an effect on plants similar to the effect of drought on plants. Additionally, under freezing temperatures, plant cells lose water as a result of ice formation within the plant. Accordingly, crop damage from drought, heat, salinity, and cold stress, is predominantly due to dehydration.

Because plants are typically exposed to conditions of reduced water availability during their life cycle, most plants have evolved protective mechanisms against desiccation caused by abiotic stresses. However, if the severity and duration of desiccation conditions are too great, the effects on development, growth, plant size, and yield of most crop plants are profound. Developing plants efficient in water use is therefore a strategy that has the potential to significantly improve human life on a worldwide scale.

Traditional plant breeding strategies are relatively slow and require abiotic stress-tolerant founder lines for crossing with other germplasm to develop new abiotic stress-resistant lines. Limited germplasm resources for such founder lines and incompatibility in crosses between distantly related plant species represent significant problems encountered in conventional breeding. Breeding for tolerance has been largely unsuccessful.

Many agricultural biotechnology companies have attempted to identify genes that could confer tolerance to abiotic stress responses, in an effort to develop transgenic abiotic stress-tolerant crop plants. Although some genes that are involved in stress responses, biomass or water use efficiency in plants have been characterized, the characterization and cloning of plant genes that confer stress tolerance and/or water use efficiency remains largely incomplete and fragmented. To date, success at developing transgenic abiotic stress-tolerant crop plants has been limited, and no such plants have been commercialized. There is a need, therefore, to identify additional genes that have the capacity to increase yield of crop plants.

In order to develop transgenic abiotic stress-tolerant crop plants, it is necessary to assay a number of parameters in model plant systems, greenhouse studies of crop plants, and in field trials. For example, water use efficiency (WUE), is a parameter often correlated with drought tolerance. Studies of a plant's response to desiccation, osmotic shock, and temperature extremes are also employed to determine the plant's tolerance or resistance to abiotic stresses. When testing for the impact of the presence of a transgene on a plant's stress tolerance, the ability to standardize soil properties, temperature, water and nutrient availability and light intensity is an intrinsic advantage of greenhouse or plant growth chamber environments compared to the field.

WUE has been defined and measured in multiple ways. One approach is to calculate the ratio of whole plant dry weight, to the weight of water consumed by the plant throughout its life. Another variation is to use a shorter time interval when biomass accumulation and water use are measured. Yet another approach is to use measurements from restricted parts of the plant, for example, measuring only aerial growth and water use. WUE also has been defined as the ratio of CO₂ uptake to water vapor loss from a leaf or portion of a leaf, often measured over a very short time period (e.g. seconds/minutes). The ratio of ¹³C/¹²C fixed in plant tissue, and measured with an isotope ratio mass-spectrometer, also has been used to estimate WUE in plants using C₃ photosynthesis.

An increase in WUE is informative about the relatively improved efficiency of growth and water consumption, but this information taken alone does not indicate whether one of these two processes has changed or both have changed. In selecting traits for improving crops, an increase in WUE due to a decrease in water use, without a change in growth would have particular merit in an irrigated agricultural system where the water input costs were high. An increase in WUE driven mainly by an increase in growth without a corresponding jump in water use would have applicability to all agricultural systems. In many agricultural systems where water supply is not limiting, an increase in growth, even if it came at the expense of an increase in water use (i.e. no change in WUE), could also increase yield. Therefore, new methods to increase both WUE and biomass accumulation are required to improve agricultural productivity.

Grain yield improvements by conventional breeding have nearly reached a plateau in maize. Because the harvest index, the ratio of yield biomass to the total cumulative biomass at harvest, in maize has remained essentially unchanged during selection for grain yield over the last hundred or so years, the yield improvements have been realized from the increased total biomass production per unit land area. This increased total biomass has been achieved by increasing planting density, which has led to adaptive phenotypic alterations, such as a reduction in leaf angle and tassel size, the former to reduce shading of lower leaves and the latter perhaps to increase harvest index.

Concomitant with measurements of parameters that correlate with abiotic stress tolerance are measurements of parameters that indicate the potential impact of a transgene on crop yield. For forage crops like alfalfa, silage corn, and hay, the plant biomass correlates with the total yield. For grain crops, however, other parameters have been used to estimate yield, such as plant size, as measured by total plant dry weight, above-ground dry weight, above-ground fresh weight, leaf area, stem volume, plant height, rosette diameter, leaf length, root length, root mass, tiller number, and leaf number. Plant size at an early developmental stage will typically correlate with plant size later in development. A larger plant with a greater leaf area can typically absorb more light and carbon dioxide than a smaller plant and therefore will likely gain a greater weight during the same period. This is in addition to the potential continuation of the micro-environmental or genetic advantage that the plant had to achieve the larger size initially. There is a strong genetic component to plant size and growth rate, and so for a range of diverse genotypes plant size under one environmental condition is likely to correlate with size under another. In this way a standard environment is used to approximate the diverse and dynamic environments encountered at different locations and times by crops in the field.

Population increases and climate change have brought the possibility of global food, feed, and fuel shortages into sharp focus in recent years. Agriculture consumes 70% of water used by people, at a time when rainfall in many parts of the world is declining. In addition, as land use shifts from farms to cities and suburbs, fewer hectares of arable land are available to grow agricultural crops. Agricultural biotechnology has attempted to meet humanity's growing needs through genetic modifications of plants that could increase crop yield, for example, by conferring better tolerance to abiotic stress responses or by increasing biomass.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a lower water potential have the potential to conduct more photosynthesis and thereby to produce more biomass and economic yield in many agricultural systems.

Agricultural biotechnologists have used assays in model plant systems, greenhouse studies of crop plants, and field trials in their efforts to develop transgenic plants that exhibit increased yield, either through increases in abiotic stress tolerance or through increased biomass.

An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Agricultural biotechnologists also use measurements of other parameters that indicate the potential impact of a transgene on crop yield. For forage crops like alfalfa, silage corn, and hay, the plant biomass correlates with the total yield. For grain crops, however, other parameters have been used to estimate yield, such as plant size, as measured by total plant dry weight, above-ground dry weight, above-ground fresh weight, leaf area, stem volume, plant height, rosette diameter, leaf length, root length, root mass, tiller number, and leaf number. Plant size at an early developmental stage will typically correlate with plant size later in development. A larger plant with a greater leaf area can typically absorb more light and carbon dioxide than a smaller plant and therefore will likely gain a greater weight during the same period. There is a strong genetic component to plant size and growth rate, and so for a range of diverse genotypes plant size under one environmental condition is likely to correlate with size under another. In this way a standard environment is used to approximate the diverse and dynamic environments encountered at different locations and times by crops in the field.

Harvest index, the ratio of seed yield to above-ground dry weight, is relatively stable under many environmental conditions and so a robust correlation between plant size and grain yield is possible. Plant size and grain yield are intrinsically linked, because the majority of grain biomass is dependent on current or stored photosynthetic productivity by the leaves and stem of the plant. Therefore, selecting for plant size, even at early stages of development, has been used as to screen for plants that may demonstrate increased yield when exposed to field testing. As with abiotic stress tolerance, measurements of plant size in early development, under standardized conditions in a growth chamber or greenhouse, are standard practices to measure potential yield advantages conferred by the presence of a transgene.

Fatty acids are crucial components of many processes related to growth and development and stress tolerance of plants. Fatty acids are sources of energy and as well being physical components of both intracellular membrane structures and extracellular structures, such as waxes in leaf cuticles. Fatty acid synthesis is strictly regulated in plants. FIG. 16 sets forth a summary diagram of fatty acid biosynthesis in plants.

Plant sterols comprise a group of compounds related to cholesterol, including campesterol, sitosterol and stigmasterol that are components of membrane bilayers. Sterol concentration and partitioning in the lipid bilayer influences the physical properties of the membranes such as fluidity and phase transitions. Cell membranes are sites for perturbation during environmental stress of plants. Brassinosteroids are a class of plant growth regulator that are synthesized from plant sterol precursors such as campesterol. Application of brassinosteroids to plants causes a diverse set of responses related to cell growth and development, including ethylene production, proton transport and cellulose microfibril orientation. Brassinosteroid biosynthesis mutants of Arabidopsis, pea and tomato are dwarf, indicating that brassinosteroid concentration regulates cell elongation in plants.

Plant sterols are synthesized from squalene, and the biochemical steps related to squalene synthesis from isopentenyl pyrophosphate are summarized in FIG. 23. Three enzymes act sequentially to produce plant sterols: geranyltranstransferase (EC 2.5.1.10, also denoted as farnesyl diphosphate synthase or FPS), squalene synthase (EC 2.5.1.21, also denoted as SQS or farnesyl-diphosphate farnesyltransferase), and squalene epoxidase (EC 1.14.99.7, also denoted as squalene monooxigenase).

There is a need, therefore, to identify additional genes expressed in stress tolerant plants and/or plants that are efficient in water use that have the capacity to confer stress tolerance and/or increased water use efficiency to the host plant and to other plant species. Newly generated stress tolerant plants and/or plants with increased water use efficiency will have many advantages, such as an increased range in which the crop plants can be cultivated, by for example, decreasing the water requirements of a plant species. Other desirable advantages include increased resistance to lodging, the bending of shoots or stems in response to wind, rain, pests, or disease.

The present inventors have found that transforming a plant with certain polynucleotides results in enhancement of the plant's growth and response to environmental stress, and accordingly the yield of the agricultural products of the plant is increased, when the polynucleotides are present in the plant as transgenes. The polynucleotides capable of mediating such enhancements have been isolated from Physcomitrella patens, Brassica napus, Zea mays, Glycine max, Linum usitatissimum, Oryza sativa, Helianthus annuus, Arabidopsis thaliana, Hordeum vulgare or Triticum aestivum, and the sequences thereof are set forth in the Sequence Listing as indicated in Table 1.

The term “table 1” used in this specification is to be taken to specify the content of table 1A, table 1B, table 10, table 1D, table 1E, table 1F and/or table 1G. The term “table 1A” used in this specification is to be taken to specify the content of table 1A. The term “table 1B” used in this specification is to be taken to specify the content of table 1B. The term “table 10” used in this specification is to be taken to specify the content of table 10. The term “table 1D” used in this specification is to be taken to specify the content of table 1D. The term “table 1E” used in this specification is to be taken to specify the content of table 1E. The term “table 1F” used in this specification is to be taken to specify the content of table 1F. The term “table 1G” used in this specification is to be taken to specify the content of table 1G.

In one preferred embodiment, the term “table 1” means table 1A. In another preferred embodiment, the term “table 1” means table 1B. In another preferred embodiment, the term “table 1” means table 10. In another preferred embodiment, the term “table 1” means table 1D. In another preferred embodiment, the term “table 1” means table 1E. In another preferred embodiment, the term “table 1” means table 1F. In another preferred embodiment, the term “table 1” means table 1G.

TABLE 1A

| | | Polynucleotide | Amino acid |
|---|---|---|---|
| Gene Name | Organism | SEQ ID NO | SEQ ID NO |
| GM47143343 | G. max | 1 | 2 |
| EST431 | P. patens | 3 | 4 |
| EST253 | P. patens | 5 | 6 |
| TA54298452 | T. aestivum | 7 | 8 |
| GM59742369 | G. max | 9 | 10 |
| LU61585372 | L. usitatissimum | 11 | 12 |
| BN44703759 | B. napus | 13 | 14 |
| GM59703946 | G. max | 15 | 16 |
| GM59589775 | G. max | 17 | 18 |
| LU61696985 | L. usitatissimum | 19 | 20 |
| ZM62001130 | Z. mays | 21 | 22 |
| HA66796355 | H. annuus | 23 | 24 |
| LU61684898 | L. usitatissimum | 25 | 26 |
| LU61597381 | L. usitatissimum | 27 | 28 |
| EST272 | P. patens | 29 | 30 |
| BN42920374 | B. napus | 31 | 32 |
| BN45700248 | B. napus | 33 | 34 |
| BN47678601 | B. napus | 35 | 36 |
| GMsj02a06 | G. max | 37 | 38 |
| GM50305602 | G. max | 39 | 40 |
| EST500 | P. patens | 41 | 42 |
| EST401 | P. patens | 43 | 44 |
| BN51391539 | B. napus | 45 | 46 |
| GM59762784 | G. max | 47 | 48 |
| BN44099508 | B. napus | 49 | 50 |
| BN45789913 | B. napus | 51 | 52 |
| BN47959187 | B. napus | 53 | 54 |
| BN51418316 | B. napus | 55 | 56 |
| GM59691587 | G. max | 57 | 58 |
| ZM62219224 | Z. mays | 59 | 60 |
| EST591 | P. patens | 61 | 62 |
| BN51345938 | B. napus | 63 | 64 |
| BN51456960 | B. napus | 65 | 66 |
| BN43562070 | B. napus | 67 | 68 |
| TA60004809 | T. aestivum | 69 | 70 |
| ZM62079719 | Z. mays | 71 | 72 |
| BN42110642 | B. napus | 73 | 74 |
| GM59794180 | G. max | 75 | 76 |
| GMsp52b07 | G. max | 77 | 78 |
| ZM57272608 | Z. mays | 79 | 80 |
| EST336 | P. patens | 81 | 82 |
| BN43012559 | B. napus | 83 | 84 |
| BN44705066 | B. napus | 85 | 86 |
| GM50962576 | G. max | 87 | 88 |
| GMsk93h09 | G. max | 89 | 90 |
| GMso31a02 | G. max | 91 | 92 |
| LU61649369 | L. usitatissimum | 93 | 94 |
| LU61704197 | L. usitatissimum | 95 | 96 |
| ZM57508275 | Z. mays | 97 | 98 |
| ZM59288476 | Z. mays | 99 | 100 |

In another embodiment, the invention provides a transgenic plant transformed with an expression cassette comprising an isolated polynucleotide encoding a mitogen activated protein kinase comprising a protein kinase domain of SEQ ID NO:2; SEQ ID NO:4; SEQ ID NO:6; SEQ ID NO:8; SEQ ID NO:10; SEQ ID NO:12; SEQ ID NO:14; SEQ ID NO:16; SEQ ID NO:18; SEQ ID NO:20; SEQ ID NO:22; SEQ ID NO:24; SEQ ID NO:26; SEQ ID NO:28; SEQ ID NO:30; SEQ ID NO:32; SEQ ID NO:34; SEQ ID NO:36; or SEQ ID NO:38.

In another embodiment, the invention provides a transgenic plant transformed with an expression cassette comprising an isolated polynucleotide encoding a calcium dependent protein kinase comprising a protein kinase domain of SEQ ID NO:40; SEQ ID NO:42; SEQ ID NO:44; SEQ ID NO:46; SEQ ID NO:48; SEQ ID NO:50; SEQ ID NO:52; SEQ ID NO:54; SEQ ID NO:56; SEQ ID NO:58; SEQ ID NO:60; SEQ ID NO:62; SEQ ID NO:64; SEQ ID NO:66; SEQ ID NO:68; SEQ ID NO:70; or SEQ ID NO:72.

In another embodiment, the invention provides a transgenic plant transformed with an expression cassette comprising an isolated polynucleotide encoding a cyclin dependent protein kinase comprising a protein kinase domain of SEQ ID NO:74; SEQ ID NO:76; SEQ ID NO:78; or SEQ ID NO:80.

In another embodiment, the invention provides a transgenic plant transformed with an expression cassette comprising an isolated polynucleotide encoding a probable serine/threonine-specific protein kinase comprising a protein kinase domain of SEQ ID NO:82; SEQ ID NO:84; SEQ ID NO:86; SEQ ID NO:88; SEQ ID NO:90; SEQ ID NO:92; SEQ ID NO:94; SEQ ID NO:96; SEQ ID NO:98; SEQ ID NO:100.

TABLE 1B

| | | Polynucleotide | Amino acid |
|---|---|---|---|
| Gene Name | Organism | SEQ ID NO | SEQ ID NO |
| BN42194524 | B. napus | 101 | 102 |
| ZM68498581 | Z. mays | 103 | 104 |
| BN42062606 | B. napus | 105 | 106 |
| BN42261838 | B. napus | 107 | 108 |
| BN43722096 | B. napus | 109 | 110 |
| GM50585691 | G. max | 111 | 112 |
| GMsa56c07 | G. max | 113 | 114 |
| GMsb20d04 | G. max | 115 | 116 |
| GMsg04a02 | G. max | 117 | 118 |
| GMsp36c10 | G. max | 119 | 120 |
| GMsp82f11 | G. max | 121 | 122 |
| GMss66f03 | G. max | 123 | 124 |
| LU61748885 | L. usitatissimum | 125 | 126 |
| OS36582281 | O. sativa | 127 | 128 |
| OS40057356 | O. sativa | 129 | 130 |
| ZM57588094 | Z. mays | 131 | 132 |
| ZM67281604 | Z. mays | 133 | 134 |
| ZM68466470 | Z. mays | 135 | 136 |

In another embodiment, the invention provides a transgenic plant transformed with an expression cassette comprising an isolated polynucleotide encoding a full-length polypeptide having phospholipid hydroperoxide glutathione peroxidase activity, wherein the polypeptide comprises a glutathione peroxidase domain of SEQ ID NO:102; SEQ ID NO:104; SEQ ID NO:106; SEQ ID NO:108; SEQ ID NO:110; SEQ ID NO:112; SEQ ID NO:114; SEQ ID NO:116; SEQ ID NO:118; SEQ ID NO:120; SEQ ID NO:122; SEQ ID NO:124; SEQ ID NO:126; SEQ ID NO:128; SEQ ID NO:130; SEQ ID NO:132; SEQ ID NO:134; or SEQ ID NO:136.

TABLE 1C

| | | Polynucleotide | Amino acid |
|---|---|---|---|
| Gene Name | Organism | SEQ ID NO | SEQ ID NO |
| BN45660154_5 | B. napus | 137 | 138 |
| BN45660154_8 | B. napus | 139 | 140 |
| ZM58885021 | Z. mays | 141 | 142 |
| BN46929759 | B. napus | 143 | 144 |
| BN43100775 | B. napus | 145 | 146 |
| GM59673822 | G. max | 147 | 148 |
| ZM59314493 | Z. mays | 149 | 150 |
| GMsk21ga12 | G. max | 151 | 152 |
| ZM62043790 | Z. mays | 153 | 154 |
| GMsk21g122 | G. max | 155 | 156 |
| AT5G60750 | A. thaliana | 157 | 158 |
| BN47819599 | B. napus | 159 | 160 |
| ZM65102675 | Z. mays | 161 | 162 |
| BN51278543 | B. napus | 163 | 164 |
| GM59587627 | G. max | 165 | 166 |
| GMsae76c10 | G. max | 167 | 168 |
| ZM68403475 | Z. mays | 169 | 170 |
| ZMTD140063555 | Z. mays | 171 | 172 |
| BN43069781 | B. napus | 173 | 174 |
| BN48622391 | B. napus | 175 | 176 |
| GM50247805 | G. max | 177 | 178 |
| ZM62208861 | Z. mays | 179 | 180 |
| GM49819537 | G. max | 181 | 182 |
| BN42562310 | B. napus | 183 | 184 |
| GM47121078 | G. max | 185 | 186 |
| GMsf89h03 | G. max | 187 | 188 |
| HA66670700 | H. annuus | 189 | 190 |
| GM50390979 | G. max | 191 | 192 |
| GM597200141 | G. max | 193 | 194 |
| GMsab62c11 | G. max | 195 | 196 |
| GMsl42e03 | G. max | 197 | 198 |
| GMss72c01 | G. max | 199 | 200 |
| HV100766 | H. vulgare | 201 | 202 |
| EST397 | P. patens | 203 | 204 |
| ZM57926241 | Z. mays | 205 | 206 |

TABLE 1D

| | | Polynucleotide | Amino acid |
|---|---|---|---|
| Gene Name | Organism | SEQ ID NO | SEQ ID NO |
| EST285 | P. patens | 207 | 208 |
| BN42471769 | B. napus | 209 | 210 |
| ZM100324 | Z. mays | 211 | 212 |
| BN42817730 | B. napus | 213 | 214 |
| BN45236208 | B. napus | 215 | 216 |
| BN46730374 | B. napus | 217 | 218 |
| BN46832560 | B. napus | 219 | 220 |
| BN46868821 | B. napus | 221 | 222 |
| GM48927342 | G. max | 223 | 224 |
| GM48955695 | G. max | 225 | 226 |
| GM48958569 | G. max | 227 | 228 |
| GM50526381 | G. max | 229 | 230 |
| HA66511283 | H. annuus | 231 | 232 |
| HA66563970 | H. annuus | 233 | 234 |
| HA66692703 | H. annuus | 235 | 236 |
| HA66822928 | H. annuus | 237 | 238 |
| LU61569679 | L. usitatissimum | 239 | 240 |
| LU61703351 | L. usitatissimum | 241 | 242 |
| LU61962194 | L. usitatissimum | 243 | 244 |
| TA54564073 | T. aestivum | 245 | 246 |
| TA54788773 | T. aestivum | 247 | 248 |
| TA56412836 | T. aestivum | 249 | 250 |
| ZM65144673 | Z. mays | 251 | 252 |

TABLE 1E

| | | Polynucleotide | Amino Acid |
|---|---|---|---|
| Gene Name | Organism | SEQ ID NO | SEQ ID NO |
| EST314 | P. patens | 253 | 254 |
| EST322 | P. patens | 255 | 256 |
| EST589 | P. patens | 257 | 258 |
| BN45899621 | B. napus | 259 | 260 |
| BN51334240 | B. napus | 261 | 262 |
| BN51345476 | B. napus | 263 | 264 |
| BN42856089 | B. napus | 265 | 266 |
| BN43206527 | B. napus | 267 | 268 |
| GMsf85h09 | G. max | 269 | 270 |
| GMsj98e01 | G. max | 271 | 272 |
| GMsu65h07 | G. max | 273 | 274 |
| HA66777473 | H. annuus | 275 | 276 |
| LU61781371 | L. usitatissimum | 277 | 278 |
| LU61589678 | L. usitatissimum | 279 | 280 |
| LU61857781 | L. usitatissimum | 281 | 282 |
| TA55079288 | T. aestivum | 283 | 284 |
| ZM59400933 | Z. mays | 285 | 286 |

In one embodiment, the invention provides the novel isolated polynucleotides and proteins of Table 1.

In another embodiment, the invention provides a transgenic plant transformed with an expression cassette comprising an isolated polynucleotide encoding a full-length polypeptide comprising a TCP family transcription factor domain of SEQ ID NO:138; SEQ ID NO:140; SEQ ID NO:142; or SEQ ID NO:144.

In another embodiment, the invention provides a transgenic plant transformed with an expression cassette comprising an isolated polynucleotide encoding a ribosomal protein S6 kinase polypeptide comprising a kinase domain of SEQ ID NO:146; SEQ ID NO:148; or SEQ ID NO:150.

In another embodiment, the invention provides a transgenic plant transformed with an expression cassette comprising an isolated polynucleotide encoding a full-length polypeptide comprising a CAAX amino terminal protease family protein domain of SEQ ID NO:158; SEQ ID NO:160; or SEQ ID NO:162.

In another embodiment, the invention provides a transgenic plant transformed with an expression cassette comprising an isolated polynucleotide encoding a DNA binding protein comprising a metallopeptidase family M24 domain of SEQ ID NO:164; SEQ ID NO:166; SEQ ID NO:168; or SEQ ID NO:170; or SEQ ID NO:172.

In another embodiment, the invention provides a transgenic plant transformed with an expression cassette comprising an isolated polynucleotide encoding a rev interacting protein mis3 selected from the group consisting of SEQ ID NO:176; SEQ ID NO:178; and SEQ ID NO:180.

In another embodiment, the invention provides a transgenic plant transformed with an expression cassette comprising an isolated polynucleotide encoding a GRF1 interacting factor comprising an SSXT protein (N terminal region) domain of SEQ ID NO:182; SEQ ID NO:184; SEQ ID NO:186; or SEQ ID NO:188.

In another embodiment, the invention provides a transgenic plant transformed with an expression cassette comprising an isolated polynucleotide encoding a eukaryotic translation initiation factor 4A comprising a helicase of SEQ ID NO:190; SEQ ID NO:192; SEQ ID NO:194; or SEQ ID NO:196; SEQ ID NO:198; or SEQ ID NO:200.

In another embodiment, the invention provides a transgenic plant transformed with an expression cassette comprising an isolated polynucleotide encoding a full-length TGF beta receptor interacting protein comprising a WD domain of SEQ ID NO:152; SEQ ID NO:154; or SEQ ID NO:156.

In another embodiment, the invention provides a transgenic plant transformed with an expression cassette comprising an isolated polynucleotide having a sequence selected from the group consisting of SEQ ID NO:173; SEQ ID NO:201; SEQ ID NO:203; and SEQ ID NO:205.

In one embodiment, the invention provides a transgenic plant transformed with an expression cassette comprising an isolated polynucleotide encoding an AP2 domain containing protein.

In one embodiment, the invention provides a transgenic plant transformed with an expression cassette comprising an isolated polynucleotide encoding a brassinosteroid biosynthetic LKB-like protein comprising a LKB-like transmembrane domain of SEQ ID NO:254.

In another embodiment, the invention provides a transgenic plant transformed with an expression cassette comprising an isolated polynucleotide encoding a RING box protein comprising a RING box domain of SEQ ID NO:256.

In another embodiment, the invention provides a transgenic plant transformed with an expression cassette comprising an isolated polynucleotide encoding a serine/threonine protein phosphatase comprising a protein phosphatase domain of SEQ ID NO:258; SEQ ID NO:260; SEQ ID NO:262; SEQ ID NO:264; SEQ ID NO:266; SEQ ID NO:268; SEQ ID NO:270; SEQ ID NO:272; SEQ ID NO:274; SEQ ID NO:276; SEQ ID NO:278; SEQ ID NO:280; SEQ ID NO:282; SEQ ID NO:284; SEQ ID NO:286.

The present inventors have found that there are three critical components that must be optimized to achieve improvement in plant yield through the modification of fatty acid metabolism—the subcellular targeting of the protein, the level of gene expression and the regulatory properties of the protein. When targeted as described herein, the fatty acid metabolic polynucleotides and polypeptides set forth in Table 1F and Table 1G are capable of improving yield of transgenic plants.

TABLE 1F

| | | Polynucleotide | Amino acid SEQ |
|---|---|---|---|
| Gene Name | Organism | SEQ ID NO | ID NO |
| b1805 | Escherichia coli | 287 | 288 |
| YER015W | Saccharomyces | 289 | 290 |
| | cerevisiae | | |
| GM59544909 | G. max | 291 | 292 |
| GM59627238 | G. max | 293 | 294 |
| GM59727707 | G. max | 295 | 296 |
| ZM57432637 | Z. mays | 297 | 298 |
| ZM58913368 | Z. mays | 299 | 300 |
| ZM62001931 | Z. mays | 301 | 302 |
| ZM65438309 | Z. mays | 303 | 304 |
| GM59610424 | G. max | 305 | 306 |
| GM59661358 | G. max | 307 | 308 |
| GMst55d11 | G. max | 309 | 310 |
| ZM65362798 | Z. mays | 311 | 312 |
| ZM62261160 | Z. mays | 313 | 314 |
| ZM62152441 | Z. mays | 315 | 316 |
| b1091 | E. coli | 317 | 318 |
| b0185 | E. coli | 319 | 320 |
| b3256 | E. coli | 321 | 322 |
| BN49370246 | B. napus | 323 | 324 |
| GM59606041 | G. max | 325 | 326 |
| GM59537012 | G. max | 327 | 328 |
| b3255 | E. coli | 329 | 330 |
| BN49342080 | B. napus | 331 | 332 |
| BN45576739 | B. napus | 333 | 334 |
| b1095 | E. coli | 335 | 336 |
| GM48933354 | G. max | 337 | 338 |
| ZM59397765 | Zea mays | 339 | 340 |
| GM59563409 | G. max | 341 | 342 |
| B1093 | E. coli | 343 | 344 |
| slr0886 | Synechocystis | 345 | 346 |
| | PCC6803 | | |
| BN44033445 | B. napus | 347 | 348 |
| BN43251017 | B. napus | 349 | 350 |
| BN42133443 | B. napus | 351 | 352 |
| GM49771427 | G. max | 353 | 354 |
| GM48925912 | G. max | 355 | 356 |
| GM51007060 | G. max | 357 | 358 |
| GM59598120 | G. max | 359 | 360 |
| GM59619826 | G. max | 361 | 362 |
| GMsaa65f11 | G. max | 363 | 364 |
| GMsf29g01 | G. max | 365 | 366 |
| GMsn33h01 | G. max | 367 | 368 |
| GMsp73h12 | G. max | 369 | 370 |
| GMst67g06 | G. max | 371 | 372 |
| GMsu14e09 | G. max | 373 | 374 |
| GMsu65c05 | G. max | 375 | 376 |
| HV62626732 | H. vulgare | 377 | 378 |
| LU61764715 | L. usitatissimum | 379 | 380 |
| OS32620492 | O. sativa | 381 | 382 |
| ZM57377353 | Z. mays | 383 | 384 |
| ZM58204125 | Z. mays | 385 | 386 |
| ZM58594846 | Z. mays | 387 | 388 |
| ZM62192824 | Z. mays | 389 | 390 |
| ZM65173545 | Z. mays | 391 | 392 |
| ZM65173829 | Z. mays | 393 | 394 |
| ZM57603160 | Z. mays | 395 | 396 |
| slr1364 | Synechocystis | 397 | 398 |
| | PCC6803 | | |
| BN51403883 | B. napus | 399 | 400 |
| ZM65220870 | Z. mays | 401 | 402 |

In one embodiment, the invention provides a transgenic plant transformed with an expression cassette comprising, in operative association, an isolated polynucleotide encoding a promoter capable of enhancing gene expression in leaves; and an isolated polynucleotide encoding a full-length polypeptide which is a long-chain-fatty-acid-CoA ligase subunit of acyl-CoA synthetase; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; and an isolated polynucleotide encoding a full-length beta-ketoacyl-acyl carrier protein (hereinafter “ACP”) synthase polypeptide, wherein the transgenic plant demonstrates increased yield as compared to a wild type plant of the same variety which does not comprise the expression cassette.

In another embodiment, the invention provides a transgenic plant transformed with an expression cassette comprising in operative association, an isolated polynucleotide encoding a promoter capable of enhancing gene expression in leaves; an isolated polynucleotide encoding a mitochondrial transit peptide; and an isolated polynucleotide encoding a subunit of an acetyl-CoA carboxylase complex, wherein the transgenic plant demonstrates increased yield as compared to a wild type plant of the same variety which does not comprise the expression cassette. In accordance with this embodiment, the acetyl-CoA carboxylase subunit may be an acetyl-CoA carboxylase, a biotin carboxylase, or a biotin carboxyl carrier protein.

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length 3-oxoacyl-[ACP] synthase II polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter and an isolated polynucleotide encoding a full-length 3-oxoacyl-[ACP] reductase polypeptide; wherein the transgenic plant demonstrates increased yield as compared to a wild type plant of the same variety which does not comprise the expression cassette. The promoter employed in the expression vector of this embodiment may optionally be capable of enhancing expression in leaves. Morover, the expression vector of this embodiment may optionally comprise a mitochondrial or chloroplast transit peptide.

In another embodiment, the invention provides a transgenic plant transformed with an expression cassette comprising, in operative association, an isolated polynucleotide encoding a promoter, an isolated polynucleotide encoding a mitochondrial transit peptide, and an isolated polynucleotide encoding a full-length biotin synthetase polypeptide, wherein the transgenic plant demonstrates increased yield as compared to a wild type plant of the same variety which does not comprise the expression cassette.

TABLE 1G

| | | Polynucleotide | Amino acid SEQ |
|---|---|---|---|
| Gene Name | Organism | SEQ ID NO | ID NO |
| B0421 | Escherichia coli | 413 | 414 |
| YJL167W | Saccharomyces | 415 | 416 |
| | cerevisiae | | |
| BN42777400 | Brassica napus | 417 | 418 |
| BN43165280 | B. napus | 419 | 420 |
| GMsf33b12 | Glycine max | 421 | 422 |
| GMsa58c11 | G. max | 423 | 424 |
| GM48958315 | G. max | 425 | 426 |
| TA55347042 | T. aestivum | 427 | 428 |
| TA59981866 | T. aestivum | 429 | 430 |
| ZM68702208 | Zea mays | 431 | 432 |
| ZM62161138 | Z. mays | 433 | 434 |
| SQS1 | synthetic | 435 | 436 |
| SQS2 | synthetic | 437 | 438 |
| BN51386398 | B. napus | 439 | 440 |
| GM59738015 | G. max | 441 | 442 |
| ZM68433599 | Z. mays | 443 | 444 |
| YGR175C | S. cerevisiae | 445 | 446 |
| BN48837983 | B. napus | 447 | 448 |
| ZM62269276 | Z. mays | 449 | 450 |

In one embodiment, the invention provides a transgenic plant transformed with an expression cassette comprising, in operative association, an isolated polynucleotide encoding a promoter capable of enhancing gene expression in leaves; and an isolated polynucleotide encoding a mitochondrial transit peptide; and an isolated polynucleotide encoding a full-length polypeptide which is a farnesyl diphosphate synthase (hereinafter “FPS”); wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; an isolated polynucleotide encoding a chloroplast transit peptide, and an isolated polynucleotide encoding a full-length squalene synthase polypeptide, wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; an isolated polynucleotide encoding a chloroplast transit peptide; and an isolated polynucleotide encoding a full-length squalene epoxidase polypeptide; wherein the transgenic plant demonstrates increased yield as compared to a wild type plant of the same variety which does not comprise the expression cassette.

In a further embodiment, the invention concerns a seed produced by the transgenic plant of the invention, wherein the seed is true breeding for a transgene comprising the polynucleotide described above. Plants derived from the seed of the invention demonstrate increased tolerance to an environmental stress, and/or increased plant growth, and/or increased yield, under normal or stress conditions as compared to a wild type variety of the plant.

In a still another aspect, the invention concerns products produced by or from the transgenic plants of the invention, their plant parts, or their seeds, such as a foodstuff, fiber, feedstuff, food supplement, feed supplement, cosmetic or pharmaceutical.

The invention further provides certain isolated polynucleotides identified in Table 1, and certain isolated polypeptides identified in Table 1. The invention is also embodied in recombinant vector comprising an isolated polynucleotide of the invention.

In yet another embodiment, the invention concerns a method of producing the aforesaid transgenic plant, wherein the method comprises transforming a plant cell with an expression vector comprising an isolated polynucleotide of the invention, and generating from the plant cell a transgenic plant that expresses the polypeptide encoded by the polynucleotide. Expression of the polypeptide in the plant results in increased tolerance to an environmental stress, and/or growth, and/or yield under normal and/or stress conditions as compared to a wild type variety of the plant.

In still another embodiment, the invention provides a method of increasing a plant's tolerance to an environmental stress, and/or growth, and/or yield. The method comprises the steps of transforming a plant cell with an expression cassette comprising an isolated polynucleotide of the invention, and generating a transgenic plant from the plant cell, wherein the transgenic plant comprises the polynucleotide.

### DETAILED DESCRIPTION OF THE PREFERRED EMBODIMENTS

Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used.

In one embodiment, the invention provides a transgenic plant that overexpresses an isolated polynucleotide identified in Table 1, or a homolog thereof. The transgenic plant of the invention demonstrates an increased tolerance to an environmental stress as compared to a wild type variety of the plant. The overexpression of such isolated nucleic acids in the plant may optionally result in an increase in plant growth or in yield of associated agricultural products, under normal or stress conditions, as compared to a wild type variety of the plant. Such yield increases may result from promotion of floral organ development, root initiation, and yield, and for modulating leaf formation, phototropism, apical dominance, fruit development and the like.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like. The transgenic plant of the invention may be male sterile or male fertile, and may further include transgenes other than those that comprise the isolated polynucleotides described herein.

As used herein, the term “variety” refers to a group of plants within a species that share constant characteristics that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of one or more isolated polynucleotides introduced into a plant variety. As also used herein, the term “wild type variety” refers to a group of plants that are analyzed for comparative purposes as a control plant, wherein the wild type variety plant is identical to the transgenic plant (plant transformed with an isolated polynucleotide in accordance with the invention) with the exception that the wild type variety plant has not been transformed with an isolated polynucleotide of the invention. The term “wild type” as used herein refers to a plant cell, seed, plant component, plant tissue, plant organ, or whole plant that has not been genetically modified with an isolated polynucleotide in accordance with the invention.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

As used herein, the term “environmental stress” refers to a sub-optimal condition associated with salinity, drought, nitrogen, temperature, metal, chemical, pathogenic, or oxidative stresses, or any combination thereof. The terms “water use efficiency” and “WUE” refer to the amount of organic matter produced by a plant divided by the amount of water used by the plant in producing it, i.e., the dry weight of a plant in relation to the plant's water use. As used herein, the term “drought” refers to an environmental condition where the amount of water available to support plant growth or development is less than optimal. As used herein, the term “fresh weight” refers to everything in the plant including water. As used herein, the term “dry weight” refers to everything in the plant other than water, and includes, for example, carbohydrates, proteins, oils, and mineral nutrients.

Any plant species may be transformed to create a transgenic plant in accordance with the invention. The transgenic plant of the invention may be a dicotyledonous plant or a monocotyledonous plant. For example and without limitation, transgenic plants of the invention may be derived from any of the following diclotyledonous plant families: Leguminosae, including plants such as pea, alfalfa and soybean; Umbelliferae, including plants such as carrot and celery; Solanaceae, including the plants such as tomato, potato, aubergine, tobacco, and pepper; Cruciferae, Brassicaceae, particularly the genus Brassica, which includes plant such as oilseed rape, beet, cabbage, cauliflower and broccoli); and A. thaliana; Compositae, which includes plants such as lettuce; Malvaceae, which includes cotton; Fabaceae, which includes plants such as peanut, and the like. Transgenic plants of the invention may be derived from monocotyledonous plants, such as, for example, wheat, barley, sorghum, millet, rye, triticale, maize, rice, oats and sugarcane. Transgenic plants of the invention are also embodied as trees such as apple, pear, quince, plum, cherry, peach, nectarine, apricot, papaya, mango, and other woody species including coniferous and deciduous trees such as poplar, pine, sequoia, cedar, oak, and the like. Especially preferred are Arabidopsis thaliana, Nicotiana tabacum, oilseed rape, soybean, corn (maize), canola, cotton, wheat, linseed, potato and tagetes.

In one embodiment, the invention provides a transgenic plant transformed with an expression cassette comprising an isolated polynucleotide encoding mitogen activated protein kinase. The transgenic plant of this embodiment may comprise any polynucleotide encoding a mitogen activated protein kinase. Preferably, the transgenic plant of this embodiment comprises a polynucleotide encoding a full-length polypeptide having mitogen activated protein kinase activity, wherein the polypeptide comprises a domain selected from the group consisting of a domain having a sequence comprising amino acids 32 to 319 of SEQ ID NO:2; amino acids 42 to 329 of SEQ ID NO:4; amino acids 32 to 319 of SEQ ID NO:6; amino acids 32 to 310 of SEQ ID NO:8; amino acids 32 to 319 of SEQ ID NO:10; amino acids 32 to 319 of SEQ ID NO:12; amino acids 28 to 318 of SEQ ID NO:14; amino acids 32 to 326 of SEQ ID NO:16; amino acids 38 to 325 of SEQ ID NO:18; amino acids 44 to 331 of SEQ ID NO:20; amino acids 40 to 357 of SEQ ID NO:22; amino acids 60 to 346 of SEQ ID NO:24; amino acids 74 to 360 of SEQ ID NO:26; amino acids 47 to 334 of SEQ ID NO:28; amino acids 38 to 325 of SEQ ID NO:30; amino acids 32 to 319 of SEQ ID NO:32; amino acids 41 to 327 of SEQ ID NO:34; amino acids 43 to 329 of SEQ ID NO:36; and amino acids 58 to 344 of SEQ ID NO:38. Mitogen-activated protein kinases are characterized by the T-loop portion of their protein kinase domain which contains the amino acid motif TDY or TEY. This motif is a phosphorylation target of mitogen-activated protein kinase kinases, which are the next step in this type of signal transduction pathway. All of the domains described herein as being a part of a mitogen-activated protein kinase contain such a motif in register with the overall alignment provided in FIG. 1. More preferably, the transgenic plant of this embodiment comprises a polynucleotide encoding a mitogen activated protein kinase having a sequence comprising amino acids 1 to 368 of SEQ ID NO:2; amino acids 1 to 376 of SEQ ID NO:4; amino acids 1 to 368 of SEQ ID NO:6; amino acids 1 to 369 of SEQ ID NO:8; amino acids 1 to 371 of SEQ ID NO:10; amino acids 1 to 375 of SEQ ID NO:12; amino acids 1 to 523 of SEQ ID NO:14; amino acids 1 to 563 of SEQ ID NO:16; amino acids 1 to 373 of SEQ ID NO:18; amino acids 1 to 377 of SEQ ID NO:20; amino acids 1 to 404 of SEQ ID NO:22; amino acids 1 to 394 of SEQ ID NO:24; amino acids 1 to 415 of SEQ ID NO:26; amino acids 1 to 381 of SEQ ID NO:28; amino acids 1 to 376 of SEQ ID NO:30; amino acids 1 to 368 of SEQ ID NO:32; amino acids 1 to 372 of SEQ ID NO:34; amino acids 1 to 374 of SEQ ID NO:36; or amino acids 1 to 372 of SEQ ID NO:38.

In another embodiment, the invention provides a transgenic plant transformed with an expression cassette comprising an isolated polynucleotide encoding calcium dependent protein kinase. Plant-derived calcium-dependent protein kinases are characterized, in part, by the fusion of a protein kinase domain with a calmodulin-like calcium-binding domain. The calmodulin-like domain contains one or more calcium-binding EF hand structural motifs. All polypeptides listed herein as being a calcium-dependent protein kinase contain motifs characteristic of protein kinase domains and EF hand motifs.

The transgenic plant of this embodiment may comprise any polynucleotide encoding a calcium dependent protein kinase. Preferably, the transgenic plant of this embodiment comprises a polynucleotide encoding a full-length polypeptide having calcium dependent protein kinase activity, wherein the polypeptide comprises a protein kinase domain selected from the group consisting of a domain having a sequence comprising amino acids 59 to 317 of SEQ ID NO:40; amino acids 111 to 369 of SEQ ID NO:42; amino acids 126 to 386 of SEQ ID NO:44; amino acids 79 to 337 of SEQ ID NO:46; amino acids 80 to 338 of SEQ ID NO:48; amino acids 125 to 287 of SEQ ID NO:50; amino acids 129 to 391 of SEQ ID NO:52; amino acids 111 to 371 of SEQ ID NO:54; amino acids 61 to 319 of SEQ ID NO:56; amino acids 86 to 344 of SEQ ID NO:58; amino acids 79 to 337 of SEQ ID NO:60; amino acids 78 to 336 of SEQ ID NO:62; amino acids 90 to 348 of SEQ ID NO:64; amino acids 56 to 314 of SEQ ID NO:66; amino acids 67 to 325 of SEQ ID NO:68; amino acids 81 to 339 of SEQ ID NO:70; and amino acids 83 to 341 of SEQ ID NO:72 and at least one EF hand domain having a sequence selected from the group consisting of amino acids 364 to 392 of SEQ ID NO:40; amino acids 416 to 444 of SEQ ID NO:42; amino acids 433 to 461 of SEQ ID NO:44; amino acids 384 to 412 of SEQ ID NO:46; amino acids 385 to 413 of SEQ ID NO:48; amino acids 433 to 461 of SEQ ID NO:50; amino acids 436 to 463 of SEQ ID NO:52; amino acids 418 to 446 of SEQ ID NO:54; amino acids 366 to 394 of SEQ ID NO:56; amino acids 391 to 419 of SEQ ID NO:58; amino acids 384 to 412 of SEQ ID NO:60; amino acids 418 to 446 of SEQ ID NO:62; amino acids 395 to 423 of SEQ ID NO:64; amino acids 372 to 400 of SEQ ID NO:68; amino acids 388 to 416 of SEQ ID NO:72; amino acids 452 to 480 of SEQ ID NO:42; amino acids 470 to 498 of SEQ ID NO:44; amino acids 420 to 448 of SEQ ID NO:46; amino acids 421 to 449 of SEQ ID NO:48; amino acids 470 to 498 of SEQ ID NO:50; amino acids 472 to 500 of SEQ ID NO:52; amino acids 455 to 483 of SEQ ID NO:54; amino acids 402 to 430 of SEQ ID NO:56; amino acids 427 to 455 of SEQ ID NO:58; amino acids 420 to 448 of SEQ ID NO:60; amino acids 454 to 482 of SEQ ID NO:62; amino acids 444 to 472 of SEQ ID NO:68; amino acids 460 to 488 of SEQ ID NO:72; amino acids 488 to 516 of SEQ ID NO:42; amino acids 512 to 540 of SEQ ID NO:44; amino acids 456 to 484 of SEQ ID NO:46; amino acids 457 to 485 of SEQ ID NO:48; amino acids 510 to 535 of SEQ ID NO:50; amino acids 512 to 537 of SEQ ID NO:52; amino acids 497 to 525 of SEQ ID NO:54; amino acids 438 to 466 of SEQ ID NO:56; amino acids 463 to 491 of SEQ ID NO:58; amino acids 456 to 484 of SEQ ID NO:60; amino acids 522 to 550 of SEQ ID NO:42; amino acids 546 to 570 of SEQ ID NO:44; amino acids 491 to 519 of SEQ ID NO:46; amino acids 492 to 520 of SEQ ID NO:48; amino acids 542 to 570 of SEQ ID NO:50; amino acids 542 to 570 of SEQ ID NO:52; amino acids 531 to 555 of SEQ ID NO:54; amino acids 474 to 502 of SEQ ID NO:56; amino acids 497 to 525 of SEQ ID NO:58; and amino acid 490 to 518 of SEQ ID NO:60; amino acids 489 to 517 of SEQ ID NO:62; amino acids 501 to 529 of SEQ ID NO:64; amino acids 470 to 498 of SEQ ID NO:66; amino acids 479 to 507 of SEQ ID NO:68; amino acids 492 to 520 of SEQ ID NO:70; and amino acids 495 to 523 of SEQ ID NO:72. More preferably, the transgenic plant of this embodiment comprises a polynucleotide encoding a calcium dependent protein kinase having a sequence comprising amino acids 1 to 418 of SEQ ID NO:40; amino acids 1 to 575 of SEQ ID NO:42; amino acids 1 to 590 of SEQ ID NO:44; amino acids 1 to 532 of SEQ ID NO:46; amino acids 1 to 528 of SEQ ID NO:48; amino acids 1 to 578 of SEQ ID NO:50; amino acids 1 to 580 of SEQ ID NO:52; amino acids 1 to 574 of SEQ ID NO:54; amino acids 1 to 543 of SEQ ID NO:56; amino acids 1 to 549 of SEQ ID NO:58; amino acids 1 to 544 of SEQ ID NO:60; amino acids 1 to 534 of SEQ ID NO:62; amino acids 1 to 549 of SEQ ID NO:64; amino acids 1 to 532 of SEQ ID NO:66; amino acids 1 to 525 of SEQ ID NO:68; amino acids 1 to 548 of SEQ ID NO:70; or amino acids 1 to 531 of SEQ ID NO:72.

In another embodiment, the invention provides a transgenic plant transformed with an expression cassette comprising an isolated polynucleotide encoding a cyclin dependent protein kinase. The transgenic plant of this embodiment may comprise any polynucleotide encoding a cyclin dependent protein kinase. Preferably, the transgenic plant of this embodiment comprises a polynucleotide encoding a full-length polypeptide having cyclin dependent protein kinase activity, wherein the polypeptide comprises a cyclin N terminal domain having a sequence selected from the group consisting of amino acids 59 to 190 of SEQ ID NO:74; amino acids 63 to 197 of SEQ ID NO:76; amino acids 73 to 222 of SEQ ID NO:78; and amino acids 54 to 186 of SEQ ID NO:80 and a cyclin C terminal domain having a sequence selected from the group consisting of amino acids 192 to 252 of SEQ ID NO:74; amino acids 199 to 259 of SEQ ID NO:76; amino acids 224 to 284 of SEQ ID NO:78; and amino acids 188 to 248 of SEQ ID NO:80. More preferably, the transgenic plant of this embodiment comprises a polynucleotide encoding a cyclin dependent protein kinase having a sequence comprising amino acids 1 to 355 of SEQ ID NO:74; amino acids 1 to 360 of SEQ ID NO:76; amino acids 1 to 399 of SEQ ID NO:78; or amino acids 1 to 345 of SEQ ID NO:80.

In one embodiment, the invention provides a transgenic plant transformed with an expression cassette comprising an isolated polynucleotide encoding phospholipid hydroperoxide glutathione peroxidase.

The transgenic plant of this embodiment may comprise any polynucleotide encoding a phospholipid hydroperoxide glutathione peroxidase. Preferably, the transgenic plant of this embodiment comprises a polynucleotide encoding glutathione peroxidase domain having a sequence comprising amino acids 9 to 117 of SEQ ID NO:102; amino acids 17 to 125 of SEQ ID NO:104; amino acids 79 to 187 of SEQ ID NO:106; amino acids 10 to 118 of SEQ ID NO:108; amino acids 12 to 120 of SEQ ID NO:110; amino acids 9 to 117 of SEQ ID NO:112; amino acids 9 to 117 of SEQ ID NO:114; amino acids 10 to 118 of SEQ ID NO:116; amino acids 9 to 117 of SEQ ID NO:118; amino acids 77 to 185 of SEQ ID NO:120; amino acids 12 to 120 of SEQ ID NO:122; amino acids 12 to 120 of SEQ ID NO:124; amino acids 12 to 120 of SEQ ID NO:126; amino acids 12 to 120 of SEQ ID NO:128; amino acids 10 to 118 of SEQ ID NO:130; amino acids 70 to 178 of SEQ ID NO:132; amino acids 10 to 118 of SEQ ID NO:134; amino acids 24 to 132 of SEQ ID NO:136. More preferably, the transgenic plant of this embodiment comprises a polynucleotide encoding a phospholipid hydroperoxide glutathione peroxidase having a sequence comprising amino acids 1 to 169 of SEQ ID NO:102; amino acids 1 to 175 of SEQ ID NO:104; amino acids 1 to 236 of SEQ ID NO:106; amino acids 1 to 169 of SEQ ID NO:108; amino acids 1 to 176 of SEQ ID NO:110; amino acids 1 to 166 of SEQ ID NO:112; amino acids 1 to 166 of SEQ ID NO:114; amino acids 1 to 167 of SEQ ID NO:116; amino acids 1 to 166 of SEQ ID NO:118; amino acids 1 to 234 of SEQ ID NO:120; amino acids 1 to 170 of SEQ ID NO:122; amino acids 1 to 170 of SEQ ID NO:124; amino acids 1 to 169 of SEQ ID NO:126; amino acids 1 to 169 of SEQ ID NO:128; amino acids 1 to 179 of SEQ ID NO:130; amino acids 1 to 227 of SEQ ID NO:132; amino acids 1 to 168 of SEQ ID NO:134; amino acids 1 to 182 of SEQ ID NO:136.

One embodiment of the invention is a transgenic plant transformed with an expression cassette comprising an isolated polynucleotide encoding a full-length polypeptide comprising a TCP family transcription factor domain having a sequence comprising amino acids 57 to 249 of SEQ ID NO:138; amino acids 54 to 237 of SEQ ID NO:140; amino acids 43 to 323 of SEQ ID NO:142; or amino acids 41 to 262 of SEQ ID NO:144. More preferably, the transgenic plant of this embodiment comprises a polynucleotide encoding a TCP family transcription factor protein having a sequence comprising amino acids 1 to 319 of SEQ ID NO:138; amino acids 1 to 311 of SEQ ID NO:140; amino acids 1 to 400 of SEQ ID NO:142; or amino acids 1 to 321 of SEQ ID NO:144.

In another embodiment, the invention provides a transgenic plant transformed with an expression cassette comprising an isolated polynucleotide encoding a full-length S6 kinase polypeptide comprising a kinase domain having a sequence comprising amino acids 124 to 379 of SEQ ID NO:146 amino acids 150 to 406 of SEQ ID NO:148 or amino acids 152 to 408 of SEQ ID NO:150 or, alternatively, a kinase C-terminal domain having a sequence comprising amino acids 399 to 444 of SEQ ID NO:146; amino acids 426 to 468 of SEQ ID NO:148; or amino acids 428 to 471 of SEQ ID NO:150. More preferably, the transgenic plant of this embodiment comprises a polynucleotide encoding a ribosomal protein S6 kinase having a sequence comprising amino acids 1 to 455 of SEQ ID NO:146; amino acids 1 to 479 of SEQ ID NO:148; or amino acids 1 to 481 of SEQ ID NO:150.

In another embodiment, the invention provides a transgenic plant transformed with an expression cassette comprising an isolated polynucleotide encoding CAAX amino terminal protease family protein comprising a CAAX amino terminal protease domain having a sequence comprising amino acids 255 to 345 of SEQ ID NO:158; amino acids 229 to 319 of SEQ ID NO:160; or amino acids 267 to 357 of SEQ ID NO:162. More preferably, the transgenic plant of this embodiment comprises a polynucleotide encoding a CAAX amino terminal protease family protein having a sequence comprising amino acids 1 to 347 of SEQ ID NO:158; amino acids 1 to 337 of SEQ ID NO:160; or amino acids 1 to 359 of SEQ ID NO:162.

In another embodiment, the invention provides a transgenic plant transformed with an expression cassette comprising an isolated polynucleotide encoding a DNA binding protein.

The transgenic plant of this embodiment may comprise any polynucleotide encoding a DNA binding protein comprising a metallopeptidase family M24 domain having a sequence comprising amino acids 21 to 296 of SEQ ID NO:164; amino acids 20 to 295 of SEQ ID NO:166; amino acids 20 to 295 of SEQ ID NO:168; amino acids 22 to 297 of SEQ ID NO:170; or amino acids 22 to 297 of SEQ ID NO:172. More preferably, the transgenic plant of this embodiment comprises a polynucleotide encoding a DNA binding protein having a sequence comprising amino acids 1 to 390 of SEQ ID NO:164; amino acids 1 to 390 of SEQ ID NO:166; amino acids 1 to 394 of SEQ ID NO:168; amino acids 1 to 392 of SEQ ID NO:170; or amino acids 1 to 394 of SEQ ID NO:172.

In another embodiment, the invention provides a transgenic plant transformed with an expression cassette comprising an isolated polynucleotide encoding rev interacting protein m is 3.

The transgenic plant of this embodiment may comprise any polynucleotide encoding a rev interacting protein mis3. Preferably, the transgenic plant of this embodiment comprises a polynucleotide encoding a rev interacting protein mis3 having a sequence comprising amino acids 1 to 390 of SEQ ID NO:176; amino acids 1 to 389 of SEQ ID NO:178; amino acids 1 to 391 of SEQ ID NO:180.

In another embodiment, the invention provides a transgenic plant transformed with an expression cassette comprising an isolated polynucleotide encoding a GRF1 interacting factor comprising an SSXT protein (N terminal region) domain having a sequence comprising amino acids 7 to 80 of SEQ ID NO:182; amino acids 7 to 80 of SEQ ID NO:184; amino acids 7 to 80 of SEQ ID NO:186; or amino acids 6 to 79 of SEQ ID NO:188. More preferably, the transgenic plant of this embodiment comprises a polynucleotide encoding a GRF1 interacting factor having a sequence comprising amino acids 1 to 212 of SEQ ID NO:182; amino acids 1 to 203 of SEQ ID NO:184; amino acids 1 to 212 of SEQ ID NO:186; amino acids 1 to 213 of SEQ ID NO:188.

In another embodiment, the invention provides a transgenic plant transformed with an expression cassette comprising an isolated polynucleotide encoding eukaryotic translation initiation factor 4A comprising a DEAD/DEAH box helicase domain having a sequence comprising amino acids 59 to 225 of SEQ ID NO:190; amino acids 64 to 230 of SEQ ID NO:192; amino acids 58 to 224 of SEQ ID NO:194; amino acids 64 to 230 of SEQ ID NO:196; amino acids 64 to 230 of SEQ ID NO:198; amino acids 64 to 230 of SEQ ID NO:200 or a helicase conserved C-terminal domain having a sequence comprising amino acids 293 to 369 of SEQ ID NO:190; amino acids 298 to 374 of SEQ ID NO:192; amino acids 292 to 368 of SEQ ID NO:194; amino acids 298 to 374 of SEQ ID NO:196; amino acids 298 to 374 of SEQ ID NO:198; amino acids 298 to 374 of SEQ ID NO:200. More preferably, the transgenic plant of this embodiment comprises a polynucleotide encoding a eukaryotic translation initiation factor 4A having a sequence comprising amino acids 1 to 408 of SEQ ID NO:190; amino acids 1 to 413 of SEQ ID NO:192; amino acids 1 to 407 of SEQ ID NO:194; amino acids 1 to 413 of SEQ ID NO:196; amino acids 1 to 413 of SEQ ID NO:198; amino acids 1 to 413 of SEQ ID NO:200.

In another embodiment, the invention provides a transgenic plant transformed with an expression cassette comprising an isolated polynucleotide encoding TGF beta receptor interacting protein comprising a WD domain, G-beta repeat having a sequence selected from the group consisting of amino acids 42 to 80 of SEQ ID NO:154; amino acids 42 to 80 of SEQ ID NO:156; and amino acids 42 to 80 of SEQ ID NO:152; or a WD domain, G-beta repeat having a sequence selected from the group consisting of amino acids 136 to 174 of SEQ ID NO:154; amino acids 136 to 174 of SEQ ID NO:156; and amino acids 136 to 174 of SEQ ID NO:152; or a WD domain, G-beta repeat having a sequence selected from the group consisting of amino acids 181 to 219 of SEQ ID NO:154; amino acids 181 to 219 of SEQ ID NO:156; and amino acids 181 to 219 of SEQ ID NO:152; or a WD domain, G-beta repeat having a sequence selected from the group consisting of amino acids 278 to 316 of SEQ ID NO:154; amino acids 278 to 316 of SEQ ID NO:156; and amino acids 278 to 316 of SEQ ID NO:152. More preferably, the transgenic plant of this embodiment comprises a polynucleotide encoding a TGF beta receptor interacting protein having a sequence comprising amino acids 1 to 326 of SEQ ID NO:154; amino acids 1 to 326 of SEQ ID NO:156; amino acids 1 to 326 of SEQ ID NO:152.

In one embodiment, the invention provides a transgenic plant transformed with an expression cassette comprising an isolated polynucleotide encoding an AP2 domain containing protein. The transgenic plant of this embodiment may comprise any polynucleotide encoding an AP2 domain containing protein. Preferably, the transgenic plant of this embodiment comprises a polynucleotide encoding an AP2 domain having a sequence comprising amino acids 44 to 99 of SEQ ID NO:208; amino acids 36 to 91 of SEQ ID NO:210; amino acids 59 to 115 of SEQ ID NO:212; amino acids 56 to 111 of SEQ ID NO:214; amino acids 32 to 87 of SEQ ID NO:216; amino acids 10 to 65 of SEQ ID NO:218; amino acids 40 to 95 of SEQ ID NO:220; amino acids 43 to 98 of SEQ ID NO:222; amino acids 63 to 118 of SEQ ID NO:224; amino acids 34 to 89 of SEQ ID NO:226; amino acids 37 to 92 of SEQ ID NO:228; amino acids 22 to 77 of SEQ ID NO:230; amino acids 14 to 69 of SEQ ID NO:232; amino acids 42 to 97 of SEQ ID NO:234; amino acids 78 to 133 of SEQ ID NO:236; amino acids 27 to 82 of SEQ ID NO:238; amino acids 45 to 100 of SEQ ID NO:240; amino acids 41 to 96 of SEQ ID NO:242; amino acids 25 to 80 of SEQ ID NO:244; amino acids 14 to 69 of SEQ ID NO:246; amino acids 22 to 77 of SEQ ID NO:248; amino acids 130 to 186 of SEQ ID NO:250; amino acids 22 to 77 of SEQ ID NO:252. More preferably, the transgenic plant of this embodiment comprises a polynucleotide encoding an AP2 domain containing protein having a sequence comprising amino acids 1 to 231 of SEQ ID NO:208; amino acids 1 to 217 of SEQ ID NO:210; amino acids 1 to 121 of SEQ ID NO:212; amino acids 1 to 203 of SEQ ID NO:214; amino acids 1 to 210 of SEQ ID NO:216; amino acids 1 to 177 of SEQ ID NO:218; amino acids 1 to 181 of SEQ ID NO:220; amino acids 1 to 245 of SEQ ID NO:222; amino acids 1 to 233 of SEQ ID NO:224; amino acids 1 to 254 of SEQ ID NO:226; amino acids 1 to 275 of SEQ ID NO:228; amino acids 1 to 213 of SEQ ID NO:230; amino acids 1 to 266 of SEQ ID NO:232; amino acids 1 to 205 of SEQ ID NO:234; amino acids 1 to 240 of SEQ ID NO:236; amino acids 1 to 157 of SEQ ID NO:238; amino acids 1 to 211 of SEQ ID NO:240; amino acids 1 to 259 of SEQ ID NO:242; amino acids 1 to 243 of SEQ ID NO:244; amino acids 1 to 191 of SEQ ID NO:246; amino acids 1 to 287 of SEQ ID NO:248; amino acids 1 to 273 of SEQ ID NO:250; amino acids 1 to 267 of SEQ ID NO:252.

In one embodiment, the invention provides a transgenic plant transformed with an expression cassette comprising an isolated polynucleotide encoding a brassinosteroid biosynthetic protein having a sequence comprising amino acids 1 to 566 of SEQ ID NO:254.

In another embodiment, the invention provides a transgenic plant transformed with an expression cassette comprising an isolated polynucleotide encoding a RING box protein having a sequence comprising amino acids 1 to 120 of SEQ ID NO:256.

In another embodiment, the invention provides a transgenic plant transformed with an expression cassette comprising an isolated polynucleotide encoding a serine/threonine protein phosphatase. The transgenic plant of this embodiment may comprise any polynucleotide encoding a serine/threonine-specific protein phosphatase. Serine/threonine-specific protein phosphatases contain the characteristic signature sequence [L/I/V/M/N][K/R]GNHE. All polypeptides described herein as being serine/threonine-specific protein phosphatases and provided in FIG. 15, contain this signature sequence. Preferably, the transgenic plant of this embodiment comprises a polynucleotide encoding a calcineurin-like phosphoesterase domain having a sequence comprising amino acids 44 to 239 of SEQ ID NO:258; amino acids 43 to 238 of SEQ ID NO:260; amino acids 54 to 249 of SEQ ID NO:262; amino acids 44 to 240 of SEQ ID NO:264; amino acids 43 to 238 of SEQ ID NO:266; amino acids 54 to 249 of SEQ ID NO:268; amino acids 48 to 243 of SEQ ID NO:270; amino acids 47 to 242 of SEQ ID NO:272; amino acids 54 to 249 of SEQ ID NO:274; amino acids 48 to 243 of SEQ ID NO:276; amino acids 47 to 242 of SEQ ID NO:278; amino acids 44 to 240 of SEQ ID NO:280; amino acids 47 to 242 of SEQ ID NO:282; amino acids 47 to 243 of SEQ ID NO:284; or amino acids 60 to 255 of SEQ ID NO:286. More preferably, the transgenic plant of this embodiment comprises a polynucleotide encoding a serine/threonine protein phosphatase having a sequence comprising amino acids 1 to 304 of SEQ ID NO:258; amino acids 1 to 303 of SEQ ID NO:260; amino acids 1 to 305 of SEQ ID NO:262; amino acids 1 to 313 of SEQ ID NO:264; amino acids 1 to 306 of SEQ ID NO:266; amino acids 1 to 306 of SEQ ID NO:268; amino acids 1 to 308 of SEQ ID NO:270; amino amino acids 1 to 314 of SEQ ID NO:272; amino acids 1 to 306 of SEQ ID NO:274; amino acids 1 to 313 of SEQ ID NO:276; amino acids 1 to 305 of SEQ ID NO:278; amino acids 1 to 303 of SEQ ID NO:280; amino acids 1 to 313 of SEQ ID NO:282; amino acids 1 to 307 of SEQ ID NO:284; or amino acids 1 to 306 of SEQ ID NO:286.

In another embodiment, the invention provides a transgenic plant transformed with an expression cassette comprising an isolated polynucleotide encoding a serine/threonine-specific protein kinase. All polypeptides listed herein as being a serine/threonine-specific protein kinases contain the characteristic active-site signature sequence, of which the sequence, HRDLKLEN, is common to the polypeptides aligned in FIG. 4. The transgenic plant of this embodiment may comprise any polynucleotide encoding a serine/threonine-specific protein kinase. Preferably, the transgenic plant of this embodiment comprises a polynucleotide encoding a full-length polypeptide having serine/threonine-specific protein kinase activity, wherein the polypeptide comprises a domain selected from the group consisting of a domain having a sequence comprising amino acids 15 to 271 of SEQ ID NO:82; amino acids 4 to 260 of SEQ ID NO:84; amino acids 4 to 260 of SEQ ID NO:86; amino acids 18 to 274 of SEQ ID NO:88; amino acids 23 to 279 of SEQ ID NO:90; amino acids 5 to 261 of SEQ ID NO:92; amino acids 23 to 279 of SEQ ID NO:94; amino acids 4 to 260 of SEQ ID NO:96; amino acids 12 to 268 of SEQ ID NO:98; and amino acids 4 to 260 of SEQ ID NO:100. More preferably, the transgenic plant of this embodiment comprises a polynucleotide encoding a serine/threonine-specific protein kinase having a sequence comprising amino acids 1 to 348 of SEQ ID NO:82; amino acids 1 to 364 of SEQ ID NO:84; amino acids 1 to 354 of SEQ ID NO:86; amino acids 1 to 359 of SEQ ID NO:88; amino acids 1 to 360 of SEQ ID NO:90; amino acids 1 to 336 of SEQ ID NO:92; amino acids 1 to 362 of SEQ ID NO:94; amino acids 1 to 370 of SEQ ID NO:96; amino acids 1 to 350 of SEQ ID NO:98; or amino acids 1 to 361 of SEQ ID NO:100.

In one embodiment, the invention provides a transgenic plant transformed with an expression cassette comprising, in operative association, an isolated polynucleotide encoding a promoter capable of enhancing gene expression in leaves; and an isolated polynucleotide encoding a full-length polypeptide which is a subunit of acyl-CoA synthetase;

wherein the transgenic plant demonstrates increased yield as compared to a wild type plant of the same variety which does not comprise the expression cassette. As indicated in FIG. 16, acyl-CoA synthetase mediates the activation of long-chain fatty acids for synthesis of cellular lipids. In prokaryotes, the acyl CoA synthetase holoenzyme is a multimer of long-chain-fatty-acid-CoA ligase subunits. These ligase subunits of acyl-CoA synthetase are characterized, in part, by the presence of a cAMP binding domain signature sequence. Such signature sequences are exemplified in the long-chain-fatty-acid-CoA ligase proteins set forth in FIG. 17.

The transgenic plant of this embodiment may comprise any polynucleotide encoding an acyl-CoA synthetase long-chain-fatty-acid-CoA ligase subunit. Preferably, the transgenic plant of this embodiment comprises a polynucleotide encoding a full-length polypeptide having acyl-CoA synthetase long-chain-fatty-acid-CoA ligase subunit activity, wherein the polypeptide comprises a cAMP binding domain signature sequence selected from the group consisting of amino acids 213 to 543 of SEQ ID NO:288; amino acids 299 to 715 of SEQ ID NO:290; amino acids 173 to 504 of SEQ ID NO:292; amino acids 124 to 457 of SEQ ID NO:294; amino acids 178 to 509 of SEQ ID NO:296; amino acids 82 to 424 of SEQ ID NO:298; amino acids 207 to 388 of SEQ ID NO:300; amino acids 215 to 561 of SEQ ID NO:302; amino acids 111 to 476 of SEQ ID NO:304; amino acids 206 to 544 of SEQ ID NO:306; amino acids 192 to 531 of SEQ ID NO:308; amino acids 191 to 528 of SEQ ID NO:310; amino acids 259 to 660 of SEQ ID NO:312; amino acids 234 to 642 of SEQ ID NO:314; and amino acids 287 to 707 of SEQ ID NO:316. Most preferably, the transgenic plant of this embodiment comprises a polynucleotide encoding a long-chain-fatty-acid-CoA ligase subunit of acyl-CoA synthetase having a sequence comprising amino acids 1 to 561 of SEQ ID NO:288; amino acids 1 to 744 of SEQ ID NO:290; amino acids 1 to 518 of SEQ ID NO:292; amino acids 1 to 471 of SEQ ID NO:294; amino acids 1 to 523 of SEQ ID NO:296; amino acids 1 to 442 of SEQ ID NO:298; amino acids 1 to 555 of SEQ ID NO:300; amino acids 1 to 582 of SEQ ID NO:302; amino acids 1 to 455 of SEQ ID NO:304; amino acids 1 to 562 of SEQ ID NO:306; amino acids 1 to 547 of SEQ ID NO:308; amino acids 1 to 546 of SEQ ID NO:310; amino acids 1 to 691 of SEQ ID NO:312; amino acids 1 to 664 of SEQ ID NO:314; or amino acids 1 to 726 of SEQ ID NO:316.

In another embodiment, the invention provides a transgenic plant transformed with an expression cassette comprising, in operative association, an isolated polynucleotide encoding a promoter capable of enhancing gene expression in leaves and an isolated polynucleotide encoding a full-length beta-ketoacyl-ACP synthase polypeptide, wherein the transgenic plant demonstrates increased yield as compared to a wild type plant of the same variety which does not comprise the expression cassette. The beta-ketoacyl-ACP synthase enzyme is active in initiating fatty acid biosynthesis and has acetyl CoA:ACP transacylase activity. It selectively catalyzes the formation of acetoacetyl-ACP and specifically uses CoA thioesters rather than acyl-ACP as the primer. The enzyme has a role in feedback regulation of fatty acid synthesis. The transgenic plant of this embodiment may comprise any polynucleotide encoding a beta-ketoacyl-ACP synthase polypeptide. Preferably, the beta-ketoacyl-ACP synthase polypeptide employed in this embodiment of the invention comprises amino acids 1 to 317 of SEQ ID NO:318.

The first committed step in fatty acid biosynthesis is the conversion of acetyl-CoA to malonyl-CoA by the enzyme acetyl CoA carboxylase (ACC). Subsequent steps include the elongation reactions with two carbon donations to the chain from malonyl-CoA. The activity of ACC is regulated by phosphorylation and dephosphorylation in eukaryotes and as well has allosteric regulation by metabolites such as citrate. In prokaryotes, ACCs are multi-subunit enzymes consisting of a carboxyl transferase designated ACC alpha, a biotin-dependent carboxylase, and biotin carboxyl carrier protein, whereas eukaryotic ACCs are multidomain enzymes. Most plants have both forms of ACCs, with the prokaryotic-like form in plastids, and the eukaryotic-like form in the cytosol. Plant mitochondria are thought to lack ACC activity and to synthesize fatty acids from malonyl CoA. Subcellular compartmentalization of the enzymes involved in fatty acid metabolism is an important determinant of the final end products produced.

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a subunit of an acetyl-CoA carboxylase complex, wherein the transgenic plant demonstrates increased yield as compared to a wild type plant of the same variety which does not comprise the expression cassette. In accordance with the invention, the ACC subunit employed in this embodiment may be an ACC alpha, a biotin-dependent carboxylase, or a biotin carboxyl carrier protein. The transgenic plant of this embodiment may comprise any polynucleotide encoding an ACC alpha, a biotin-dependent carboxylase, or biotin carboxyl carrier protein which is a subunit of ACC.

When the subunit is ACC alpha, it preferably comprises amino acids 1 to 319 of SEQ ID NO:320.

When the ACC subunit is a biotin-dependent carboxylase, it is characterized, in part, by the presence of a carbamoyl-phosphate synthase subdomain signature sequence. Such signature sequences are exemplified in the biotin-dependent carboxylases set forth in FIG. 18. In accordance with the invention, the biotin-dependent carboxylase of this embodiment comprises a domain selected from the group consisting of amino acids 3 to 308 of SEQ ID NO:322; amino acids 73 to 378 of SEQ ID NO:324; amino acids 38 to 344 of SEQ ID NO:326; and amino acids 73 to 378 of SEQ ID NO:328. More preferably, the biotin-dependent carboxylase of this embodiment comprises amino acids 1 to 449 of SEQ ID NO:322; amino acids 1 to 535 of SEQ ID NO:324; amino acids 1 to 732 of SEQ ID NO:326; or amino acids 1 to 539 of SEQ ID NO:328.

When the ACC subunit is a biotin carboxyl carrier protein, it is characterized, in part, by the presence of a signature sequence surrounding an M-K dipeptide sequence, of which the lysine residue is the biotin attachment site. Such signature sequences are exemplified in the biotin carboxyl carrier proteins set forth in FIG. 19. In accordance with the invention, the biotin carboxyl carrier protein of this embodiment comprises a domain selected from the group consisting of amino acids 79 to 152 of SEQ ID NO:330; amino acids 204 to 277 of SEQ ID NO:332; and amino acids 37 to 110 of SEQ ID NO:334. More preferably, the biotin carboxyl carrier protein subunit of this embodiment comprises amino acids 1 to 156 of SEQ ID NO:330; amino acids 1 to 282 of SEQ ID NO:332; or amino acids 1 to 115 of SEQ ID NO:334.

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length 3-oxoacyl-[ACP] synthase II polypeptide; wherein the transgenic plant demonstrates increased yield as compared to a wild type plant of the same variety which does not comprise the expression cassette. The 3-oxoacyl-ACP synthase II enzymes belong to the class of beta-ketoacyl synthases, which first transfer the acyl component of an activated acyl primer to the highly conserved, active-site cysteine residue of the enzyme and then catalyze a condensation reaction with an activated malonyl donor, concomitantly releasing carbon dioxide. The 3-oxoacyl-ACP synthase II enzymes contain a conserved signature sequence which surrounds the active-site cysteine residue. Such signature sequences are exemplified in the 3-oxoacyl-ACP synthase II proteins set forth in FIG. 20.

The transgenic plant of this embodiment may comprise any polynucleotide encoding a 3-oxoacyl-ACP synthase II. Preferably, the transgenic plant of this embodiment comprises a polynucleotide encoding a full-length polypeptide having 3-oxoacyl-ACP synthase II activity, wherein the polypeptide comprises a domain selected from the group consisting of amino acids 12 to 410 of SEQ ID NO:336; amino acids 2 to 401 of SEQ ID NO:338; amino acids 55 to 456 of SEQ ID NO:340; amino acids 2 to 401 of SEQ ID NO:342. More preferably, the transgenic plant of this embodiment comprises a polynucleotide encoding a 3-oxoacyl-ACP synthase II comprising amino acids 1 to 413 of SEQ ID NO:336; amino acids 1 to 406 of SEQ ID NO:338; amino acids 1 to 461 of SEQ ID NO:340; amino acids 1 to 406 of SEQ ID NO:342.

In another embodiment, the invention provides a transgenic plant transformed with an expression cassette comprising, in operative association, an isolated polynucleotide encoding a promoter and an isolated polynucleotide encoding a full-length 3-oxoacyl-[ACP] reductase polypeptide; wherein the transgenic plant demonstrates increased yield as compared to a wild type plant of the same variety which does not comprise the expression cassette. The promoter employed in the expression vector of this embodiment may optionally be capable of enhancing expression in leaves. Moreover, the expression vector of this embodiment may optionally comprise a mitochondrial or chloroplast transit peptide.

Predicted domains of 3-oxoacyl-[ACP] reductase polypeptides include a short chain dehydrogenase (PF00106) domain. Short chain dehydrogenases are a large family of enzymes, many of which are NAD- or NADP-dependent oxidoreductases. Most dehydrogenases have two domains, one to bind the coenzyme, e.g. NAD, and the second domain to bind the substrate, which determines substrate specificity, and contains amino acids involved in catalysis. Within the coenzyme binding domain there is little primary sequence similarity, although structural similarity has been found. However, a signature sequence of short-chain dehydrogenases, which includes a YxxxK motif, has been identified. Such signature sequences are exemplified in the 3-oxoacyl-[ACP] reductase proteins set forth in FIG. 21.

The transgenic plant of this embodiment may comprise any polynucleotide encoding a 3-oxoacyl-ACP reductase. Preferably, the transgenic plant of this embodiment comprises a polynucleotide encoding a full-length polypeptide having 3-oxoacyl-ACP reductase activity, wherein the polypeptide comprises a domain selected from the group consisting of a domain having a sequence comprising amino acids 80 to 181 of SEQ ID NO:344; amino acids 85 to 186 of SEQ ID NO:346; amino acids 79 to 180 of SEQ ID NO:348; amino acids 69 to 170 of SEQ ID NO:350; amino acids 51 to 154 of SEQ ID NO:352; amino acids 156 to 257 of SEQ ID NO:354; amino acids 90 to 193 of SEQ ID NO:356; amino acids 81 to 184 of SEQ ID NO:358; amino acids 128 to 228 of SEQ ID NO:360; amino acids 96 to 197 of SEQ ID NO:362; amino acids 97 to 198 of SEQ ID NO:364; amino acids 95 to 198 of SEQ ID NO:366; amino acids 103 to 208 of SEQ ID NO:368; amino acids 103 to 208 of SEQ ID NO:370; amino acids 100 to 203 of SEQ ID NO:372; amino acids 96 to 197 of SEQ ID NO:374; amino acids 96 to 197 of SEQ ID NO:376; amino acids 89 to 192 of SEQ ID NO:378; amino acids 159 to 260 of SEQ ID NO:380; amino acids 88 to 187 of SEQ ID NO:382; amino acids 148 to 249 of SEQ ID NO:384; amino acids 98 to 202 of SEQ ID NO:386; amino acids 95 to 199 of SEQ ID NO:388; amino acids 154 to 257 of SEQ ID NO:390; amino acids 88 to 187 of SEQ ID NO:392; amino acids 100 to 201 of SEQ ID NO:394; and amino acids 88 to 187 of SEQ ID NO:396. More preferably, the transgenic plant of this embodiment comprises a polynucleotide encoding a 3-oxoacyl-ACP reductase having a sequence comprising amino acids 1 to 244 of SEQ ID NO:344; amino acids 1 to 247 of SEQ ID NO:346; amino acids 1 to 253 of SEQ ID NO:348; amino acids 1 to 243 of SEQ ID NO:350; amino acids 1 to 236 of SEQ ID NO:352; amino acids 1 to 320 of SEQ ID NO:354; amino acids 1 to 275 of SEQ ID NO:356; amino acids 1 to 260 of SEQ ID NO:358; amino acids 1 to 294 of SEQ ID NO:360; amino acids 1 to 267 of SEQ ID NO:362; amino acids 1 to 272 of SEQ ID NO:364; amino acids 1 to 280 of SEQ ID NO:366; amino acids 1 to 282 of SEQ ID NO:368; amino acids 1 to 282 of SEQ ID NO:370; amino acids 1 to 265 of SEQ ID NO:372; amino acids 1 to 264 of SEQ ID NO:374; amino acids 1 to 271 of SEQ ID NO:376; amino acids 1 to 256 of SEQ ID NO:378; amino acids 1 to 323 of SEQ ID NO:380; amino acids 1 to 249 of SEQ ID NO:382; amino acids 1 to 312 of SEQ ID NO:384; amino acids 1 to 246 of SEQ ID NO:386; amino acids 1 to 258 of SEQ ID NO:388; amino acids 1 to 320 of SEQ ID NO:390; amino acids 1 to 253 of SEQ ID NO:392; amino acids 1 to 273 of SEQ ID NO:394; or amino acids 1 to 253 of SEQ ID NO:396.

In another embodiment, the invention provides a transgenic plant transformed with an expression cassette comprising, in operative association, an isolated polynucleotide encoding a promoter, an isolated polynucleotide encoding a mitochondrial transit peptide, and an isolated polynucleotide encoding a full-length biotin synthetase polypeptide, wherein the transgenic plant demonstrates increased yield as compared to a wild type plant of the same variety which does not comprise the expression cassette.

Biotin synthetases catalyze the last step of biotin biosynthesis, converting 9-mercaptothiobiotin to biotin. The structure of biotin synthetases includes a predicted radical SAM superfamily domain (PF04055). These domains in the radical SAM superfamily are important in catalyzing diverse reactions including unusual methylations, isomerization, sulphur insertion, ring formation, anaerobic oxidation and protein radical formation. Evidence exists that these proteins generate a radical species by reductive cleavage of S-adenosylmethionine (SAM) through an unusual Fe—S center. Three cysteine residues arranged in a CxxxCxxC pattern are an essential component of such Fe—S centers. All polypeptides listed herein as have this predicted motif as a part of their predicted radical SAM superfamily domain. Such signature sequences are exemplified in the biotin sythetase proteins set forth in FIG. 22.

The transgenic plant of this embodiment may comprise any polynucleotide encoding a biotin synthetase. Preferably, the transgenic plant of this embodiment comprises a polynucleotide encoding a full-length polypeptide having biotin synthetase activity, wherein the polypeptide comprises a domain selected from the group consisting of a domain having a sequence comprising amino acids 78 to 300 of SEQ ID NO:398; amino acids 82 to 301 of SEQ ID NO:400; and amino acids 79 to 298 of SEQ ID NO:402. More preferably, the transgenic plant of this embodiment comprises a polynucleotide encoding a biotin synthetase having a sequence comprising amino acids 1 to 362 of SEQ ID NO:398; amino acids 1 to 304 of SEQ ID NO:400; or amino acids 1 to 372 of SEQ ID NO:402.

The invention further provides a seed which is true breeding for the expression cassettes (also referred to herein as “transgenes”) described herein, wherein transgenic plants grown from said seed demonstrate increased yield as compared to a wild type variety of the plant. The invention also provides a product produced by or from the transgenic plants expressing the polynucleotide, their plant parts, or their seeds. The product can be obtained using various methods well known in the art. As used herein, the word “product” includes, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs. The invention further provides an agricultural product produced by any of the transgenic plants, plant parts, and plant seeds. Agricultural products include, but are not limited to, plant extracts, proteins, amino acids, carbohydrates, fats, oils, polymers, vitamins, and the like.

The invention also provides an isolated polynucleotide which has a sequence selected from the group consisting of SEQ ID NO:291; SEQ ID NO:293; SEQ ID NO:295; SEQ ID NO:297; SEQ ID NO:299; SEQ ID NO:301; SEQ ID NO:303; SEQ ID NO:311; SEQ ID NO:313; SEQ ID NO:315; SEQ ID NO:331; SEQ ID NO:333; SEQ ID NO:337; SEQ ID NO:339; SEQ ID NO:341; SEQ ID NO:347; SEQ ID NO:349; SEQ ID NO:351; SEQ ID NO:353; SEQ ID NO:355; SEQ ID NO:357; SEQ ID NO:359; SEQ ID NO:361; SEQ ID NO:363; SEQ ID NO:365; SEQ ID NO:367; SEQ ID NO:369; SEQ ID NO:371; SEQ ID NO:373; SEQ ID NO:375; SEQ ID NO:377; SEQ ID NO:379; SEQ ID NO:383; SEQ ID NO:385; SEQ ID NO:387; SEQ ID NO:389; SEQ ID NO:391; SEQ ID NO:393; SEQ ID NO:395; SEQ ID NO:399; and SEQ ID NO:401. Also encompassed by the isolated polynucleotide of the invention is an isolated polynucleotide encoding a polypeptide having an amino acid sequence selected from the group consisting of SEQ ID NO:292; SEQ ID NO:294; SEQ ID NO:296; SEQ ID NO:298; SEQ ID NO:300; SEQ ID NO:302; SEQ ID NO:304; SEQ ID NO:312; SEQ ID NO:314; SEQ ID NO:316; SEQ ID NO:332; SEQ ID NO:334; SEQ ID NO:338; SEQ ID NO:340; SEQ ID NO:342; SEQ ID NO:348; SEQ ID NO:350; SEQ ID NO:352; SEQ ID NO:354; SEQ ID NO:356; SEQ ID NO:358; SEQ ID NO:360; SEQ ID NO:362; SEQ ID NO:364; SEQ ID NO:366; SEQ ID NO:368; SEQ ID NO:370; SEQ ID NO:372; SEQ ID NO:374; SEQ ID NO:376; SEQ ID NO:378; SEQ ID NO:380; SEQ ID NO:384; SEQ ID NO:386; SEQ ID NO:388; SEQ ID NO:390; SEQ ID NO:392; SEQ ID NO:394; SEQ ID NO:396; SEQ ID NO:400; and SEQ ID NO:402. A polynucleotide of the invention can be isolated using standard molecular biology techniques and the sequence information provided herein, for example, using an automated DNA synthesizer.

In one embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and polynucleotide encoding a full-length FPS polypeptide, wherein the transgenic plant demonstrates increased yield as compared to a wild type plant of the same variety which does not comprise the expression cassette.

Gene B0421 (SEQ ID NO:414) and gene YJL167W (SEQ ID NO:416) encode FPS. As indicated in FIG. 23, FPS catalyzes the synthesis of farnesyl diphosphate (an important precursor of sterols and terpenoids) from isopentenyl diphosphate and dimethylallyl diphosphate. Previous reports on high expression of FPS in A. thaliana plants indicated that the gene caused a cell death/senescence-like phenotype with less-vigorous growth compared to wild-type plants, with the onset and severity of the phenotype corresponding to the level of FPS activity. A. thaliana has two genes encoding three isoforms of farnesyl diphosphate synthase: FPS1L, FPS1S, and FPS2. When FPS1L is targeted to the mitochondria in Arabidopsis, chlorosis and cell death under continuous light occur. This overexpression in mitochondria causes an altered leaf cytokinin profile, and renders the plant more sensitive to oxidative stress induced by continuous light.

In contrast to these published observations, we observed that if gene B0421 (SEQ ID NO:414) was expressed under control of the USP promoter and the protein was targeted to the mitochondria, the plants were larger under water limiting growth conditions. Moreover, if gene YJL167W (SEQ ID NO:416) was expressed under control of the USP promoter and the protein was targeted to the mitochondria, the plants were larger under well watered growth conditions.

The transgenic plant of this embodiment may comprise any polynucleotide encoding an FPS polypeptide. A predicted domain of FPS proteins is a polyprenyl synthetase (PF00348). The polyprenyl synthetase domain is characterized, in part, by the presence of two signature sequences. Such signature sequences are exemplified in the FPS proteins set forth in FIG. 24. Preferably, the transgenic plant of this embodiment comprises a polynucleotide encoding a full-length polypeptide having FPS activity, wherein the polypeptide comprises a polyprenyl synthetase domain comprising a pair of signature sequences, wherein one member of the pair is selected from the group consisting of amino acids 81 to 125 of SEQ ID NO:414; amino acids 97 to 139 of SEQ ID NO:416; amino acids 76 to 120 of SEQ ID NO:418; amino acids 116 to 160 of SEQ ID NO:420; amino acids 90 to 132 of SEQ ID NO:422; amino acids 7 to 51 of SEQ ID NO:424; amino acids 46 to 90 of SEQ ID NO:426; amino acids 7 to 49 of SEQ ID NO:428; amino acids 19 to 61 of SEQ ID NO:430; amino acids 7 to 49 of SEQ ID NO:432; and amino acids 98 to 140 of SEQ ID NO:434; and the other member of the pair of signature sequences is selected from the group consisting of amino acids 193 to 227 of SEQ ID NO:414; amino acids 210 to 244 of SEQ ID NO:416; amino acids 191 to 224 of SEQ ID NO:418; amino acids 224 to 257 of SEQ ID NO:420; amino acids 203 to 236 of SEQ ID NO:422; amino acids 115 to 148 of SEQ ID NO:424; amino acids 158 to 191 of SEQ ID NO:426; amino acids 108 to 141 of SEQ ID NO:428; amino acids 132 to 165 of SEQ ID NO:430; amino acids 108 to 141 of SEQ ID NO:432; and amino acids 211 to 244 of SEQ ID NO:434. Most preferably, the transgenic plant of this embodiment comprises a polynucleotide encoding an FPS polypeptide having a sequence comprising amino acids 1 to 299 of SEQ ID NO:414; amino acids 1 to 352 of SEQ ID NO:416; amino acids 1 to 294 of SEQ ID NO:418; amino acids 1 to 274 of SEQ ID NO:420; amino acids 1 to 342 of SEQ ID NO:422; amino acids 1 to 222 of SEQ ID NO:424; amino acids 1 to 261 of SEQ ID NO:426; amino acids 1 to 161 of SEQ ID NO:428; amino acids 1 to 174 of SEQ ID NO:430; amino acids 1 to 245 of SEQ ID NO:432; or amino acids 1 to 350 of SEQ ID NO:434.

In another embodiment, the invention provides a transgenic plant transformed with an expression cassette comprising, in operative association, an isolated polynucleotide encoding a promoter capable of enhancing gene expression in leaves, an isolated polynucleotide encoding a chloroplast transit peptide, and an isolated polynucleotide encoding a full-length squalene synthase polypeptide, wherein the transgenic plant demonstrates increased yield as compared to a wild type plant of the same variety which does not comprise the expression cassette. Gene SQS1 (SEQ ID NO:436) encodes SQS, which catalyzes the conversion of two molecules of farnesyl diphosphate into squalene, which is the first committed step in sterol biosynthesis.

The transgenic plant of this embodiment may comprise any polynucleotide encoding a SQS polypeptide. Preferably, the transgenic plant of this embodiment comprises a polynucleotide encoding a full-length polypeptide having SQS activity, wherein the polypeptide comprises a squalene synthetase domain which comprises a pair of SQS signature sequences. Such signature sequences are exemplified in the SQS polypeptides set forth in FIG. 25. Preferably, the polynucleotide encodes a SQS polypeptide comprising a squalene synthetase domain comprising a pair of signature sequences, wherein one member of the pair has a sequence selected from the group consisting of amino acids 201 to 216 of SEQ ID NO:436; amino acids 201 to 216 of SEQ ID NO:438; amino acids 168 to 183 of SEQ ID NO:440; amino acids 168 to 183 of SEQ ID NO:442; and amino acids 164 to 179 of SEQ ID NO:444; and the other member of the pair of signature sequences has a sequence selected from the group consisting of amino acids 234 to 262 of SEQ ID NO:436; amino acids 234 to 262 of SEQ ID NO:438; amino acids 203 to 231 of SEQ ID NO:440; amino acids 201 to 229 of SEQ ID NO:442; and amino acids 197 to 225 of SEQ ID NO:444. More preferably, the polynucleotide encodes a SQS polypeptide comprising a squalene synthetase domain selected from the group consisting of amino acids 95 to 351 of SEQ ID NO:436; amino acids 95 to 351 of SEQ ID NO:438; amino acids 62 to 320 of SEQ ID NO:440; amino acids 62 to 318 of SEQ ID NO:442; and amino acids 58 to 314 of SEQ ID NO:444. Most preferably, the polynucleotide encodes a SQS polypeptide comprising amino acids 1 to 436 of SEQ ID NO:436; amino acids 1 to 436 of SEQ ID NO:438; amino acids 1 to 357 of SEQ ID NO:440; amino acids 1 to 413 of SEQ ID NO:442; or amino acids 1 to 401 of SEQ ID NO:444.

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length squalene epoxidase polypeptide; wherein the transgenic plant demonstrates increased yield as compared to a wild type plant of the same variety which does not comprise the expression cassette. Gene YGR175C (SEQ ID NO:446) encodes squalene epoxidase, which catalyzes the first oxygenation step in sterol biosynthesis, the conversion of squalene into oxidosqualene, a precursor of cyclic triterpenoids such as membrane sterols, brassinosteroid phytohormones, and non-steroidal triterpenoids. Squalene epoxidase may be one of the rate-limiting steps in this pathway. Like other flavin-dependent enzymes, squalene epoxidase enzymes are characterized, in part, by the presence of a flavin adenine dinucleotide (FAD) cofactor binding domain and a substrate-binding domain. The active site is at the interface of these two domains. These domains are characterized by two distinctive sequence motifs. One of these motifs forms a loop at the interface between the FAD and the substrate-binding domains and has the sequence, D-R-I-v-G-E-I-m-Q-P-g-G (SEQ ID NO:461) in YGR175C (SEQ ID NO:446). Those amino acid residues represented in uppercase are highly conserved among squalene epoxidases. The other motif, G-D-x-x-N-M-R-H-P-1-t-g-g-G-M-t-V (SEQ ID NO:462), includes an FAD binding site (334GD335) and part of the potential substrate binding residues identified in squalene epoxidase from rat. This motif also forms a loop near the FAD cofactor at the interface between the two squalene epoxidase domains and is located opposite to the first motif. Such conserved motifs are exemplified in the squalene epoxidase proteins set forth in FIG. 26.

The transgenic plant of this embodiment may comprise any polynucleotide encoding a squalene epoxidase. Preferably, the transgenic plant of this embodiment comprises a polynucleotide encoding a full-length polypeptide having squalene epoxidase activity, wherein the polypeptide comprises a domain comprising a pair of FAD-dependent enzyme motifs, wherein one member of the pair has a sequence selected from the group consisting of amino acids 55 to 66 of SEQ ID NO:446; amino acids 79 to 90 of SEQ ID NO:448; and amino acids 98 to 109 of SEQ ID NO:450; and the other member of the pair has a sequence selected from the group consisting of amino acids 334 to 350 of SEQ ID NO:446; amino acids 331 to 347 of SEQ ID NO:448; and amino acids 347 to 363 of SEQ ID NO:450. More preferably, the polynucleotide encodes a a full-length polypeptide having squalene epoxidase activity, wherein the polypeptide comprises a domain selected from the group consisting of amino acids 20 to 488 of SEQ ID NO:446; amino acids 44 to 483 of SEQ ID NO:448; or amino acids 63 to 500 of SEQ ID NO:450. Most preferably, the transgenic plant of this embodiment comprises a polynucleotide encoding a squalene epoxidase comprising amino acids 1 to 496 of SEQ ID NO:446; amino acids 1 to 512 of SEQ ID NO:448; or amino acids 1 to 529 of SEQ ID NO:450.

The invention also provides an isolated polynucleotide which has a sequence selected from the group consisting of SEQ ID NO:417; SEQ ID NO:419; SEQ ID NO:421; SEQ ID NO:423; SEQ ID NO:425; SEQ ID NO:427; SEQ ID NO:429; SEQ ID NO:431; SEQ ID NO:435; SEQ ID NO:437; SEQ ID NO:439; SEQ ID NO:447; and SEQ ID NO:449. Also encompassed by the isolated polynucleotide of the invention is an isolated polynucleotide encoding a polypeptide having an amino acid sequence selected from the group consisting of SEQ ID NO:418; SEQ ID NO:420; SEQ ID NO:422; SEQ ID NO:424; SEQ ID NO:426; SEQ ID NO:428; SEQ ID NO:430; SEQ ID NO:432; SEQ ID NO:436; SEQ ID NO:438; SEQ ID NO:440; SEQ ID NO:448; and SEQ ID NO:450. A polynucleotide of the invention can be isolated using standard molecular biology techniques and the sequence information provided herein, for example, using an automated DNA synthesizer.

The invention further provides a recombinant expression vector which comprises an expression cassette selected from the group consisting of a) an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length FPS polypeptide; b) an expression cassette comprising, in operative association, an isolated polynucleotide encoding a promoter capable of enhancing gene expression in leaves; and an isolated polynucleotide encoding a full-length SQS polypeptide; and c) an expression cassette comprising in operative association, an isolated polynucleotide encoding a promoter capable of enhancing gene expression in leaves; an isolated polynucleotide encoding a chloroplast transit peptide; and an isolated polynucleotide encoding a full-length squalene epoxidase polypeptide.

In another embodiment, the recombinant expression vector of the invention comprises an isolated polynucleotide having a sequence selected from the group consisting of SEQ ID NO:417; SEQ ID NO:419; SEQ ID NO:421; SEQ ID NO:423; SEQ ID NO:425; SEQ ID NO:427; SEQ ID NO:429; SEQ ID NO:431; SEQ ID NO:435; SEQ ID NO:437; SEQ ID NO:439; SEQ ID NO:447; and SEQ ID NO:449. In addition, the recombinant expression vector of the invention comprises an isolated polynucleotide encoding a polypeptide having an amino acid sequence selected from the group consisting of SEQ ID NO:418; SEQ ID NO:420; SEQ ID NO:422; SEQ ID NO:424; SEQ ID NO:426; SEQ ID NO:428; SEQ ID NO:430; SEQ ID NO:432; SEQ ID NO:436; SEQ ID NO:438; SEQ ID NO:440; SEQ ID NO:448; and SEQ ID NO:450.

The invention further provides a seed produced by a transgenic plant expressing polynucleotide listed in Table 1, wherein the seed contains the polynucleotide, and wherein the plant is true breeding for increased growth and/or yield under normal or stress conditions and/or increased tolerance to an environmental stress as compared to a wild type variety of the plant. The invention also provides a product produced by or from the transgenic plants expressing the polynucleotide, their plant parts, or their seeds. The product can be obtained using various methods well known in the art. As used herein, the word “product” includes, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs. The invention further provides an agricultural product produced by any of the transgenic plants, plant parts, and plant seeds. Agricultural products include, but are not limited to, plant extracts, proteins, amino acids, carbohydrates, fats, oils, polymers, vitamins, and the like.

In a preferred embodiment, an isolated polynucleotide of the invention comprises a polynucleotide having a sequence selected from the group consisting of the polynucleotide sequences listed in Table 1. These polynucleotides may comprise sequences of the coding region, as well as 5′ untranslated sequences and 3′ untranslated sequences.

A polynucleotide of the invention can be isolated using standard molecular biology techniques and the sequence information provided herein, for example, using an automated DNA synthesizer.

“Homologs” are defined herein as two nucleic acids or polypeptides that have similar, or substantially identical, nucleotide or amino acid sequences, respectively. Homologs include allelic variants, analogs, and orthologs, as defined below. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. The term homolog further encompasses nucleic acid molecules that differ from one of the nucleotide sequences shown in Table 1 due to degeneracy of the genetic code and thus encode the same polypeptide. As used herein, a “naturally occurring” nucleic acid molecule refers to an RNA or DNA molecule having a nucleotide sequence that occurs in nature (e.g., encodes a natural polypeptide).

To determine the percent sequence identity of two amino acid sequences (e.g., one of the polypeptide sequences of Table 1 and a homolog thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence then the molecules are identical at that position. The same type of comparison can be made between two nucleic acid sequences.

Preferably, the isolated amino acid homologs, analogs, and orthologs of the polypeptides of the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence identified in Table 1. In another preferred embodiment, an isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence shown in Table 1.

For the purposes of the invention, the percent sequence identity between two nucleic acid or polypeptide sequences is determined using Align 2.0 (Myers and Miller, CABIOS (1989) 4:11-17) with all parameters set to the default settings or the Vector NTI 9.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

Nucleic acid molecules corresponding to homologs, analogs, and orthologs of the polypeptides listed in Table 1 can be isolated based on their identity to said polypeptides, using the polynucleotides encoding the respective polypeptides or primers based thereon, as hybridization probes according to standard hybridization techniques under stringent hybridization conditions. As used herein with regard to hybridization for DNA to a DNA blot, the term “stringent conditions” refers to hybridization overnight at 60° C. in 10×Denhart's solution, 6×SSC, 0.5% SDS, and 100 g/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 g/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art. Preferably, an isolated nucleic acid molecule of the invention that hybridizes under stringent or highly stringent conditions to a nucleotide sequence listed in Table 1 corresponds to a naturally occurring nucleic acid molecule.

There are a variety of methods that can be used to produce libraries of potential homologs from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential sequences. Methods for synthesizing degenerate oligonucleotides are known in the art.

The isolated polynucleotides employed in the invention may be optimized, that is, genetically engineered to increase its expression in a given plant or animal. To provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) to eliminate sequences that cause destabilization, inappropriate polyadenylation, degradation and termination of RNA, or that form secondary structure hairpins or RNA splice sites; or 5) elimination of antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

The invention further provides a recombinant expression vector which comprise an expression cassette selected from the group consisting of a) an expression cassette comprising, in operative association, an isolated polynucleotide encoding a promoter capable of enhancing gene expression in leaves; and an isolated polynucleotide encoding a full-length polypeptide which is a subunit of acyl-CoA synthetase; b) an expression cassette comprising, in operative association, an isolated polynucleotide encoding a promoter capable of enhancing gene expression in leaves; and an isolated polynucleotide encoding a full-length beta-ketoacyl-ACP synthase polypeptide; c) an expression cassette comprising in operative association, an isolated polynucleotide encoding a promoter capable of enhancing gene expression in leaves; an isolated polynucleotide encoding a mitochondrial transit peptide; and an isolated polynucleotide encoding a subunit of an acetyl-CoA carboxylase complex, d) an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length 3-oxoacyl-[ACP] synthase II polypeptide; e) an expression cassette comprising, in operative association, an isolated polynucleotide encoding a promoter; an isolated polynucleotide encoding a full-length 3-oxoacyl-[ACP] reductase polypeptide, and optionally a mitochondrial or chloroplast transit peptide; and f) an expression cassette comprising, in operative association, an isolated polynucleotide encoding a promoter, an isolated polynucleotide encoding a mitochondrial transit peptide, and an isolated polynucleotide encoding a full-length biotin synthetase polypeptide.

In another embodiment, the recombinant expression vector of the invention comprises an isolated polynucleotide having a sequence selected from the group consisting of SEQ ID NO:291; SEQ ID NO:293; SEQ ID NO:295; SEQ ID NO:297; SEQ ID NO:299; SEQ ID NO:301; SEQ ID NO:303; SEQ ID NO:311; SEQ ID NO:313; SEQ ID NO:315; SEQ ID NO:331; SEQ ID NO:333; SEQ ID NO:337; SEQ ID NO:339; SEQ ID NO:341; SEQ ID NO:347; SEQ ID NO:349; SEQ ID NO:351; SEQ ID NO:353; SEQ ID NO:355; SEQ ID NO:357; SEQ ID NO:359; SEQ ID NO:361; SEQ ID NO:363; SEQ ID NO:365; SEQ ID NO:367; SEQ ID NO:369; SEQ ID NO:371; SEQ ID NO:373; SEQ ID NO:375; SEQ ID NO:377; SEQ ID NO:379; SEQ ID NO:383; SEQ ID NO:385; SEQ ID NO:387; SEQ ID NO:389; SEQ ID NO:391; SEQ ID NO:393; SEQ ID NO:395; SEQ ID NO:399; and SEQ ID NO:401. In addition, the recombinant expression vector of the invention comprises an isolated polynucleotide encoding a polypeptide having an amino acid sequence selected from the group consisting of SEQ ID NO:292; SEQ ID NO:294; SEQ ID NO:296; SEQ ID NO:298; SEQ ID NO:300; SEQ ID NO:302; SEQ ID NO:304; SEQ ID NO:312; SEQ ID NO:314; SEQ ID NO:316; SEQ ID NO:332; SEQ ID NO:334; SEQ ID NO:338; SEQ ID NO:340; SEQ ID NO:342; SEQ ID NO:348; SEQ ID NO:350; SEQ ID NO:352; SEQ ID NO:354; SEQ ID NO:356; SEQ ID NO:358; SEQ ID NO:360; SEQ ID NO:362; SEQ ID NO:364; SEQ ID NO:366; SEQ ID NO:368; SEQ ID NO:370; SEQ ID NO:372; SEQ ID NO:374; SEQ ID NO:376; SEQ ID NO:378; SEQ ID NO:380; SEQ ID NO:384; SEQ ID NO:386; SEQ ID NO:388; SEQ ID NO:390; SEQ ID NO:392; SEQ ID NO:394; SEQ ID NO:396; SEQ ID NO:400; and SEQ ID NO:402.

Additionally, optimized nucleic acids can be created. Preferably, an optimized nucleic acid encodes a polypeptide that has a function similar to those of the polypeptides listed in Table 1 and/or modulates a plant's growth and/or yield under normal and/or water-limited conditions and/or tolerance to an environmental stress, and more preferably increases a plant's growth and/or yield under normal and/or water-limited conditions and/or tolerance to an environmental stress upon its overexpression in the plant. As used herein, “optimized” refers to a nucleic acid that is genetically engineered to increase its expression in a given plant or animal. To provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) to eliminate sequences that cause destabilization, inappropriate polyadenylation, degradation and termination of RNA, or that form secondary structure hairpins or RNA splice sites; or 5) elimination of antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

An isolated polynucleotide of the invention can be optimized such that its distribution frequency of codon usage deviates, preferably, no more than 25% from that of highly expressed plant genes and, more preferably, no more than about 10%. In addition, consideration is given to the percentage G+C content of the degenerate third base (monocotyledons appear to favor G+C in this position, whereas dicotyledons do not). It is also recognized that the XCG (where X is A, T, C, or G) nucleotide is the least preferred codon in dicots, whereas the XTA codon is avoided in both monocots and dicots. Optimized nucleic acids of this invention also preferably have CG and TA doublet avoidance indices closely approximating those of the chosen host plant. More preferably, these indices deviate from that of the host by no more than about 10-15%.

The invention further provides an isolated recombinant expression vector comprising a polynucleotide as described above, wherein expression of the vector in a host cell results in the plant's increased growth and/or yield under normal or water-limited conditions and/or increased tolerance to environmental stress as compared to a wild type variety of the host cell. Accordingly, the isolated recombinant expression vector of the invention may be used to increase expression of nucleotides and polypeptides of Table 1 and thus to modulate floral organ development, root initiation, and yield in plants. When the nucleotides and polypeptides of Table 1 are expressed in a cereal plant of interest, the result is improved yield of the plant. In one embodiment, the invention provides a transgenic plant that overexpresses an isolated polynucleotide identified in Table 1 in the subcellular compartment and tissue indicated herein. The transgenic plant of the invention demonstrates an improved yield as compared to a wild type variety of the plant. As used herein, the term “improved yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured plant product. Alternatively, the increased plant yield can comprise about a 1.001 fold, 1.01 fold, 1.1 fold, 2 fold, 4 fold, 8 fold, 16 fold or 32 fold increase in measured plant products. For example, an increase in the bu/acre yield of soybeans or corn derived from a crop comprising plants which are transgenic for the nucleotides and polypeptides of Table 1, as compared with the bu/acre yield from untreated soybeans or corn cultivated under the same conditions, would be considered an improved yield. By increased yield it is also intended at least one of an increase in total seed numbers, an increase in total seed weight, an increase in root biomass and an increase in harvest index as compared to a wild-type variety of the crop plant that does not contain the recombinant expression vector of the invention.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. As used herein with respect to a recombinant expression vector, “operatively linked” is intended to mean that the nucleotide sequence of interest is linked to the regulatory sequence(s) in a manner which allows for expression of the nucleotide sequence (e.g., in a bacterial or plant host cell when the vector is introduced into the host cell). The term “regulatory sequence” is intended to include promoters, enhancers, and other expression control elements (e.g., polyadenylation signals). Such regulatory sequences are well known in the art. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides encoded by nucleic acids as described herein.

The recombinant expression vector of the invention also include one or more regulatory sequences, selected on the basis of the host cells to be used for expression, which is in operative association with the isolated polynucleotide to be expressed. As used herein with respect to a recombinant expression vector, “in operative association” or “operatively linked” means that the polynucleotide of interest is linked to the regulatory sequence(s) in a manner which allows for expression of the polynucleotide when the vector is introduced into the host cell (e.g., in a bacterial or plant host cell). The term “regulatory sequence” is intended to include promoters, enhancers, and other expression control elements (e.g., polyadenylation signals).

Plant gene expression should be operatively linked to an appropriate promoter conferring gene expression in a timely, cell specific, or tissue specific manner. Promoters useful in the expression cassettes of the invention include any promoter that is capable of initiating transcription in a plant cell. Such promoters include, but are not limited to, those that can be obtained from plants, plant viruses, and bacteria that contain genes that are expressed in plants, such as Agrobacterium and Rhizobium.

The promoter may be constitutive, inducible, developmental stage-preferred, cell type-preferred, tissue-preferred, or organ-preferred. Constitutive promoters are active under most conditions. Examples of constitutive promoters include the CaMV 19S and 35S promoters, the sX CaMV 35S promoter, the Sep1 promoter, the rice actin promoter, the Arabidopsis actin promoter, the ubiquitin promoter, pEmu, the figwort mosaic virus 35S promoter, the Smas promoter, the super promoter (U.S. Pat. No. 5,955,646), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

Inducible promoters are preferentially active under certain environmental conditions, such as the presence or absence of a nutrient or metabolite, heat or cold, light, pathogen attack, anaerobic conditions, and the like. For example, the hsp80 promoter from Brassica is induced by heat shock; the PPDK promoter is induced by light; the PR-1 promoters from tobacco, Arabidopsis, and maize are inducible by infection with a pathogen; and the Adh1 promoter is induced by hypoxia and cold stress. Plant gene expression can also be facilitated via an inducible promoter (For a review, see Gatz, 1997, Annu. Rev. Plant Physiol. Plant Mol. Biol. 48:89-108). Chemically inducible promoters are especially suitable if gene expression is wanted to occur in a time specific manner. Examples of such promoters are a salicylic acid inducible promoter (PCT Application No. WO 95/19443), a tetracycline inducible promoter (Gatz et al., 1992, Plant J. 2: 397-404), and an ethanol inducible promoter (PCT Application No. WO 93/21334).

In one preferred embodiment of the present invention, the inducible promoter is a stress-inducible promoter. For the purposes of the invention, stress-inducible promoters are preferentially active under one or more of the following stresses: sub-optimal conditions associated with salinity, drought, nitrogen, temperature, metal, chemical, pathogenic, and oxidative stresses. Stress inducible promoters include, but are not limited to, Cor78 (Chak et al., 2000, Planta 210:875-883; Hovath et al., 1993, Plant Physiol. 103:1047-1053), Cori5a (Artus et al., 1996, PNAS 93(23):13404-09), Rci2A (Medina et al., 2001, Plant Physiol. 125:1655-66; Nylander et al., 2001, Plant Mol. Biol. 45:341-52; Navarre and Goffeau, 2000, EMBO J. 19:2515-24; Capel et al., 1997, Plant Physiol. 115:569-76), Rd22 (Xiong et al., 2001, Plant Cell 13:2063-83; Abe et al., 1997, Plant Cell 9:1859-68; Iwasaki et al., 1995, Mol. Gen. Genet. 247:391-8), cDet6 (Lang and Palve, 1992, Plant Mol. Biol. 20:951-62), ADH1 (Hoeren et al., 1998, Genetics 149:479-90), KAT1 (Nakamura et al., 1995, Plant Physiol. 109:371-4), KST1 (Müller-Röber et al., 1995, EMBO 14:2409-16), Rha1 (Terryn et al., 1993, Plant Cell 5:1761-9; Terryn et al., 1992, FEBS Lett. 299(3):287-90), ARSK1 (Atkinson et al., 1997, GenBank Accession # L22302, and PCT Application No. WO 97/20057), PtxA (Plesch et al., GenBank Accession # X67427), SbHRGP3 (Ahn et al., 1996, Plant Cell 8:1477-90), GH3 (Liu et al., 1994, Plant Cell 6:645-57), the pathogen inducible PRP1-gene promoter (Ward et al., 1993, Plant. Mol. Biol. 22:361-366), the heat inducible hsp80-promoter from tomato (U.S. Pat. No. 5,187,267), cold inducible alpha-amylase promoter from potato (PCT Application No. WO 96/12814), or the wound-inducible pinII-promoter (European Patent No. 375091). For other examples of drought, cold, and salt-inducible promoters, such as the RD29A promoter, see Yamaguchi-Shinozalei et al., 1993, Mol. Gen. Genet. 236:331-340.

Developmental stage-preferred promoters are preferentially expressed at certain stages of development. Tissue and organ preferred promoters include those that are preferentially expressed in certain tissues or organs, such as leaves, roots, seeds, or xylem. Examples of tissue-preferred and organ-preferred promoters include, but are not limited to fruit-preferred, ovule-preferred, male tissue-preferred, seed-preferred, integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Seed-preferred promoters are preferentially expressed during seed development and/or germination. For example, seed-preferred promoters can be embryo-preferred, endosperm-preferred, and seed coat-preferred (See Thompson et al., 1989, BioEssays 10:108). Examples of seed-preferred promoters include, but are not limited to, cellulose synthase (celA), Cim1, gamma-zein, globulin-1, maize 19 kD zein (cZ19B1), and the like.

Other suitable tissue-preferred or organ-preferred promoters include the napin-gene promoter from rapeseed (U.S. Pat. No. 5,608,152), the USP-promoter from Vicia faba (Baeumlein et al., 1991, Mol. Gen. Genet. 225(3): 459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980), or the legumin B4 promoter (LeB4; Baeumlein et al., 1992, Plant Journal, 2(2): 233-9), as well as promoters conferring seed specific expression in monocot plants like maize, barley, wheat, rye, rice, etc. Suitable promoters to note are the Ipt2 or Ipt1-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15 kD zein promoter, the 22 kD zein promoter, the 27 kD zein promoter, the —zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

As set forth above, certain embodiments of the invention employ promoters that are capable of enhancing gene expression in leaves. In some embodiments, the promoter is a leaf-specific promoter. Any leaf-specific promoter may be employed in these embodiments of the invention. Many such promoters are known, for example, the USP promoter from Vicia faba (SEQ ID NO:403 or SEQ ID NO:404, Baeumlein et al. (1991) Mol. Gen. Genet. 225, 459-67), promoters of light-inducible genes such as ribulose-1.5-bisphosphate carboxylase (rbcS promoters), promoters of genes encoding chlorophyll a/b-binding proteins (Cab), Rubisco activase, B-subunit of chloroplast glyceraldehyde 3-phosphate dehydrogenase from A. thaliana, (Kwon et al. (1994) Plant Physiol. 105, 357-67) and other leaf specific promoters such as those identified in Aleman, I. (2001) Isolation and characterization of leaf-specific promoters from alfalfa (Medicago sativa), Masters thesis, New Mexico State University, Los Cruces, N. Mex., and the like.

In other embodiments of the invention, a root or shoot specific promoter is employed. For example, the Super promoter (SEQ ID NO:405) provides high level expression in both root and shoots (Ni et al. (1995) Plant J. 7: 661-676). Other root specific promoters include, without limitation, the TobRB7 promoter (Yamamoto et al. (1991) Plant Cell 3, 371-382), the rolD promoter (Leach et al. (1991) Plant Science 79, 69-76); CaMV 35S Domain A (Benfey et al. (1989) Science 244, 174-181), and the like.

In other embodiments, a constitutive promoter is employed. Constitutive promoters are active under most conditions. Examples of constitutive promoters suitable for use in these embodiments include the parsley ubiquitin promoter described in WO 2003/102198 (SEQ ID NO:406, (SEQ ID NO:452)); the CaMV 19S and 35S promoters, the sX CaMV 35S promoter, the Sep1 promoter, the rice actin promoter, the Arabidopsis actin promoter, the maize ubiquitin promoter, pEmu, the figwort mosaic virus 35S promoter, the Smas promoter, the super promoter (U.S. Pat. No. 5,955,646), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

In accordance with the invention, a chloroplast transit sequence refers to a nucleotide sequence that encodes a chloroplast transit peptide. Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); ferredoxin (Jansen et al. (1988) Curr. Genetics 13:517-522) (SEQ ID NO:460); nitrite reductase (Back et al (1988) MGG 212:20-26) and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481. As defined herein, a mitochondrial transit sequence refers to a nucleotide sequence that encodes a mitochondrial presequence and directs the protein to mitochondria. Examples of mitochondrial presequences include groups consisting of ATPase subunits, ATP synthase subunits, Rieske-FeS protein, Hsp60, malate dehydrogenase, citrate synthase, aconitase, isocitrate dehydrogenase, pyruvate dehydrogenase, malic enzyme, glycine decarboxylase, serine hydroxymethyl transferase, isovaleryl-CoA dehydrogenase and superoxide dismutase. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; Faivre-Nitschke et al (2001) Eur J Biochem 268 1332-1339; Däschner et al. (1999) 39:1275-1282 (SEQ ID NO:456 and SEQ ID NO:458) and Shah et al. (1986) Science 233:478-481.

Additional flexibility in controlling heterologous gene expression in plants may be obtained by using DNA binding domains and response elements from heterologous sources (i.e., DNA binding domains from non-plant sources). An example of such a heterologous DNA binding domain is the LexA DNA binding domain (Brent and Ptashne, 1985, Cell 43:729-736).

In a preferred embodiment of the present invention, the polynucleotides listed in Table 1 are expressed in plant cells from higher plants (e.g., the spermatophytes, such as crop plants). A polynucleotide may be “introduced” into a plant cell by any means, including transfection, transformation or transduction, electroporation, particle bombardment, agroinfection, and the like. Suitable methods for transforming or transfecting plant cells are disclosed, for example, using particle bombardment as set forth in U.S. Pat. Nos. 4,945,050; 5,036,006; 5,100,792; 5,302,523; 5,464,765; 5,120,657; 6,084,154; and the like. More preferably, the transgenic corn seed of the invention may be made using Agrobacterium transformation, as described in U.S. Pat. Nos. 5,591,616; 5,731,179; 5,981,840; 5,990,387; 6,162,965; 6,420,630, U.S. patent application publication number 2002/0104132, and the like. Transformation of soybean can be performed using for example a technique described in European Patent No. EP 0424047, U.S. Pat. No. 5,322,783, European Patent No. EP 0397 687, U.S. Pat. No. 5,376,543, or U.S. Pat. No. 5,169,770. A specific example of wheat transformation can be found in PCT Application No. WO 93/07256. Cotton may be transformed using methods disclosed in U.S. Pat. Nos. 5,004,863; 5,159,135; 5,846,797, and the like. Rice may be transformed using methods disclosed in U.S. Pat. Nos. 4,666,844; 5,350,688; 6,153,813; 6,333,449; 6,288,312; 6,365,807; 6,329,571, and the like. Canola may be transformed, for example, using methods such as those disclosed in U.S. Pat. Nos. 5,188,958; 5,463,174; 5,750,871; EP1566443; WO02/00900; and the like. Other plant transformation methods are disclosed, for example, in U.S. Pat. Nos. 5,932,782; 6,153,811; 6,140,553; 5,969,213; 6,020,539, and the like. Any plant transformation method suitable for inserting a transgene into a particular plant may be used in accordance with the invention.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and may be transiently expressed or transiently active.

Another aspect of the invention pertains to an isolated polypeptide having a sequence selected from the group consisting of the polypeptide sequences listed in Table 1. An “isolated” or “purified” polypeptide is free of some of the cellular material when produced by recombinant DNA techniques, or chemical precursors or other chemicals when chemically synthesized. The language “substantially free of cellular material” includes preparations of a polypeptide in which the polypeptide is separated from some of the cellular components of the cells in which it is naturally or recombinantly produced. In one embodiment, the language “substantially free of cellular material” includes preparations of a polypeptide of the invention having less than about 30% (by dry weight) of contaminating polypeptides, more preferably less than about 20% of contaminating polypeptides, still more preferably less than about 10% of contaminating polypeptides, and most preferably less than about 5% contaminating polypeptides.

The determination of activities and kinetic parameters of enzymes is well established in the art. Experiments to determine the activity of any given altered enzyme must be tailored to the specific activity of the wild-type enzyme, which is well within the ability of one skilled in the art. Overviews about enzymes in general, as well as specific details concerning structure, kinetics, principles, methods, applications and examples for the determination of many enzyme activities are abundant and well known to one skilled in the art.

The invention is also embodied in a method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of: (a) introducing into a plant cell an expression vector comprising at least one polynucleotide listed in Table 1, and (b) generating from the plant cell a transgenic plant that expresses the polynucleotide, wherein expression of the polynucleotide in the transgenic plant results in the plant's increased growth and/or yield under normal or water-limited conditions and/or increased tolerance to environmental stress as compared to a wild type variety of the plant. The plant cell may be, but is not limited to, a protoplast, gamete producing cell, and a cell that regenerates into a whole plant. As used herein, the term “transgenic” refers to any plant, plant cell, callus, plant tissue, or plant part, that contains at least one recombinant polynucleotide listed in Table 1. In many cases, the recombinant polynucleotide is stably integrated into a chromosome or stable extra-chromosomal element, so that it is passed on to successive generations.

The present invention also provides a method of increasing a plant's growth and/or yield under normal or water-limited conditions and/or increasing a plant's tolerance to an environmental stress comprising the steps of increasing the expression of at least one polynucleotide listed in Table 1 in the plant. Expression of a protein can be increased by any method known to those of skill in the art.

The effect of the genetic modification on plant growth and/or yield and/or stress tolerance can be assessed by growing the modified plant under normal and/or less than suitable conditions and then analyzing the growth characteristics and/or metabolism of the plant. Such analysis techniques are well known to one skilled in the art, and include dry weight, wet weight, polypeptide synthesis, carbohydrate synthesis, lipid synthesis, evapotranspiration rates, general plant and/or crop yield, flowering, reproduction, seed setting, seed weight, seed number, root growth, respiration rates, photosynthesis rates, metabolite composition, etc., using methods known to those of skill in biotechnology.

In one embodiment the invention relates to subject mater summarized as follows:

Item 1 A transgenic plant transformed with an expression cassette comprising a polynucleotide encoding a full-length polypeptide having mitogen activated protein kinase activity, wherein the polypeptide comprises a domain having a sequence selected from the group consisting of amino acids 32 to 319 of SEQ ID NO:2; amino acids 42 to 329 of SEQ ID NO:4; amino acids 32 to 319 of SEQ ID NO:6; amino acids 32 to 310 of SEQ ID NO:8; amino acids 32 to 319 of SEQ ID NO:10; amino acids 32 to 319 of SEQ ID NO:12; amino acids 28 to 318 of SEQ ID NO:14; amino acids 32 to 326 of SEQ ID NO:16; amino acids 38 to 325 of SEQ ID NO:18; amino acids 44 to 331 of SEQ ID NO:20; amino acids 40 to 357 of SEQ ID NO:22; amino acids 60 to 346 of SEQ ID NO:24; amino acids 74 to 360 of SEQ ID NO:26; and amino acids 47 to 334 of SEQ ID NO:28 amino acids 47 to 334 of SEQ ID NO:28; amino acids 38 to 325 of SEQ ID NO:30; amino acids 32 to 319 of SEQ ID NO:32; amino acids 41 to 327 of SEQ ID NO:34; amino acids 43 to 329 of SEQ ID NO:36; and amino acids 58 to 344 of SEQ ID NO:38.

Item 2 The transgenic plant of item 1, wherein the polypeptide comprises amino acids 1 to 368 of SEQ ID NO:2; amino acids 1 to 376 of SEQ ID NO:4; amino acids 1 to 368 of SEQ ID NO:6; amino acids 1 to 369 of SEQ ID NO:8; amino acids 1 to 371 of SEQ ID NO:10; amino acids 1 to 375 of SEQ ID NO:12; amino acids 1 to 523 of SEQ ID NO:14; amino acids 1 to 494 of SEQ ID NO:16; amino acids 1 to 373 of SEQ ID NO:18; amino acids 1 to 377 of SEQ ID NO:20; amino acids 1 to 404 of SEQ ID NO:22; amino acids 1 to 394 of SEQ ID NO:24; amino acids 1 to 415 of SEQ ID NO:26; amino acids 1 to 381 of SEQ ID NO:28 amino acids 1 to 381 of SEQ ID NO:28; amino acids 1 to 376 of SEQ ID NO:30; amino acids 1 to 368 of SEQ ID NO:32; amino acids 1 to 372 of SEQ ID NO:34; amino acids 1 to 374 of SEQ ID NO:36; or amino acids 1 to 372 of SEQ ID NO:38.

Item 3 A transgenic plant transformed with an expression cassette comprising an isolated polynucleotide encoding a full-length polypeptide having calcium dependent protein kinase activity, wherein the polypeptide comprises:
a) a protein kinase domain selected from the group consisting of a domain having a sequence comprising amino acids 59 to 317 of SEQ ID NO:40; amino acids 111 to 369 of SEQ ID NO:42; amino acids 126 to 386 of SEQ ID NO:44; amino acids 79 to 337 of SEQ ID NO:46; amino acids 80 to 338 of SEQ ID NO:48; amino acids 125 to 287 of SEQ ID NO:50; amino acids 129 to 391 of SEQ ID NO:52; amino acids 111 to 371 of SEQ ID NO:54; amino acids 61 to 319 of SEQ ID NO:56; amino acids 86 to 344 of SEQ ID NO:58; amino acids 79 to 337 of SEQ ID NO:60; amino acids 78 to 336 of SEQ ID NO:62; amino acids 90 to 348 of SEQ ID NO:64; amino acids 56 to 314 of SEQ ID NO:66; amino acids 67 to 325 of SEQ ID NO:68; amino acids 81 to 339 of SEQ ID NO:70; and amino acids 83 to 341 of SEQ ID NO:72; and
b) at least one EF hand domain having a sequence selected from the group consisting of amino acids 364 to 392 of SEQ ID NO:40; amino acids 416 to 444 of SEQ ID NO:42; amino acids 433 to 461 of SEQ ID NO:44; amino acids 384 to 412 of SEQ ID NO:46; amino acids 385 to 413 of SEQ ID NO:48; amino acids 433 to 461 of SEQ ID NO:50; amino acids 436 to 463 of SEQ ID NO:52; amino acids 418 to 446 of SEQ ID NO:54; amino acids 366 to 394 of SEQ ID NO:56; amino acids 391 to 419 of SEQ ID NO:58; amino acids 384 to 412 of SEQ ID NO:60; amino acids 418 to 446 of SEQ ID NO:62; amino acids 395 to 423 of SEQ ID NO:64; amino acids 372 to 400 of SEQ ID NO:68; amino acids 388 to 416 of SEQ ID NO:72; amino acids 452 to 480 of SEQ ID NO:42; amino acids 470 to 498 of SEQ ID NO:44; amino acids 420 to 448 of SEQ ID NO:46; amino acids 421 to 449 of SEQ ID NO:48; amino acids 470 to 498 of SEQ ID NO:50; amino acids 472 to 500 of SEQ ID NO:52; amino acids 455 to 483 of SEQ ID NO:54; amino acids 402 to 430 of SEQ ID NO:56; amino acids 427 to 455 of SEQ ID NO:58; amino acids 420 to 448 of SEQ ID NO:60; amino acids 454 to 482 of SEQ ID NO:62; amino acids 444 to 472 of SEQ ID NO:68; amino acids 460 to 488 of SEQ ID NO:72; amino acids 488 to 516 of SEQ ID NO:42; amino acids 512 to 540 of SEQ ID NO:44; amino acids 456 to 484 of SEQ ID NO:46; amino acids 457 to 485 of SEQ ID NO:48; amino acids 510 to 535 of SEQ ID NO:50; amino acids 512 to 537 of SEQ ID NO:52; amino acids 497 to 525 of SEQ ID NO:54; amino acids 438 to 466 of SEQ ID NO:56; amino acids 463 to 491 of SEQ ID NO:58; amino acids 456 to 484 of SEQ ID NO:60; amino acids 522 to 550 of SEQ ID NO:42; amino acids 546 to 570 of SEQ ID NO:44; amino acids 491 to 519 of SEQ ID NO:46; amino acids 492 to 520 of SEQ ID NO:48; amino acids 542 to 570 of SEQ ID NO:50; amino acids 542 to 570 of SEQ ID NO:52; amino acids 531 to 555 of SEQ ID NO:54; amino acids 474 to 502 of SEQ ID NO:56; amino acids 497 to 525 of SEQ ID NO:58; and amino acid 490 to 518 of SEQ ID NO:60; amino acids 489 to 517 of SEQ ID NO:62; amino acids 501 to 529 of SEQ ID NO:64; amino acids 470 to 498 of SEQ ID NO:66; amino acids 479 to 507 of SEQ ID NO:68; amino acids 492 to 520 of SEQ ID NO:70; and amino acids 495 to 523 of SEQ ID NO:72.

Item 4 The transgenic plant of item 3, wherein the polypeptide has a sequence comprising amino acids 1 to 418 of SEQ ID NO:40; amino acids 1 to 575 of SEQ ID NO:42; amino acids 1 to 590 of SEQ ID NO:44; amino acids 1 to 532 of SEQ ID NO:46; amino acids 1 to 528 of SEQ ID NO:48; amino acids 1 to 578 of SEQ ID NO:50; amino acids 1 to 580 of SEQ ID NO:52; amino acids 1 to 574 of SEQ ID NO:54; amino acids 1 to 543 of SEQ ID NO:56; amino acids 1 to 549 of SEQ ID NO:58; amino acids 1 to 544 of SEQ ID NO:60; amino acids 1 to 534 of SEQ ID NO:62; amino acids 1 to 549 of SEQ ID NO:64; amino acids 1 to 532 of SEQ ID NO:66; amino acids 1 to 525 of SEQ ID NO:68; amino acids 1 to 548 of SEQ ID NO:70; or amino acids 1 to 531 of SEQ ID NO:72.

Item 5 A transgenic plant transformed with an expression cassette comprising an isolated polynucleotide a full-length polypeptide having cyclin dependent protein kinase activity, wherein the polypeptide comprises:
a) a cyclin N terminal domain having a sequence selected from the group consisting of amino acids 59 to 190 of SEQ ID NO:74; amino acids 63 to 197 of SEQ ID NO:76; amino acids 73 to 222 of SEQ ID NO:78; and amino acids 54 to 186 of SEQ ID NO:80 and
b) a cyclin C terminal domain having a sequence selected from the group consisting of amino acids 192 to 252 of SEQ ID NO:74; amino acids 199 to 259 of SEQ ID NO:76; amino acids 224 to 284 of SEQ ID NO:78; and amino acids 188 to 248 of SEQ ID NO:80.

Item 6 The transgenic plant of item 5, wherein the polypeptide has a sequence comprising amino acids 1 to 355 of SEQ ID NO:74; amino acids 1 to 360 of SEQ ID NO:76; amino acids 1 to 399 of SEQ ID NO:78; or amino acids 1 to 345 of SEQ ID NO:80.

Item 7 A transgenic plant transformed with an expression cassette comprising an isolated polynucleotide a full-length polypeptide having serine/threonine-specific protein kinase activity, wherein the polypeptide comprises a domain selected from the group consisting of a domain having a sequence comprising amino acids 15 to 271 of SEQ ID NO:82; amino acids 4 to 260 of SEQ ID NO:84; amino acids 4 to 260 of SEQ ID NO:86; amino acids 18 to 274 of SEQ ID NO:88; amino acids 23 to 279 of SEQ ID NO:90; amino acids 5 to 261 of SEQ ID NO:92; amino acids 23 to 279 of SEQ ID NO:94; amino acids 4 to 260 of SEQ ID NO:96; amino acids 12 to 268 of SEQ ID NO:98; and amino acids 4 to 260 of SEQ ID NO:100.

Item 8 The transgenic plant of item 7, wherein the polypeptide has a sequence comprising amino acids 1 to 348 of SEQ ID NO:82; amino acids 1 to 364 of SEQ ID NO:84; amino acids 1 to 354 of SEQ ID NO:86; amino acids 1 to 359 of SEQ ID NO:88; amino acids 1 to 360 of SEQ ID NO:90; amino acids 1 to 336 of SEQ ID NO:92; amino acids 1 to 362 of SEQ ID NO:94; amino acids 1 to 370 of SEQ ID NO:96; amino acids 1 to 350 of SEQ ID NO:98; or amino acids 1 to 361 of SEQ ID NO:100.

Item 9 An isolated polynucleotide having a sequence selected from the group consisting of the polynucleotide sequences set forth in Table 1.

Item 10 An isolated polypeptide having a sequence selected from the group consisting of the polypeptide sequences set forth in Table 1.

Item 11 A method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of:

(a) introducing into a plant cell an expression vector comprising at least one polynucleotide listed in Table 1, and
(b) generating from the plant cell a transgenic plant that expresses the polynucleotide, wherein expression of the polynucleotide in the transgenic plant results in the plant having increased growth or yield under normal or water-limited conditions or increased tolerance to environmental stress, as compared to a wild type variety of the plant.

Item 12 A method of increasing a plant's growth or yield under normal or water-limited conditions or increasing a plant's tolerance to an environmental stress comprising the steps of;

(a) introducing into a plant cell an expression vector comprising at least one polynucleotide listed in Table 1, and
(b) generating from the plant cell a transgenic plant that expresses the polynucleotide, wherein expression of the polynucleotide in the transgenic plant results in the plant having increased growth or yield under normal or water-limited conditions or increased tolerance to environmental stress, as compared to a wild type variety of the plant.

Item 13 A transgenic plant transformed with an expression cassette comprising an isolated polynucleotide encoding a full-length polypeptide having phospholipid hydroperoxide glutathione peroxidase activity, wherein the polypeptide comprises a glutathione peroxidase domain selected from the group consisting of 9 to 117 of SEQ ID NO:102; amino acids 17 to 125 of SEQ ID NO:104; amino acids 79 to 187 of SEQ ID NO:106; amino acids 10 to 118 of SEQ ID NO:108; amino acids 12 to 120 of SEQ ID NO:110; amino acids 9 to 117 of SEQ ID NO:112; amino acids 9 to 117 of SEQ ID NO:114; amino acids 10 to 118 of SEQ ID NO:116; amino acids 9 to 117 of SEQ ID NO:118; amino acids 77 to 185 of SEQ ID NO:120; amino acids 12 to 120 of SEQ ID NO:122; amino acids 12 to 120 of SEQ ID NO:124; amino acids 12 to 120 of SEQ ID NO:126; amino acids 12 to 120 of SEQ ID NO:128; amino acids 10 to 118 of SEQ ID NO:130; amino acids 70 to 178 of SEQ ID NO:132; amino acids 10 to 118 of SEQ ID NO:134; and amino acids 24 to 132 of SEQ ID NO:136.

Item 14 An isolated polynucleotide having a sequence selected from the group consisting of the polynucleotide sequences set forth in Table 1.

Item 15 An isolated polypeptide having a sequence selected from the group consisting of the polypeptide sequences set forth in Table 1.

Item 16 A method of producing a transgenic plant comprising at least one polynucleotide listed in Table 1, wherein expression of the polynucleotide in the plant results in the plant's increased growth or yield under normal or water-limited conditions or increased tolerance to an environmental stress as compared to a wild type variety of the plant comprising the steps of:

(a) introducing into a plant cell an expression vector comprising at least one polynucleotide listed in Table 1, and
(b) generating from the plant cell a transgenic plant that expresses the polynucleotide, wherein expression of the polynucleotide in the transgenic plant results in the plant's increased growth or yield under normal or water-limited conditions or increased tolerance to environmental stress as compared to a wild type variety of the plant.

Item 17 A method of increasing a plant's growth or yield under normal or water-limited conditions or increasing a plant's tolerance to an environmental stress comprising the steps of:
(a) introducing into a plant cell an expression vector comprising at least one polynucleotide listed in Table 1, and
(b) generating from the plant cell a transgenic plant that expresses the polynucleotide, wherein expression of the polynucleotide in the transgenic plant results in the plant's increased growth or yield under normal or water-limited conditions or increased tolerance to environmental stress, as compared to a wild type variety of the plant.

Item 18 A transgenic plant transformed with an expression cassette comprising an isolated polynucleotide encoding a full-length polypeptide comprising a TCP family transcription factor domain having a sequence selected from the group consisting of amino acids 57 to 249 of SEQ ID NO:138; amino acids 54 to 237 of SEQ ID NO:140; amino acids 43 to 323 of SEQ ID NO:142; or amino acids 41 to 262 of SEQ ID NO:144.

Item 19 A transgenic plant transformed with an expression cassette comprising an isolated polynucleotide encoding a full-length ribosomal protein S6 kinase polypeptide comprising:
a) a kinase domain having a sequence selected from the group consisting of amino acids 124 to 379 of SEQ ID NO:146; amino acids 150 to 406 of SEQ ID NO:148; and amino acids 152 to 408 of SEQ ID NO:150 or
b) a kinase C-terminal domain having a sequence selected from the group consisting of amino acids 399 to 444 of SEQ ID NO:146; amino acids 426 to 468 of SEQ ID NO:148; and amino acids 428 to 471 of SEQ ID NO:150.

Item 20 A transgenic plant transformed with an expression cassette comprising an isolated polynucleotide encoding a full-length polypeptide comprising a CAAX amino terminal protease domain having a sequence selected from the group consisting of amino acids 255 to 345 of SEQ ID NO:158; amino acids 229 to 319 of SEQ ID NO:160; and amino acids 267 to 357 of SEQ ID NO:162.

Item 21 A transgenic plant transformed with an expression cassette comprising an isolated polynucleotide encoding a full-length DNA binding protein comprising a metallopeptidase family M24 domain having a sequence selected from the group consisting of amino acids 21 to 296 of SEQ ID NO:164; amino acids 20 to 295 of SEQ ID NO:166; amino acids 20 to 295 of SEQ ID NO:168; amino acids 22 to 297 of SEQ ID NO:170; and amino acids 22 to 297 of SEQ ID NO:172.

Item 22 A transgenic plant transformed with an expression cassette comprising an isolated polynucleotide encoding a rev interacting protein mis3 having a sequence comprising amino acids 1 to 390 of SEQ ID NO:176; amino acids 1 to 389 of SEQ ID NO:178; or amino acids 1 to 391 of SEQ ID NO:180.

Item 23 A transgenic plant transformed with an expression cassette comprising an isolated polynucleotide encoding a GRF1 interacting factor comprising an SSXT protein (N terminal region) domain having a sequence selected from the group consisting of amino acids 7 to 80 of SEQ ID NO:182; amino acids 7 to 80 of SEQ ID NO:184; amino acids 7 to 80 of SEQ ID NO:186; and amino acids 6 to 79 of SEQ ID NO:188.

Item 24 A transgenic plant transformed with an expression cassette comprising an isolated polynucleotide encoding eukaryotic translation initiation factor 4A comprising:
a) a DEAD/DEAH box helicase domain having a sequence selected from the group consisting of amino acids 59 to 225 of SEQ ID NO:190; amino acids 64 to 230 of SEQ ID NO:192; amino acids 58 to 224 of SEQ ID NO:194; amino acids 64 to 230 of SEQ ID NO:196; amino acids 64 to 230 of SEQ ID NO:198; and amino acids 64 to 230 of SEQ ID NO:200; or
b) a helicase conserved C-terminal domain having a sequence comprising amino acids 293 to 369 of SEQ ID NO:190; amino acids 298 to 374 of SEQ ID NO:192; amino acids 292 to 368 of SEQ ID NO:194; amino acids 298 to 374 of SEQ ID NO:196; amino acids 298 to 374 of SEQ ID NO:198; and amino acids 298 to 374 of SEQ ID NO:200.

Item 25 A transgenic plant transformed with an expression cassette comprising an isolated polynucleotide encoding TGF beta receptor interacting protein comprising a WD domain, G-beta repeat having a sequence selected from the group consisting of amino acids 42 to 80 of SEQ ID NO:154; amino acids 42 to 80 of SEQ ID NO:156; and amino acids 42 to 80 of SEQ ID NO:152; or a WD domain, G-beta repeat having a sequence selected from the group consisting of amino acids 136 to 174 of SEQ ID NO:154; amino acids 136 to 174 of SEQ ID NO:156; and amino acids 136 to 174 of SEQ ID NO:152; or a WD domain, G-beta repeat having a sequence selected from the group consisting of amino acids 181 to 219 of SEQ ID NO:154; amino acids 181 to 219 of SEQ ID NO:156; and amino acids 181 to 219 of SEQ ID NO:152; or a WD domain, G-beta repeat having a sequence selected from the group consisting of amino acids 278 to 316 of SEQ ID NO:154; amino acids 278 to 316 of SEQ ID NO:156; and amino acids 278 to 316 of SEQ ID NO:152.

Item 26 A transgenic plant transformed with an expression cassette comprising an isolated polynucleotide having a sequence selected from the group consisting of SEQ ID NO:173; SEQ ID NO:201; SEQ ID NO:203; and SEQ ID NO:205.

Item 27 An isolated polynucleotide having a sequence selected from the group consisting of the polynucleotide sequences set forth in Table 1.

Item 28 An isolated polypeptide having a sequence selected from the group consisting of the polypeptide sequences set forth in Table 1.

Item 29 A method of producing a transgenic plant comprising at least one polynucleotide listed in Table 1, wherein expression of the polynucleotide in the plant results in the plant's increased growth and/or yield under normal or water-limited conditions or increased tolerance to an environmental stress as compared to a wild type variety of the plant comprising the steps of:

(a) introducing into a plant cell an expression vector comprising at least one polynucleotide listed in Table 1, and
(b) generating from the plant cell a transgenic plant that expresses the polynucleotide, wherein expression of the polynucleotide in the transgenic plant results in the plant's increased growth or yield under normal or water-limited conditions and/or increased tolerance to environmental stress as compared to a wild type variety of the plant.

Item 30 A method of increasing a plant's growth or yield under normal or water-limited conditions or increasing a plant's tolerance to an environmental stress comprising the steps of:

(a) introducing into a plant cell an expression vector comprising at least one polynucleotide listed in Table 1, and
(b) generating from the plant cell a transgenic plant that expresses the polynucleotide, wherein expression of the polynucleotide in the transgenic plant results in the plant's increased growth or yield under normal or water-limited conditions and/or increased tolerance to environmental stress as compared to a wild type variety of the plant.

Item 31 A transgenic plant transformed with an expression cassette comprising an isolated polynucleotide encoding a full-length polypeptide comprising an AP2 domain having a sequence at least 64% identical to amino acids 44 to 99 of SEQ ID NO:208.

Item 32 The transgenic plant of item 31, wherein the polypeptide has a sequence selected from the group consisting of SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, and SEQ ID NO: 252.

Item 33 An isolated polynucleotide having a sequence selected from the group consisting of the polynucleotide sequences set forth in Table 1.

Item 34 An isolated polypeptide having a sequence selected from the group consisting of the polypeptide sequences set forth in Table 1.

Item 35 A method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of:

(a) introducing into a plant cell an expression vector comprising at least one polynucleotide listed in Table 1, and
(b) generating from the plant cell a transgenic plant that expresses the polynucleotide, wherein expression of the polynucleotide in the transgenic plant results in the plant's increased growth and/or yield under normal or water-limited conditions and/or increased tolerance to environmental stress as compared to a wild type variety of the plant.

Item 36 A method of increasing a plant's growth and/or yield under normal or water-limited conditions and/or increasing a plant's tolerance to an environmental stress comprising the steps of increasing the expression of at least one polynucleotide listed in Table 1 in the plant.

Item 37 A transgenic plant transformed with an expression cassette comprising a polynucleotide encoding a full-length brassinosteroid biosynthetic LKB-like polypeptide selected from the group consisting of amino acids 1 to 566 of SEQ ID NO:254, CAN79299, AAK15493, P93472, AAM47602, and AAL91175.

Item 38 A transgenic plant transformed with an expression cassette comprising a polynucleotide encoding a full-length RING-box polypeptide comprising amino acids 1 to 120 of SEQ ID NO:256.

Item 39 A transgenic plant transformed with an expression cassette comprising an isolated polynucleotide encoding a full-length polypeptide having serine/threonine protein phosphatase activity, wherein the polypeptide comprises a calcineurin-like phosphoesterase domain having a sequence selected from the groups consisting of amino amino acids 44 to 239 of SEQ ID NO:258; amino acids 43 to 238 of SEQ ID NO:260; amino acids 54 to 249 of SEQ ID NO:262; amino acids 44 to 240 of SEQ ID NO:264; amino acids 43 to 238 of SEQ ID NO:266; amino acids 54 to 249 of SEQ ID NO:268; amino acids 48 to 243 of SEQ ID NO:270; amino acids 47 to 242 of SEQ ID NO:272; amino acids 54 to 249 of SEQ ID NO:274; amino acids 48 to 243 of SEQ ID NO:276; amino acids 47 to 242 of SEQ ID NO:278; amino acids 44 to 240 of SEQ ID NO:280; amino acids 47 to 242 of SEQ ID NO:282; amino acids 47 to 243 of SEQ ID NO:284; and amino acids 60 to 255 of SEQ ID NO:286.

Item 40 The transgenic plant of item 39, wherein the polypeptide has a sequence comprising amino acids 1 to 304 of SEQ ID NO:258; amino acids 1 to 303 of SEQ ID NO:260; amino acids 1 to 305 of SEQ ID NO:262; amino acids 1 to 313 of SEQ ID NO:264; amino acids 1 to 306 of SEQ ID NO:266; amino acids 1 to 306 of SEQ ID NO:268; amino acids 1 to 308 of SEQ ID NO:270; amino acids 1 to 314 of SEQ ID NO:272; amino acids 1 to 306 of SEQ ID NO:274; amino acids 1 to 313 of SEQ ID NO:276; amino acids 1 to 305 of SEQ ID NO:278; amino acids 1 to 303 of SEQ ID NO:280; amino acids 1 to 313 of SEQ ID NO:282; amino acids 1 to 307 of SEQ ID NO:284; or amino acids 1 to 306 of SEQ ID NO:286.

Item 41 An isolated polynucleotide having a sequence selected from the group consisting of the polynucleotide sequences set forth in Table 1.

Item 42 An isolated polypeptide having a sequence selected from the group consisting of the polypeptide sequences set forth in Table 1.

Item 43 A method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of:

(a) introducing into a plant cell an expression vector comprising at least one polynucleotide listed in Table 1, and
(b) generating from the plant cell a transgenic plant that expresses the polynucleotide, wherein expression of the polynucleotide in the transgenic plant results in the plant having increased growth or yield under normal or water-limited conditions or increased tolerance to environmental stress, as compared to a wild type variety of the plant.

Item 44 A method of increasing a plant's growth or yield under normal or water-limited conditions or increasing a plant's tolerance to an environmental stress comprising the steps of;

(a) introducing into a plant cell an expression vector comprising at least one polynucleotide listed in Table 1, and
(b) generating from the plant cell a transgenic plant that expresses the polynucleotide, wherein expression of the polynucleotide in the transgenic plant results in the plant having increased growth or yield under normal or water-limited conditions or increased tolerance to environmental stress, as compared to a wild type variety of the plant.

Item 45 A transgenic plant transformed with an expression cassette comprising, in operative association,
a) an isolated polynucleotide encoding a promoter capable of enhancing gene expression in leaves; and
b) an isolated polynucleotide encoding a full-length polypeptide which is a long-chain-fatty-acid-CoA ligase subunit of acyl-CoA synthetase;
   wherein the transgenic plant demonstrates increased yield as compared to a wild type plant of the same variety which does not comprise the expression cassette.

Item 46 The transgenic plant of item 45, wherein the long-chain-fatty-acid-CoA ligase comprises a domain selected from the group amino acids 213 to 543 of SEQ ID NO:288; amino acids 299 to 715 of SEQ ID NO:290; amino acids 173 to 504 of SEQ ID NO:292; amino acids 124 to 457 of SEQ ID NO:294; amino acids 178 to 509 of SEQ ID NO:296; amino acids 82 to 424 of SEQ ID NO:298; amino acids 207 to 388 of SEQ ID NO:300; amino acids 215 to 561 of SEQ ID NO:302; amino acids 111 to 476 of SEQ ID NO:304; amino acids 206 to 544 of SEQ ID NO:306; amino acids 192 to 531 of SEQ ID NO:308; amino acids 191 to 528 of SEQ ID NO:310; amino acids 259 to 660 of SEQ ID NO:312; amino acids 234 to 642 of SEQ ID NO:314; and amino acids 287 to 707 of SEQ ID NO:316.

Item 47 The transgenic plant of 2, wherein the long-chain-fatty-acid-CoA ligase comprises amino acids 1 to 561 of SEQ ID NO:288; amino acids 1 to 744 of SEQ ID NO:290; amino acids 1 to 518 of SEQ ID NO:292; amino acids 1 to 471 of SEQ ID NO:294; amino acids 1 to 523 of SEQ ID NO:296; amino acids 1 to 442 of SEQ ID NO:298; amino acids 1 to 555 of SEQ ID NO:300; amino acids 1 to 582 of SEQ ID NO:302; amino acids 1 to 455 of SEQ ID NO:304; amino acids 1 to 562 of SEQ ID NO:306; amino acids 1 to 547 of SEQ ID NO:308; amino acids 1 to 546 of SEQ ID NO:310; amino acids 1 to 691 of SEQ ID NO:312; amino acids 1 to 664 of SEQ ID NO:314; or amino acids 1 to 726 of SEQ ID NO:316.

Item 48 The transgenic plant of item 45, further defined as a species selected from the group consisting of maize, wheat, rice, soybean, cotton, oilseed rape, and canola.

Item 49 A seed which is true breeding for a transgene comprising, in operative association,
a) an isolated polynucleotide encoding a promoter capable of enhancing gene expression in leaves; and
b) an isolated polynucleotide encoding a full-length polypeptide which is a long-chain-fatty-acid-CoA ligase subunit of acyl-CoA synthetase;
   wherein a transgenic plant grown from said seed demonstrates increased tolerance to drought as compared to a wild type plant of the same variety which does not comprise the expression cassette.

Item 50 The seed of item 49, wherein the long-chain-fatty-acid-CoA ligase comprises a domain selected from the group amino acids 213 to 543 of SEQ ID NO:288; amino acids 299 to 715 of SEQ ID NO:290; amino acids 173 to 504 of SEQ ID NO:292; amino acids 124 to 457 of SEQ ID NO:294; amino acids 178 to 509 of SEQ ID NO:296; amino acids 82 to 424 of SEQ ID NO:298; amino acids 207 to 388 of SEQ ID NO:300; amino acids 215 to 561 of SEQ ID NO:302; amino acids 111 to 476 of SEQ ID NO:304; amino acids 206 to 544 of SEQ ID NO:306; amino acids 192 to 531 of SEQ ID NO:308; amino acids 191 to 528 of SEQ ID NO:310; amino acids 259 to 660 of SEQ ID NO:312; amino acids 234 to 642 of SEQ ID NO:314; and amino acids 287 to 707 of SEQ ID NO:316.

Item 51 The seed of item 50, wherein the long-chain-fatty-acid-CoA ligase comprises amino acids 1 to 561 of SEQ ID NO:288; amino acids 1 to 744 of SEQ ID NO:290; amino acids 1 to 518 of SEQ ID NO:292; amino acids 1 to 471 of SEQ ID NO:294; amino acids 1 to 523 of SEQ ID NO:296; amino acids 1 to 442 of SEQ ID NO:298; amino acids 1 to 555 of SEQ ID NO:300; amino acids 1 to 582 of SEQ ID NO:302; amino acids 1 to 455 of SEQ ID NO:304; amino acids 1 to 562 of SEQ ID NO:306; amino acids 1 to 547 of SEQ ID NO:308; amino acids 1 to 546 of SEQ ID NO:310; amino acids 1 to 691 of SEQ ID NO:312; amino acids 1 to 664 of SEQ ID NO:314; or amino acids 1 to 726 of SEQ ID NO:316.

Item 52 A method of producing a transgenic plant having enhanced yield as compared to a wild type plant of the same variety, the method comprising the steps of:
a) transforming a plant cell with an expression vector comprising, in operative association,
   i) an isolated polynucleotide encoding a promoter capable of enhancing gene expression in leaves; and
   ii) an isolated polynucleotide encoding a full-length polypeptide which is a long-chain-fatty-acid-CoA ligase subunit of acyl-CoA synthetase;
b) regenerating transgenic plants from the transformed plant cell; and
c) selecting higher-yielding plants from the regenerated transgenic plants.

Item 53 A transgenic plant transformed with an expression cassette comprising, in operative association,
a) an isolated polynucleotide encoding a promoter capable of enhancing gene expression in leaves; and
b) an isolated polynucleotide encoding a full-length beta-ketoacyl-ACP synthase polypeptide;
   wherein the transgenic plant demonstrates increased yield as compared to a wild type plant of the same variety which does not comprise the expression cassette.

Item 54 The transgenic plant of item 53, wherein the beta-ketoacyl-ACP synthase polypeptide comprises amino acids 1 to 379 of SEQ ID NO:318.

Item 55 The transgenic plant of item 53, further defined as a species selected from the group consisting of maize, wheat, rice, soybean, cotton, oilseed rape, and canola.

Item 56 A seed which is true breeding for a transgene comprising, in operative association,
a) an isolated polynucleotide encoding a promoter capable of enhancing gene expression in leaves; and
b) an isolated polynucleotide encoding a full-length beta-ketoacyl-ACP synthase polypeptide;
   wherein a transgenic plant grown from said seed demonstrates increased yield as compared to a wild type plant of the same variety which does not comprise the expression cassette.

Item 57 The seed of item 56, wherein the beta-ketoacyl-ACP synthase amino acids 1 to 379 of SEQ ID NO:318.

Item 58 A method of producing a transgenic plant having enhanced yield as compared to a wild type plant of the same variety, the method comprising the steps of:
a) transforming a plant cell with an expression vector comprising, in operative association,
   i) an isolated polynucleotide encoding a promoter capable of enhancing gene expression in leaves; and
   ii) an isolated polynucleotide encoding a full-length beta-ketoacyl-ACP synthase polypeptide;
b) regenerating transgenic plants from the transformed plant cell; and
c) selecting higher-yielding plants from the regenerated transgenic plants.

Item 59 A transgenic plant transformed with an expression cassette comprising, in operative association,
a) an isolated polynucleotide encoding a promoter capable of enhancing gene expression in leaves;
b) an isolated polynucleotide encoding a mitochondrial transit peptide; and
c) an isolated polynucleotide encoding a full-length polypeptide which is a subunit of acetyl-CoA carboxylase;
   wherein the transgenic plant demonstrates increased yield as compared to a wild type plant of the same variety which does not comprise the expression cassette.

Item 60 The transgenic plant of item 59, wherein the acetyl-CoA carboxylase subunit is selected from the group consisting of acetyl-CoA carboxylase alpha, biotin-dependent carboxylase, and biotin carboxyl carrier protein.

Item 61 The transgenic plant of item 60, wherein the acetyl-CoA carboxylase subunit is acetyl-CoA carboxylase alpha.

Item 62 The transgenic plant of item 61, wherein the acetyl-CoA carboxylase alpha comprises amino acids 1 to 319 of SEQ ID NO:320.

Item 63 The transgenic plant of item 60, wherein the acetyl-CoA carboxylase subunit is biotin-dependent carboxylase.

Item 64 The transgenic plant of item 63, wherein the biotin-dependent carboxylase comprises a domain selected from the group consisting of amino acids 3 to 308 of SEQ ID NO:322; amino acids 73 to 378 of SEQ ID NO:324; amino acids 38 to 344 of SEQ ID NO:326; and amino acids 73 to 378 of SEQ ID NO:328.

Item 65 The transgenic plant of item 64, wherein the biotin-dependent carboxylase comprises amino acids 1 to 449 of SEQ ID NO:322; amino acids 1 to 535 of SEQ ID NO:324; amino acids 1 to 732 of SEQ ID NO:326; or amino acids 1 to 539 of SEQ ID NO:328.

Item 66 The transgenic plant of item 60, wherein the acetyl-CoA carboxylase subunit is biotin carboxyl carrier protein.

Item 67 The transgenic plant of item 66, wherein the biotin carboxyl carrier protein comprises a domain selected from the group consisting of amino acids 79 to 152 of SEQ ID NO:330; amino acids 204 to 277 of SEQ ID NO:332; and amino acids 37 to 110 of SEQ ID NO:334.

Item 68 The transgenic plant of item 67, wherein the biotin carboxyl carrier protein subunit comprises amino acids 1 to 156 of SEQ ID NO:330; amino acids 1 to 282 of SEQ ID NO:332; or amino acids 1 to 115 of SEQ ID NO:334.

Item 69 The transgenic plant of item 66, further defined as a species selected from the group consisting of maize, wheat, rice, soybean, cotton, oilseed rape, and canola.

Item 70 A seed which is true breeding for a transgene comprising, in operative association,
a) an isolated polynucleotide encoding a promoter capable of enhancing gene expression in leaves;
b) an isolated polynucleotide encoding a mitochondrial transit peptide; and
c) an isolated polynucleotide encoding a full-length polypeptide which is a subunit of acetyl-CoA carboxylase;
   wherein a transgenic plant grown from said seed demonstrates increased yield as compared to a wild type plant of the same variety which does not comprise the expression cassette.

Item 71 The seed of item 70, wherein the acetyl-CoA carboxylase subunit is selected from the group consisting of acetyl-CoA carboxylase alpha, biotin-dependent carboxylase, and biotin carboxyl carrier protein.

Item 72 The seed of item 71, wherein the acetyl-CoA carboxylase subunit is acetyl-CoA carboxylase alpha.

Item 73 The seed of item 72, wherein the acetyl-CoA carboxylase alpha comprises amino acids 1 to 319 of SEQ ID NO:320.

Item 74 The seed of item 71, wherein the acetyl-CoA carboxylase subunit is biotin-dependent carboxylase.

Item 75 The seed of item 74, wherein the biotin-dependent carboxylase comprises a domain selected from the group consisting of amino acids 3 to 308 of SEQ ID NO:322; amino acids 73 to 378 of SEQ ID NO:324; amino acids 38 to 344 of SEQ ID NO:326; and amino acids 73 to 378 of SEQ ID NO:328.

Item 76 The seed of item 75, wherein the biotin-dependent carboxylase comprises amino acids 1 to 449 of SEQ ID NO:322; amino acids 1 to 535 of SEQ ID NO:324; amino acids 1 to 732 of SEQ ID NO:326; or amino acids 1 to 539 of SEQ ID NO:328.

Item 77 The seed of item 71, wherein the acetyl-CoA carboxylase subunit is biotin carboxyl carrier protein.

Item 78 The seed of item 77, wherein the biotin carboxyl carrier protein comprises a domain selected from the group consisting of amino acids 79 to 152 of SEQ ID NO:330; amino acids 204 to 277 of SEQ ID NO:332; and amino acids 37 to 110 of SEQ ID NO:334.

Item 79 The seed of item 78, wherein the biotin carboxyl carrier protein subunit comprises amino acids 1 to 156 of SEQ ID NO:330; amino acids 1 to 282 of SEQ ID NO:332; or amino acids 1 to 115 of SEQ ID NO:334.

Item 80 A method of producing a transgenic plant having enhanced yield as compared to a wild type plant of the same variety, the method comprising the steps of:
a) transforming a plant cell with an expression vector comprising, in operative association,
   i) an isolated polynucleotide encoding a promoter capable of enhancing gene expression in leaves;
   ii) an isolated polynucleotide encoding a mitochondrial transit peptide; and
   iii) an isolated polynucleotide encoding a full-length polypeptide which is a subunit of acetyl-CoA carboxylase;
b) regenerating transgenic plants from the transformed plant cell; and
c) selecting higher-yielding plants from the regenerated transgenic plants.

Item 81 A transgenic plant transformed with an expression cassette comprising, in operative association,
a) an isolated polynucleotide encoding a promoter capable of enhancing gene expression in leaves;
b) an isolated polynucleotide encoding a mitochondrial transit peptide; and
c) an isolated polynucleotide encoding a full-length 3-oxoacyl-[ACP] synthase II polypeptide;
   wherein the transgenic plant demonstrates increased yield as compared to a wild type plant of the same variety which does not comprise the expression cassette.

Item 82 The transgenic plant of item 81, wherein the 3-oxoacyl-ACP synthase II polypeptide comprises a domain selected from the group consisting of amino acids 12 to 410 of SEQ ID NO:336; amino acids 2 to 401 of SEQ ID NO:338; amino acids 55 to 456 of SEQ ID NO:340; and amino acids 2 to 401 of SEQ ID NO:342.

Item 83 The transgenic plant of item 82, wherein the 3-oxoacyl-ACP synthase II comprising amino acids 1 to 413 of SEQ ID NO:336; amino acids 1 to 406 of SEQ ID NO:338; amino acids 1 to 461 of SEQ ID NO:340; amino acids 1 to 406 of SEQ ID NO:342.

Item 84 The transgenic plant of item 81, further defined as a species selected from the group consisting of maize, wheat, rice, soybean, cotton, oilseed rape, and canola.

Item 85 A seed which is true breeding for a transgene comprising, in operative association,
a) an isolated polynucleotide encoding a promoter capable of enhancing gene expression in leaves;
b) an isolated polynucleotide encoding a mitochondrial transit peptide; and
c) an isolated polynucleotide encoding a full-length 3-oxoacyl-[ACP] synthase II polypeptide;
   wherein a transgenic plant grown from said seed demonstrates increased yield as compared to a wild type plant of the same variety which does not comprise the transgene.

Item 86 The seed of item 85, wherein the 3-oxoacyl-ACP synthase II polypeptide comprises a domain selected from the group consisting of amino acids 12 to 410 of SEQ ID NO:336; amino acids 2 to 401 of SEQ ID NO:338; amino acids 55 to 456 of SEQ ID NO:340; and amino acids 2 to 401 of SEQ ID NO:342.

Item 87 The seed of item 86, wherein the 3-oxoacyl-ACP synthase II comprising amino acids 1 to 413 of SEQ ID NO:336; amino acids 1 to 406 of SEQ ID NO:338; amino acids 1 to 461 of SEQ ID NO:340; amino acids 1 to 406 of SEQ ID NO:342.

Item 88 A method of producing a transgenic plant having enhanced yield as compared to a wild type plant of the same variety, the method comprising the steps of:
a) transforming a plant cell with an expression vector comprising, in operative association,
   i) an isolated polynucleotide encoding a promoter capable of enhancing gene expression in leaves;
   ii) an isolated polynucleotide encoding a mitochondrial transit peptide; and
   iii) an isolated polynucleotide encoding a full-length 3-oxoacyl-[ACP] synthase II polypeptide;
b) regenerating transgenic plants from the transformed plant cell; and
c) selecting higher-yielding plants from the regenerated transgenic plants.

Item 89 A transgenic plant transformed with an expression cassette comprising, in operative association,
a) an isolated polynucleotide encoding a promoter; and
b) an isolated polynucleotide encoding a full-length 3-oxoacyl-[ACP] reductase polypeptide;
   wherein the transgenic plant demonstrates increased yield as compared to a wild type plant of the same variety which does not comprise the expression cassette.

Item 90 The transgenic plant of item 89, wherein the promoter is capable of enhancing expression in leaves.

Item 91 The transgenic plant of item 89, wherein the expression vector further comprises a mitochondrial transit peptide.

Item 92 The transgenic plant of item 89, wherein the expression vector further comprises a chloroplast transit peptide.

Item 93 The transgenic plant of item 89, wherein the 3-oxoacyl-[ACP] reductase polypeptide comprises a domain selected from the group consisting of amino acids 80 to 181 of SEQ ID NO:344; amino acids 85 to 186 of SEQ ID NO:346; amino acids 79 to 180 of SEQ ID NO:348; amino acids 69 to 170 of SEQ ID NO:350; amino acids 51 to 154 of SEQ ID NO:352; amino acids 156 to 257 of SEQ ID NO:354; amino acids 90 to 193 of SEQ ID NO:356; amino acids 81 to 184 of SEQ ID NO:358; amino acids 128 to 228 of SEQ ID NO:360; amino acids 96 to 197 of SEQ ID NO:362; amino acids 97 to 198 of SEQ ID NO:364; amino acids 95 to 198 of SEQ ID NO:366; amino acids 103 to 208 of SEQ ID NO:368; amino acids 103 to 208 of SEQ ID NO:370; amino acids 100 to 203 of SEQ ID NO:372; amino acids 96 to 197 of SEQ ID NO:374; amino acids 96 to 197 of SEQ ID NO:376; amino acids 89 to 192 of SEQ ID NO:378; amino acids 159 to 260 of SEQ ID NO:380; amino acids 88 to 187 of SEQ ID NO:382; amino acids 148 to 249 of SEQ ID NO:384; amino acids 98 to 202 of SEQ ID NO:386; amino acids 95 to 199 of SEQ ID NO:388; amino acids 154 to 257 of SEQ ID NO:390; amino acids 88 to 187 of SEQ ID NO:392; amino acids 100 to 201 of SEQ ID NO:394; and amino acids 88 to 187 of SEQ ID NO:396.

Item 94 The transgenic plant of item 93, wherein the 3-oxoacyl-ACP reductase polypeptide comprises amino acids 1 to 244 of SEQ ID NO:344; amino acids 1 to 247 of SEQ ID NO:346; amino acids 1 to 253 of SEQ ID NO:348; amino acids 1 to 243 of SEQ ID NO:350; amino acids 1 to 236 of SEQ ID NO:352; amino acids 1 to 320 of SEQ ID NO:354; amino acids 1 to 275 of SEQ ID NO:356; amino acids 1 to 260 of SEQ ID NO:358; amino acids 1 to 294 of SEQ ID NO:360; amino acids 1 to 267 of SEQ ID NO:362; amino acids 1 to 272 of SEQ ID NO:364; amino acids 1 to 280 of SEQ ID NO:366; amino acids 1 to 282 of SEQ ID NO:368; amino acids 1 to 282 of SEQ ID NO:370; amino acids 1 to 265 of SEQ ID NO:372; amino acids 1 to 264 of SEQ ID NO:374; amino acids 1 to 271 of SEQ ID NO:376; amino acids 1 to 256 of SEQ ID NO:378; amino acids 1 to 323 of SEQ ID NO:380; amino acids 1 to 249 of SEQ ID NO:382; amino acids 1 to 312 of SEQ ID NO:384; amino acids 1 to 246 of SEQ ID NO:386; amino acids 1 to 258 of SEQ ID NO:388; amino acids 1 to 320 of SEQ ID NO:390; amino acids 1 to 253 of SEQ ID NO:392; amino acids 1 to 273 of SEQ ID NO:394; or amino acids 1 to 253 of SEQ ID NO:396.

Item 95 The transgenic plant of item 89, further defined as a species selected from the group consisting of maize, wheat, rice, soybean, cotton, oilseed rape, and canola.

Item 96 A seed which is true breeding for a transgene comprising, in operative association,
a) an isolated polynucleotide encoding a promoter; and
b) an isolated polynucleotide encoding a full-length 3-oxoacyl-[ACP] reductase polypeptide;
   wherein a transgenic plant grown from said seed demonstrates increased yield as compared to a wild type plant of the same variety which does not comprise the expression cassette.

Item 97 The seed of item 96, wherein the promoter is capable of enhancing expression in leaves.

Item 98 The seed of item 97, wherein the expression vector further comprises a mitochondrial transit peptide.

Item 99 The seed of item 96, wherein the expression vector further comprises a chloroplast transit peptide.

Item 100 The seed of item 96, wherein the 3-oxoacyl-[ACP] reductase polypeptide comprises a domain selected from the group consisting of amino acids 80 to 181 of SEQ ID NO:344; amino acids 85 to 186 of SEQ ID NO:346; amino acids 79 to 180 of SEQ ID NO:348; amino acids 69 to 170 of SEQ ID NO:350; amino acids 51 to 154 of SEQ ID NO:352; amino acids 156 to 257 of SEQ ID NO:354; amino acids 90 to 193 of SEQ ID NO:356; amino acids 81 to 184 of SEQ ID NO:358; amino acids 128 to 228 of SEQ ID NO:360; amino acids 96 to 197 of SEQ ID NO:362; amino acids 97 to 198 of SEQ ID NO:364; amino acids 95 to 198 of SEQ ID NO:366; amino acids 103 to 208 of SEQ ID NO:368; amino acids 103 to 208 of SEQ ID NO:370; amino acids 100 to 203 of SEQ ID NO:372; amino acids 96 to 197 of SEQ ID NO:374; amino acids 96 to 197 of SEQ ID NO:376; amino acids 89 to 192 of SEQ ID NO:378; amino acids 159 to 260 of SEQ ID NO:380; amino acids 88 to 187 of SEQ ID NO:382; amino acids 148 to 249 of SEQ ID NO:384; amino acids 98 to 202 of SEQ ID NO:386; amino acids 95 to 199 of SEQ ID NO:388; amino acids 154 to 257 of SEQ ID NO:390; amino acids 88 to 187 of SEQ ID NO:392; amino acids 100 to 201 of SEQ ID NO:394; and amino acids 88 to 187 of SEQ ID NO:396.

Item 101 The seed of item 100, wherein the 3-oxoacyl-ACP reductase polypeptide comprises amino acids 1 to 244 of SEQ ID NO:344; amino acids 1 to 247 of SEQ ID NO:346; amino acids 1 to 253 of SEQ ID NO:348; amino acids 1 to 243 of SEQ ID NO:350; amino acids 1 to 236 of SEQ ID NO:352; amino acids 1 to 320 of SEQ ID NO:354; amino acids 1 to 275 of SEQ ID NO:356; amino acids 1 to 260 of SEQ ID NO:358; amino acids 1 to 294 of SEQ ID NO:360; amino acids 1 to 267 of SEQ ID NO:362; amino acids 1 to 272 of SEQ ID NO:364; amino acids 1 to 280 of SEQ ID NO:366; amino acids 1 to 282 of SEQ ID NO:368; amino acids 1 to 282 of SEQ ID NO:370; amino acids 1 to 265 of SEQ ID NO:372; amino acids 1 to 264 of SEQ ID NO:374; amino acids 1 to 271 of SEQ ID NO:376; amino acids 1 to 256 of SEQ ID NO:378; amino acids 1 to 323 of SEQ ID NO:380; amino acids 1 to 249 of SEQ ID NO:382; amino acids 1 to 312 of SEQ ID NO:384; amino acids 1 to 246 of SEQ ID NO:386; amino acids 1 to 258 of SEQ ID NO:388; amino acids 1 to 320 of SEQ ID NO:390; amino acids 1 to 253 of SEQ ID NO:392; amino acids 1 to 273 of SEQ ID NO:394; or amino acids 1 to 253 of SEQ ID NO:396.

Item 102 A method of producing a transgenic plant having enhanced yield as compared to a wild type plant of the same variety, the method comprising the steps of:
a) transforming a plant cell with an expression vector comprising, in operative association,
   i) an isolated polynucleotide encoding a promoter; and
   ii) an isolated polynucleotide encoding a full-length 3-oxoacyl-[ACP] reductase polypeptide;
b) regenerating transgenic plants from the transformed plant cell; and
c) selecting higher-yielding plants from the regenerated transgenic plants.

Item 103 The method of item 102, wherein the promoter is capable of enhancing expression in leaves.

Item 104 The method of item 103, wherein the expression vector further comprises a mitochondrial transit peptide.

Item 105 The method of item 102, wherein the expression vector further comprises a chloroplast transit peptide.

Item 106 A transgenic plant transformed with an expression cassette comprising, in operative association,
a) an isolated polynucleotide encoding a promoter;
b) an isolated polynucleotide encoding a mitochondrial transit peptide, and
c) an isolated polynucleotide encoding a full-length biotin synthetase polypeptide;
   wherein the transgenic plant demonstrates increased yield as compared to a wild type plant of the same variety which does not comprise the expression cassette.

Item 107 The transgenic plant of item 105, wherein the biotin synthetase comprises a domain selected from the group consisting of amino acids 78 to 300 of SEQ ID NO:398; amino acids 82 to 301 of SEQ ID NO:400; and amino acids 79 to 298 of SEQ ID NO:402.

Item 108 The transgenic plant of item 107, wherein the biotin synthetase comprises amino acids 1 to 362 of SEQ ID NO:398; amino acids 1 to 304 of SEQ ID NO:400; or amino acids 1 to 372 of SEQ ID NO:402.

Item 109 The transgenic plant of item 106, further defined as a species selected from the group consisting of maize, wheat, rice, soybean, cotton, oilseed rape, and canola.

Item 110 A seed which is true breeding for a transgene comprising, in operative association,
a) an isolated polynucleotide encoding a promoter;
b) an isolated polynucleotide encoding a mitochondrial transit peptide, and
c) an isolated polynucleotide encoding a full-length biotin synthetase polypeptide;
   wherein a transgenic plant grown from said seed demonstrates increased yield as compared to a wild type plant of the same variety which does not comprise the expression cassette.

Item 111 The seed of item 110, wherein the biotin synthetase comprises a domain selected from the group consisting of amino acids 78 to 300 of SEQ ID NO:398; amino acids 82 to 301 of SEQ ID NO:400; and amino acids 79 to 298 of SEQ ID NO:402.

Item 112 The seed of item 111, wherein the biotin synthetase comprises amino acids 1 to 362 of SEQ ID NO:398; amino acids 1 to 304 of SEQ ID NO:400; or amino acids 1 to 372 of SEQ ID NO:402.

Item 113 A method of producing a transgenic plant having enhanced yield as compared to a wild type plant of the same variety, the method comprising the steps of:
a) transforming a plant cell with an expression vector comprising, in operative association,
   i) an isolated polynucleotide encoding a promoter;
   ii) an isolated polynucleotide encoding a mitochondrial transit peptide, and
   iii) an isolated polynucleotide encoding a full-length biotin synthetase polypeptide;
b) regenerating transgenic plants from the transformed plant cell; and
c) selecting higher-yielding plants from the regenerated transgenic plants.

Item 114 An isolated polynucleotide having a sequence selected from the group consisting of SEQ ID NO:291; SEQ ID NO:293; SEQ ID NO:295; SEQ ID NO:297; SEQ ID NO:299; SEQ ID NO:301; SEQ ID NO:303; SEQ ID NO:311; SEQ ID NO:313; SEQ ID NO:315; SEQ ID NO:331; SEQ ID NO:333; SEQ ID NO:337; SEQ ID NO:339; SEQ ID NO:341; SEQ ID NO:347; SEQ ID NO:349; SEQ ID NO:351; SEQ ID NO:353; SEQ ID NO:355; SEQ ID NO:357; SEQ ID NO:359; SEQ ID NO:361; SEQ ID NO:363; SEQ ID NO:365; SEQ ID NO:367; SEQ ID NO:369; SEQ ID NO:371; SEQ ID NO:373; SEQ ID NO:375; SEQ ID NO:377; SEQ ID NO:379; SEQ ID NO:383; SEQ ID NO:385; SEQ ID NO:387; SEQ ID NO:389; SEQ ID NO:391; SEQ ID NO:393; SEQ ID NO:395; SEQ ID NO:399; and SEQ ID NO:401.

Item 115 An isolated polynucleotide encoding a polypeptide having an amino acid sequence selected from the group consisting of SEQ ID NO:292; SEQ ID NO:294; SEQ ID NO:296; SEQ ID NO:298; SEQ ID NO:300; SEQ ID NO:302; SEQ ID NO:304; SEQ ID NO:312; SEQ ID NO:314; SEQ ID NO:316; SEQ ID NO:332; SEQ ID NO:334; SEQ ID NO:338; SEQ ID NO:340; SEQ ID NO:342; SEQ ID NO:348; SEQ ID NO:350; SEQ ID NO:352; SEQ ID NO:354; SEQ ID NO:356; SEQ ID NO:358; SEQ ID NO:360; SEQ ID NO:362; SEQ ID NO:364; SEQ ID NO:366; SEQ ID NO:368; SEQ ID NO:370; SEQ ID NO:372; SEQ ID NO:374; SEQ ID NO:376; SEQ ID NO:378; SEQ ID NO:380; SEQ ID NO:384; SEQ ID NO:386; SEQ ID NO:388; SEQ ID NO:390; SEQ ID NO:392; SEQ ID NO:394; SEQ ID NO:396; SEQ ID NO:400; and SEQ ID NO:402.

Item 116 A method of high-throughput screening of transgenic plants for yield-related phenotypes, the method comprising the steps of:
a) forming at least one pool of transgenic plants, each transgenic plant comprising a transgene in an expression cassette;
b) growing the pooled transgenic plants under well watered and water limited growth conditions in a primary screen;
c) selecting transgenic plants that demonstrate an undiminished biomass under water limited growth conditions in the primary screen;
d) determining the molecular identity of each element in the expression cassette in each selected transgenic plant;
e) growing the transgenic plants selected in step c) under well watered and water limited growth conditions in a secondary screen;
f) selecting transgenic plants that demonstrate an undiminished biomass under water limited growth conditions in the secondary screen;
g) growing the transgenic plants selected in step f) under well watered and water limited growth conditions in a tertiary screen; and
h) selecting transgenic plants that demonstrate an undiminished biomass under water limited growth conditions in the tertiary screen;
   wherein:
the well watered growth conditions consist of watering to soil saturation twice a week and determining biomass and health index on days 17 and 21 after sowing; and the water limited growth conditions consist of watering to soil saturation on days 0, 8, and 19 after sowing, and determining biomass and health index on days 20 and 27 after sowing.

Item 117 A transgenic plant transformed with an expression cassette comprising, in operative association,
a) an isolated polynucleotide encoding a promoter capable of enhancing gene expression in leaves;
b) an isolated polynucleotide encoding a mitochondrial transit peptide; and
c) an isolated polynucleotide encoding a full-length farnesyl diphosphate synthase polypeptide;
   wherein the transgenic plant demonstrates increased yield as compared to a wild type plant of the same variety which does not comprise the expression cassette.

Item 118 The transgenic plant of item 117, wherein the farnesyl diphosphate synthase polypeptide comprises a polyprenyl synthetase domain comprising a pair of signature sequences, wherein:
a) one member of the pair is selected from the group consisting of amino acids 81 to 125 of SEQ ID NO:414; amino acids 97 to 139 of SEQ ID NO:416; amino acids 76 to 120 of SEQ ID NO:418; amino acids 116 to 160 of SEQ ID NO:420; amino acids 90 to 132 of SEQ ID NO:422; amino acids 7 to 51 of SEQ ID NO:424; amino acids 46 to 90 of SEQ ID NO:426; amino acids 7 to 49 of SEQ ID NO:428; amino acids 19 to 61 of SEQ ID NO:430; amino acids 7 to 49 of SEQ ID NO:432; and amino acids 98 to 140 of SEQ ID NO:434; and
b) the other member of the pair of signature sequences is selected from the group consisting of amino acids 193 to 227 of SEQ ID NO:414; amino acids 210 to 244 of SEQ ID NO:416; amino acids 191 to 224 of SEQ ID NO:418; amino acids 224 to 257 of SEQ ID NO:420; amino acids 203 to 236 of SEQ ID NO:422; amino acids 115 to 148 of SEQ ID NO:424; amino acids 158 to 191 of SEQ ID NO:426; amino acids 108 to 141 of SEQ ID NO:428; amino acids 132 to 165 of SEQ ID NO:430; amino acids 108 to 141 of SEQ ID NO:432; and amino acids 211 to 244 of SEQ ID NO:434.

Item 119 The transgenic plant of item 117, wherein the farnesyl diphosphate synthase polypeptide has a sequence comprising amino acids 1 to 299 of SEQ ID NO:414; amino acids 1 to 352 of SEQ ID NO:416; amino acids 1 to 294 of SEQ ID NO:418; amino acids 1 to 274 of SEQ ID NO:420; amino acids 1 to 342 of SEQ ID NO:422; amino acids 1 to 222 of SEQ ID NO:424; amino acids 1 to 261 of SEQ ID NO:426; amino acids 1 to 161 of SEQ ID NO:428; amino acids 1 to 174 of SEQ ID NO:430; amino acids 1 to 245 of SEQ ID NO:432; or amino acids 1 to 350 of SEQ ID NO:434.

Item 120 The transgenic plant of item 117, further defined as a species selected from the group consisting of maize, wheat, rye, oat, triticale, rice, barley, soybean, peanut, cotton, rapeseed, canola, manihot, pepper, sunflower, tagetes, potato, tobacco, eggplant, tomato, Vicia species, pea, alfalfa, coffee, cacao, tea, Salix species, oil palm, coconut, perennial grasses, and a forage crop plant.

Item 121 A seed which is true breeding for a transgene comprising, in operative association,
a) an isolated polynucleotide encoding a promoter capable of enhancing gene expression in leaves;
b) an isolated polynucleotide encoding a mitochondrial transit peptide; and
c) an isolated polynucleotide encoding a full-length farnesyl diphosphate synthase polypeptide;
   wherein a transgenic plant grown from said seed demonstrates increased yield as compared to a wild type plant of the same variety which does not comprise the expression cassette.

Item 122 The seed of item 121, wherein he farnesyl diphosphate synthase polypeptide comprises a polyprenyl synthetase domain comprising a pair of signature sequences, wherein:
a) one member of the pair is selected from the group consisting of amino acids 81 to 125 of SEQ ID NO:414; amino acids 97 to 139 of SEQ ID NO:416; amino acids 76 to 120 of SEQ ID NO:418; amino acids 116 to 160 of SEQ ID NO:420; amino acids 90 to 132 of SEQ ID NO:422; amino acids 7 to 51 of SEQ ID NO:424; amino acids 46 to 90 of SEQ ID NO:426; amino acids 7 to 49 of SEQ ID NO:428; amino acids 19 to 61 of SEQ ID NO:430; amino acids 7 to 49 of SEQ ID NO:432; and amino acids 98 to 140 of SEQ ID NO:434; and
b) the other member of the pair of signature sequences is selected from the group consisting of amino acids 193 to 227 of SEQ ID NO:414; amino acids 210 to 244 of SEQ ID NO:416; amino acids 191 to 224 of SEQ ID NO:418; amino acids 224 to 257 of SEQ ID NO:420; amino acids 203 to 236 of SEQ ID NO:422; amino acids 115 to 148 of SEQ ID NO:424; amino acids 158 to 191 of SEQ ID NO:426; amino acids 108 to 141 of SEQ ID NO:428; amino acids 132 to 165 of SEQ ID NO:430; amino acids 108 to 141 of SEQ ID NO:432; and amino acids 211 to 244 of SEQ ID NO:434.

Item 123 The seed of item 121, wherein the farnesyl diphosphate synthase polypeptide has a sequence comprising amino acids 1 to 299 of SEQ ID NO:414; amino acids 1 to 352 of SEQ ID NO:416; amino acids 1 to 294 of SEQ ID NO:418; amino acids 1 to 274 of SEQ ID NO:420; amino acids 1 to 342 of SEQ ID NO:422; amino acids 1 to 222 of SEQ ID NO:424; amino acids 1 to 261 of SEQ ID NO:426; amino acids 1 to 161 of SEQ ID NO:428; amino acids 1 to 174 of SEQ ID NO:430; amino acids 1 to 245 of SEQ ID NO:432; or amino acids 1 to 350 of SEQ ID NO:434.

Item 124 The seed of item 121, further defined as a species selected from the group consisting of maize, wheat, rye, oat, triticale, rice, barley, soybean, peanut, cotton, rapeseed, canola, manihot, pepper, sunflower, tagetes, potato, tobacco, eggplant, tomato, Vicia species, pea, alfalfa, coffee, cacao, tea, Salix species, oil palm, coconut, perennial grasses, and a forage crop plant.

Item 125 A method of increasing yield of a plant, the method comprising the steps of:
a) transforming a plant cell with an expression vector comprising, in operative association,
   i) an isolated polynucleotide encoding a promoter capable of enhancing gene expression in leaves;
   ii) an isolated polynucleotide encoding a mitochondrial transit peptide; and
   iii) an isolated polynucleotide encoding a full-length farnesyl diphosphate synthase polypeptide;
b) regenerating transgenic plants from the transformed plant cell; and
c) selecting drought-tolerant plants from the regenerated transgenic plants.

Item 126 A transgenic plant transformed with an expression cassette comprising, in operative association,
a) an isolated polynucleotide encoding a promoter;
b) an isolated polynucleotide encoding a chloroplast transit peptide; and
c) an isolated polynucleotide encoding a full-length squalene synthase polypeptide;
   wherein the transgenic plant demonstrates increased yield as compared to a wild type plant of the same variety which does not comprise the expression cassette.

Item 127 The transgenic plant of item 126, wherein the squalene synthase polypeptide comprises a squalene synthetase domain comprising a pair of signature sequences, wherein:
a) one member of the pair has a sequence selected from the group consisting of amino acids 201 to 216 of SEQ ID NO:436; amino acids 201 to 216 of SEQ ID NO:438; amino acids 168 to 183 of SEQ ID NO:440; amino acids 168 to 183 of SEQ ID NO:442; and amino acids 164 to 179 of SEQ ID NO:444; and
b) the other member of the pair of signature sequences has a sequence selected from the group consisting of amino acids 234 to 262 of SEQ ID NO:436; amino acids 234 to 262 of SEQ ID NO:438; amino acids 203 to 231 of SEQ ID NO:440; amino acids 201 to 229 of SEQ ID NO:442; and amino acids 197 to 225 of SEQ ID NO:444.

Item 128 The transgenic plant of item 126, wherein the squalene synthase polypeptide comprises a squalene synthetase domain selected from the group consisting of amino acids 95 to 351 of SEQ ID NO:436; amino acids 95 to 351 of SEQ ID NO:438; amino acids 62 to 320 of SEQ ID NO:440; amino acids 62 to 318 of SEQ ID NO:442; and amino acids 58 to 314 of SEQ ID NO:444.

Item 129 The transgenic plant of item 126, wherein the squalene synthase polypeptide comprises amino acids 1 to 436 of SEQ ID NO:436; amino acids 1 to 436 of SEQ ID NO:438; amino acids 1 to 357 of SEQ ID NO:440; amino acids 1 to 413 of SEQ ID NO:442; or amino acids 1 to 401 of SEQ ID NO:444.

Item 130 The transgenic plant of item 126, further defined as a species selected from the group consisting of maize, wheat, rye, oat, triticale, rice, barley, soybean, peanut, cotton, rapeseed, canola, manihot, pepper, sunflower, tagetes, potato, tobacco, eggplant, tomato, Vicia species, pea, alfalfa, coffee, cacao, tea, Salix species, oil palm, coconut, perennial grasses, and a forage crop plant.

Item 131 A seed which is true breeding for a transgene comprising, in operative association,
a) an isolated polynucleotide encoding a promoter;
b) an isolated polynucleotide encoding a chloroplast transit peptide; and
c) an isolated polynucleotide encoding a full-length squalene synthase polypeptide;
   wherein a transgenic plant grown from said seed demonstrates increased yield as compared to a wild type plant of the same variety which does not comprise the expression cassette.

Item 132 The seed of item 131, wherein the squalene synthase polypeptide comprises a squalene synthetase domain comprising a pair of signature sequences, wherein:
a) one member of the pair has a sequence selected from the group consisting of amino acids 201 to 216 of SEQ ID NO:436; amino acids 201 to 216 of SEQ ID NO:438; amino acids 168 to 183 of SEQ ID NO:440; amino acids 168 to 183 of SEQ ID NO:442; and amino acids 164 to 179 of SEQ ID NO:444; and
b) the other member of the pair of signature sequences has a sequence selected from the group consisting of amino acids 234 to 262 of SEQ ID NO:436; amino acids 234 to 262 of SEQ ID NO:438; amino acids 203 to 231 of SEQ ID NO:440; amino acids 201 to 229 of SEQ ID NO:442; and amino acids 197 to 225 of SEQ ID NO:444.

Item 133 The seed of item 131, wherein the squalene synthase polypeptide comprises a squalene synthetase domain selected from the group consisting of amino acids 95 to 351 of SEQ ID NO:436; amino acids 95 to 351 of SEQ ID NO:438; amino acids 62 to 320 of SEQ ID NO:440; amino acids 62 to 318 of SEQ ID NO:442; and amino acids 58 to 314 of SEQ ID NO:444.

Item 134 The seed of item 131, wherein the squalene synthase polypeptide comprises amino acids 1 to 436 of SEQ ID NO:436; amino acids 1 to 436 of SEQ ID NO:438; amino acids 1 to 357 of SEQ ID NO:440; amino acids 1 to 413 of SEQ ID NO:442; or amino acids 1 to 401 of SEQ ID NO:444.

Item 135 The seed of item 131, further defined as a species selected from the group consisting of maize, wheat, rye, oat, triticale, rice, barley, soybean, peanut, cotton, rapeseed, canola, manihot, pepper, sunflower, tagetes, potato, tobacco, eggplant, tomato, Vicia species, pea, alfalfa, coffee, cacao, tea, Salix species, oil palm, coconut, perennial grasses, and a forage crop plant.

Item 136 A method of producing a transgenic plant having enhanced yield as compared to a wild type plant of the same variety, the method comprising the steps of:
a) transforming a plant cell with an expression vector comprising, in operative association,
   i) an isolated polynucleotide encoding a promoter;
   ii) an isolated polynucleotide encoding a chloroplast transit peptide; and
   iii) an isolated polynucleotide encoding a full-length squalene synthase polypeptide;
b) regenerating transgenic plants from the transformed plant cell; and
c) selecting higher-yielding plants from the regenerated transgenic plants.

Item 137 A transgenic plant transformed with an expression cassette comprising, in operative association,
a) an isolated polynucleotide encoding a promoter;
b) an isolated polynucleotide encoding a chloroplast transit peptide; and
c) an isolated polynucleotide encoding a full-length squalene epoxidase polypeptide;
   wherein the transgenic plant demonstrates increased yield as compared to a wild type plant of the same variety which does not comprise the expression cassette.

Item 138 The transgenic plant of item 137, wherein the squalene epoxidase polypeptide comprises a domain comprising a pair of FAD-dependent enzyme motifs, wherein:
a) one member of the pair has a sequence selected from the group consisting of amino acids 55 to 66 of SEQ ID NO:446; amino acids 79 to 90 of SEQ ID NO:448; and amino acids 98 to 109 of SEQ ID NO:450; and
b) the other member of the pair has a sequence selected from the group consisting of amino acids 334 to 350 of SEQ ID NO:446; amino acids 331 to 347 of SEQ ID NO:448; and amino acids 347 to 363 of SEQ ID NO:450.

Item 139 The transgenic plant of item 137, wherein the squalene epoxidase polypeptide comprises a domain selected from the group consisting of amino acids 20 to 488 of SEQ ID NO:446; amino acids 44 to 483 of SEQ ID NO:448; and amino acids 63 to 500 of SEQ ID NO:450.

Item 140 The transgenic plant of item 137, wherein the squalene epoxidase polypeptide amino acids 1 to 496 of SEQ ID NO:446; amino acids 1 to 512 of SEQ ID NO:448; or amino acids 1 to 529 of SEQ ID NO:450.

Item 141 The transgenic plant of item 137, further defined as a species selected from the group consisting of maize, wheat, rye, oat, triticale, rice, barley, soybean, peanut, cotton, rapeseed, canola, manihot, pepper, sunflower, tagetes, potato, tobacco, eggplant, tomato, Vicia species, pea, alfalfa, coffee, cacao, tea, Salix species, oil palm, coconut, perennial grasses, and a forage crop plant.

Item 142 A seed which is true breeding for a transgene comprising, in operative association,
a) an isolated polynucleotide encoding a promoter;
b) an isolated polynucleotide encoding a chloroplast transit peptide; and
c) an isolated polynucleotide encoding a full-length squalene epoxidase polypeptide;
   wherein a transgenic plant grown from said seed demonstrates increased yield as compared to a wild type plant of the same variety which does not comprise the expression cassette.

Item 143 The seed of item 142, wherein the squalene epoxidase polypeptide comprises a domain comprising a pair of FAD-dependent enzyme motifs, wherein:
a) one member of the pair has a sequence selected from the group consisting of amino acids 55 to 66 of SEQ ID NO:446; amino acids 79 to 90 of SEQ ID NO:448; and amino acids 98 to 109 of SEQ ID NO:450; and
b) the other member of the pair has a sequence selected from the group consisting of amino acids 334 to 350 of SEQ ID NO:446; amino acids 331 to 347 of SEQ ID NO:448; and amino acids 347 to 363 of SEQ ID NO:450.

Item 144 The seed of item 142, wherein the squalene epoxidase polypeptide comprises a domain selected from the group consisting of amino acids 20 to 488 of SEQ ID NO:446; amino acids 44 to 483 of SEQ ID NO:448; and amino acids 63 to 500 of SEQ ID NO:450.

Item 145 The seed of item 142, wherein the squalene epoxidase polypeptide amino acids 1 to 496 of SEQ ID NO:446; amino acids 1 to 512 of SEQ ID NO:448; or amino acids 1 to 529 of SEQ ID NO:450.

Item 146 The seed of item 142, further defined as a species selected from the group consisting of maize, wheat, rye, oat, triticale, rice, barley, soybean, peanut, cotton, rapeseed, canola, manihot, pepper, sunflower, tagetes, potato, tobacco, eggplant, tomato, Vicia species, pea, alfalfa, coffee, cacao, tea, Salix species, oil palm, coconut, perennial grasses, and a forage crop plant.

Item 147 A method of producing a transgenic plant having enhanced yield as compared to a wild type plant of the same variety, the method comprising the steps of:

a) transforming a plant cell with an expression vector comprising, in operative association,
i) an isolated polynucleotide encoding a promoter;
ii) an isolated polynucleotide encoding a chloroplast transit peptide; and
iii) an isolated polynucleotide encoding a full-length squalene epoxidase polypeptide;
   b) regenerating transgenic plants from the transformed plant cell; and
   c) selecting higher-yielding plants from the regenerated transgenic plants.

Item 148 An isolated polynucleotide having a sequence selected from the group consisting of SEQ ID NO:417; SEQ ID NO:419; SEQ ID NO:421; SEQ ID NO:423; SEQ ID NO:425; SEQ ID NO:427; SEQ ID NO:429; SEQ ID NO:431; SEQ ID NO:435; SEQ ID NO:437; SEQ ID NO:439; SEQ ID NO:447; and SEQ ID NO:449.

Item 149 An isolated polynucleotide encoding a polypeptide having an amino acid sequence selected from the group consisting of SEQ ID NO:418; SEQ ID NO:420; SEQ ID NO:422; SEQ ID NO:424; SEQ ID NO:426; SEQ ID NO:428; SEQ ID NO:430; SEQ ID NO:432; SEQ ID NO:436; SEQ ID NO:438; SEQ ID NO:440; SEQ ID NO:448; and SEQ ID NO:450.

The invention is further illustrated by the following examples, which are not to be construed in any way as imposing limitations upon the scope thereof.

### Example 1

### Characterization of cDNAs

cDNAs were isolated from proprietary libraries of the respective plant species using known methods. Sequences were processed and annotated using bioinformatics analyses. The degrees of amino acid identity and similarity of the isolated sequences to the respective closest known public sequences are indicated in Tables 2A through 11A, Tables 2B through 19B, Tables 2C through 16C, Tables 2D through 24D and Tables 2E through 4E (Pairwise Comparison was used: gap penalty: 10; gap extension penalty: 0.1; score matrix: blosum62).

TABLE 2A
Comparison of GM47143343 (SEQ ID NO: 2) to known
mitogen activated protein kinases

| Public Database | | |
|---|---|---|
| Accession # | Species | Sequence Identity (%) |
| AAD32204 | Prunus armeniaca | 88.60% |
| NP_179409 | A. thaliana | 85.90% |
| BAA04870 | A. thaliana | 85.60% |
| CAN70944 | Vitis vinifera | 82.90% |
| ABO84371 | M. truncatula | 82.90% |

TABLE 3A
Comparison of EST431 (SEQ ID NO: 4) to known mitogen
activated protein kinases

| Public Database Accession # | Species | Sequence Identity (%) |
|---|---|---|
| CAN75543 | V. vinifera | 78.20% |
| NP_001065156 | O. sativa | 77.80% |
| AAR11450 | Z. mays | 77.10% |
| ABB69023 | B. napus | 76.60% |
| AAN65180 | Petroselinum | 76.40% |
| | crispum | |

TABLE 4A
Comparison of EST253 (SEQ ID NO: 6) to known
mitogen activated protein kinase

| Public Database | | |
|---|---|---|
| Accession # | Species | Sequence Identity (%) |
| CAH05024 | Papaver rhoeas | 67.40% |
| Q40517 | Nicotiana tabacum | 67.00% |
| CAN70091 | V. vinifera | 66.80% |
| ABA00652 | Gossypium hirsutum | 66.50% |
| AAF73257 | Pisum sativum | 66.20% |

TABLE 5A
Comparison of EST272 (SEQ ID NO: 30) to known
mitogen activated protein kinase

| Public Database Accession # | Species | Sequence Identity (%) |
|---|---|---|
| NP_001065156 | O. sativa | 69.90% |
| BAB93532 | S. tuberosum | 68.80% |
| Q40353 | M. sativa | 67.70% |
| BAB93531 | S. tuberosum | 66.70% |
| Q06060 | Pisum sativum | 65.80% |

TABLE 6A
Comparison of GM50305602 (SEQ ID NO: 40) to known
calcium dependent protein kinases

| Public Database Accession # | Species | Sequence Identity (%) |
|---|---|---|
| NP_564066 | A. thaliana | 60.90% |
| AAO42812 | A. thaliana | 60.70% |
| BAE98496 | A. thaliana | 59.70% |
| NP_177612 | A. thaliana | 58.00% |
| AAA99794 | A. thaliana | 56.80% |

TABLE 7A
Comparison of EST500 (SEQ ID NO: 42) to known
calcium dependent protein kinases

| Public Database | | |
|---|---|---|
| Accession # | Species | Sequence Identity (%) |
| AAB70706 | Tortula ruralis | 90.00% |
| BAA13232 | Z. mays | 64.80% |
| CAN78387 | V. vinifera | 64.70% |
| AAL68972 | Cucurbita maxima | 64.60% |
| EAY87105 | O. sativa | 64.40% |

TABLE 8A
Comparison of EST401 (SEQ ID NO: 44) to known
calcium dependent protein kinases

| Public Database | | Sequence |
|---|---|---|
| Accession # | Species | Identity (%) |
| AAL30819 | N. tabacum | 64.80% |
| CAN69589 | V. vinifera | 64.30% |
| NP_179379 | A. thaliana | 64.00% |
| AAX81331 | N. tabacum | 64.00% |
| AAX14494 | M. truncatula | 63.70% |

TABLE 9A
Comparison of EST591 (SEQ ID NO: 62) to known
calcium-dependent protein kinases

| Public Database Accession # | Species | Sequence Identity (%) |
|---|---|---|
| NP_001044575 | O. sativa | 61.90% |
| CAN62888 | V. vinifera | 60.90% |
| BAA13440 | Ipomoea batatas | 59.30% |
| CAA65500 | Medicago sativa | 57.50% |
| ABD98803 | T. aestivum | 57.30% |

TABLE 10A
Comparison of BN42110642 (SEQ ID NO: 74) to known
cyclin dependent protein kinases

| Public Database Accession # | Species | Sequence Identity (%) |
|---|---|---|
| NP_190576 | A. thaliana | 74.70% |
| NP_201527 | A. thaliana | 61.30% |
| CAN59802 | V. vinifera | 50.90% |
| BAE80325 | Camellia sinensis | 50.30% |
| AAO72990 | Populus alba | 49.70% |

TABLE 11A
Comparison of EST336 (SEQ ID NO: 82) to known
serine/threonine-specific protein kinases

| Public Database | | |
|---|---|---|
| Accession # | Species | Sequence Identity (%) |
| CAA19877 | A. thaliana | 79.70% |
| NP_567945 | A. thaliana | 79.30% |
| CAN62745 | V. vinifera | 79.10% |
| EAZ21035 | O. sativa | 76.80% |
| ABA40436 | Solanum tuberosum | 76.00% |

The full-length DNA sequence of the GM47143343 (SEQ ID NO: 2), EST431 (SEQ ID NO:4), EST253 (SEQ ID NO:6), and EST272 (SEQ ID NO:30) were blasted against proprietary databases of canola, soybean, rice, maize, linseed, sunflower, and wheat cDNAs at an e value of e-10 (Altschul et al., 1997, Nucleic Acids Res. 25: 3389-3402). All the contig hits were analyzed for the putative full length sequences, and the longest clones representing the putative full length contigs were fully sequenced. One homolog from wheat, one homolog from corn, four homologs from soybean, four homologs from linseed, four homologs from canola, and one homolog from sunflower were identified. The degree of amino acid identity of these sequences to the closest known public sequences is indicated in Tables 12A through 26A (Pairwise Comparison was used: gap penalty: 10; gap extension penalty: 0.1; score matrix: blosum62).

TABLE 12A
Comparison of TA54298452 (SEQ ID NO: 8) to known
mitogen activated protein kinases

| Public Database Accession # | Species | Sequence Identity (%) |
|---|---|---|
| CAJ85945 | Festuca | 95.10% |
| | arundinacea | |
| CAG23921 | F. arundinacea | 94.60% |
| CAD54741 | O. sativa | 94.00% |
| ABH01191 | O. sativa | 93.80% |
| CAB61889 | O. sativa | 93.50% |

TABLE 13A
Comparison of GM59742369 (SEQ ID NO: 10) to known
mitogen activated protein kinases

| Public Database Accession # | Species | Sequence Identity (%) |
|---|---|---|
| AAF73257 | P. sativum | 93.80% |
| ABA00652 | G. hirsutum | 88.20% |
| Q40517 | N. tabacum | 87.90% |
| CAN70091 | V. vinifera | 87.90% |
| CAH05024 | Papaver rhoeas | 85.50% |

TABLE 14A
Comparison of LU61585372 (SEQ ID NO: 12) to known
mitogen activated protein kinases

| Public Database Accession # | Species | Sequence Identity (%) |
|---|---|---|
| CAN70091 | V. vinifera | 87.50% |
| ABA00652 | G. hirsutum | 87.20% |
| Q40517 | N. tabacum | 86.70% |
| CAH05024 | P. rhoeas | 84.60% |
| AAF73257 | P. sativum | 84.50% |

TABLE 15A
Comparison of BN44703759 (SEQ ID NO: 14) to known
mitogen activated protein kinases

| Public Database | | |
|---|---|---|
| Accession # | Species | Sequence Identity (%) |
| NP_565989 | A. thaliana | 80.10% |
| ABG54347 | synthetic construct | 77.50% |
| ABF69963 | Musa acuminata | 67.70% |
| NP_001043642 | O. sativa | 66.60% |
| NP_001056342 | O. sativa | 64.30% |

TABLE 16A
Comparison of GM59703946 (SEQ ID NO: 16) to known
mitogen activated protein kinases

| Public Database | | |
|---|---|---|
| Accession # | Species | Sequence Identity (%) |
| CAO71082 | V. vinifera | 88.10% |
| AAL32607 | A. thaliana | 80.70% |
| NP_197402 | A. thaliana | 80.70% |
| NP_197402 | A. thaliana | 80.70% |
| ABG54343 | synthetic | 77.80% |
| | construct | |

TABLE 17A
Comparison of GM59589775 (SEQ ID NO: 18) to known
mitogen activated protein kinases

| Public Database | | |
|---|---|---|
| Accession # | Species | Sequence Identity (%) |
| Q40353 | Medicago sativa | 91.20% |
| CAN75543 | V. vinifera | 88.00% |
| AAN65180 | Petroselinum | 87.70% |
| | crispum | |
| BAE46985 | N. tabacum | 84.80% |
| BAA04867 | A. thaliana | 83.60% |

TABLE 18A
Comparison of LU61696985 (SEQ ID NO: 20) to known
mitogen activated protein kinases

| Public Database | | |
|---|---|---|
| Accession # | Species | Sequence Identity (%) |
| AAZ57337 | Cucumis sativus | 86.20% |
| ABM67698 | C. sinensis | 85.70% |
| AAV34677 | B. napus | 83.60% |
| ABJ89813 | Nicotiana attenuata | 83.30% |
| BAE44363 | S. tuberosum | 83.30% |

TABLE 19A
Comparison of ZM62001130 (SEQ ID NO: 22) to known
mitogen activated protein kinases

| Public Database | | |
|---|---|---|
| Accession # | Species | Sequence Identity (%) |
| BAA74733 | Z. mays | 91.20% |
| AAW65993 | Saccharum | 87.40% |
| | officinarum | |
| AAK01710 | O. sativa | 83.70% |
| CAA56314 | A. sativa | 83.70% |
| ABH01189 | O. sativa | 83.40% |

TABLE 20A
Comparison of HA66796355 (SEQ ID NO: 24) to known
mitogen activated protein kinases

| Public Database | | |
|---|---|---|
| Accession # | Species | Sequence Identity (%) |
| ABB16418 | N. tabacum | 92.40% |
| Q40532 | N. tabacum | 92.10% |
| ABB16417 | N. tabacum | 90.90% |
| AAQ14867 | G. max | 90.70% |
| AAP20420 | L. esculentum | 90.20% |

TABLE 21A
Comparison of LU61684898 (SEQ ID NO: 26) to known
mitogen activated protein kinases

| Public Database | | |
|---|---|---|
| Accession # | Species | Sequence Identity (%) |
| AAQ14867 | G. max | 87.70% |
| ABB16418 | N. tabacum | 86.80% |
| Q06060 | P. sativum | 86.70% |
| Q40532 | N. tabacum | 86.30% |
| ABE83899 | M. truncatula | 86.30% |

TABLE 22A
Comparison of LU61597381 (SEQ ID NO: 28) to known
mitogen activated protein kinases

| Public Database | | |
|---|---|---|
| Accession # | Species | Sequence Identity (%) |
| AAN65180 | P. crispum | 82.40% |
| CAN75543 | V. vinifera | 80.10% |
| BAE46985 | N. tabacum | 78.80% |
| Q40353 | M. sativa | 78.50% |
| NP_001065156 | O. sativa | 78.50% |

TABLE 23A
Comparison of BN42920374 (SEQ ID NO: 32) to known
mitogen activated protein kinases

| Public Database | | |
|---|---|---|
| Accession # | Species | Sequence Identity (%) |
| NP_179409 | A. thaliana | 96.50% |
| BAA04870 | A. thaliana | 95.40% |
| ABG54334 | synthetic | 91.50% |
| AAD32204 | P. armeniaca | 85.90% |
| Q40517 | N. tabacum | 81.50% |

TABLE 24A
Comparison of BN45700248 (SEQ ID NO: 34) to known
mitogen activated protein kinases

| Public Database | | |
|---|---|---|
| Accession # | Species | Sequence Identity (%) |
| NP_182131 | A. thaliana | 96.20% |
| ABG54339 | synthetic | 91.10% |
| AAC62906 | A. thaliana | 88.20% |
| AAN65180 | P. crispum | 79.70% |
| CAN75543 | Vitis vinifera | 79.20% |

TABLE 25A
Comparison of BN47678601 (SEQ ID NO: 36) to known
mitogen activated protein kinases

| Public Database | | |
|---|---|---|
| Accession # | Species | Sequence Identity (%) |
| ABB69023 | B. napus | 98.70% |
| BAA04867 | A. thaliana | 93.60% |
| ABG54331 | synthetic | 88.90% |
| NP_192046 | A. thaliana | 88.30% |
| ABG54338 | synthetic | 82.50% |

TABLE 26A
Comparison of GMsj02a06 (SEQ ID NO: 38) to known
mitogen activated protein kinases

| Public Database | | |
|---|---|---|
| Accession # | Species | Sequence Identity (%) |
| AAQ14867 | G. max | 91.60% |
| Q07176 | M. sativa | 88.20% |
| ABE83899 | M. truncatula | 88.20% |
| Q06060 | P. sativum | 87.10% |
| AAP20420 | L. esculentum | 84.30% |

The full-length DNA sequences of the GM50305602 (SEQ ID NO: 40), EST500 (SEQ ID NO:42), and EST401 (SEQ ID NO:44) were blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Eight homologs from canola, two homologs from soybean, two homologs from corn, and one homolog from wheat were identified. The degree of amino acid identity of these sequences to the closest known public sequences is indicated in Tables 27A through 39A (Pairwise Comparison was used: gap penalty: 10; gap extension penalty: 0.1; score matrix: blosum62).

TABLE 27A
Comparison of BN51391539 (SEQ ID NO: 46) to known
calcium dependent protein kinases

| Public Database | | |
|---|---|---|
| Accession # | Species | Sequence Identity (%) |
| AAL38596 | A. thaliana | 91.00% |
| CAN61364 | V. vinifera | 72.90% |
| ABE79749 | M. truncatula | 72.70% |
| EAZ12734 | O. sativa | 72.30% |
| CAF18446 | T. aestivum | 70.90% |

TABLE 28A
Comparison of GM59762784 (SEQ ID NO: 48) to known
calcium dependent protein kinases

| Public Database | | |
|---|---|---|
| Accession # | Species | Sequence Identity (%) |
| CAA65500 | M. sativa | 79.10% |
| ABE72958 | M. truncatula | 78.80% |
| AAB80693 | G. max | 77.70% |
| AAP03014 | G. max | 77.10% |
| AAD28192 | S. tuberosum | 76.50% |

TABLE 29A
Comparison of BN44099508 (SEQ ID NO: 50) to known
calcium dependent protein kinases

| Public Database | | |
|---|---|---|
| Accession # | Species | Sequence Identity (%) |
| NP_181647 | A. thaliana | 93.80% |
| NP_191235 | A. thaliana | 90.50% |
| ABD33022 | M. truncatula | 78.90% |
| BAC16472 | O. sativa | 74.40% |
| NP_001050179 | O. sativa | 70.80% |

TABLE 30A
Comparison of BN45789913 (SEQ ID NO: 52) to known
calcium dependent protein kinases

| Public Database Accession # | Species | Sequence Identity (%) |
|---|---|---|
| NP_197831 | A. thaliana | 91.90% |
| NP_190506 | A. thaliana | 85.50% |
| AAD28759 | A. thaliana | 70.70% |
| AAM91611 | A. thaliana | 70.50% |
| AAL30818 | N. tabacum | 68.00% |

TABLE 31A
Comparison of BN47959187 (SEQ ID NO: 54) to known calcium
dependent protein kinases

| Public Database Accession # | Species | Sequence Identity (%) |
|---|---|---|
| NP_179379 | A. thaliana | 89.80% |
| NP_195331 | A. thaliana | 74.90% |
| AAX14494 | M. truncatula | 74.50% |
| AAL30819 | N. tabacum | 73.90% |
| CAA18501 | A. thaliana | 73.60% |

TABLE 32A
Comparison of BN51418316 (SEQ ID NO: 56) to known
calcium dependent protein kinases

| Public Database Accession # | Species | Sequence Identity (%) |
|---|---|---|
| NP_564066 | A. thaliana | 90.30% |
| AAO42812 | A. thaliana | 90.10% |
| BAA04829 | A. thaliana | 84.50% |
| NP_177612 | A. thaliana | 81.40% |
| EAZ04388 | O. sativa | 65.80% |

TABLE 33A
Comparison of GM59691587 (SEQ ID NO: 58) to known calcium
dependent protein kinases

| Public Database | | |
|---|---|---|
| Accession # | Species | Sequence Identity (%) |
| AAC49405 | Vigna radiata | 87.10% |
| AAL68972 | Cucurbita maxima | 86.60% |
| BAF57913 | S. tuberosum | 85.60% |
| BAF57914 | S. tuberosum | 85.50% |
| CAN78387 | V. vinifera | 85.20% |

TABLE 34A
Comparison of ZM62219224 (SEQ ID NO: 60) to known
calcium dependent protein kinases

| Public Database Accession # | Species | Sequence Identity (%) |
|---|---|---|
| CAA57156 | O. sativa | 86.60% |
| BAC19839 | O. sativa | 86.40% |
| AAC05270 | O. sativa | 85.70% |
| EAY88372 | O. sativa | 85.10% |
| AAN17388 | O. sativa | 82.80% |

TABLE 35A
Comparison of BN51345938 (SEQ ID NO: 64) to known
calcium dependent protein kinases

| Public Database Accession # | Species | Sequence Identity (%) |
|---|---|---|
| AAZ32753 | B. napus | 97.10% |
| AAZ32752 | B. rapa | 96.90% |
| NP_565411 | A. thaliana | 86.00% |
| AAZ32751 | B. oleracea | 85.90% |
| NP_195257 | A. thaliana | 82.00% |

TABLE 36A
Comparison of BN51456960 (SEQ ID NO: 66) to known
calcium dependent protein kinases

| Public Database Accession # | Species | Sequence Identity (%) |
|---|---|---|
| NP_568281 | A. thaliana | 94.20% |
| NP_197446 | A. thaliana | 89.70% |
| BAE99123 | A. thaliana | 82.70% |
| CAG27839 | Nicotiana | 80.80% |
| | plumbaginifolia | |
| AAP72282 | Cicer arietinum | 77.60% |

TABLE 37A
Comparison of BN43562070 (SEQ ID NO: 68) to known
calcium dependent protein kinases

| Public Database Accession # | Species | Sequence Identity (%) |
|---|---|---|
| NP_196779 | A. thaliana | 95.70% |
| AAL59948 | A. thaliana | 95.50% |
| NP_197437 | A. thaliana | 93.00% |
| ABE77685 | M. truncatula | 81.10% |
| CAN62888 | V. vinifera | 79.10% |

TABLE 38A
Comparison of TA60004809 (SEQ ID NO: 70) to known
calcium dependent protein kinases

| Public Database Accession # | Species | Sequence Identity (%) |
|---|---|---|
| ABK63287 | T. aestivum | 96.50% |
| CAA57156 | O. sativa | 82.70% |
| EAY88372 | O. sativa | 81.20% |
| AAN17388 | O. sativa | 78.50% |
| NP_001048842 | O. sativa | 61.10% |

TABLE 39A
Comparison of ZM62079719 (SEQ ID NO: 72) to known
calcium dependent protein kinases

| Public Database Accession # | Species | Sequence Identity (%) |
|---|---|---|
| BAA12715 | Z. mays | 97.20% |
| NP_001059775 | O. sativa | 92.30% |
| CAA57157 | O. sativa | 92.30% |
| ABC59619 | T. aestivum | 90.10% |
| ABD98803 | T. aestivum | 89.90% |

The full-length DNA sequence of the BN42110642 (SEQ ID NO:74) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Two homologs from soybean and one homolog from corn were identified. The degree of amino acid identity of these sequences to the closest known public sequences is indicated in Tables 40A through 42A (Pairwise Comparison was used: gap penalty: 10; gap extension penalty: 0.1; score matrix: blosum62).

TABLE 40A
Comparison of GM59794180 (SEQ ID NO: 76) to known
cyclin dependent protein kinases

| Public Database Accession # | Species | Sequence Identity (%) |
|---|---|---|
| ABP03744 | M. truncatula | 73.90% |
| NP_177178 | A. thaliana | 60.90% |
| CAA58285 | A. thaliana | 60.30% |
| S51650 | A. thaliana | 58.10% |
| AAL47479 | Helianthus | 56.30% |
| | tuberosus | |

TABLE 41A
Comparison of GMsp52b07 (SEQ ID NO: 78) to known
cyclin dependent protein kinases

| Public Database Accession # | Species | Sequence Identity (%) |
|---|---|---|
| AAS13371 | G. max | 90.80% |
| CAB40540 | M. sativa | 72.80% |
| CAA61334 | M. sativa | 72.20% |
| BAA33153 | P. sativum | 70.80% |
| BAE93057 | N. tabacum | 58.70% |

TABLE 42A
Comparison of ZM57272608 (SEQ ID NO: 80) to known
cyclin dependent protein kinases

| Public Database Accession # | Species | Sequence Identity (%) |
|---|---|---|
| EAZ04741 | O. sativa | 64.60% |
| NP_001060304 | O. sativa | 64.60% |
| AAV28532 | S. officinarum | 47.40% |
| AAV28533 | S. officinarum | 47.00% |
| ABB36799 | Z. mays | 46.70% |

The full-length DNA sequence of the EST336 (SEQ ID NO: 82) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Two homologs from canola, two homologs from maize, two homologs from linseed, and three homologs from soybean were identified. The degree of amino acid identity of these sequences to the closest known public sequences is indicated in Tables 43A through 51A (Pairwise Comparison was used: gap penalty: 10; gap extension penalty: 0.1; score matrix: blosum62).

TABLE 43A
Comparison of BN43012559 (SEQ ID NO: 84) to known
serine/threonine-specific protein kinases

| Public Database Accession # | Species | Sequence Identity (%) |
|---|---|---|
| NP_196476 | A. thaliana | 90.20% |
| CAA78106 | A. thaliana | 89.60% |
| AAM65503 | A. thaliana | 88.00% |
| NP_201170 | A. thaliana | 87.50% |
| BAE99712 | A. thaliana | 87.30% |

TABLE 44A
Comparison of BN44705066 (SEQ ID NO: 86) to known
serine/threonine-specific protein kinases

| Public Database Accession # | Species | Sequence Identity (%) |
|---|---|---|
| AAA33004 | B. napus | 94.70% |
| AAA33003 | B. napus | 94.70% |
| NP_172563 | A. thaliana | 92.80% |
| AAM67112 | A. thaliana | 90.30% |
| NP_176290 | A. thaliana | 71.90% |

TABLE 45A
Comparison of GM50962576 (SEQ ID NO: 88) to known
serine/threonine-specific protein kinases

| Public Database Accession # | Species | Sequence Identity (%) |
|---|---|---|
| CAN62745 | V. vinifera | 89.80% |
| NP_567945 | A. thaliana | 89.30% |
| CAA19877 | A. thaliana | 87.90% |
| EAY83693 | O. sativa | 81.30% |
| NP_001050653 | O. sativa | 58.30% |

TABLE 46A
Comparison of GMsk93h09 (SEQ ID NO: 90) to known
serine/threonine-specific protein kinases

| Public Database Accession # | Species | Sequence Identity (%) |
|---|---|---|
| CAN62745 | V. vinifera | 83.70% |
| ABA40436 | S. tuberosum | 82.50% |
| AAF27340 | Vicia faba | 82.50% |
| NP_201489 | A. thaliana | 81.50% |
| NP_001050653 | O. sativa | 55.40% |

TABLE 47A
Comparison of GMso31a02 (SEQ ID NO: 92) to known
serine/threonine-specific protein kinases

| Public Database Accession # | Species | Sequence Identity (%) |
|---|---|---|
| Q75V63 | O. sativa | 78.90% |
| NP_001065412 | O. sativa | 78.90% |
| AAA34017 | G. max | 78.50% |
| AAA33979 | G. max | 77.00% |
| CAN62023 | V. vinifera | 75.10% |

TABLE 48A
Comparison of LU61649369 (SEQ ID NO: 94) to known
serine/threonine-specific protein kinases

| Public Database Accession # | Species | Sequence Identity (%) |
|---|---|---|
| NP_201489 | A. thaliana | 83.10% |
| CAN62745 | V. vinifera | 82.10% |
| NP_567945 | A. thaliana | 81.60% |
| CAA19877 | A. thaliana | 80.70% |
| NP_001050653 | O. sativa | 55.70% |

TABLE 49A
Comparison of LU61704197 (SEQ ID NO: 96) to known
serine/threonine-specific protein kinases

| Public Database Accession # | Species | Sequence Identity (%) |
|---|---|---|
| CAN78793 | V. vinifera | 84.90% |
| ABG81507 | C. sinensis | 83.90% |
| AAL89456 | N. tabacum | 83.20% |
| CAE54588 | Fagus sylvatica | 83.00% |
| AAV41842 | M. truncatula | 82.20% |

TABLE 50A
Comparison of ZM57508275 (SEQ ID NO: 98) to known
serine/threonine-specific protein kinases

| Public Database Accession # | Species | Sequence Identity (%) |
|---|---|---|
| EAY91961 | O. sativa | 94.60% |
| CAN62745 | Vitis vinifera | 82.60% |
| CAA19877 | A. thaliana | 81.20% |
| NP_001051371 | O. sativa | 74.70% |
| NP_001050653 | O. sativa | 58.60% |

TABLE 51A
Comparison of ZM59288476 (SEQ ID NO: 100) to known
serine/threonine-specific protein kinases

| Public Database Accession # | Species | Sequence Identity (%) |
|---|---|---|
| ABD72268 | O. sativa | 91.20% |
| NP_001052827 | O. sativa | 74.90% |
| AAU43772 | Z. mays | 73.50% |
| NP_001044930 | O. sativa | 64.40% |
| NP_001047099 | O. sativa | 54.10% |

TABLE 2B
Comparison of BN42194524 (SEQ ID NO: 102) to known phospholipid
hydroperoxide glutathione peroxidases

| Public Database Accession # | Species | Sequence Identity (%) |
|---|---|---|
| AAP59427 | Lycopersicon | 76.90% |
| | esculentum | |
| CAN60579 | V. vinifera | 76.90% |
| AAL40914 | Momordica | 76.30% |
| | charantia | |
| CAD31839 | Cicer arietinum | 71.60% |
| NP_001053524 | O. sativa | 71.20% |

The full-length DNA sequence of the BN42194524 (SEQ ID NO: 102) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Four homologs from corn, three homologs from canola, seven homologs from soybean, one homolog from linseed, and two homologs from rice were identified. The degree of amino acid identity of these sequences to the closest known public sequences is indicated in Tables 19B and 20B (Pairwise Comparison was used: gap penalty: 10; gap extension penalty: 0.1; score matrix: blosum62).

TABLE 3B
Comparison of ZM68498581 (SEQ ID NO: 104) to known phospholipid
hydroperoxide glutathione peroxidases

| Public Database Accession # | Species | Sequence Identity (%) |
|---|---|---|
| AAT42154 | Z. mays | 93.70% |
| AAT42166 | Sorghum bicolor | 92.60% |
| AAS47590 | S. italica | 91.40% |
| AAM88847 | Z. mays | 88.60% |
| NP_001053524 | O. sativa | 88.00% |

TABLE 4B
Comparison of BN42062606 (SEQ ID NO: 106) to known phospholipid
hydroperoxide glutathione peroxidases

| Public Database Accession # | Species | Sequence Identity (%) |
|---|---|---|
| NP_180080 | A. thaliana | 87.00% |
| S71250 | A. thaliana | 84.90% |
| NP_194915 | A. thaliana | 80.10% |
| Q9SZ54 | A. thaliana | 77.50% |
| AAM12502 | B. napus | 73.20% |

TABLE 5B
Comparison of BN42261838 (SEQ ID NO: 108) to known phospholipid
hydroperoxide glutathione peroxidases

| Public Database | | |
|---|---|---|
| Accession # | Species | Sequence Identity (%) |
| BAA24226 | A. thaliana | 94.70% |
| AAQ03092 | Malus × domestica | 88.80% |
| AAT42166 | S. bicolor | 87.00% |
| AAT42154 | Z. mays | 87.00% |
| AAS47590 | Setaria italica | 86.40% |

TABLE 6B
Comparison of BN43722096 (SEQ ID NO: 110) to known phospholipid
hydroperoxide glutathione peroxidases

| Public Database Accession # | Species | Sequence Identity (%) |
|---|---|---|
| NP_191867 | A. thaliana | 83.90% |
| NP_566128 | A. thaliana | 81.20% |
| A84924 | A. thaliana | 77.30% |
| BAC55016 | H. vulgare | 66.50% |
| AAT42166 | S. bicolor | 65.90% |

TABLE 7B
Comparison of GM50585691 (SEQ ID NO: 112) to known phospholipid
hydroperoxide glutathione peroxidases

| Public Database | | |
|---|---|---|
| Accession # | Species | Sequence Identity (%) |
| Q06652 | C. sinensis | 82.00% |
| CAE46896 | C. sinensis | 81.40% |
| AAQ03092 | Malus × domestica | 81.00% |
| BAA24226 | A. thaliana | 79.90% |
| CAJ43709 | Plantago major | 79.80% |

TABLE 8B
Comparison of GMsa56c07 (SEQ ID NO: 114) to known phospholipid
hydroperoxide glutathione peroxidases

| Public Database | | |
|---|---|---|
| Accession # | Species | Sequence Identity (%) |
| Q06652 | Citrus sinensis | 85.00% |
| CAE46896 | C. sinensis | 84.40% |
| AAQ03092 | Malus × domestica | 82.70% |
| NP_001053524 | O. sativa | 82.10% |
| CAJ43709 | P. major | 81.50% |

TABLE 9B
Comparison of GMsb20d04 (SEQ ID NO: 116) to known phospholipid
hydroperoxide glutathione peroxidases

| Public Database | | |
|---|---|---|
| Accession # | Species | Sequence Identity (%) |
| AAQ03092 | Malus × domestica | 87.50% |
| Q06652 | C. sinensis | 87.50% |
| CAE46896 | C. sinensis | 86.90% |
| AAT42166 | S. bicolor | 85.10% |
| AAS47590 | S. italica | 85.10% |

TABLE 10B
Comparison of GMsg04a02 (SEQ ID NO: 118) to known
phospholipid hydroperoxide glutathione peroxidases

| Public Database | | |
|---|---|---|
| Accession # | Species | Sequence Identity (%) |
| CAD31839 | C. arietinum | 88.00% |
| AAP81673 | Lotus corniculatus | 85.60% |
| AAL40914 | Momordica charantia | 83.80% |
| CAN60579 | V. vinifera | 83.20% |
| AAT42166 | S. bicolor | 76.20% |

TABLE 11B
Comparison of GMsp36c10 (SEQ ID NO: 120) to known
phospholipid hydroperoxide glutathione peroxidases

| Public Database | | |
|---|---|---|
| Accession # | Species | Sequence Identity (%) |
| O24296 | P. sativum | 80.10% |
| ABE93916 | M. truncatula | 79.20% |
| CAL59721 | M. sativa | 79.20% |
| NP_194915 | A. thaliana | 70.80% |
| AAC78466 | Zantedeschia | 69.30% |
| | aethiopica | |

TABLE 12B
Comparison of GMsp82f11 (SEQ ID NO: 122) to known
phospholipid hydroperoxide glutathione peroxidases

| Public Database | | |
|---|---|---|
| Accession # | Species | Sequence Identity (%) |
| NP_566128 | A. thaliana | 72.90% |
| NP_191867 | A. thaliana | 71.40% |
| AAQ03092 | Malus x domestica | 70.80% |
| AAM88847 | Z. mays | 69.40% |
| A84924 | A. thaliana | 69.00% |

TABLE 13B
Comparison of GMss66f03 (SEQ ID NO: 124) to known
phospholipid hydroperoxide glutathione peroxidases

| Public Database | | |
|---|---|---|
| Accession # | Species | Sequence Identity (%) |
| NP_566128 | A. thaliana | 71.20% |
| NP_191867 | A. thaliana | 69.10% |
| A84924 | A. thaliana | 67.30% |
| CAJ43709 | P. major | 66.50% |
| CAJ00224 | Capsicum chinense | 65.90% |

TABLE 14B
Comparison of LU61748885 (SEQ ID NO: 126) to known
phospholipid hydroperoxide glutathione peroxidases

| Public Database | | Sequence |
|---|---|---|
| Accession # | Species | Identity (%) |
| ABN59534 | Populus trichocarpa x Populus | 75.90% |
| | deltoides | |
| ABE92132 | M. truncatula | 73.80% |
| NP_564813 | A. thaliana | 71.80% |
| AAQ03092 | Malus x domestica | 70.60% |
| Q06652 | C. sinensis | 70.60% |

TABLE 15B
Comparison of OS36582281 (SEQ ID NO: 128) to known
phospholipid hydroperoxide glutathione peroxidases

| Public Database | | |
|---|---|---|
| Accession # | Species | Sequence Identity (%) |
| NP_001050145 | O. sativa | 76.50% |
| NP_566128 | A. thaliana | 72.90% |
| NP_191867 | A. thaliana | 69.10% |
| A84924 | A. thaliana | 68.40% |
| AAT42166 | S. bicolor | 64.70% |

TABLE 16B
Comparison of OS40057356 (SEQ ID NO: 130) to known
phospholipid hydroperoxide glutathione peroxidases

| Public Database | | |
|---|---|---|
| Accession # | Species | Sequence Identity (%) |
| NP_001053524 | O. sativa | 86.70% |
| CAD41644 | O. sativa | 85.30% |
| EAY95121 | O. sativa | 84.80% |
| AAS47590 | S. italica | 82.70% |
| AAT42166 | S. bicolor | 82.30% |

TABLE 17B
Comparison of ZM57588094 (SEQ ID NO: 132) to known
phospholipid hydroperoxide glutathione peroxidases

| Public Database | | |
|---|---|---|
| Accession # | Species | Sequence Identity (%) |
| BAD72440 | O. sativa | 73.50% |
| NP_001057006 | O. sativa | 71.80% |
| EAY99944 | O. sativa | 71.60% |
| CAN70486 | V. vinifera | 68.70% |
| NP_194915 | A. thaliana | 68.50% |

TABLE 18B
Comparison of ZM67281604 (SEQ ID NO: 134) to known
phospholipid hydroperoxide glutathione peroxidases

| Public Database | | |
|---|---|---|
| Accession # | Species | Sequence Identity (%) |
| AAS82602 | Z. mays | 95.50% |
| AAT42166 | S. bicolor | 95.20% |
| AAS47590 | S. italica | 94.00% |
| AAT42154 | Z. mays | 92.90% |
| AAQ64633 | T. monococcum | 92.30% |

TABLE 19B
Comparison of ZM68466470 (SEQ ID NO: 136) to known
phospholipid hydroperoxide glutathione peroxidases

| Public Database | | |
|---|---|---|
| Accession # | Species | Sequence Identity (%) |
| AAP59427 | L. esculentum | 50.30% |
| YP_570594 | Rhodopseudomonas | 49.70% |
| | palustris | |
| ZP_01061463 | Flavobacterium sp. MED217 | 47.50% |
| NP_948965 | Rhodopseudomonas | 47.50% |
| | palustris | |
| YP_578461 | Nitrobacter hamburgensis | 47.00% |

TABLE 2C
Comparison of BN45660154_5 (SEQ ID NO: 138) to known
TCP family transcription factors

| Public Database | | |
|---|---|---|
| Accession # | Species | Sequence Identity (%) |
| NP_189337 | A. thaliana | 79.90% |
| NP_001045247 | O. sativa | 44.00% |
| EAZ14676 | O. sativa | 41.20% |
| EAY77036 | O. sativa | 40.40% |
| NP_198919 | A. thaliana | 40.00% |

TABLE 3C
Comparison of BN45660154_8 (SEQ ID NO: 140) to known
TCP family transcription factors

| Public Database | | |
|---|---|---|
| Accession # | Species | Sequence Identity (%) |
| NP_189337 | A. thaliana | 81.30% |
| NP_001045247 | O. sativa | 44.50% |
| EAZ14676 | O. sativa | 41.40% |
| EAY77036 | O. sativa | 41.20% |
| NP_198919 | A. thaliana | 40.60% |

TABLE 4C
Comparison of ZM58885021 (SEQ ID NO: 142) to known
TCP family transcription factors

| Public Database | | |
|---|---|---|
| Accession # | Species | Sequence Identity (%) |
| EAZ24612 | O. sativa | 83.50% |
| NP_001048115 | O. sativa | 83.30% |
| BAD37305 | O. sativa | 67.80% |
| EAZ36344 | O. sativa | 60.40% |
| EAY87524 | O. sativa | 60.40% |

TABLE 5C
Comparison of BN43100775 (SEQ ID NO: 146) to known
ribosomal protein S6 kinases

| Public Database | | |
|---|---|---|
| Accession # | Species | Sequence Identity (%) |
| BAA07661 | A. thaliana | 84.80% |
| AAM61496 | A. thaliana | 83.50% |
| NP_187484 | A. thaliana | 66.20% |
| NP_001050027 | O. sativa | 65.70% |
| CAA56313 | Avena sativa | 64.50% |

TABLE 6C
Comparison of GM59673822 (SEQ ID NO: 148) to known
ribosomal protein S6 kinases

| Public Database | | |
|---|---|---|
| Accession # | Species | Sequence Identity (%) |
| NP_001050027 | O. sativa | 68.30% |
| BAA07661 | A. thaliana | 68.00% |
| CAB89082 | Asparagus officinalis | 67.60% |
| AAM61496 | A. thaliana | 66.50% |
| CAA56313 | A. sativa | 66.20% |

TABLE 7C
Comparison of AT5G60750 (SEQ ID NO: 158) to known
CAAX amino terminal protease family proteins

| Public Database | | |
|---|---|---|
| Accession # | Species | Sequence Identity (%) |
| NP_568928 | A. thaliana | 100.00% |
| BAB09848 | A. thaliana | 85.90% |
| ABE87113 | M. truncatula | 57.90% |
| EAZ01098 | O. sativa | 51.90% |
| NP_001057716 | O. sativa | 51.90% |

TABLE 8C
Comparison of BN51278543 (SEQ ID NO: 164) to
known DNA binding proteins

| Public Database | | |
|---|---|---|
| Accession # | Species | Sequence Identity (%) |
| AAK25936 | A. thaliana | 87.50% |
| NP_850679 | A. thaliana | 85.80% |
| ABJ97690 | Solanum tuberosum | 77.90% |
| NP_190748 | A. thaliana | 77.20% |
| ABF66654 | Ammopiptanthus | 75.80% |
| | mongolicus | |

TABLE 9C
Comparison of BN4306781 (SEQ ID NO: 174) to
proteins of unknown function

| Public Database | | |
|---|---|---|
| Accession # | Species | Sequence Identity (%) |
| NP_563630 | A. thaliana | 62.20% |
| NP_566063 | A. thaliana | 55.60% |
| AAL24177 | A. thaliana | 55.30% |
| ABB16971 | S. tuberosum | 52.10% |
| NP_192045 | A. thaliana | 48.10% |

TABLE 10C
Comparison of BN48622391 (SEQ ID NO: 176) to
known rev interacting proteins mis3

| Public Database | | |
|---|---|---|
| Accession # | Species | Sequence Identity (%) |
| NP_196459 | A. thaliana | 80.90% |
| AAM64563 | A. thaliana | 80.90% |
| NP_001064737 | O. sativa | 67.50% |
| EAY78750 | O. sativa | 60.50% |
| EAZ16285 | O. sativa | 60.20% |

TABLE 11C
Comparison of ZM57926241 (SEQ ID NO: 206) to
known CCCH type zinc finger proteins

| Public Database | | |
|---|---|---|
| Accession # | Species | Sequence Identity (%) |
| NP_001042276 | O. sativa | 74.90% |
| EAY72862 | O. sativa | 74.70% |
| EAY96854 | O. sativa | 67.20% |
| NP_001054861 | O. sativa | 67.10% |
| EAZ10869 | O. sativa | 57.40% |

TABLE 12C
Comparison of GM49819537 (SEQ ID NO: 182) to
known GRF1 interacting factors

| Public Database | | |
|---|---|---|
| Accession # | Species | Sequence Identity (%) |
| NP_198216 | A. thaliana | 65.20% |
| NP_001051174 | O. sativa | 51.10% |
| ABQ01228 | Z. mays | 50.40% |
| EAZ28484 | O. sativa | 40.80% |
| EAY91764 | O. sativa | 40.20% |

TABLE 13C
Comparison of HA66670700 (SEQ ID NO: 190) to
known eukaryotic translation initiation factor 4A proteins

| Public Database | | Sequence |
|---|---|---|
| Accession # | Species | Identity (%) |
| CAN62124 | V. vinifera | 88.60% |
| P41380 | Nicotiana plumbaginifolia | 88.00% |
| NP_001043673 | O. sativa | 88.00% |
| NP_001050506 | O. sativa | 87.50% |
| ABC55720 | Z. mays | 87.00% |

TABLE 14C
Comparison of HV100766 (SEQ ID NO: 202) to
known amino acid transporters

| Public Database | | |
|---|---|---|
| Accession # | Species | Sequence Identity (%) |
| NP_001060901 | O. sativa | 89.50% |
| CAD89802 | O. sativa | 87.70% |
| NP_198894 | A. thaliana | 76.70% |
| NP_851109 | A. thaliana | 76.50% |
| NP_564217 | A. thaliana | 76.10% |

TABLE 15C
Comparison of EST397 (SEQ ID NO: 204) to
known cyclic nucleotide gated ion channels

| Public Database | | |
|---|---|---|
| Accession # | Species | Sequence Identity (%) |
| NP_194785 | A. thaliana | 52.20% |
| NP_180393 | A. thaliana | 51.80% |
| CAN83465 | V. vinifera | 51.50% |
| Q9S9N5 | A. thaliana | 51.50% |
| NP_173051 | A. thaliana | 51.50% |

TABLE 16C
Comparison of ZM62043790 (SEQ ID NO: 154) to
known TGF beta receptor interacting proteins

| Public Database | | |
|---|---|---|
| Accession # | Species | Sequence Identity (%) |
| NP_001055036 | O. sativa | 89.30% |
| CAN80198 | V. vinifera | 80.10% |
| AAK49947 | Phaseolus | 79.50% |
| | vulgaris | |
| EAY97288 | O. sativa | 78.70% |
| ABO78477 | M. truncatula | 77.90% |

The full-length DNA sequence of the BN45660154_{—}5 (SEQ ID NO: 138), BN45660154_{—}8 (SEQ ID NO:140), and ZM58885021 (SEQ ID NO:142) were blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. One homologs from canola was identified. The degree of amino acid identity of these sequences to the closest known public sequences is indicated in Table 17C (Pairwise Comparison was used: gap penalty: 10; gap extension penalty: 0.1; score matrix: blosum62).

TABLE 17C
Comparison of BN46929759 (SEQ ID NO: 144) to
known TCP family transcription factors

| Public Database | | |
|---|---|---|
| Accession # | Species | Sequence Identity (%) |
| NP_564973 | A. thaliana | 82.80% |
| EAY87524 | O. sativa | 45.60% |
| EAZ24612 | O. sativa | 42.90% |
| NP_001048115 | O. sativa | 42.80% |
| BAD37305 | O. sativa | 42.60% |

The full-length DNA sequence of the BN43100775 (SEQ ID NO: 146) and GM59673822 (SEQ ID NO:148) were blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. One homolog from corn was identified. The degree of amino acid identity of these sequences to the closest known public sequences is indicated in Table 18C (Pairwise Comparison was used: gap penalty: 10; gap extension penalty: 0.1; score matrix: blosum62).

TABLE 18C
Comparison of ZM59314493 (SEQ ID NO: 150) to
known ribosomal protein S6 kinases

| Public Database | | |
|---|---|---|
| Accession # | Species | Sequence Identity (%) |
| NP_001050027 | O. sativa | 87.70% |
| CAA56313 | O. sativa | 85.20% |
| EAZ41107 | O. sativa | 75.70% |
| EAZ05158 | O. sativa | 75.70% |
| AAQ93804 | Z. mays | 73.40% |

The full-length DNA sequence of the AT5G60750 (SEQ ID NO: 158) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. One homolog from canola and one homolog from corn were identified. The degree of amino acid identity of these sequences to the closest known public sequences is indicated in Tables 19C and 20C (Pairwise Comparison was used: gap penalty: 10; gap extension penalty: 0.1; score matrix: blosum62).

TABLE 19C
Comparison of BN47819599 (SEQ ID NO: 160) to
known CAAX amino terminal protease family proteins

| Public Database | | |
|---|---|---|
| Accession # | Species | Sequence Identity (%) |
| AAM65055 | A. thaliana | 86.20% |
| NP_563943 | A. thaliana | 83.10% |
| AAF43926 | A. thaliana | 82.00% |
| NP_973823 | A. thaliana | 65.90% |
| NP_001077532 | A. thaliana | 61.40% |

TABLE 20C
Comparison of ZM65102675 (SEQ ID NO: 162) to
known CAAX amino terminal protease family proteins

| Public Database | | |
|---|---|---|
| Accession # | Species | Sequence Identity (%) |
| EAZ01098 | O. sativa | 75.30% |
| NP_001057716 | O. sativa | 75.30% |
| ABE87113 | M. truncatula | 55.90% |
| NP_568928 | A. thaliana | 53.70% |
| BAB09848 | A. thaliana | 52.20% |

The full-length DNA sequence of the BN51278543 (SEQ ID NO: 164) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Two homologs from soybean and two homologs from corn were identified. The degree of amino acid identity of these sequences to the closest known public sequences is indicated in Tables 21C through 24C (Pairwise Comparison was used: gap penalty: 10; gap extension penalty: 0.1; score matrix: blosum62).

TABLE 21C
Comparison of GM59587627 (SEQ ID NO: 166) to
known DNA binding protein

| Public Database | | |
|---|---|---|
| Accession # | Species | Sequence Identity (%) |
| ABF66654 | Ammopiptanthus | 91.40% |
| | mongolicus | |
| ABJ97690 | S. tuberosum | 87.70% |
| EAY97646 | O. sativa | 84.60% |
| NP_001055274 | O. sativa | 84.30% |
| AAB80919 | O. sativa | 82.80% |

TABLE 22C
Comparison of GMsae76c10 (SEQ ID NO: 168) to
known DNA binding proteins

| Public Database | | Sequence |
|---|---|---|
| Accession # | Species | Identity (%) |
| ABF66654 | A. mongolicus | 94.20% |
| ABJ97690 | S. tuberosum | 86.10% |
| EAY97646 | O. sativa | 83.90% |
| NP_001055274 | O. sativa | 83.60% |
| AAF91445 | Atriplex hortensis | 82.20% |

TABLE 23C
Comparison of ZM68403475 (SEQ ID NO: 170) to
known DNA binding proteins

| Public Database | | Sequence |
|---|---|---|
| Accession # | Species | Identity (%) |
| EAY97646 | O. sativa | 90.60% |
| NP_001055274 | O. sativa | 90.40% |
| AAB80919 | O. sativa | 88.60% |
| ABF66654 | A. mongolicus | 84.70% |
| ABJ97690 | S. tuberosum | 82.20% |

TABLE 24C
Comparison of ZMTD146063555 (SEQ ID NO: 172) to
known DNA binding proteins

| Public Database | | Sequence |
|---|---|---|
| Accession # | Species | Identity (%) |
| EAY97646 | O. sativa | 90.90% |
| NP_001055274 | O. sativa | 90.60% |
| AAB80919 | O. sativa | 88.80% |
| ABF66654 | A. mongolicus | 84.00% |
| ABJ97690 | S. tuberosum | 81.50% |

The full-length DNA sequence of BN48622391 (SEQ ID NO:176) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. One homolog from soybean and one homolog from corn were identified. The degree of amino acid identity of these sequences to the closest known public sequences is indicated in Tables 25C and 26C (Pairwise Comparison was used: gap penalty: 10; gap extension penalty: 0.1; score matrix: blosum62).

TABLE 25C
Comparison of GM50247805 (SEQ ID NO: 178) to
known rev interacting proteins

| Public Database | | Sequence |
|---|---|---|
| Accession # | Species | Identity (%) |
| NP_196459 | A. thaliana | 72.0% |
| AAM64563 | A. thaliana | 71.7% |
| NP_001064737 | O. sativa | 70.9% |
| BAD82278 | O. sativa | 62.3% |
| EAY75588 | O. sativa | 62.0% |

TABLE 26C
Comparison of ZM62208861 (SEQ ID NO: 180) to
known rev interacting proteins

| Public Database | | Sequence |
|---|---|---|
| Accession # | Species | Identity (%) |
| NP_001064737 | O. sativa | 82.90% |
| EAZ16285 | O. sativa | 74.30% |
| EAY78750 | O. sativa | 74.10% |
| BAD82278 | O. sativa | 72.80% |
| EAY75588 | O. sativa | 72.80% |

The full-length DNA sequence of the GM49819537 (SEQ ID NO: 182) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. One homolog from canola and two homologs from soybean were identified. The degree of amino acid identity of these sequences to the closest known public sequences is indicated in Tables 27C through 29C (Pairwise Comparison was used: gap penalty: 10; gap extension penalty: 0.1; score matrix: blosum62).

TABLE 27C
Comparison of BN42562310 (SEQ ID NO: 184) to
known GRF1 interacting factors

| Public Database | | Sequence |
|---|---|---|
| Accession # | Species | Identity (%) |
| NP_198216 | A. thaliana | 94.80% |
| NP_001051174 | O. sativa | 50.40% |
| ABQ01228 | Z. mays | 48.70% |
| EAY91764 | O. sativa | 40.80% |
| EAZ28484 | O. sativa | 40.50% |

TABLE 28C
Comparison of GM47121078 (SEQ ID NO: 186) to
known GRF1 interacting factors

| Public Database | | Sequence |
|---|---|---|
| Accession # | Species | Identity (%) |
| NP_198216 | A. thaliana | 65.20% |
| NP_001051174 | O. sativa | 51.10% |
| ABQ01228 | Z. mays | 50.40% |
| EAZ28484 | O. sativa | 40.80% |
| EAY91764 | O. sativa | 40.20% |

TABLE 29C
Comparison of GMsf89h03 (SEQ ID NO: 188) to
known GRF1 interacting factors

| Public Database | | Sequence |
|---|---|---|
| Accession # | Species | Identity (%) |
| AAB62864 | A. thaliana | 62.90% |
| NP_567194 | A. thaliana | 62.50% |
| NP_563619 | A. thaliana | 56.30% |
| ABQ01229 | Z. mays | 50.00% |
| NP_001068275 | O. sativa | 50.00% |

The full-length DNA sequence of the HA66670700 (SEQ ID NO: 190) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Five homologs from soybean were identified. The degree of amino acid identity of these sequences to the closest known public sequences is indicated in Tables 30C through 34C (Pairwise Comparison was used: gap penalty: 10; gap extension penalty: 0.1; score matrix: blosum62).

TABLE 30C
Comparison of GM50390979 (SEQ ID NO: 192) to known
eukaryotic translation initiation factor 4A proteins

| Public Database | | Sequence |
|---|---|---|
| Accession # | Species | Identity (%) |
| CAN61608 | V. vinifera | 95.20% |
| Q40465 | N. tabacum | 94.40% |
| P41382 | N. tabacum | 94.20% |
| ABE81297 | M. truncatula | 94.20% |
| Q40467 | N. tabacum | 93.70% |

TABLE 31C
Comparison of GM59720014 (SEQ ID NO: 194) to known
eukaryotic translation initiation factor 4A proteins

| Public Database | | Sequence |
|---|---|---|
| Accession # | Species | Identity (%) |
| CAA76677 | P. sativum | 90.70% |
| CAN62124 | V. vinifera | 90.00% |
| P41380 | N. plumbaginifolia | 87.20% |
| NP_001043673 | O. sativa | 86.70% |
| NP_001050506 | O. sativa | 86.20% |

TABLE 32C
Comparison of GMsab62c11 (SEQ ID NO: 196) to known
eukaryotic translation initiation factor 4A proteins

| Public Database | | Sequence |
|---|---|---|
| Accession # | Species | Identity (%) |
| CAN61608 | V. vinifera | 95.40% |
| P41382 | N. tabacum | 94.20% |
| AAR23806 | H. annuus | 94.20% |
| Q40468 | N. tabacum | 94.20% |
| Q40471 | N. tabacum | 93.90% |

TABLE 33C
Comparison of GMsl42e03 (SEQ ID NO: 198) to known
eukaryotic translation initiation factor 4A proteins

| Public Database | | Sequence |
|---|---|---|
| Accession # | Species | Identity (%) |
| CAN61608 | V. vinifera | 95.60% |
| ABN09109 | M. truncatula | 94.90% |
| AAR23806 | H. annuus | 94.70% |
| AAN74635 | P. sativum | 94.40% |
| Q40468 | N. tabacum | 94.40% |

TABLE 34C
Comparison of GMss72c01 (SEQ ID NO: 200) to known
eukaryotic translation initiation factor 4A proteins

| Public Database | | Sequence |
|---|---|---|
| Accession # | Species | Identity (%) |
| CAN61608 | V. vinifera | 95.40% |
| P41382 | N. tabacum | 94.40% |
| Q40465 | N. tabacum | 94.20% |
| ABE81297 | M. truncatula | 94.20% |
| Q40467 | N. tabacum | 93.90% |

The full-length DNA sequence of the ZM62043790 (SEQ ID NO: 154) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Two homologs from soybean were identified. The degree of amino acid identity of these sequences to the closest known public sequences is indicated in Tables 19C and 20C (Pairwise Comparison was used: gap penalty: 10; gap extension penalty: 0.1; score matrix: blosum62).

TABLE 35C
Comparison of GMsk21g122 (SEQ ID NO: 156) to
known TGF beta receptor interacting proteins

| Public Database | | Sequence |
|---|---|---|
| Accession # | Species | Identity (%) |
| AAK49947 | Phaseolus | 93.60% |
| | vulgaris | |
| ABO78477 | M. truncatula | 90.50% |
| CAN80198 | V. vinifera | 89.30% |
| NP_001055036 | O. sativa | 82.80% |
| AAK43862 | A. thaliana | 81.40% |

TABLE 36C
Comparison of GMsk21ga12 (SEQ ID NO: 152) to
known TGF beta receptor interacting proteins

| Public Database | | Sequence |
|---|---|---|
| Accession # | Species | Identity (%) |
| AAK49947 | P. vulgaris | 94.20% |
| ABO78477 | M. truncatula | 90.50% |
| CAN80198 | V. vinifera | 90.20% |
| NP_001055036 | O. sativa | 82.30% |
| AAK43862 | A. thaliana | 80.80% |

TABLE 2D
Comparison of EST285 (SEQ ID NO: 208) to known AP2
domain containing proteins

| Public Database | | Sequence |
|---|---|---|
| Accession # | Species | Identity (%) |
| ABA43687 | P. patens | 39.50% |
| ABE80929 | Medicago | 38.00% |
| | truncatula | |
| NP_181113 | A. thaliana | 37.30% |
| ABK28523 | A. thaliana | 37.20% |
| NP_174636 | A. thaliana | 37.00% |

TABLE 3D
Comparison of ZM100324 (SEQ ID NO: 212) to known AP2
domain containing proteins

| Public Database | | Sequence |
|---|---|---|
| Accession # | Species | Identity (%) |
| AAX28957 | H. vulgare | 56.20% |
| BAC20185 | Prunus avium | 51.10% |
| ABD72616 | A. thaliana | 49.40% |
| AAT65201 | Glycine soja | 47.90% |
| AAY21898 | Chorispora | 45.80% |
| | bungeana | |

The full-length DNA sequence of the EST285 (SEQ ID NO: 208) and ZM100324 (SEQ ID NO:212) were blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Six homologs from canola, four homologs from soybean, four homologs from sunflower, three homologs from linseed, three homologs from wheat, and one homolog from corn were identified. The degree of amino acid identity of these sequences to the closest known public sequences is indicated in Tables 19D and 20D (Pairwise Comparison was used: gap penalty: 10; gap extension penalty: 0.1; score matrix: blosum62).

TABLE 4D
Comparison of BN42471769 (SEQ ID NO: 210) to known AP2
domain containing proteins

| | | Sequence Identity |
|---|---|---|
| Public Database Accession # | Species | (%) |
| NP_197953 | A. thaliana | 80.40% |
| NP_196720 | A. thaliana | 59.50% |
| BAD01554 | Cucumis melo | 52.30% |
| ABE80929 | M. truncatula | 48.90% |
| NP_195006 | A. thaliana | 47.40% |

TABLE 5D
Comparison of BN42817730 (SEQ ID NO: 214) to known AP2
domain containing proteins

| | | Sequence Identity |
|---|---|---|
| Public Database Accession # | Species | (%) |
| ABA54282 | B. napus | 73.00% |
| AAW28084 | B. napus | 73.00% |
| ABA54281 | B. napus | 72.50% |
| ABA54280 | B. napus | 72.00% |
| NP_181113 | A. thaliana | 71.20% |

TABLE 6D
Comparison of BN45236208 (SEQ ID NO: 216) to known AP2
domain containing proteins

| | | Sequence Identity |
|---|---|---|
| Public Database Accession # | Species | (%) |
| NP_173609 | A. thaliana | 73.80% |
| AAM63137 | A. thaliana | 73.50% |
| NP_177887 | A. thaliana | 58.50% |
| BAD43987 | A. thaliana | 56.90% |
| NP_175104 | A. thaliana | 50.20% |

TABLE 7D
Comparison of BN46730374 (SEQ ID NO: 218) to known AP2
domain containing proteins

| Public Database | | |
|---|---|---|
| Accession # | Species | Sequence Identity (%) |
| NP_173355 | A. thaliana | 74.10% |
| AAF82238 | A. thaliana | 73.80% |
| ABB36646 | G. max | 51.00% |
| BAF47194 | Daucus carota | 49.00% |
| NP_680184 | A. thaliana | 42.40% |

TABLE 8D
Comparison of BN46832560 (SEQ ID NO: 220) to known AP2
domain containing proteins

| | | Sequence Identity |
|---|---|---|
| Public Database Accession # | Species | (%) |
| NP_193408 | A. thaliana | 85.80% |
| NP_181113 | A. thaliana | 66.20% |
| ABK28523 | A. thaliana | 65.80% |
| AAW28084 | B. napus | 64.60% |
| ABA54282 | B. napus | 64.10% |

TABLE 9D
Comparison of BN46868821 (SEQ ID NO: 222) to known AP2
domain containing proteins

| | | Sequence Identity |
|---|---|---|
| Public Database Accession # | Species | (%) |
| NP_177844 | A. thaliana | 83.50% |
| ABK28471 | A. thaliana | 83.10% |
| NP_195006 | A. thaliana | 46.60% |
| NP_565609 | A. thaliana | 45.60% |
| NP_188249 | A. thaliana | 44.20% |

TABLE 10D
Comparison of GM48927342 (SEQ ID NO: 224) to known AP2
domain containing proteins

| | | Sequence Identity |
|---|---|---|
| Public Database Accession # | Species | (%) |
| BAD01554 | C. melo | 48.60% |
| NP_196720 | A. thaliana | 47.70% |
| NP_188249 | A. thaliana | 45.80% |
| NP_177844 | A. thaliana | 44.60% |
| ABE80929 | M. truncatula | 44.50% |

TABLE 11D
Comparison of GM48955695 (SEQ ID NO: 226) to known AP2
domain containing proteins

| Public Database Accession # | Species | Sequence Identity (%) |
|---|---|---|
| ABB36646 | G. max | 39.80% |
| NP_173355 | A. thaliana | 39.10% |
| BAF47194 | D. carota | 38.30% |
| AAF82238 | A. thaliana | 37.10% |
| EAZ07208 | O. sativa | 35.10% |

TABLE 12D
Comparison of GM48958569 (SEQ ID NO: 228) to known AP2
domain containing proteins

| Public Database Accession # | Species | Sequence Identity (%) |
|---|---|---|
| ABK28850 | M. truncatula | 75.20% |
| ABQ85893 | P. sativum | 69.40% |
| ABE86412 | M. truncatula | 54.30% |
| ABE86412 | M. truncatula | 54.30% |
| EAZ03158 | O. sativa | 42.60% |

TABLE 13D
Comparison of GM50526381 (SEQ ID NO: 230) to known AP2
domain containing proteins

| Public Database Accession # | Species | Sequence Identity (%) |
|---|---|---|
| ABB36646 | G. max | 56.00% |
| NP_173355 | A. thaliana | 46.40% |
| BAF47194 | D. carota | 45.50% |
| AAF82238 | A. thaliana | 45.50% |
| NP_680184 | A. thaliana | 42.70% |

TABLE 14D
Comparison of HA66511283 (SEQ ID NO: 232) to known AP2
domain containing proteins

| Public Database | | |
|---|---|---|
| Accession # | Species | Sequence Identity (%) |
| AAS82861 | H. annuus | 36.90% |
| CAB93939 | Catharanthus roseus | 31.40% |
| AAN77051 | L. esculentum | 31.00% |
| NP_001042107 | O. sativa | 30.30% |
| ABQ59087 | Populus alba x Populus x | 30.10% |
| | berolinensis | |

TABLE 15D
Comparison of HA66563970 (SEQ ID NO: 234) to known AP2
domain containing proteins

| | | Sequence Identity |
|---|---|---|
| Public Database Accession # | Species | (%) |
| ABQ42205 | G. max | 47.80% |
| CAH67505 | O. sativa | 45.80% |
| NP_001053487 | O. sativa | 45.50% |
| ABA54281 | B. napus | 45.50% |
| ABA54280 | B. napus | 45.50% |

TABLE 16D
Comparison of HA66692703 (SEQ ID NO: 236) to known AP2
domain containing proteins

| | | Sequence Identity |
|---|---|---|
| Public Database Accession # | Species | (%) |
| AAS20427 | C. annuum | 52.00% |
| AAO34704 | L. esculentum | 49.50% |
| AAR87866 | L. esculentum | 49.50% |
| BAD01556 | C. melo | 48.40% |
| ABE84970 | M. truncatula | 44.30% |

TABLE 17D
Comparison of HA66822928 (SEQ ID NO: 238) to known
AP2 domain containing proteins

| Public Database | | |
|---|---|---|
| Accession # | Species | Sequence Identity (%) |
| AAY89658 | G. max | 56.40% |
| ABB36645 | G. max | 56.40% |
| CAN64037 | V. vinifera | 56.10% |
| AAQ08000 | G. hirsutum | 55.80% |
| NP_179915 | A. thaliana | 53.90% |

TABLE 18D
Comparison of LU61569679 (SEQ ID NO: 240) to
known AP2 domain containing proteins

| Public Database | | Sequence |
|---|---|---|
| Accession # | Species | Identity (%) |
| NP_177681 | A. thaliana | 50.90% |
| ABK59671 | A. thaliana | 50.40% |
| CAN60823 | V. vinifera | 44.50% |
| CAN66064 | V. vinifera | 43.30% |
| ABP02847 | M. truncatula | 35.90% |

TABLE 19D
Comparison of LU61703351 (SEQ ID NO: 242) to
known AP2 domain containing proteins

| Public Database | | |
|---|---|---|
| Accession # | Species | Sequence Identity (%) |
| ABK59671 | A. thaliana | 42.90% |
| NP_177681 | A. thaliana | 42.30% |
| CAN60823 | V. vinifera | 38.20% |
| CAN66064 | V. vinifera | 35.90% |
| EAZ08049 | O. sativa | 32.50% |

TABLE 20D
Comparison of LU61962194 (SEQ ID NO: 244) to
known AP2 domain containing proteins

| Public Database | | |
|---|---|---|
| Accession # | Species | Sequence Identity (%) |
| CAN63728 | V. vinifera | 50.00% |
| ABC69353 | M. truncatula | 49.60% |
| AAQ96342 | V. aestivalis | 47.20% |
| CAN80071 | V. vinifera | 46.50% |
| AAD09248 | Stylosanthes | 46.10% |
| | hamata | |

TABLE 21D
Comparison of TA54564073 (SEQ ID NO: 246) to
known AP2 domain containing proteins

| Public Database | | |
|---|---|---|
| Accession # | Species | Sequence Identity (%) |
| AAX13289 | T. aestivum | 75.20% |
| ABA08426 | T. aestivum | 72.00% |
| AAY44604 | T. aestivum | 67.60% |
| AAU29412 | Hordeum | 67.40% |
| | brevisubulatum | |
| AAL01124 | T. aestivum | 67.30% |

TABLE 22D
Comparison of TA54788773 (SEQ ID NO: 248) to
known AP2 domain containing proteins

| Public Database | | |
|---|---|---|
| Accession # | Species | Sequence Identity (%) |
| ABB51574 | C. annuum | 31.70% |
| EAZ36121 | O. sativa | 17.00% |
| CAD56466 | T. aestivum | 15.20% |
| AAX13280 | T. aestivum | 14.90% |
| EAY87770 | O. sativa | 14.80% |

TABLE 23D
Comparison of TA56412836 (SEQ ID NO: 250) to
known AP2 domain containing proteins

| Public Database | | Sequence Identity |
|---|---|---|
| Accession # | Species | (%) |
| EAY86936 | O. sativa | 72.80% |
| NP_001047614 | O. sativa | 72.80% |
| CAC39058 | O. sativa | 72.50% |
| ABQ52686 | Thinopyrum | 72.40% |
| | intermedium | |
| ABQ52687 | T. aestivum | 67.80% |

TABLE 24D
Comparison of ZM65144673 (SEQ ID NO: 252) to
known AP2 domain containing proteins

| Public Database | | Sequence Identity |
|---|---|---|
| Accession # | Species | (%) |
| ABP65298 | O. sativa | 63.30% |
| EAY87770 | O. sativa | 53.40% |
| NP_001048319 | O. sativa | 52.10% |
| EAZ36121 | O. sativa | 51.90% |
| AAF23899 | O. sativa | 50.70% |

TABLE 2E
Comparison of EST314 (SEQ ID NO: 254) to known
brassinosteroid biosynthetic LKB-like proteins

| Public Database | | |
|---|---|---|
| Accession # | Species | Sequence Identity (%) |
| CAN79299 | Vitis vinifera | 74.20% |
| AAK15493 | Pisum sativum | 73.90% |
| P93472 | P. sativum | 73.50% |
| AAM47602 | Gossypium hirsutum | 73.50% |
| AAL91175 | A. thaliana | 72.30% |

TABLE 3E
Comparison of EST322 (SEQ ID NO: 256) to
known RING-box proteins

| Public Database | | |
|---|---|---|
| Accession # | Species | Sequence Identity (%) |
| EDK43882 | Lodderomyces | 46.50% |
| | elongisporus | |
| AAT10276 | Fragaria x ananassa | 25.50% |
| CAF93382 | Tetraodon nigroviridis | 24.90% |
| XP_001249317 | Bos taurus | 24.70% |
| XP_637131 | Dictyostelium discoideum | 24.70% |

TABLE 4E
Comparison of EST589 (SEQ ID NO: 258) to known
serine/threonine protein phosphatases

| Public Database | | |
|---|---|---|
| Accession # | Species | Sequence Identity (%) |
| NP_001062774 | O. sativa | 89.30% |
| NP_200337 | A. thaliana | 89.20% |
| CAA80312 | A. thaliana | 88.90% |
| XP_799172 | Strongylocentrotus purpuratus | 84.40% |
| NP_988943 | Xenopus tropicalis | 83.70% |

The full-length DNA sequence of the serine/threonine protein phosphatase EST589 (SEQ ID NO:258) was blasted against proprietary databases of canola, soybean, rice, maize, linseed, sunflower, and wheat cDNAs at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Five homologs from canola, three homologs from soybean, one homolog from sunflower, three homologs from linseed, one homolog from wheat and one homolog from corn were identified. The degree of amino acid identity of these sequences to the closest known public sequences is indicated in Tables 5E through 18E (Pairwise Comparison was used: gap penalty: 10; gap extension penalty: 0.1; score matrix: blosum62).

TABLE 5E
Comparison of BN45899621 (SEQ ID NO: 260) to
known serine/threonine protein phosphatases

| Public Database | | |
|---|---|---|
| Accession # | Species | Sequence Identity (%) |
| NP_188632 | A. thaliana | 97.00% |
| NP_175454 | A. thaliana | 96.70% |
| BAE98396 | A. thaliana | 96.40% |
| AAM21172 | P. sativum | 94.70% |
| CAA87385 | Malus x domestica | 94.70% |

TABLE 6E
Comparison of BN51334240 (SEQ ID NO: 262) to
known serine/threonine protein phosphatases

| Public Database | | |
|---|---|---|
| Accession # | Species | Sequence Identity (%) |
| NP_200337 | A. thaliana | 93.10% |
| CAA80312 | A. thaliana | 92.80% |
| NP_194402 | A. thaliana | 92.50% |
| NP_001062774 | O. sativa | 90.60% |
| XP_001435846 | Paramecium tetraurelia | 81.10% |

TABLE 7E
Comparison of BN51345476 (SEQ ID NO: 264) to
known serine/threonine protein phosphatases

| Public Database | | |
|---|---|---|
| Accession # | Species | Sequence Identity (%) |
| P23778 | B. napus | 94.90% |
| Q06009 | Medicago sativa | 94.60% |
| S12986 | B. napus | 94.60% |
| NP_565974 | A. thaliana | 86.60% |

TABLE 8E
Comparison of BN42856089 (SEQ ID NO: 266) to
known serine/threonine protein phosphatases

| Public Database | | |
|---|---|---|
| Accession # | Species | Sequence Identity (%) |
| NP_172514 | A. thaliana | 97.10% |
| AAM65099 | A. thaliana | 95.80% |
| AAQ67226 | Lycopersicon esculentum | 95.40% |
| BAA92697 | V. faba | 95.10% |
| NP_176192 | A. thaliana | 79.40% |

TABLE 9E
Comparison of BN43206527 (SEQ ID NO: 268) to
known serine/threonine protein phosphatases

| Public Database | | Sequence |
|---|---|---|
| Accession # | Species | Identity (%) |
| NP_172514 | A. thaliana | 97.40% |
| AAM65099 | A. thaliana | 96.10% |
| AAQ67226 | L. esculentum | 95.10% |
| BAA92697 | V. faba | 94.10% |
| NP_176192 | A. thaliana | 79.70% |

TABLE 10E
Comparison of GMsf85h09 (SEQ ID NO: 270) to
known serine/threonine protein phosphatases

| Public Database | | Sequence |
|---|---|---|
| Accession # | Species | Identity (%) |
| NP_200337 | A. thaliana | 93.80% |
| CAA80312 | A. thaliana | 93.20% |
| NP_001062774 | O. sativa | 92.90% |
| NP_194402 | A. thaliana | 86.90% |
| NP_988943 | X. tropicalis | 82.80% |

TABLE 11E
Comparison of GMsj98e01 (SEQ ID NO: 272) to
known serine/threonine protein phosphatases

| Public Database | | Sequence |
|---|---|---|
| Accession # | Species | Identity (%) |
| BAA92699 | V. faba | 94.60% |
| Q06009 | M. sativa | 93.90% |
| CAN78260 | V. vinifera | 92.70% |
| NP_565974 | A. thaliana | 81.80% |

TABLE 12E
Comparison of GMsu65h07 (SEQ ID NO: 274) to
known serine/threonine protein phosphatases

| Public Database | | Sequence |
|---|---|---|
| Accession # | Species | Identity (%) |
| BAA92697 | V. faba | 98.70% |
| CAC11129 | F. sylvatica | 98.40% |
| AAQ67226 | L. esculentum | 97.40% |
| BAA92698 | V. faba | 96.70% |
| Q9ZSE4 | Hevea brasiliensis | 96.40% |

TABLE 13E
Comparison of HA66777473 (SEQ ID NO: 276) to
known serine/threonine protein phosphatases

| Public Database | | Sequence |
|---|---|---|
| Accession # | Species | Identity (%) |
| CAN78260 | V. vinifera | 93.30% |
| ABE78681 | Medicago truncatula | 91.70% |
| Q06009 | M. sativa | 90.70% |
| BAA92699 | V. faba | 90.40% |
| NP_001051627 | O. sativa | 52.90% |

TABLE 14E
Comparison of LU61781371 (SEQ ID NO: 278) to
known serine/threonine protein phosphatases

| Public Database | | Sequence |
|---|---|---|
| Accession # | Species | Identity (%) |
| NP_200337 | A. thaliana | 95.10% |
| CAA80312 | A. thaliana | 94.40% |
| NP_001062774 | O. sativa | 92.80% |
| NP_194402 | A. thaliana | 86.90% |

TABLE 15E
Comparison of LU61589678 (SEQ ID NO: 280) to
known serine/threonine protein phosphatases

| Public Database | | Sequence |
|---|---|---|
| Accession # | Species | Identity (%) |
| AAM21172 | P. sativum | 97.40% |
| CAA87385 | Malus × domestica | 97.40% |
| NP_175454 | A. thaliana | 96.00% |
| NP_188632 | A. thaliana | 95.70% |
| BAE98396 | A. thaliana | 95.70% |

TABLE 16E
Comparison of LU61857781 (SEQ ID NO: 282) to
known serine/threonine protein phosphatases

| Public Database | | Sequence |
|---|---|---|
| Accession # | Species | Identity (%) |
| CAN78260 | V. vinifera | 97.10% |
| ABE78681 | M. truncatula | 95.20% |
| Q9XGH7 | Nicotiana tabacum | 94.60% |
| NP_565974 | A. thaliana | 82.90% |

TABLE 17E
Comparison of TA55079288 (SEQ ID NO: 284) to
known serine/threonine protein phosphatases

| Public Database | | Sequence |
|---|---|---|
| Accession # | Species | Identity (%) |
| ABE78681 | M. truncatula | 92.90% |
| Q9XGH7 | N. tabacum | 92.60% |
| CAN78260 | V. vinifera | 92.40% |
| NP_001051627 | O. sativa | 56.00% |

TABLE 18E
Comparison of ZM59400933 (SEQ ID NO: 286) to
known serine/threonine protein phosphatases

| Public Database | | Sequence |
|---|---|---|
| Accession # | Species | Identity (%) |
| AAC72838 | O. sativa | 95.80% |
| AAA91806 | O. sativa | 94.40% |
| BAA92697 | V. faba | 92.80% |
| NP_001057926 | O. sativa | 82.80% |
| NP_001046300 | O. sativa | 78.90% |

### Example 2

### Characterization of Genes

Lead genes b1805 (SEQ ID NO:287), YER015W (SEQ ID NO:289), b1091 (SEQ ID NO:317), b0185 (SEQ ID NO:319), b3256 (SEQ ID NO:321), b3255 (SEQ ID NO:329), b1095 (SEQ ID NO:335), b1093 (SEQ ID NO:343), slr0886 (SEQ ID NO:345), and slr1364 (SEQ ID NO:397) were cloned using standard recombinant techniques. The functionality of each lead gene was predicted by comparing the amino acid sequence of the gene with other genes of known functionality. Homolog cDNAs were isolated from proprietary libraries of the respective species using known methods. Sequences were processed and annotated using bioinformatics analyses. The degrees of amino acid identity of the isolated sequences to the respective closest known public sequences (Pairwise Comparison was used: gap penalty: 10; gap extension penalty: 0.1; score matrix: blosum. 62) were used in the selection of homologous sequences as indicated in Tables 2F through 11F

TABLE 2F
Comparison of b1805 (SEQ ID NO: 288) to
known long-chain-fatty-acid-CoA ligase subunits
of acyl-CoA synthetase

| Public Database | | Sequence |
|---|---|---|
| Accession # | Species | Identity (%) |
| YP_407739 | Shigella boydii | 99.60% |
| NP_288241 | Escherichia coli | 99.50% |
| YP_310302 | Shigella sonnei | 99.50% |
| ZP_00709029 | Escherichia coli | 98.10% |

TABLE 3F
Comparison of YER015W (SEQ ID NO: 290) to
known long-chain-fatty-acid-CoA ligase
subunits of acyl-CoA synthetase

| Public Database | | Sequence |
|---|---|---|
| Accession # | Species | Identity (%) |
| XP_001643054 | Vanderwaltozyma polyspora | 66.10% |
| XP_447210 | Candida glabrata | 65.40% |
| XP_452045 | Kluyveromyces lactis | 52.30% |
| NP_984148 | Ashbya gossypii | 47.80% |

The b1805 (SEQ ID NO:287), and YER015W (SEQ ID NO:289) genes, from E. coli and S. cerevisiae, respectively, encode a subunit of acyl-CoA synthetase (long-chain-fatty-acid-CoA ligase, EC 6.2.1.3). The full-length DNA sequences of these genes were blasted against proprietary databases of soybean and maize cDNAs at an e value of e-10 (Altschul et al., 1997, Nucleic Acids Res. 25: 3389-3402). Six homologs from soybean, and seven homologs from corn were identified. The amino acid relatedness of these sequences is indicated in the alignments shown in FIG. 17.

TABLE 4F
Comparison of b1091 (SEQ ID NO: 318) to
known beta-ketoacyl-ACP synthases

| Public Database | | Sequence |
|---|---|---|
| Accession # | Species | Identity (%) |
| NP_287225 | Escherichia coli | 83.60% |
| YP_403645 | Shigella dysenteriae | 83.40% |
| NP_707007 | Shigella flexneri | 83.40% |
| ZP_00735938 | Escherichia coli | 83.40% |
| 1MZS | Escherichia coli | 83.40% |

TABLE 5F
Comparison of b0185 (SEQ ID NO: 320) to
known acetyl-CoA carboxylase complex alpha subunits

| Public Database | | Sequence |
|---|---|---|
| Accession # | Species | Identity (%) |
| YP_539241 | Escherichia coli | 99.70% |
| YP_309224 | Shigella sonnei | 99.70% |
| YP_406731 | Shigella boydii | 99.70% |
| ZP_00920451 | Shigella dysenteriae | 99.70% |

TABLE 6F
Comparison of b3256 (SEQ ID NO: 322) to known
biotin carboxylase subunits of acetyl CoA carboxylase

| Public Database | | Sequence |
|---|---|---|
| Accession # | Species | Identity (%) |
| ZP_00721902 | Escherichia coli | 99.80% |
| NP_312155 | Escherichia coli | 99.80% |
| NP_838758 | Shigella flexneriT | 99.80% |
| ZP_00923176 | Escherichia coli | 99.80% |

The b3256 gene (SEQ ID NO:321) from E. coli encodes a biotin-dependent carboxylase subunit of ACC. The full-length DNA sequence of this gene was blasted against a proprietary database of canola and soybean cDNAs at an e value of e⁻¹⁰ (Altschul et al., supra). One homolog from canola and two homologs from soybean were identified. The amino acid relatedness of these sequences is indicated in the alignments shown in FIG. 18.

TABLE 7F
Comparison of b3255 (SEQ ID NO: 330) to known biotin carboxyl
carrier protein subunits of acetyl-CoA carboxylase

| | | Sequence |
|---|---|---|
| Public Database Accession # | Species | Identity (%) |
| YP_404913 | Shigella dysenteriae | 99.40% |
| YP_001573179 | Salmonella enterica | 93.60% |
| NP_457755 | Salmonella enterica | 92.90% |
| YP_001456152 | Citrobacter koseri | 92.40% |

The b3255 gene (SEQ ID NO:329) from E. coli encodes a biotin carboxyl carrier protein subunit of ACC. The full-length DNA sequence of this gene was blasted against a proprietary database of canola cDNAs at an e value of e⁻¹⁰ (Altschul et al., supra). Two homologs from canola were identified. The amino acid relatedness of these sequences is indicated in the alignments shown in FIG. 19.

TABLE 8F
Comparison of b1095 (SEQ ID NO: 336) to known
3-oxoacyl-[acyl-carrier-protein] synthase II

| | | Sequence |
|---|---|---|
| Public Database Accession # | Species | Identity (%) |
| YP_310075 | Shigella sonnei | 99.80% |
| YP_540234 | Escherichia coli | 99.80% |
| ZP_01702199 | Escherichia coli | 99.80% |
| 1B3N | Escherichia coli | 99.80% |

The b1095 (SEQ ID NO:335) gene encodes a 3-oxoacyl-[acyl-carrier-protein] synthase II in E coli. The full-length DNA sequence of the b1095 was blasted against a proprietary database of soybean cDNAs at an e value of e⁻¹⁰ (Altschul et al., supra). Three homologs from soybean were identified. The amino acid relatedness of these sequences is indicated in the alignments shown in FIG. 20.

TABLE 9F
Comparison of b1093 (SEQ ID NO: 344) to known
3-oxoacyl-[acyl-carrier-protein] reductases

| | | Sequence |
|---|---|---|
| Public Database Accession # | Species | Identity (%) |
| NP_287227 | Escherichia coli | 99.60% |
| AAA23739 | Escherichia coli | 99.60% |
| 1Q7C | Escherichia coli | 99.60% |
| YP_403643 | Shigella dysenteriae | 99.60% |

TABLE 10F
Comparison of slr0886 (SEQ ID NO: 346) to known
3-oxoacyl-[acyl-carrier-protein] reductases

| | | Sequence |
|---|---|---|
| Public Database Accession # | Species | Identity (%) |
| YP_001519901 | Acaryochloris marina | 80.60% |
| YP_324264 | Anabaena variabilis | 78.90% |
| NP_485934 | Nostoc sp. PCC 7120 | 78.50% |
| ZP_01631414 | Nodularia spumigena | 77.00% |

Genes b1093 (SEQ ID NO:343) and slr0886 (SEQ ID NO:345) encode 3-oxoacyl-ACP reductases in E. coli and Synechocystis sp. pcc6803, respectively. The full-length DNA sequences of these genes were blasted against proprietary databases of canola, soybean, rice, maize, and linseed cDNAs at an e value of e⁻¹⁰ (Altschul et al., supra). Three homologs from canola, seven homologs from maize, one homolog from linseed, one homolog from rice, one homolog from barley and twelve homologs from soybean were identified. The amino acid relatedness of these sequences is indicated in the alignments shown in FIG. 21.

TABLE 11F
Comparison of slr1364 (SEQ ID NO: 398) to known biotin synthetases

| | | Sequence |
|---|---|---|
| Public Database Accession # | Species | Identity (%) |
| ZP_00514954 | Crocosphaera watsonii | 74.80% |
| ZP_01728784 | Cyanothece sp. | 74.80% |
| YP_723094 | Trichodesmium erythraeum | 73.00% |
| CAO89443 | Microcystis aeruginosa | 72.50% |

The full-length DNA sequence of slr1364 (SEQ ID NO:397) encodes a biotin synthetase from Synechocystis sp. pcc6803. The full-length DNA sequences of this gene were blasted against proprietary databases of canola and maize cDNAs at an e value of e⁻¹⁰ (Altschul et al., supra). One homolog each from canola and maize was identified. The amino acid relatedness of these sequences is indicated in the alignments shown in FIG. 22.

### Example 3

### Characterization of Genes

Sterol pathway genes B0421 (SEQ ID NO:413), YJL167W (SEQ ID NO:415), SQS1 (SEQ ID NO:435), and YGR175c (SEQ ID NO:443) were cloned using standard recombinant techniques. The functionality of each sterol pathway gene was predicted by comparing the amino acid sequence of the gene with other genes of known functionality. Homolog cDNAs were isolated from proprietary libraries of the respective species using known methods. Sequences were processed and annotated using bioinformatics analyses. The degrees of amino acid identity of the isolated sequences to the respective closest known public sequences are indicated in Tables 2G through 5G (Pairwise Comparison was used: gap penalty: 11; gap extension penalty: 1; score matrix: blosum62). The degrees of amino acid identity and similarity of the isolated sequences to the respective closest known public sequences were used in the selection of homologous sequences as described below.

TABLE 2G
Comparison of B0421 (SEQ ID NO: 414)
to known farnesyl diphosphate synthases

| | | Sequence |
|---|---|---|
| Public Database Accession # | Species | Identity (%) |
| 1RQI | Escherichia coli | 99.70% |
| ZP_00921756 | Shigella dysenteriae | 99.70% |
| ZP_01700053 | Escherichia coli | 99.70% |
| ZP_00710166 | Escherichia coli | 99.70% |

TABLE 3G
Comparison of YJL167W (SEQ ID NO: 416)
to known farnesyl diphosphate synthases

| | | Sequence |
|---|---|---|
| Public Database Accession # | Species | Identity (%) |
| EDN63217 | Saccharomyces cerevisiae | 99.70% |
| XP_001646858 | Vanderwaltozyma | 77.60% |
| | polyspora | |
| XP_448787 | Candida glabrata | 77.60% |
| XP_451300 | Kluyveromyces lactis | 74.50% |

The B0421 (SEQ ID NO:414), and YJL167W (SEQ ID NO:416) genes, from E. coli and S. cerevisiae, respectively, encode FPS. The full-length DNA sequences of these genes were blasted against proprietary databases of soybean and maize cDNAs at an e value of e-10 (Altschul et al., 1997, Nucleic Acids Res. 25: 3389-3402). Two homologs from canola, three homologs from soybean, two homologs from wheat and two homologs from corn were identified. The amino acid relatedness of these sequences is indicated in the alignments shown in FIG. 24.

TABLE 4G
Comparison of SQS1 (SEQ ID NO: 436) to known squalene synthases

| | | Sequence |
|---|---|---|
| Public Database Accession # | Species | Identity (%) |
| A9RRG4 | Physcomitrella patens | 76.68% |
| O22107 | Glycine max | 46.07% |
| Q84LE3 | Lotus japonicus | 45.98% |
| O22106 | Zea mays | 45.29% |
| Q6Z368 | Oryza sativa | 40.22% |

SQS1 (SEQ ID NO:435) and SQS2 (SEQ ID NO:437) are synthetic squalene synthase genes. The full-length DNA sequence of this gene was blasted against proprietary databases of canola and maize cDNAs at an e value of e⁻¹⁰ (Altschul et al., supra). One homolog each from canola, soybean and maize was identified. The amino acid relatedness of these sequences is indicated in the alignments shown in FIG. 25.

TABLE 5G
Comparison of YGR175C (SEQ ID NO: 444)
to known squalene expoxidases

| | | Sequence |
|---|---|---|
| Public Database Accession # | Species | Identity (%) |
| AAA34592 | Saccharomyces | 99.80% |
| | cerevisiae | |
| EDN61765 | Saccharomyces | 99.60% |
| | cerevisiae | |
| XP_445667 | Candida glabrata | 83.70% |
| XP_001646877 | Vanderwaltozyma | 77.30% |
| | polyspora | |

The full-length DNA sequence of YGR175C (SEQ ID NO:444) encodes a squalene expoxidase from S. cerevisiae. The full-length DNA sequence of this gene was blasted against proprietary databases of canola and maize cDNAs at an e value of e⁻¹⁰ (Altschul et al., supra). One homolog each from canola and maize was identified. The amino acid relatedness of these sequences is indicated in the alignments shown in FIG. 26.

### Example 4

### Overexpression of Lead Genes in Plants

The polynucleotides of Table 1F were ligated into an expression cassette using known methods. Three different promoters were used to control expression of the transgenes in Arabidopsis: the USP promoter from Vicia faba (SEQ ID NO:403 was used for expression of genes from Escherichia coli or SEQ ID NO:404 was used for expression of genes from Saccharomyces cerevisiae); the super promoter (SEQ ID NO:405); and the parsley ubiquitin promoter (SEQ ID NO:406). For targeted expression, the mitochondrial transit peptide from an Arabidopsis thaliana gene encoding mitochondrial isovaleryl-CoA dehydrogenase designated “Mit” in Tables 12F-24F. SEQ ID NO:407 was used for expression of genes from Escherichia coli or SEQ ID NO:408 was used for expression of genes from Saccharomyces cerevisiae. In addition, for targeted expression, the chloroplast transit peptide of an Spinacia oleracea gene encoding ferredoxin nitrite reductase designated “Chlor” in Tables 12F-22F (SEQ ID NO:409) was used.

The Arabidopsis ecotype C24 was transformed with constructs containing the lead genes described in Example 2 using known methods. Seeds from T2 transformed plants were pooled on the basis of the promoter driving the expression, gene source species and type of targeting (chloroplastic, mitochondrial and cytoplasmic). The seed pools were used in the primary screens for biomass under well watered and water limited growth conditions. Hits from pools in the primary screen were selected, molecular analysis performed and seed collected. The collected seeds were then used for analysis in secondary screens where a larger number of individuals for each transgenic event were analyzed. If plants from a construct were identified in the secondary screen as having increased biomass compared to the controls, it passed to the tertiary screen. In this screen, over 100 plants from all transgenic events for that construct were measured under well watered and drought growth conditions. The data from the transgenic plants were compared to wild type Arabidopsis plants or to plants grown from a pool of randomly selected transgenic Arabidopsis seeds using standard statistical procedures.

Plants that were grown under well watered conditions were watered to soil saturation twice a week. Images of the transgenic plants were taken at 17 and 21 days using a commercial imaging system. Alternatively, plants were grown under water limited growth conditions by watering to soil saturation infrequently which allowed the soil to dry between watering treatments. In these experiments, water was given on days 0, 8, and 19 after sowing. Images of the transgenic plants were taken at 20 and 27 days using a commercial imaging system.

Image analysis software was used to compare the images of the transgenic and control plants grown in the same experiment. The images were used to determine the relative size or biomass of the plants as pixels and the color of the plants as the ratio of dark green to total area. The latter ratio, termed the health index, was a measure of the relative amount of chlorophyll in the leaves and therefore the relative amount of leaf senescence or yellowing and was recorded at day 27 only. Variation exists among transgenic plants that contain the various lead genes, due to different sites of DNA insertion and other factors that impact the level or pattern of gene expression.

Tables 12F to 24F show the comparison of measurements of the Arabidopsis plants. Percent change indicates the measurement of the transgenic relative to the control plants as a percentage of the control non-transgenic plants; p value is the statistical significance of the difference between transgenic and control plants based on a T-test comparison of all independent events where NS indicates not significant at the 5% level of probabilty; No. of events indicates the total number of independent transgenic events tested in the experiment; No. of positive events indicates the total number of independent transgenic events that were larger than the control in the experiment; No. of negative events indicates the total number of independent transgenic events that were smaller than the control in the experiment. NS indicates not significant at the 5% level of probability.

### A. Long-Chain-Fatty-Acid-CoA Ligase Subunits of Acyl-CoA Synthetase

The gene designated b1805 (SEQ ID NO:287), encoding the long-chain-fatty-acid-CoA ligase subunit of acyl-CoA synthetase, was expressed in Arabidopsis using three different constructs controlled by the USP promoter: constructs with no subcellular targeting, constructs targeted to the chloroplast, and constructs targeted to mitochondria. The b1805 gene (SEQ ID NO:287) was also expressed in Arabidopsis using the Super promoter, without subcellular targeting. Table 12F sets forth biomass and health index data obtained from the Arabidopsis plants transformed with these constructs and tested under water-limiting conditions. Table 13F sets forth biomass and health index data obtained from the Arabidopsis plants transformed with b1805 (SEQ ID NO:287) under control of the Super promoter, without subcellular targeting, and tested under well-watered conditions.

TABLE 12F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| b1805 | Super | none | Biomass at | −7.1 | NS | 6 | 1 | 5 |
| | | | day 20 | | | | | |
| b1805 | Super | none | Biomass at | −7.0 | NS | 6 | 1 | 5 |
| | | | day 27 | | | | | |
| b1805 | Super | none | Health index | −10.1 | 0.0037 | 6 | 2 | 4 |
| b1805 | USP | Mit | Biomass at | 53.0 | 0.0000 | 8 | 8 | 0 |
| | | | day 20 | | | | | |
| b1805 | USP | Mit | Biomass at | 20.3 | 0.0000 | 8 | 8 | 0 |
| | | | day 27 | | | | | |
| b1805 | USP | Mit | Health index | 19.8 | 0.0000 | 8 | 8 | 0 |
| b1805 | USP | none | Biomass at | 28.0 | 0.0001 | 5 | 4 | 1 |
| | | | day 20 | | | | | |
| b1805 | USP | none | Biomass at | 16.8 | 0.0024 | 5 | 4 | 1 |
| | | | day 27 | | | | | |
| b1805 | USP | none | Health index | 14.6 | 0.0000 | 5 | 4 | 1 |
| b1805 | USP | Chlor | Biomass at | 4.8 | NS | 5 | 3 | 2 |
| | | | day 20 | | | | | |
| b1805 | USP | Chlor | Biomass at | 3.5 | NS | 5 | 2 | 3 |
| | | | day 27 | | | | | |
| b1805 | USP | Chlor | Health index | −2.4 | NS | 5 | 3 | 2 |

Table 12F shows that Arabidopsis plants expressing b1805 (SEQ ID NO:287) without subcellular targeting or with targeting to mitochondria that were grown under water limiting conditions were significantly larger than the control plants that did not express b1805 (SEQ ID NO:287). In addition, these transgenic plants were darker green in color than the controls. This data indicates that the plants produced more chlorophyll or had less chlorophyll degradation during stress than the control plants. Table 12F also shows that the majority of independent transgenic events were larger than the controls. In addition, Table 12F shows that Arabidopsis plants expressing the b1805 gene with subcellular targeting to the chloroplast that were grown under water limiting conditions were similar in biomass and Health Index to the control plants that did not express the b1805 gene at two measuring times. Table 12F indicates that when transgenic Arabidopsis plants containing b1805 (SEQ ID NO:287) with no subcellular targeting under control of the Super promoter were grown under water limiting conditions, the transgenic plants were smaller than the control plants that did not express the b1805 gene at two measuring times indicating that these plants were more sensitive to water deprivation.

TABLE 13F

| | | | | | | | No. of | No. of |
|---|---|---|---|---|---|---|---|---|
| | | | | % | | No. of | Positive | Negative |
| Gene | Promoter | Targeting | Measurement | Change | p-Value | Events | Events | Events |
| b1805 | Super | none | Biomass at | 17.0 | 0.0000 | 6 | 6 | 0 |
| | | | day 17 | | | | | |
| b1805 | Super | none | Biomass at | 11.0 | 0.0000 | 6 | 6 | 0 |
| | | | day 21 | | | | | |
| b1805 | Super | none | Health index | −3.3 | NS | 6 | 1 | 5 |

Table 13F shows that Arabidopsis plants containing the b1805 gene (SEQ ID NO:287) in an expression cassette with no subcellular targeting under control of the Super promoter were significantly larger than control plants if grown under well watered conditions. Table 13F shows that the majority of independent transgenic events were larger than the controls in the well watered environment.

The gene designated YER015W (SEQ ID NO:289), encoding the long-chain-fatty-acid-CoA ligase subunit of acyl-CoA synthetase, was expressed in Arabidopsis using the USP promoter, with subcellular targeting to the mitochondria. Table 14F sets forth biomass and health index data obtained from Arabidopsis plants transformed with this construct.

TABLE 14F

| | | | | | | | No. of | No. of |
|---|---|---|---|---|---|---|---|---|
| | | | | % | | No. of | Positive | Negative |
| Gene | Promoter | Targeting | Measurement | Change | p-Value | Events | Events | Events |
| YER015W | USP | Mito | Biomass at | 23.5 | 0.0000 | 6 | 6 | 0 |
| | | | 17 days | | | | | |
| YER015W | USP | Mito | Biomass at | 16.7 | 0.0000 | 6 | 6 | 0 |
| | | | 21 days | | | | | |
| YER015W | USP | Mito | Health Index | 6.8 | 0.09 | 6 | 5 | 1 |

Table 14F shows that Arabidopsis plants that were grown under well watered conditions were significantly larger than the control plants that did not express YER015W (SEQ ID NO:290). Table 14F also shows that all independent transgenic events were larger than the controls in the well watered environment.

Tables 12F, 13F and 14F indicate that expression of a long-chain-fatty-acid-CoA ligase subunit of acyl-CoA synthetase will increase growth of plants, resulting in plants with larger biomass. The amount of water that the plants receive also influences growth and the plants with different constructs do not respond to the same extent to this stress. The promoter and the subcellular targeting used in the construct determines whether the plant is relatively more or less sensitive to the water deprivation.

### B. Beta-Ketoacyl-ACP Synthase

The b1091 gene (SEQ ID NO:317), which encodes a beta-ketoacyl-ACP synthase, was expressed in Arabidopsis using two constructs that had no subcellular targeting signal. In one construct, transcription was controlled by the USP promoter and in the second by the Super promoter. Table 15F sets forth biomass and health index data obtained from Arabidopsis plants transformed with these constructs and grown under well watered conditions.

TABLE 15F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| b1091 | Super | None | Biomass at | −7.5 | 0.0458 | 5 | 1 | 4 |
| | | | day 17 | | | | | |
| b1091 | Super | None | Biomass at | −7.8 | 0.0109 | 5 | 1 | 4 |
| | | | day 21 | | | | | |
| b1091 | Super | None | Health index | −1.5 | NS | 5 | 2 | 3 |
| b1091 | USP | None | Biomass at | 8.2 | 0.0031 | 6 | 5 | 1 |
| | | | day 17 | | | | | |
| b1091 | USP | None | Biomass at | 7.4 | 0.0002 | 6 | 6 | 0 |
| | | | day 21 | | | | | |
| b1091 | USP | None | Health index | −2.5 | NS | 6 | 3 | 3 |

Table 15F shows that Arabidopsis plants with the USP promoter controlling expression of b1091 (SEQ ID NO:317) were significantly larger than the control plants. Table 15F also shows that the majority of independent transgenic events with the USP promoter and b1091 (SEQ ID NO:317) were larger than the controls. In contrast, plants with the Super promoter controlling expression of b1091 (SEQ ID NO:317) were smaller than controls.

### C. Acetyl-CoA Carboxylase Complex Subunits

The b0185 gene (SEQ ID NO:319), which encodes an acetyl-CoA carboxylase complex alpha subunit, was expressed in Arabidopsis using an expression cassette that targeted the protein to the mitochondria and was controlled by the USP promoter. Table 16F sets forth biomass and health index data obtained from Arabidopsis plants transformed with these constructs and grown under water-limiting conditions.

TABLE 16F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| b0185 | USP | Mit | Biomass at | 8.0 | 0.0306 | 7 | 5 | 2 |
| | | | day 20 | | | | | |
| b0185 | USP | Mit | Biomass at | 2.4 | 0.4640 | 7 | 4 | 3 |
| | | | day 27 | | | | | |
| b0185 | USP | Mit | Health index | 12.1 | 0.0008 | 7 | 5 | 2 |

Table 16F shows that Arabidopsis plants containing the b0185 gene (SEQ ID NO:319) under control of the USP promoter that were grown under water limiting conditions were significantly larger than control plants that did not express b0185 (SEQ ID NO:319) at day 20. Table 16F shows that the majority of independent transgenic events were larger than the controls, indicating better adaptation to the stress environment. In addition, the transgenic plants were darker green in color than the controls at day 27. This indicates that the plants produced more chlorophyll or had less chlorophyll degradation during stress than the control plants.

The b3256 gene (SEQ ID NO:321), which encodes a biotin carboxylase subunit of acetyl CoA carboxylase, was expressed in Arabidopsis using an expression cassette that targeted the protein to the mitochondria and was controlled by the USP promoter. Table 17F sets forth biomass and health index data obtained from Arabidopsis plants transformed with these constructs and grown under water-limiting conditions.

TABLE 17F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| b3256 | USP | Mit | Biomass at | 12.3 | 0.0012 | 7 | 5 | 2 |
| | | | day 20 | | | | | |
| b3256 | USP | Mit | Biomass at | 8.3 | 0.0080 | 7 | 6 | 1 |
| | | | day 27 | | | | | |
| b3256 | USP | Mit | Health index | 1.2 | NS | 7 | 4 | 3 |

Table 17F shows that Arabidopsis plants that were grown under water limiting conditions were significantly larger than control plants that did not express the b3256 gene, at two measuring times. Table 17F shows that the majority of independent transgenic events were larger than the controls indicating better adaptation to the stress environment.

The b3255 gene (SEQ ID NO:329), which encodes a biotin carboxyl carrier protein subunit of acetyl CoA carboxylase, was expressed in Arabidopsis using two expression cassettes: in one cassette, the protein was targeted to the mitochondria and was controlled by the USP promoter. In the second cassette, b3255 (SEQ ID NO:329) was not targeted subcellularly, and was expressed under control of the Super promoter. Table 18F sets forth biomass and health index data obtained from Arabidopsis plants transformed with these constructs and grown under water-limiting conditions.

TABLE 18F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| B3255 | Super | None | Biomass at | 8.1 | NS | 6 | 4 | 2 |
| | | | day 20 | | | | | |
| B3255 | Super | None | Biomass at | 6.8 | NS | 6 | 3 | 3 |
| | | | day 27 | | | | | |
| B3255 | Super | None | Health index | 0.3 | NS | 6 | 3 | 3 |
| B3255 | USP | Mit | Biomass at | 25.4 | 0.0000 | 5 | 5 | 0 |
| | | | day 20 | | | | | |
| B3255 | USP | Mit | Biomass at | 7.4 | 0.0759 | 5 | 3 | 2 |
| | | | day 27 | | | | | |
| B3255 | USP | Mit | Health index | 9.1 | 0.0180 | 5 | 4 | 1 |

Table 18F shows that Arabidopsis plants comprising the b3255 gene (SEQ ID NO:329) under control of the USP promoter that were grown under water limiting conditions were larger than the control plants that did not express the b3255 gene (SEQ ID NO:329), at two measuring times. In addition, the transgenic plants were darker green in color than the controls. This indicates that the plants produced more chlorophyll or had less chlorophyll degradation during stress than the control plants. Table 18F shows that the majority of independent transgenic events were larger than the controls indicating better adaptation to the stress environment.

Table 18F further shows that when b3255 (SEQ ID NO:329) was expressed in Arabidopsis using an expression cassette that had no subcellular targeting, under control of the Super promoter and grown under water limiting conditions, the resulting Arabidopsis plants were similar in size and health index to the control plants that did not express the b3255 (SEQ ID NO:329), at two measuring times.

Table 19 sets forth biomass and health index data obtained from Arabidopsis plants transformed with these constructs and grown under well watered conditions. Table 19F shows that Arabidopsis plants expressing b3255 (SEQ ID NO:329) with no subcellular targeting that were grown under well watered conditions were larger than the control plants with the USP promoter but smaller if expression was controlled by the super promoter.

TABLE 19F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| B3255 | Super | None | Biomass at | −6.9 | 0.0331 | 6 | 2 | 4 |
| | | | day 17 | | | | | |
| B3255 | Super | None | Biomass at | −6.7 | 0.0145 | 6 | 2 | 4 |
| | | | day 21 | | | | | |
| B3255 | Super | None | Health index | −3.7 | NS | 6 | 2 | 4 |
| B3255 | USP | None | Biomass at | 13.4 | 0.0000 | 6 | 5 | 1 |
| | | | day 17 | | | | | |
| B3255 | USP | None | Biomass at | 6.4 | 0.0040 | 6 | 5 | 1 |
| | | | day 21 | | | | | |
| B3255 | USP | None | Health index | −6.1 | NS | 6 | 2 | 4 |

D. 3-oxoacyl-[acyl-carrier-protein]Synthase II

The b1095 (SEQ ID NO:335) gene, which encodes a 3-oxoacyl-[acyl-carrier-protein] synthase II, was expressed in Arabidopsis using an expression cassette that targeted the protein to the mitochondria, under control of the USP promoter. Table 20F sets forth biomass and health index data obtained from Arabidopsis plants transformed with this construct and grown under water-limiting conditions.

TABLE 20F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| b1095 | USP | Mit | Biomass at | 10.9 | 0.0073 | 7 | 5 | 2 |
| | | | day 20 | | | | | |
| b1095 | USP | Mit | Biomass at | 16.4 | 0.0001 | 7 | 6 | 1 |
| | | | day 27 | | | | | |
| b1095 | USP | Mit | Health index | −4.9 | NS | 7 | 2 | 5 |

Table 20F shows that Arabidopsis plants that were grown under water limiting conditions were significantly larger than the control plants that did not express b1095 (SEQ ID NO:335) at two measuring times. Table 20F shows that the majority of independent transgenic events were larger than the controls indicating better adaptation to the stress environment.

E. 3-oxoacyl-ACP Reductase

Gene b1093 (SEQ ID NO:343), which encodes a 3-oxoacyl-ACP reductase, was expressed in Arabidopsis using an expression cassette that targeted the protein to the mitochondria and was controlled by the USP promoter. Table 21F sets forth biomass and health index data obtained from Arabidopsis plants transformed with this construct and grown under water-limitina conditions.

TABLE 21F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| b1093 | USP | Mit | Biomass at | 25.1 | 0.0000 | 7 | 6 | 1 |
| | | | day 20 | | | | | |
| b1093 | USP | Mit | Biomass at | 14.4 | 0.0000 | 7 | 6 | 1 |
| | | | day 27 | | | | | |
| b1093 | USP | Mit | Health index | 16.6 | 0.0000 | 7 | 6 | 1 |

Table 21F shows that Arabidopsis plants containing b1093 (SEQ ID NO:343) targeted to mitochondria under control of the USP promoter and grown under water limiting conditions were significantly larger than the control plants that did not express b1093 (SEQ ID NO:343), at two measuring times. In addition, the transgenic plants were darker green in color than the controls. This indicates that the plants produced more chlorophyll or had less chlorophyll degradation during stress than the control plants. Table 21F shows that six of the seven independent transgenic events were larger than the controls indicating better adaptation to the stress environment.

The slr0886 gene (SEQ ID NO:345), which also encodes a 3-oxoacyl-ACP reductase, was expressed in Arabidopsis using three different constructs controlled by the PCUbi promoter: the constructs either had no subcellular targeting or they were targetted to the mitochondria or to the chloroplast. Table 22F sets forth biomass and health index data obtained from Arabidopsis plants transformed with these constructs and grown under water-limiting conditions, and Table 23F sets forth biomass and health index data for the untargeted construct under well-watered conditions.

TABLE 22F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| slr0886 | PCUbi | None | Biomass at | 38.5 | 0.0000 | 5 | 4 | 1 |
| | | | day 20 | | | | | |
| slr0886 | PCUbi | None | Biomass at | 20.9 | 0.0000 | 5 | 4 | 1 |
| | | | day 27 | | | | | |
| slr0886 | PCUbi | None | Health index | 10.0 | 0.0310 | 5 | 4 | 1 |
| slr0886 | PCUbi | Mit | Biomass at | 15.2 | 0.0014 | 5 | 5 | 0 |
| | | | day 20 | | | | | |
| slr0886 | PCUbi | Mit | Biomass at | 14.3 | 0.0000 | 5 | 4 | 1 |
| | | | day 27 | | | | | |
| slr0886 | PCUbi | Mit | Health index | 7.3 | NS | 5 | 3 | 2 |
| slr0886 | PCUbi | Chlor | Biomass at | 37.8 | 0.0000 | 6 | 6 | 0 |
| | | | day 20 | | | | | |
| slr0886 | PCUbi | Chlor | Biomass at | 11.4 | 0.0048 | 6 | 6 | 0 |
| | | | day 27 | | | | | |
| slr0886 | PCUbi | Chlor | Health index | 17.4 | 0.0000 | 6 | 5 | 1 |

Table 22F shows that all Arabidopsis plants expressing slr0886 (SEQ ID NO:345) that were grown under water limiting conditions were significantly larger than the control plants that did not express slr0886 (SEQ ID NO:345) at two measuring times. In addition, the transgenic plants were darker green in color than the controls. Table 22F shows that the majority of the independent transgenic events were larger than the controls, indicating better adaptation to the stress environment.

TABLE 23F

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| slr0886 | PCUbi | None | Biomass at | 20.4 | 0.0000 | 6 | 6 | 0 |
| | | | day 17 | | | | | |
| slr0886 | PCUbi | None | Biomass at | 12.3 | 0.0000 | 6 | 5 | 1 |
| | | | day 21 | | | | | |
| slr0886 | PCUbi | None | Health index | 5.2 | NS | 6 | 6 | 0 |

Table 23F shows that Arabidopsis plants expressing slr0886 (SEQ ID NO:345) with no subcellular targeting that were grown under well watered conditions were significantly larger than the control plants that did not express slr0886 (SEQ ID NO:345), at two measuring times.

### F. Biotin Synthetase

The slr1364 gene (SEQ ID NO:397), which encodes a biotin synthetase, was expressed in Arabidopsis using the PCUbi promoter with no subcellular targeting or with subcellular targeting to the mitochondria. Table 24F sets forth biomass and health index data obtained from Arabidopsis plants transformed with these constructs and grown under water-limiting conditions.

TABLE 24F

| | | | | | | | No. of | No. of |
|---|---|---|---|---|---|---|---|---|
| | | | | % | | No. of | Positive | Negative |
| Gene | Promoter | Targeting | Measurement | Change | p-Value | Events | Events | Events |
| slr1364 | PCUbi | None | Biomass at | 0.0 | NS | 5 | 1 | 4 |
| | | | 20 days | | | | | |
| slr1364 | PCUbi | None | Biomass at | −9.2 | 0.0048 | 5 | 1 | 4 |
| | | | 27 days | | | | | |
| slr1364 | PCUbi | None | Health Index | −1.8 | NS | 5 | 2 | 3 |
| slr1364 | PCUbi | Mit | Biomass at | 4.9 | 0.0223 | 6 | 6 | 0 |
| | | | 20 days | | | | | |
| slr1364 | PCUbi | Mit | Biomass at | 2.6 | NS | 6 | 3 | 3 |
| | | | 27 days | | | | | |
| slr1364 | PCUbi | Mit | Health Index | 6.3 | 0.0033 | 6 | 5 | 1 |

Table 24F shows that Arabidopsis plants that expressed slr1364 (SEQ ID NO:397) using the PCUbi promoter with subcellular targeting to the mitochondria were significantly larger under water limited conditions than the control plants that did not express slr1364 (SEQ ID NO:397) at two measuring times. Arabidopsis plants that expressed slr1364 (SEQ ID NO:397) with no subcellular targeting were smaller under water limited conditions than the control plants.

### Example 5

### Overexpression of Sterol Pathway Genes in Plants

The polynucleotides of Table 1G were ligated into an expression cassette using known methods. Three different promoters were used to control expression of the transgenes in Arabidopsis: the USP promoter from Vicia faba (SEQ ID NO:451 was used for expression of genes from E. coli or SEQ ID NO:452 was used for expression of genes from S. cerevisiae); the super promoter (SEQ ID NO:453); and the parsley ubiquitin promoter (SEQ ID NO:454). For selective targeting of the polypeptides, the mitochondrial transit peptide from an A. thaliana gene encoding mitochondrial isovaleryl-CoA dehydrogenase designated “Mit” in Tables 6G-9G. SEQ ID NO:456 was used for expression of genes from E. coli or SEQ ID NO:458 was used for expression of genes from S. cerevisiae. In addition, for targeted expression, the chloroplast transit peptide of an Spinacia oleracea gene encoding ferredoxin nitrite reductase designated “Chlor” in Tables 8G-9G (SEQ ID NO:460) was used.

The Arabidopsis ecotype C24 was transformed with constructs containing the sterol pathway genes described in Example 3 using known methods. Seeds from T2 transformed plants were pooled on the basis of the promoter driving the expression, gene source species and type of targeting (chloroplastic, mitochondrial and cytoplasmic). The seed pools were used in the primary screens for biomass under well watered and water limited growth conditions. Hits from pools in the primary screen were selected, molecular analysis performed and seed collected. The collected seeds were then used for analysis in secondary screens where a larger number of individuals for each transgenic event were analyzed. If plants from a construct were identified in the secondary screen as having increased biomass compared to the controls, it passed to the tertiary screen. In this screen, over 100 plants from all transgenic events for that construct were measured under well watered and drought growth conditions. The data from the transgenic plants were compared to wild type Arabidopsis plants or to plants grown from a pool of randomly selected transgenic Arabidopsis seeds using standard statistical procedures.

Plants that were grown under well watered conditions were watered to soil saturation twice a week. Images of the transgenic plants were taken at 17 and 21 days using a commercial imaging system. Alternatively, plants were grown under water limited growth conditions by watering to soil saturation infrequently which allowed the soil to dry between watering treatments. In these experiments, water was given on days 0, 8, and 19 after sowing. Images of the transgenic plants were taken at 20 and 27 days using a commercial imaging system.

Image analysis software was used to compare the images of the transgenic and control plants grown in the same experiment. The images were used to determine the relative size or biomass of the plants as pixels and the color of the plants as the ratio of dark green to total area. The latter ratio, termed the health index, was a measure of the relative amount of chlorophyll in the leaves and therefore the relative amount of leaf senescence or yellowing and was recorded at day 27 only. Variation exists among transgenic plants that contain the various sterol pathway genes, due to different sites of DNA insertion and other factors that impact the level or pattern of gene expression.

Tables 6G to 9G show the comparison of measurements of the Arabidopsis plants. Percent change indicates the measurement of the transgenic relative to the control plants as a percentage of the control non-transgenic plants; p value is the statistical significance of the difference between transgenic and control plants based on a T-test comparison of all independent events where NS indicates not significant at the 5% level of probabilty; No. of events indicates the total number of independent transgenic events tested in the experiment; No. of positive events indicates the total number of independent transgenic events that were larger than the control in the experiment; No. of negative events indicates the total number of independent transgenic events that were smaller than the control in the experiment. NS indicates not significant at the 5% level of probability.

a. Farnesyl Diphosphate Synthase (FPS)

The FPS designated B0421 (SEQ ID NO:414) was expressed in Arabidopsis using a construct wherein FPS expression is controlled by the USP promoter and the FPS protein is targeted to mitochondria. Table 6G sets forth biomass and health index data obtained from the Arabidopsis plants transformed with these constructs and tested under water-limiting conditions.

TABLE 6G

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| B0421 | USP | Mit | Biomass at | 18.8 | 0.0000 | 7 | 7 | 0 |
| | | | day 20 | | | | | |
| B0421 | USP | Mit | Biomass at | 11.4 | 0.0007 | 7 | 6 | 1 |
| | | | day 27 | | | | | |
| B0421 | USP | Mit | Health index | 12.6 | 0.0002 | 7 | 6 | 1 |

Table 6G shows that Arabidopsis plants expressing B0421 (SEQ ID NO:414) with targeting to mitochondria that were grown under water limiting conditions were significantly larger than the control plants that did not express B0421 (SEQ ID NO:414). In addition, these transgenic plants were darker green in color than the controls. These data indicate that the plants produced more chlorophyll or had less chlorophyll degradation during stress than the control plants. Table 6G also shows that the majority of independent transgenic events were larger than the controls.

The FPS designated YJL167W (SEQ ID NO:416) was expressed in Arabidopsis using a construct wherein FPS expression is controlled by the USP promoter and the FPS protein is targeted to mitochondria. Table 7G sets forth biomass and health index data obtained from Arabidopsis plants transformed with these constructs and tested under well-watered conditions.

TABLE 7G

| | | | | | | | No. of | No. of |
|---|---|---|---|---|---|---|---|---|
| | | | | % | | No. of | Positive | Negative |
| Gene | Promoter | Targeting | Measurement | Change | p-Value | Events | Events | Events |
| YJL167W | USP | Mit | Biomass at | 16.1 | 0.0000 | 6 | 6 | 0 |
| | | | day 17 | | | | | |
| YJL167W | USP | Mit | Biomass at | 9.7 | 0.0000 | 6 | 6 | 0 |
| | | | day 21 | | | | | |
| YJL167W | USP | Mit | Health index | 14.1 | 0.0095 | 6 | 4 | 2 |

Table 7G shows that Arabidopsis plants that were grown under well watered conditions were significantly larger than the control plants that did not express YJL167W (SEQ ID NO:416). Table 7G also shows that all independent transgenic events were larger than the controls in the well watered environment.

### B. Squalene Epoxidase

The YGR175C gene (SEQ ID NO:444), which encodes squalene epoxidase, was expressed in Arabidopsis using three constructs. In one, transcription is controlled by the PCUbi promoter and the protein translated from the resulting transcript is targeted to the chloroplast. Trancription in the other two constructs is controlled by the USP promoter. One of these USP promoter-containing constructs also has a chloroplast targeting sequence in operative association with the gene and the other construct has a mitochondrial targeting sequence in operative association with the gene. Table 8G sets forth biomass and health index data obtained from Arabidopsis plants transformed with these constructs and tested under water-limiting conditions.

TABLE 8G

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| YGR175C | PCUbi | Chlor | Biomass at | 38.2 | 0.0000 | 12 | 11 | 1 |
| | | | day 20 | | | | | |
| YGR175C | PCUbi | Chlor | Biomass at | 37.6 | 0.0000 | 12 | 12 | 0 |
| | | | day 27 | | | | | |
| YGR175C | PCUbi | Chlor | Health index | 13.9 | 0.0001 | 12 | 11 | 1 |
| YGR175C | USP | Chlor | Biomass at | 28.5 | 0.0000 | 8 | 7 | 1 |
| | | | day 20 | | | | | |
| YGR175C | USP | Chlor | Biomass at | 12.9 | 0.0089 | 8 | 5 | 3 |
| | | | day 27 | | | | | |
| YGR175C | USP | Chlor | Health index | 24.3 | 0.0000 | 8 | 8 | 0 |
| YGR175C | USP | Mit | Biomass at | −5.7 | NS | 6 | 2 | 4 |
| | | | day 20 | | | | | |
| YGR175C | USP | Mit | Biomass at | −8.0 | 0.0480 | 6 | 3 | 3 |
| | | | day 27 | | | | | |
| YGR175C | USP | Mit | Health index | 1.3 | NS | 6 | 5 | 1 |

Table 8G shows that Arabidopsis plants with the either the USP or PCUbi promoter controlling expression of YGR175C (SEQ ID NO:446) were significantly larger than the control plants when the protein was also targeted to the chloroplast. In addition, these transgenic plants were darker green in color than the controls. These data indicate that the plants produced more chlorophyll or had less chlorophyll degradation during stress than the control plants. Table 8G also shows that the majority of independent transgenic events were larger than the controls. In contrast, no increase in size or green color was observed for transgenic plants with a mitochondrial targeting sequence in operative association with YGR175C (SEQ ID NO:446). These observations suggest that the subcellular localization of the protein is important for conferring increased plant size and darker green color.

Table 9G sets forth biomass and health index data obtained from Arabidopsis plants transformed with these constructs and tested under well-watered conditions.

TABLE 9G

| | | | | | | | No of | No. of |
|---|---|---|---|---|---|---|---|---|
| | | | | % | | No. of | Positive | Negative |
| Gene | Promoter | Targeting | Measurement | Change | p-Value | Events | Events | Events |
| YGR175C | PCUbi | Chlor | Biomass at | 21.0 | 0.0000 | 10 | 9 | 1 |
| | | | day 17 | | | | | |
| YGR175C | PCUbi | Chlor | Biomass at | 17.7 | 0.0000 | 10 | 9 | 1 |
| | | | day 21 | | | | | |
| YGR175C | PCUbi | Chlor | Health index | 4.0 | NS | 10 | 5 | 5 |
| YGR175C | USP | Chlor | Biomass at | 5.1 | NS | 6 | 3 | 3 |
| | | | day 17 | | | | | |
| YGR175C | USP | Chlor | Biomass at | 3.5 | NS | 6 | 3 | 3 |
| | | | day 21 | | | | | |
| YGR175C | USP | Chlor | Health index | 7.1 | NS | 6 | 4 | 2 |
| YGR175C | USP | Mit | Biomass at | 7.9 | NS | 6 | 4 | 2 |
| | | | day 17 | | | | | |
| YGR175C | USP | Mit | Biomass at | 3.7 | NS | 6 | 4 | 2 |
| | | | day 21 | | | | | |
| YGR175C | USP | Mit | Health index | 3.7 | NS | 6 | 2 | 4 |

Table 9G shows that Arabidopsis plants grown under well-watered conditions with the either the PCUbi promoter controlling expression of YGR175C (SEQ ID NO:446) were significantly larger than the control plants when the protein was also targeted to the chloroplast. Table 9G also shows that the majority of independent transgenic events were larger than the controls when the PCUbi promoter/chloroplast transit peptide combination was present in the construct used for transformation. In contrast, no increase in size was observed for transgenic plants with the USP promoter controlling transcription of the transgene, when the plants were grown under well-watered conditions. In addition, none of these constructs had a significant effect on the amount of green color of the plants when grown under well-watered conditions. These observations indicate the importance of expression level and subcellular targeting to create the increased growth phenotype under either well watered or water limiting growth conditions.

### Example 6

### Well-Watered Arabidopsis Plants

The polynucleotides of Table 1 are ligated into a binary vector containing a selectable marker. The resulting recombinant vector contains the corresponding gene in the sense orientation under a constitutive promoter. The recombinant vectors are transformed into an Agrobacterium tumefaciens strain according to standard conditions. A. thaliana ecotype Col-0 or C24 are grown and transformed according to standard conditions. T1 and T2 plants are screened for resistance to the selection agent conferred by the selectable marker gene. T3 seeds are used in greenhouse or growth chamber experiments. Approximately 3-5 days prior to planting, seeds are refrigerated for stratification. Seeds are then planted, fertilizer is applied and humidity is maintained using transparent domes. Plants are grown in a greenhouse at 22 C with photoperiod of 16 hours light/8 hours dark. Plants are watered twice a week.

At 19 and 22 days, plant area, leaf area, biomass, color distribution, color intensity, and growth rate for each plant are measured using a commercially available imaging system. Biomass is calculated as the total plant leaf area at the last measuring time point. Growth rate is calculated as the plant leaf area at the last measuring time point minus the plant leaf area at the first measuring time point divided by the plant leaf area at the first measuring time point. Health index is calculated as the dark green leaf area divided by the total plant leaf area.

### Example 7

### Water Stress Tolerant Arabidopsis Plants

The polynucleotides of Table 1 are ligated into a binary vector containing a selectable marker. The resulting recombinant vector contains the corresponding gene in the sense orientation under a constitutive promoter. The recombinant vectors are transformed into an Agrobacterium tumefaciens strain according to standard conditions. A. thaliana ecotype Col-0 or C24 are grown and transformed according to standard conditions. T1 and T2 plants are screened for resistance to the selection agent conferred by the selectable marker gene, and positive plants were transplanted into soil and grown in a growth chamber for 3 weeks. Soil moisture was maintained throughout this time at approximately 50% of the maximum water-holding capacity of soil.

The total water lost (transpiration) by the plant during this time was measured. After 3 weeks, the entire above-ground plant material was collected, dried at 65 C for 2 days and weighed. The ratio of above-ground plant dry weight (DW) to plant water use is water use efficiency (WUE). Tables 52A through 64A, Tables 25D and 26D, Tables 19E through 24E present WUE and DW for independent transformation events (lines) of transgenic plants overexpressing representative Mitogen activated protein kinase, calcium-dependent protein kinase, cyclin-dependent protein kinase and serine/threonine specific protein kinase polynucleotides of Table 1. Least square means (TR), percent improvement for the line (% Delta), and significant value (p-value) of a line compared to wild-type controls (WT) from an Analysis of Variance are presented. The percent improvement of each transgene-containing line as compared to wild-type control plants for WUE and DW is also presented/calculated.

TABLE 52A
DW analysis of A. thaliana lines overexpressing EST431 (SEQ ID NO: 4)

| Event | WT DW | TR DW | % | |
|---|---|---|---|---|
| ID | mean | mean | Delta | p-value |
| 1 | 0.098 | 0.102 | 4% | 0.8299 |
| 2 | 0.098 | 0.158 | 61% | 0.0053 |
| 3 | 0.098 | 0.094 | −5% | 0.8315 |
| 4 | 0.098 | 0.085 | −13% | 0.5566 |
| 5 | 0.098 | 0.083 | −16% | 0.4913 |
| 6 | 0.098 | 0.104 | 6% | 0.7769 |
| 7 | 0.098 | 0.094 | −5% | 0.806 |
| 8 | 0.098 | 0.107 | 9% | 0.6464 |
| 9 | 0.098 | 0.125 | 27% | 0.1644 |

TABLE 53A
WUE analysis of A. thaliana lines overexpressing EST431
(SEQ ID NO: 4)

| Event | WT WUE | TR WUE | % | |
|---|---|---|---|---|
| ID | mean | mean | Delta | p-value |
| 1 | 1.49 | 1.65 | 11% | 0.4566 |
| 2 | 1.49 | 2.33 | 56% | 0.0005 |
| 3 | 1.49 | 1.38 | −8% | 0.6575 |
| 4 | 1.49 | 1.38 | −7% | 0.6787 |
| 5 | 1.49 | 1.52 | 2% | 0.9083 |
| 6 | 1.49 | 1.67 | 12% | 0.4102 |
| 7 | 1.49 | 1.58 | 6% | 0.671 |
| 8 | 1.49 | 1.65 | 11% | 0.4698 |
| 9 | 1.49 | 1.69 | 13% | 0.3753 |

TABLE 54A
DW analysis of A. thaliana lines overexpressing EST253
(SEQ ID NO: 6)

| Event | WT DW | TR DW | % | |
|---|---|---|---|---|
| ID | mean | mean | Delta | p-value |
| 1 | 0.114 | 0.178 | 56% | 0.0006 |
| 2 | 0.114 | 0.183 | 61% | 0.0002 |
| 3 | 0.114 | 0.186 | 64% | 0.0003 |
| 4 | 0.114 | 0.172 | 50% | 0.0017 |
| 5 | 0.114 | 0.167 | 47% | 0.007 |
| 6 | 0.114 | 0.148 | 30% | 0.0587 |
| 7 | 0.114 | 0.185 | 62% | 0.0004 |
| 8 | 0.114 | 0.160 | 40% | 0.0115 |
| 9 | 0.114 | 0.164 | 44% | 0.0105 |

TABLE 55A
WUE analysis of A. thaliana lines overexpressing EST253
(SEQ ID NO: 6)

| Event | WT WUE | TR WUE | % | |
|---|---|---|---|---|
| ID | mean | mean | Delta | p-value |
| 1 | 1.96 | 2.30 | 17% | 0.0412 |
| 2 | 1.96 | 2.16 | 10% | 0.2303 |
| 3 | 1.96 | 2.32 | 18% | 0.0469 |
| 4 | 1.96 | 2.28 | 16% | 0.0574 |
| 5 | 1.96 | 2.22 | 13% | 0.1446 |
| 6 | 1.96 | 2.04 | 4% | 0.6433 |
| 7 | 1.96 | 2.26 | 15% | 0.0986 |
| 8 | 1.96 | 2.17 | 11% | 0.1991 |
| 9 | 1.96 | 2.02 | 3% | 0.7458 |

TABLE 56A
DW analysis of A. thaliana lines overexpressing EST272
(SEQ ID NO: 30).

| Event | WT DW | TR DW | | |
|---|---|---|---|---|
| ID | mean | mean | % Delta | p-value |
| 1 | 0.1779 | 0.2223 | 25% | 0.0928 |
| 2 | 0.1779 | 0.2608 | 47% | 0.0007 |
| 3 | 0.1779 | 0.284 | 60% | 0.0001 |
| 4 | 0.1779 | 0.2898 | 63% | <0.0001 |
| 5 | 0.1779 | 0.2483 | 40% | 0.0085 |
| 6 | 0.1779 | 0.2518 | 42% | 0.0024 |
| 7 | 0.1779 | 0.1997 | 12% | 0.4674 |
| 8 | 0.1779 | 0.2486 | 40% | 0.0035 |
| 9 | 0.1779 | 0.2422 | 36% | 0.0077 |
| 10 | 0.1779 | 0.255 | 43% | 0.0015 |

TABLE 57A
WUE analysis of A. thaliana lines overexpressing EST272
(SEQ ID NO: 30).

| Event | WT WUE | TR WUE | | |
|---|---|---|---|---|
| ID | mean | mean | % Delta | p-value |
| 1 | 1.8947 | 2.0651 | 9% | 0.3094 |
| 2 | 1.8947 | 2.0777 | 10% | 0.2271 |
| 3 | 1.8947 | 2.253 | 19% | 0.0344 |
| 4 | 1.8947 | 2.1471 | 13% | 0.0971 |
| 5 | 1.8947 | 1.9713 | 4% | 0.6467 |
| 6 | 1.8947 | 1.958 | 3% | 0.6748 |
| 7 | 1.8947 | 1.8884 | 0% | 0.9738 |
| 8 | 1.8947 | 2.0853 | 10% | 0.2086 |
| 9 | 1.8947 | 2.0011 | 6% | 0.4812 |
| 10 | 1.8947 | 2.466 | 30% | 0.0003 |

TABLE 58A
DW analysis of A. thaliana lines overexpressing
EST591 (SEQ ID NO: 62)

| Event | WT DW | TR DW | | |
|---|---|---|---|---|
| ID | mean | mean | % Delta | p-value |
| 1 | 0.114 | 0.0744 | −35% | 0.0272 |
| 11 | 0.114 | 0.128 | 27% | 0.3893 |
| 14 | 0.114 | 0.1552 | 31% | 0.0215 |
| 15 | 0.114 | 0.197 | 71% | 0.0029 |
| 17 | 0.114 | 0.1974 | 31% | <.0001 |
| 2 | 0.114 | 0.1444 | 51% | 0.0875 |
| 3 | 0.114 | 0.1488 | 12% | 0.0511 |
| 5 | 0.114 | 0.1949 | 36% | <.0001 |
| 6 | 0.114 | 0.149 | 73% | 0.0498 |
| 8 | 0.114 | 0.1724 | 73% | 0.0013 |

TABLE 59A
WUE analysis of A. thaliana lines overexpressing
EST591 (SEQ ID NO: 62)

| | | TR | | |
|---|---|---|---|---|
| Event | WT WUE | WUE | | |
| ID | mean | mean | % Delta | p-value |
| 1 | 1.9696 | 1.7367 | −11% | 0.1758 |
| 2 | 1.9696 | 2.0929 | 7% | 0.472 |
| 3 | 1.9696 | 2.4553 | 25% | 0.0055 |
| 5 | 1.9696 | 2.3519 | 20% | 0.0108 |
| 6 | 1.9696 | 2.0568 | 5% | 0.6109 |
| 8 | 1.9696 | 2.124 | 8% | 0.3682 |
| 11 | 1.9696 | 1.8794 | −4% | 0.5673 |
| 14 | 1.9696 | 2.2768 | 16% | 0.0753 |
| 15 | 1.9696 | 2.1498 | 10% | 0.4941 |
| 17 | 1.9696 | 2.1415 | 9% | 0.3167 |

TABLE 60A
DW analysis of A. thaliana lines overexpressing
EST500 (SEQ ID NO: 42)

| Event | WT | TR | % | |
|---|---|---|---|---|
| ID | mean | mean | Delta | p-value |
| 1 | 0.091 | 0.121 | 33% | 0.3656 |
| 2 | 0.091 | 0.131 | 44% | 0.2757 |
| 3 | 0.091 | 0.114 | 26% | 0.4848 |
| 4 | 0.091 | 0.148 | 63% | 0.1002 |
| 5 | 0.091 | 0.152 | 67% | 0.0739 |
| 6 | 0.091 | 0.169 | 86% | 0.025 |
| 7 | 0.091 | 0.150 | 65% | 0.0842 |
| 8 | 0.091 | 0.154 | 70% | 0.0634 |
| 9 | 0.091 | 0.098 | 8% | 0.8416 |
| 10 | 0.091 | 0.113 | 24% | 0.5393 |
| 11 | 0.091 | 0.108 | 18% | 0.7555 |

TABLE 61A
WUE analysis of A. thaliana lines overexpressing
EST500 (SEQ ID NO: 42)

| Event | WT | TR | % | |
|---|---|---|---|---|
| ID | mean | mean | Delta | p-value |
| 1 | 1.92 | 1.82 | −5% | 0.743 |
| 2 | 1.92 | 2.66 | 39% | 0.0261 |
| 3 | 1.92 | 2.42 | 26% | 0.0948 |
| 4 | 1.92 | 2.33 | 21% | 0.1925 |
| 5 | 1.92 | 2.25 | 17% | 0.2665 |
| 6 | 1.92 | 2.27 | 19% | 0.2374 |
| 7 | 1.92 | 2.17 | 13% | 0.4063 |
| 8 | 1.92 | 2.11 | 10% | 0.5302 |
| 9 | 1.92 | 1.71 | −11% | 0.5171 |
| 10 | 1.92 | 1.82 | −5% | 0.7606 |
| 11 | 1.92 | 1.67 | −13% | 0.6203 |

TABLE 62A
DW analysis of A. thaliana lines overexpressing
EST401 (SEQ ID NO: 44)

| Event | WT DW | TR DW | % | |
|---|---|---|---|---|
| ID | mean | mean | Delta | p-value |
| 2 | 0.110 | 0.147 | 33% | 0.007 |
| 3 | 0.110 | 0.156 | 41% | 0.0008 |
| 4 | 0.110 | 0.137 | 24% | 0.0466 |
| 5 | 0.110 | 0.132 | 20% | 0.1048 |
| 6 | 0.110 | 0.137 | 24% | 0.045 |
| 7 | 0.110 | 0.125 | 13% | 0.2645 |
| 8 | 0.110 | 0.117 | 6% | 0.6177 |
| 9 | 0.110 | 0.141 | 28% | 0.0405 |
| 10 | 0.110 | 0.140 | 27% | 0.0272 |
| 11 | 0.110 | 0.124 | 13% | 0.2979 |

TABLE 63A
WUE analysis of A. thaliana lines overexpressing
EST401 (SEQ ID NO: 44)

| Event | WT WUE | TR WUE | % | |
|---|---|---|---|---|
| ID | mean | mean | Delta | p-value |
| 2 | 1.62 | 2.05 | 27% | 0.0439 |
| 3 | 1.62 | 2.06 | 27% | 0.0386 |
| 4 | 1.62 | 2.08 | 29% | 0.0303 |
| 5 | 1.62 | 1.87 | 16% | 0.2362 |
| 6 | 1.62 | 1.92 | 18% | 0.1607 |
| 7 | 1.62 | 2.00 | 23% | 0.078 |
| 8 | 1.62 | 1.88 | 16% | 0.2145 |
| 9 | 1.62 | 2.04 | 26% | 0.0739 |
| 10 | 1.62 | 2.31 | 42% | 0.0014 |
| 11 | 1.62 | 2.19 | 35% | 0.0078 |

TABLE 64A
DW analysis of A. thaliana lines overexpressing
EST336 (SEQ ID NO: 82)

| Event | WT DW | TR DW | | |
|---|---|---|---|---|
| ID | mean | mean | % Delta | p-value |
| 1 | 0.114 | 0.1758 | 54% | 0.0032 |
| 2 | 0.114 | 0.1724 | 51% | 0.0052 |
| 3 | 0.114 | 0.2143 | 88% | <.0001 |
| 4 | 0.114 | 0.1608 | 41% | 0.0145 |
| 5 | 0.114 | 0.1516 | 33% | 0.0684 |
| 6 | 0.114 | 0.1492 | 31% | 0.0876 |
| 7 | 0.114 | 0.1412 | 24% | 0.1855 |
| 8 | 0.114 | 0.15 | 32% | 0.0585 |
| 9 | 0.114 | 0.157 | 38% | 0.0377 |

TABLE 25D
DW analysis of A. thaliana lines overexpressing
EST285 (SEQ ID NO: 208)

| Event | WT DW | TR DW | % | |
|---|---|---|---|---|
| ID | mean | mean | Delta | p-value |
| 1 | 0.110 | 0.103 | −7% | 0.6618 |
| 2 | 0.110 | 0.108 | −3% | 0.8751 |
| 3 | 0.110 | 0.129 | 17% | 0.2879 |
| 4 | 0.110 | 0.161 | 45% | 0.0059 |
| 5 | 0.110 | 0.076 | −32% | 0.0797 |
| 6 | 0.110 | 0.159 | 44% | 0.008 |
| 7 | 0.110 | 0.144 | 31% | 0.059 |
| 8 | 0.110 | 0.110 | −1% | 0.9642 |
| 9 | 0.110 | 0.171 | 55% | 0.0011 |
| 10 | 0.110 | 0.110 | 0% | 0.9838 |

TABLE 26D
WUE analysis of A. thaliana lines overexpressing
EST285 (SEQ ID NO: 208)

| Event | WT WUE | TR WUE | % | |
|---|---|---|---|---|
| ID | mean | mean | Delta | p-value |
| 1 | 1.62 | 1.65 | 2% | 0.8855 |
| 2 | 1.62 | 1.97 | 22% | 0.1046 |
| 3 | 1.62 | 2.27 | 40% | 0.0033 |
| 4 | 1.62 | 1.93 | 19% | 0.1536 |
| 5 | 1.62 | 1.37 | −15% | 0.3083 |
| 6 | 1.62 | 1.94 | 20% | 0.1378 |
| 7 | 1.62 | 1.87 | 16% | 0.2491 |
| 8 | 1.62 | 1.72 | 6% | 0.6425 |
| 9 | 1.62 | 2.11 | 30% | 0.027 |
| 10 | 1.62 | 1.75 | 8% | 0.6 |

TABLE 19E
DW analysis of A. thaliana lines overexpressing
EST314 (SEQ ID NO: 254)

| Event | WT DW | TR DW | | |
|---|---|---|---|---|
| ID | mean | mean | % Delta | p-value |
| 1 | 0.114 | 0.1648 | 45% | 0.0057 |
| 2 | 0.114 | 0.1564 | 37% | 0.0202 |
| 3 | 0.114 | 0.14 | 23% | 0.1502 |
| 4 | 0.114 | 0.157 | 38% | 0.0185 |
| 5 | 0.114 | 0.1422 | 25% | 0.119 |
| 6 | 0.114 | 0.1452 | 27% | 0.0851 |
| 7 | 0.114 | 0.1652 | 45% | 0.0053 |
| 8 | 0.114 | 0.1488 | 31% | 0.0553 |
| 9 | 0.114 | 0.176 | 54% | 0.0008 |
| 11 | 0.114 | 0.1784 | 56% | 0.0005 |

TABLE 20E
WUE analysis A. thaliana lines overexpressing
EST314 (SEQ ID NO: 254).

| Event | WT WUE | TR WUE | | |
|---|---|---|---|---|
| ID | mean | mean | % Delta | p-value |
| 1 | 1.9696 | 2.4723 | 26% | 0.0078 |
| 2 | 1.9696 | 2.2242 | 13% | 0.1718 |
| 3 | 1.9696 | 2.155 | 9% | 0.3185 |
| 4 | 1.9696 | 2.0887 | 6% | 0.5209 |
| 5 | 1.9696 | 1.9933 | 1% | 0.8983 |
| 6 | 1.9696 | 2.2717 | 15% | 0.1056 |
| 7 | 1.9696 | 2.001 | 2% | 0.8656 |
| 8 | 1.9696 | 1.9265 | −2% | 0.816 |
| 9 | 1.9696 | 2.3454 | 19% | 0.0449 |
| 11 | 1.9696 | 2.2909 | 16% | 0.0856 |

TABLE 21E
DW analysis of A. thaliana lines overexpressing
EST322 (SEQ ID NO: 256)

| Event | WT DW | TR DW | | |
|---|---|---|---|---|
| ID | mean | mean | % Delta | p-value |
| 1 | 0.1089 | 0.1355 | 24% | 0.1052 |
| 2 | 0.1089 | 0.0838 | −23% | 0.1568 |
| 3 | 0.1089 | 0.1884 | 73% | <.0001 |
| 4 | 0.1089 | 0.1033 | −5% | 0.8019 |
| 5 | 0.1089 | 0.048 | −56% | 0.0266 |
| 6 | 0.1089 | 0.1788 | 64% | 0.0006 |
| 7 | 0.1089 | 0.1743 | 60% | 0.0001 |
| 8 | 0.1089 | 0.1422 | 31% | 0.0436 |
| 9 | 0.1089 | 0.1518 | 39% | 0.0307 |
| 10 | 0.1089 | 0.147 | 35% | 0.0334 |

TABLE 22E
WUE analysis of A. thaliana lines overexpressing
EST322 (SEQ ID NO: 256)

| Event | WT WUE | TR WUE | | |
|---|---|---|---|---|
| ID | mean | mean | % Delta | p-value |
| 1 | 1.9868 | 1.8144 | −9% | 0.3609 |
| 2 | 1.9868 | 1.5181 | −24% | 0.0239 |
| 3 | 1.9868 | 2.183 | 10% | 0.3381 |
| 4 | 1.9868 | 1.628 | −18% | 0.1674 |
| 5 | 1.9868 | 0.9151 | −54% | 0.0009 |
| 6 | 1.9868 | 2.4043 | 21% | 0.0676 |
| 7 | 1.9868 | 2.2196 | 12% | 0.2183 |
| 8 | 1.9868 | 1.9381 | −2% | 0.7956 |
| 9 | 1.9868 | 1.8251 | −8% | 0.4752 |
| 10 | 1.9868 | 1.7922 | −10% | 0.342 |

TABLE 23E
DW analysis of A. thaliana lines overexpressing
EST589 (SEQ ID NO: 258)

| Event | WT DW | TR DW | | |
|---|---|---|---|---|
| ID | mean | mean | % Delta | p-value |
| 1 | 0.09376 | 0.1122 | 20% | 0.5855 |
| 2 | 0.09376 | 0.0808 | −14% | 0.7064 |
| 3 | 0.09376 | 0.1223 | 30% | 0.4131 |
| 4 | 0.09376 | 0.1011 | 8% | 0.8305 |
| 5 | 0.09376 | 0.1061 | 13% | 0.7196 |
| 6 | 0.09376 | 0.07416 | −21% | 0.5732 |
| 7 | 0.09376 | 0.0911 | −3% | 0.9378 |
| 8 | 0.09376 | 0.1018 | 9% | 0.8147 |
| 9 | 0.09376 | 0.09155 | −2% | 0.9484 |
| 10 | 0.09376 | 0.1457 | 55% | 0.2354 |

TABLE 24E
WUE analysis of A. thaliana lines overexpressing
EST589 (SEQ ID NO: 258)

| Event | WT WUE | TR WUE | | |
|---|---|---|---|---|
| ID | mean | mean | % Delta | p-value |
| 1 | 1.5808 | 1.6999 | 24% | 0.5956 |
| 2 | 1.5808 | 1.4025 | 3% | 0.4551 |
| 3 | 1.5808 | 1.7463 | 28% | 0.4872 |
| 4 | 1.5808 | 1.6957 | 24% | 0.6275 |
| 5 | 1.5808 | 1.5321 | 12% | 0.8363 |
| 6 | 1.5808 | 1.4906 | 9% | 0.7074 |
| 7 | 1.5808 | 1.6152 | 18% | 0.8821 |
| 8 | 1.5808 | 1.6083 | 18% | 0.907 |
| 9 | 1.5808 | 1.5863 | 16% | 0.9811 |
| 10 | 1.5808 | 1.6231 | 19% | 0.8846 |

### Example 8

### Nitrogen Stress Tolerant Arabidopsis Plants

The polynucleotides of Table 1 are ligated into a binary vector containing a selectable marker. The resulting recombinant vector contains the corresponding gene in the sense orientation under a constitutive promoter. The recombinant vectors are transformed into an A. tumefaciens strain according to standard conditions. A. thaliana ecotype Col-0 or C24 are grown and transformed according to standard conditions. T1 and T2 plants are screened for resistance to the selection agent conferred by the selectable marker gene. Plants are grown in flats using a substrate that contains no organic components. Each flat is wet with water before seedlings resistant to the selection agent are transplanted onto substrate. Plants are grown in a growth chamber set to 22 C with a 55% relative humidity with photoperiod set at 16 h light/8 h dark. A controlled low or high nitrogen nutrient solution is added to waterings on Days 12, 15, 22 and 29. Watering without nutrient solution occurs on Days 18, 25, and 32. Images of all plants in a tray are taken on days 26, 30, and 33 using a commercially available imaging system. At each imaging time point, biomass and plant phenotypes for each plant are measured including plant area, leaf area, biomass, color distribution, color intensity, and growth rate.

### Example 9

### Stress-Tolerant Rapeseed/Canola Plants

Canola cotyledonary petioles of 4 day-old young seedlings are used as explants for tissue culture and transformed according to EP1566443, the contents of which are hereby incorporated by reference. The commercial cultivar Westar (Agriculture Canada) is the standard variety used for transformation, but other varieties can be used. A. tumefaciens GV3101:pMP90RK containing a binary vector is used for canola transformation. The standard binary vector used for transformation is pSUN (WO02/00900), but many different binary vector systems have been described for plant transformation (e.g. An, G. in Agrobacterium Protocols, Methods in Molecular Biology vol 44, pp 47-62, Gartland K M A and M R Davey eds. Humana Press, Totowa, N.J.). A plant gene expression cassette comprising a selection marker gene, a plant promoter, and a polynucleotide of Table 1 is employed. Various selection marker genes can be used including the mutated acetohydroxy acid synthase (AHAS) gene disclosed in U.S. Pat. Nos. 5,767,366 and 6,225,105. A suitable promoter is used to regulate the trait gene to provide constitutive, developmental, tissue or environmental regulation of gene transcription. Seed is produced from the primary transgenic plants by self-pollination. The second-generation plants are grown in greenhouse conditions and self-pollinated. The plants are analyzed to confirm the presence of T-DNA and to determine the number of T-DNA integrations. Homozygous transgenic, heterozygous transgenic and azygous (null transgenic) plants are compared for their stress tolerance, for example, in the assays described in Examples 6 and 7, and for yield, both in the greenhouse and in field studies.

### Example 10

### Screening for Stress-Tolerant Rice Plants

Transgenic rice plants comprising a polynucleotide of Table 1 are generated using known methods. Approximately 15 to 20 independent transformants (T0) are generated. The primary transformants are transferred from tissue culture chambers to a greenhouse for growing and harvest of T1 seeds. Five events of the T1 progeny segregated 3:1 for presence/absence of the transgene are retained. For each of these events, 10 T1 seedlings containing the transgene (hetero- and homozygotes), and 10 T1 seedlings lacking the transgene (nullizygotes) are selected by visual marker screening. The selected T1 plants are transferred to a greenhouse. Each plant receives a unique barcode label to link unambiguously the phenotyping data to the corresponding plant. The selected T1 plants are grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Transgenic plants and the corresponding nullizygotes are grown side-by-side at random positions. From the stage of sowing until the stage of maturity, the plants are passed several times through a digital imaging cabinet. At each time point digital, images (2048×1536 pixels, 16 million colours) of each plant are taken from at least 6 different angles.

The data obtained in the first experiment with T1 plants are confirmed in a second experiment with T2 plants. Lines that have the correct expression pattern are selected for further analysis. Seed batches from the positive plants (both hetero- and homozygotes) in T1 are screened by monitoring marker expression. For each chosen event, the heterozygote seed batches are then retained for T2 evaluation. Within each seed batch, an equal number of positive and negative plants are grown in the greenhouse for evaluation.

Transgenic plants are screened for their improved growth and/or yield and/or stress tolerance, for example, using the assays described in Examples 6 and 7, and for yield, both in the greenhouse and in field studies.

### Example 11

### Stress-Tolerant Soybean Plants

The polynucleotides of Table 1 are transformed into soybean using the methods described in commonly owned copending international application number WO 2005/121345, the contents of which are incorporated herein by reference.

The transgenic plants generated are then screened for their improved growth under water-limited conditions and/or drought, salt, and/or cold tolerance, for example, using the assays described in Examples 6 and 7, and for yield, both in the greenhouse and in field studies.

### Example 12

### Stress-Tolerant Wheat Plants

The polynucleotides of Table 1 are transformed into wheat using the method described by Ishida et al., 1996, Nature Biotech. 14745-50. Immature embryos are co-cultivated with Agrobacterium tumefaciens that carry “super binary” vectors, and transgenic plants are recovered through organogenesis. This procedure provides a transformation efficiency between 2.5% and 20%. The transgenic plants are then screened for their improved growth and/or yield under water-limited conditions and/or stress tolerance, for example, is the assays described in Examples 6 and 7, and for yield, both in the greenhouse and in field studies.

### Example 13

### Stress-Tolerant Corn Plants

The polynucleotides of Table 1 are transformed into immature embryos of corn using Agrobacterium. After imbibition, embryos are transferred to medium without selection agent. Seven to ten days later, embryos are transferred to medium containing selection agent and grown for 4 weeks (two 2-week transfers) to obtain transformed callus cells. Plant regeneration is initiated by transferring resistant calli to medium supplemented with selection agent and grown under light at 25-27° C. for two to three weeks. Regenerated shoots are then transferred to rooting box with medium containing selection agent. Plantlets with roots are transferred to potting mixture in small pots in the greenhouse and after acclimatization are then transplanted to larger pots and maintained in greenhouse till maturity.

Using assays such as the assay described in Examples 6 and 7, each of these plants is uniquely labeled, sampled and analyzed for transgene copy number. Transgene positive and negative plants are marked and paired with similar sizes for transplanting together to large pots. This provides a uniform and competitive environment for the transgene positive and negative plants. The large pots are watered to a certain percentage of the field water capacity of the soil depending the severity of water-stress desired. The soil water level is maintained by watering every other day. Plant growth and physiology traits such as height, stem diameter, leaf rolling, plant wilting, leaf extension rate, leaf water status, chlorophyll content and photosynthesis rate are measured during the growth period. After a period of growth, the above ground portion of the plants is harvested, and the fresh weight and dry weight of each plant are taken. A comparison of the drought tolerance phenotype between the transgene positive and negative plants is then made.

Using assays such as the assay described in Example 6 and 7, the pots are covered with caps that permit the seedlings to grow through but minimize water loss. Each pot is weighed periodically and water added to maintain the initial water content. At the end of the experiment, the fresh and dry weight of each plant is measured, the water consumed by each plant is calculated and WUE of each plant is computed. Plant growth and physiology traits such as WUE, height, stem diameter, leaf rolling, plant wilting, leaf extension rate, leaf water status, chlorophyll content and photosynthesis rate are measured during the experiment. A comparison of WUE phenotype between the transgene positive and negative plants is then made.

Using assays such as the assay described in Example 6 and 7, these pots are kept in an area in the greenhouse that has uniform environmental conditions, and cultivated optimally. Each of these plants is uniquely labeled, sampled and analyzed for transgene copy number. The plants are allowed to grow under theses conditions until they reach a predefined growth stage. Water is then withheld. Plant growth and physiology traits such as height, stem diameter, leaf rolling, plant wilting, leaf extension rate, leaf water status, chlorophyll content and photosynthesis rate are measured as stress intensity increases. A comparison of the dessication tolerance phenotype between transgene positive and negative plants is then made.

Segregating transgenic corn seeds for a transformation event are planted in small pots for testing in a cycling drought assay. These pots are kept in an area in the greenhouse that has uniform environmental conditions, and cultivated optimally. Each of these plants is uniquely labeled, sampled and analyzed for transgene copy number. The plants are allowed to grow under theses conditions until they reach a predefined growth stage. Plants are then repeatedly watered to saturation at a fixed interval of time. This water/drought cycle is repeated for the duration of the experiment. Plant growth and physiology traits such as height, stem diameter, leaf rolling, leaf extension rate, leaf water status, chlorophyll content and photosynthesis rate are measured during the growth period. At the end of the experiment, the plants are harvested for above-ground fresh and dry weight. A comparison of the cycling drought tolerance phenotype between transgene positive and negative plants is then made.

In order to test segregating transgenic corn for drought tolerance under rain-free conditions, managed-drought stress at a single location or multiple locations is used. Crop water availability is controlled by drip tape or overhead irrigation at a location which has less than 10 cm rainfall and minimum temperatures greater than 5° C. expected during an average 5 month season, or a location with expected in-season precipitation intercepted by an automated “rain-out shelter” which retracts to provide open field conditions when not required. Standard agronomic practices in the area are followed for soil preparation, planting, fertilization and pest control. Each plot is sown with seed segregating for the presence of a single transgenic insertion event. A Taqman transgene copy number assay is used on leaf samples to differentiate the transgenics from null-segregant control plants. Plants that have been genotyped in this manner are also scored for a range of phenotypes related to drought-tolerance, growth and yield. These phenotypes include plant height, grain weight per plant, grain number per plant, ear number per plant, above ground dry-weight, leaf conductance to water vapor, leaf CO₂ uptake, leaf chlorophyll content, photosynthesis-related chlorophyll fluorescence parameters, water use efficiency, leaf water potential, leaf relative water content, stem sap flow rate, stem hydraulic conductivity, leaf temperature, leaf reflectance, leaf light absorptance, leaf area, days to flowering, anthesis-silking interval, duration of grain fill, osmotic potential, osmotic adjustment, root size, leaf extension rate, leaf angle, leaf rolling and survival. All measurements are made with commercially available instrumentation for field physiology, using the standard protocols provided by the manufacturers. Individual plants are used as the replicate unit per event.

In order to test non-segregating transgenic corn for drought tolerance under rain-free conditions, managed-drought stress at a single location or multiple locations is used. Crop water availability is controlled by drip tape or overhead irrigation at a location which has less than 10 cm rainfall and minimum temperatures greater than 5° C. expected during an average 5 month season, or a location with expected in-season precipitation intercepted by an automated “rain-out shelter” which retracts to provide open field conditions when not required. Standard agronomic practices in the area are followed for soil preparation, planting, fertilization and pest control. Trial layout is designed to pair a plot containing a non-segregating transgenic event with an adjacent plot of null-segregant controls. A null segregant is progeny (or lines derived from the progeny) of a transgenic plant that does not contain the transgene due to Mendelian segregation. Additional replicated paired plots for a particular event are distributed around the trial. A range of phenotypes related to drought-tolerance, growth and yield are scored in the paired plots and estimated at the plot level. When the measurement technique could only be applied to individual plants, these are selected at random each time from within the plot. These phenotypes include plant height, grain weight per plant, grain number per plant, ear number per plant, above ground dry-weight, leaf conductance to water vapor, leaf CO₂ uptake, leaf chlorophyll content, photosynthesis-related chlorophyll fluorescence parameters, water use efficiency, leaf water potential, leaf relative water content, stem sap flow rate, stem hydraulic conductivity, leaf temperature, leaf reflectance, leaf light absorptance, leaf area, days to flowering, anthesis-silking interval, duration of grain fill, osmotic potential, osmotic adjustment, root size, leaf extension rate, leaf angle, leaf rolling and survival. All measurements are made with commercially available instrumentation for field physiology, using the standard protocols provided by the manufacturers. Individual plots are used as the replicate unit per event.

To perform multi-location testing of transgenic corn for drought tolerance and yield, five to twenty locations encompassing major corn growing regions are selected. These are widely distributed to provide a range of expected crop water availabilities based on average temperature, humidity, precipitation and soil type. Crop water availability is not modified beyond standard agronomic practices. Trial layout is designed to pair a plot containing a non-segregating transgenic event with an adjacent plot of null-segregant controls. A range of phenotypes related to drought-tolerance, growth and yield are scored in the paired plots and estimated at the plot level. When the measurement technique could only be applied to individual plants, these are selected at random each time from within the plot. These phenotypes included plant height, grain weight per plant, grain number per plant, ear number per plant, above ground dry-weight, leaf conductance to water vapor, leaf CO₂ uptake, leaf chlorophyll content, photosynthesis-related chlorophyll fluorescence parameters, water use efficiency, leaf water potential, leaf relative water content, stem sap flow rate, stem hydraulic conductivity, leaf temperature, leaf reflectance, leaf light absorptance, leaf area, days to flowering, anthesis-silking interval, duration of grain fill, osmotic potential, osmotic adjustment, root size, leaf extension rate, leaf angle, leaf rolling and survival. All measurements are made with commercially available instrumentation for field physiology, using the standard protocols provided by the manufacturers. Individual plots are used as the replicate unit per event.

### BRIEF DESCRIPTION OF THE DRAWINGS

FIG. 1 shows an alignment of the disclosed amino acid sequences of mitogen activated protein kinases GM471-43343 (SEQ ID NO:2), EST431 (SEQ ID NO:4), and EST253 (SEQ ID NO:6), TA54298452 (SEQ ID NO:8), GM59742369 (SEQ ID NO:10), LU61585372 (SEQ ID NO:12), BN44703759 (SEQ ID NO:14), GM59703946 (SEQ ID NO:16), GM59589775 (SEQ ID NO:18), LU61696985 (SEQ ID NO:20), ZM62001130 (SEQ ID NO:22), HA66796355 (SEQ ID NO:24), LU61684898 (SEQ ID NO:26), LU61597381 (SEQ ID NO:28), EST272 (SEQ ID NO:30), BN42920374 (SEQ ID NO:32), BN45700248 (SEQ ID NO:34), BN47678601 (SEQ ID NO:36), and GMsj02a06 (SEQ ID NO:38). The alignment was generated using Align X of Vector NTI.

FIG. 2 shows an alignment of the disclosed amino acid sequences of calcium-dependent protein kinases GM50305602 (SEQ ID NO:40), EST500 (SEQ ID NO:42), and EST401 (SEQ ID NO:44), BN51391539 (SEQ ID NO:46), GM59762784 (SEQ ID NO:48), BN44099508 (SEQ ID NO:50), BN45789913 (SEQ ID NO:52), BN47959187 (SEQ ID NO:54), BN51418316 (SEQ ID NO:56), GM59691587 (SEQ ID NO:58), ZM62219224 (SEQ ID NO:60), EST591 (SEQ ID NO:62), BN51345938 (SEQ ID NO:64), BN51456960 (SEQ ID NO:66), BN43562070 (SEQ ID NO:68), TA60004809 (SEQ ID NO:70), ZM62079719 (SEQ ID NO:72). The alignment was generated using Align X of Vector NTI.

FIG. 3 shows an alignment of the disclosed amino acid sequences of cyclin-dependent protein kinases BN42110642 (SEQ ID NO:74), GM59794180 (SEQ ID NO:76), GMsp52b07 (SEQ ID NO:78), and ZM57272608 (SEQ ID NO:80). The alignment was generated using Align X of Vector NTI.

FIG. 4 shows an alignment of the disclosed amino acid sequences of serine/threonine specific protein kinases EST336 (SEQ ID NO:82), BN43012559 (SEQ ID NO:84), BN44705066 (SEQ ID NO:86), GM50962576 (SEQ ID NO:88), GMsk93h09 (SEQ ID NO:90), GMso31a02 (SEQ ID NO:92), LU61649369 (SEQ ID NO:94), LU61704197 (SEQ ID NO:96), ZM57508275 (SEQ ID NO:98), and ZM59288476 (SEQ ID NO:100). The alignment was generated using Align X of Vector NTI.

FIG. 5 shows an alignment of the disclosed amino acid sequences BN42194524 (SEQ ID NO:102), ZM68498581 (SEQ ID NO:104), BN42062606 (SEQ ID NO:106), BN42261838 (SEQ ID NO:108), BN43722096 (SEQ ID NO:110), GM50585691 (SEQ ID NO:112), GMsa56c07 (SEQ ID NO:114), GMsb20d04 (SEQ ID NO:116), GMsg04a02 (SEQ ID NO:118), GMsp36c10 (SEQ ID NO:120), GMsp82f11 (SEQ ID NO:122), GMss66f03 (SEQ ID NO:124), LU61748885 (SEQ ID NO:126), 0S36582281 (SEQ ID NO:128), 0S40057356 (SEQ ID NO:130), ZM57588094 (SEQ ID NO:132), ZM67281604 (SEQ ID NO:134), and ZM68466470 (SEQ ID NO:136). The alignment was generated using Align X of Vector NTI

FIG. 6 shows an alignment of the disclosed amino acid sequences BN45660154_{—}5 (SEQ ID NO:138), BN45660154_{—}8 (SEQ ID NO:140), and ZM58885021 (SEQ ID NO:142), and BN46929759 (SEQ ID NO:144). The alignment was generated using Align X of Vector NTI.

FIG. 7 shows an alignment of the disclosed amino acid sequences BN43100775 (SEQ ID NO:146), GM59673822 (SEQ ID NO:148), and ZM59314493 (SEQ ID NO:150). The alignment was generated using Align X of Vector NTI.

FIG. 8 shows an alignment of the disclosed amino acid sequences At5G60750 (SEQ ID NO:158), BN47819599 (SEQ ID NO:160), and ZM65102675 (SEQ ID NO:162). The alignment was generated using Align X of Vector NTI.

FIG. 9 shows an alignment of the disclosed amino acid sequences BN51278543 (SEQ ID NO:164), GM59587627 (SEQ ID NO:166), GMsae76c10 (SEQ ID NO:168), ZM68403475 (SEQ ID NO:170), and ZMTD14006355 (SEQ ID NO:172). The alignment was generated using Align X of Vector NTI.

FIG. 10 shows an alignment of the disclosed amino acid sequences BN48622391 (SEQ ID NO:176), GM50247805 (SEQ ID NO:178), and ZM62208861 (SEQ ID NO:180). The alignment was generated using Align X of Vector NTI.

FIG. 11 shows an alignment of the disclosed amino acid sequences GM49819537 (SEQ ID NO:182), BN42562310 (SEQ ID NO:184), GM47121078 (SEQ ID NO:186), and GMsf89h03 (SEQ ID NO:188). The alignment was generated using Align X of Vector NTI.

FIG. 12 shows an alignment of the disclosed amino acid sequences HA66670700 (SEQ ID NO:190), GM50390979 (SEQ ID NO:192), GM59720014 (SEQ ID NO:194), GMsab62c11 (SEQ ID NO:196), GMs142e03 (SEQ ID NO:198), and GMss72c01 (SEQ ID NO:200). The alignment was generated using Align X of Vector NTI.

FIG. 13 shows an alignment of the disclosed amino acid sequences ZM62043790 (SEQ ID NO:154), GMsk21g122 (SEQ ID NO:156), and GMsk21ga12 (SEQ ID NO:152). The alignment was generated using Align X of Vector NTI.

FIG. 14 shows an alignment of the disclosed amino acid sequences EST285 (SEQ ID NO:208), BN42471769 (SEQ ID NO:210), and ZM100324 (SEQ ID NO:212), BN42817730 (SEQ ID NO:214), BN45236208 (SEQ ID NO:216), BN46730374 (SEQ ID NO:218), BN46832560 (SEQ ID NO:220), BN46868821 (SEQ ID NO:222), GM48927342 (SEQ ID NO:224), GM48955695 (SEQ ID NO:226), GM48958569 (SEQ ID NO:228), GM50526381 (SEQ ID NO:230), HA66511283 (SEQ ID NO:232), HA66563970 (SEQ ID NO:234), HA66692703 (SEQ ID NO:236), HA66822928 (SEQ ID NO:238), LU61569679 (SEQ ID NO:240), LU61703351 (SEQ ID NO:242), LU61962194 (SEQ ID NO:244), TA54564073 (SEQ ID NO:246), TA54788773 (SEQ ID NO:248), TA56412836 (SEQ ID NO:250), and ZM65144673 (SEQ ID NO:252). The alignment was generated using Align X of Vector NTI

FIG. 15 shows an alignment of the disclosed amino acid sequences EST589 (SEQ ID NO:258), BN45899621 (SEQ ID NO:260), BN51334240 (SEQ ID NO:262), BN51345476 (SEQ ID NO:264), BN42856089 (SEQ ID NO:266), BN43206527 (SEQ ID NO:268), GMsf85h09 (SEQ ID NO:270), GMsj98e01 (SEQ ID NO:272), GMsu65h07 (SEQ ID NO:274), HA66777473 (SEQ ID NO:276), LU61781371 (SEQ ID NO:278), LU61589678 (SEQ ID NO:280), LU61857781 (SEQ ID NO:282), TA55079288 (SEQ ID NO:284), ZM59400933 (SEQ ID NO:286). The alignment was generated using Align X of Vector NTI.

FIG. 16 shows a flow diagram of acetyl-CoA metabolism and fatty acid biosynthesis with relation to the gene products that modify yield.

FIG. 17 shows an alignment of the amino acid sequences of the acyl-CoA synthetase long-chain-fatty-acid-CoA ligase subunits designated b1805 (SEQ ID NO:288), YER015W (SEQ ID NO:290), GM59544909 (SEQ ID NO:292), GM59627238 (SEQ ID NO:294), GM59727707 (SEQ ID NO:296), ZM57432637 (SEQ ID NO:298), ZM58913368 (SEQ ID NO:300), ZM62001931 (SEQ ID NO:302), ZM65438309 (SEQ ID NO:304), GM59610424 (SEQ ID NO:306), GM59661358 (SEQ ID NO:308), GMst55d11 (SEQ ID NO:310), ZM65362798 (SEQ ID NO:312), ZM62261160 (SEQ ID NO:314), and ZM62152441 (SEQ ID NO:316). The alignment was generated using Align X of Vector NTI.

FIG. 18 shows an alignment of the amino acid sequences of the biotin carboxylase subunits of acetyl CoA carboxylase designated b3256 (SEQ ID NO:322), BN49370246 (SEQ ID NO:324), GM59606041 (SEQ ID NO:326), GM59537012 (SEQ ID NO:328). The alignment was generated using Align X of Vector NTI.

FIG. 19 shows an alignment of the amino acid sequences of the acetyl-CoA carboxylase biotin carboxyl carrier protein subunits designated b3255 (SEQ ID NO:330), BN493-42080 (SEQ ID NO:332), BN45576739 (SEQ ID NO:334). The alignment was generated using Align X of Vector NTI.

FIG. 20 shows an alignment of the amino acid sequences b1095 (SEQ ID NO:336), GM48933354 (SEQ ID NO:338), ZM59397765 (SEQ ID NO:340), GM59563409 (SEQ ID NO:342). The alignment was generated using Align X of Vector NTI.

FIG. 21 shows an alignment of the disclosed amino acid sequences B1093 (SEQ ID NO:344), slr0886 (SEQ ID NO:346), BN44033445 (SEQ ID NO:348), BN43251017 (SEQ ID NO:350), BN42133443 (SEQ ID NO:352), GM49771427 (SEQ ID NO:354), GM48925912 (SEQ ID NO:356), GM51007060 (SEQ ID NO:358), GM59598120 (SEQ ID NO:360), GM59619826 (SEQ ID NO:362), GMsaa65f11 (SEQ ID NO:364), GMsf29g01 (SEQ ID NO:366), GMsn33h01 (SEQ ID NO:368), GMsp73h12 (SEQ ID NO:370), GMst67g06 (SEQ ID NO:372), GMsu14e09 (SEQ ID NO:374), GMsu65c05 (SEQ ID NO:376), HV62626732 (SEQ ID NO:378), LU61764715 (SEQ ID NO:380), 0S32620492 (SEQ ID NO:382), ZM57377353 (SEQ ID NO:384), ZM58204125 (SEQ ID NO:386), ZM58594846 (SEQ ID NO:388), ZM62192824 (SEQ ID NO:390), ZM65173545 (SEQ ID NO:392), ZM65173829 (SEQ ID NO:394), ZM57603160 (SEQ ID NO:396). The alignment was generated using Align X of Vector NTI.

FIG. 22 shows an alignment of the biotin synthetase amino acid sequences slr1364 (SEQ ID NO:398), BN51403883 (SEQ ID NO:400), ZM65220870 (SEQ ID NO:402). The alignment was generated using Align X of Vector NTI.

FIG. 23 shows a flow diagram of phytosterol metabolism as it relates to the present invention.

FIG. 24 shows an alignment of the amino acid sequences of the farnesyl diphosphate synthases designated B0421 (SEQ ID NO:414), YJL167W (SEQ ID NO:416), BN42777400 (SEQ ID NO:418), BN43165280 (SEQ ID NO:420), GMsf33b12 (SEQ ID NO:422), GMsa58c11 (SEQ ID NO:424), GM48958315 (SEQ ID NO:426), TA55347042 (SEQ ID NO:428), TA59981866 (SEQ ID NO:430), ZM68702208 (SEQ ID NO:432), ZM62161138 (SEQ ID NO:434). The alignment was generated using Align X of Vector NTI.

FIG. 25 shows an alignment of the amino acid sequences of the squalene synthases designated SQS1 (SEQ ID NO:436), SQS2 (SEQ ID NO:438), BN51386398 (SEQ ID NO:440), GM59738015 SEQ ID NO:442), ZM68433599 (SEQ ID NO:444), A9RRG4 (SEQ ID NO:463), O22107 (SEQ ID NO:464), Q84LE3 (SEQ ID NO:465), O22106 (SEQ ID NO:466), Q6Z368 (SEQ ID NO:467), YHR190W (SEQ ID NO:468). The alignment was generated using Align X of Vector NTI.

FIG. 26 shows an alignment of the amino acid sequences of the squalene epoxidases designated YGR175C (SEQ ID NO:446), BN48837983 (SEQ ID NO:448), ZM62269276 (SEQ ID NO:450). The alignment was generated using Align X of Vector NTI.

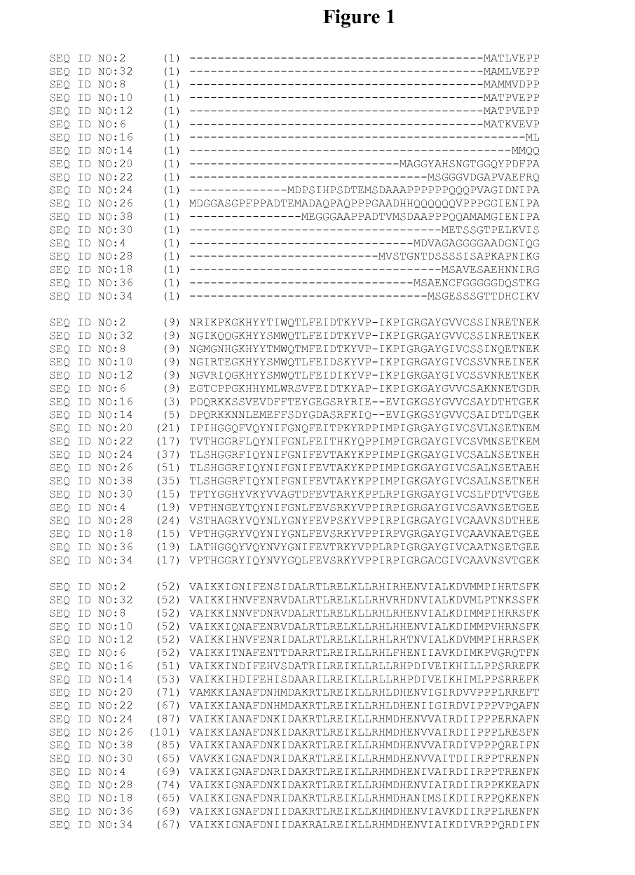

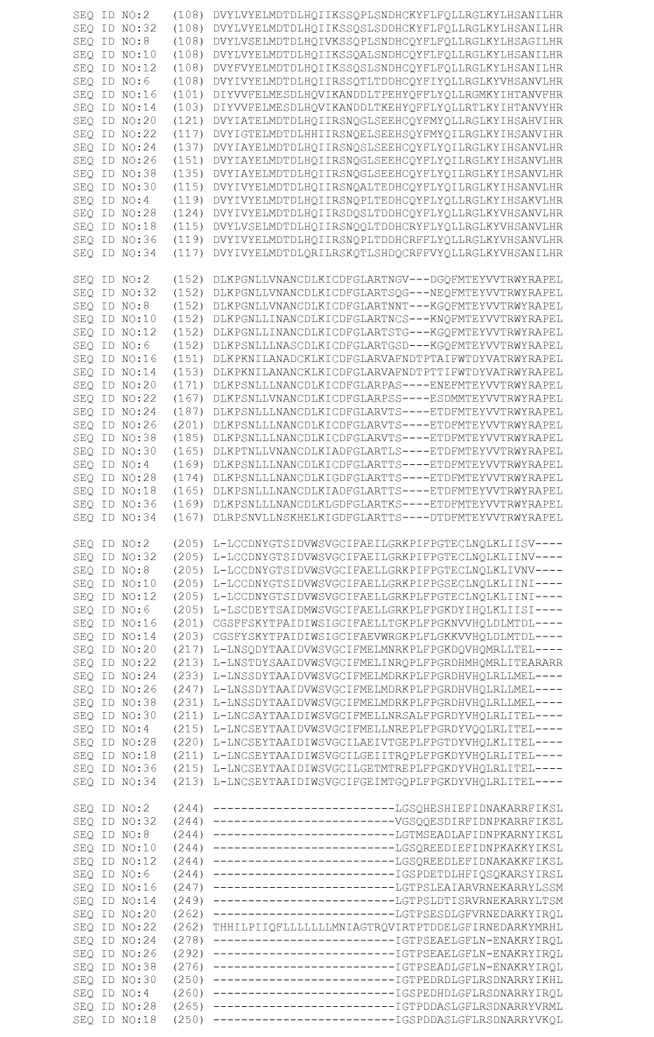

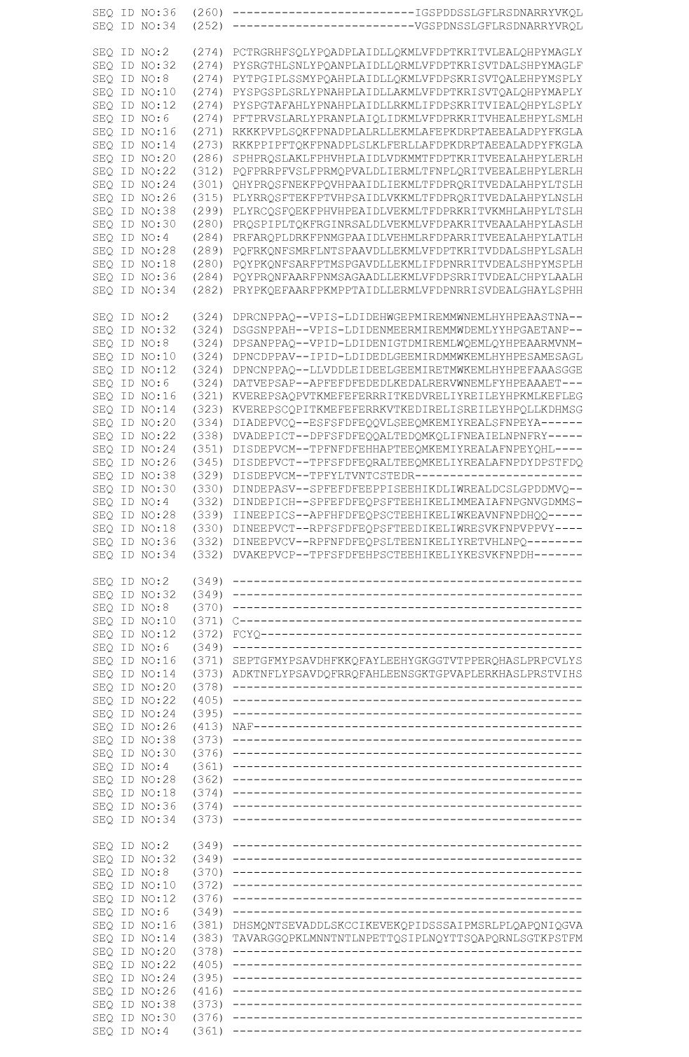

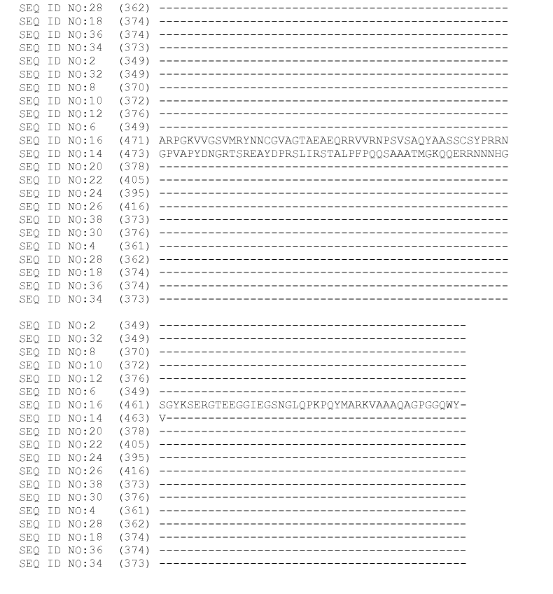

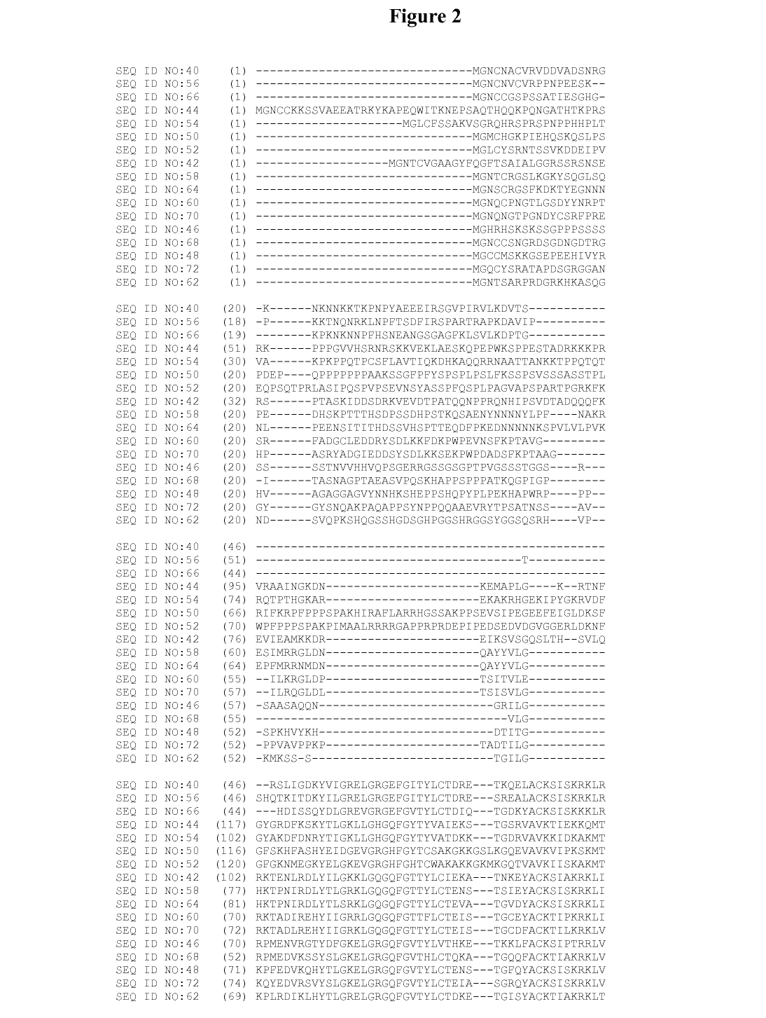

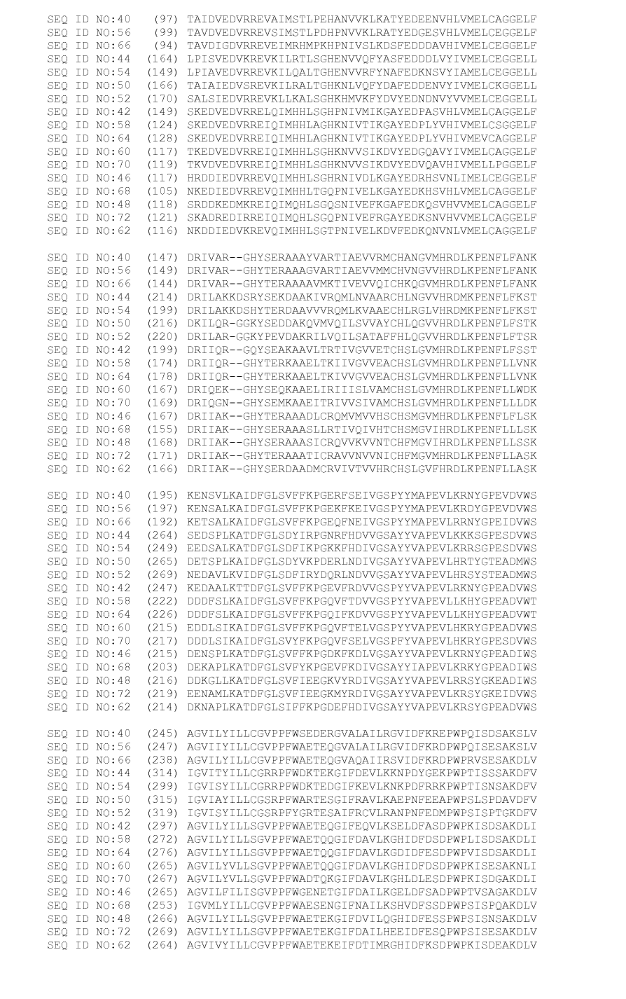

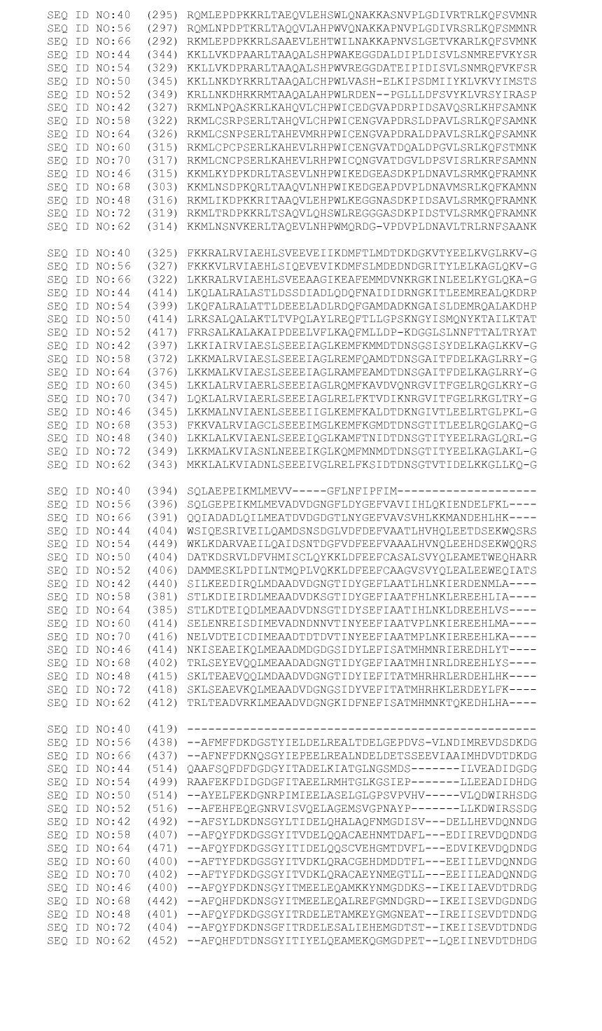

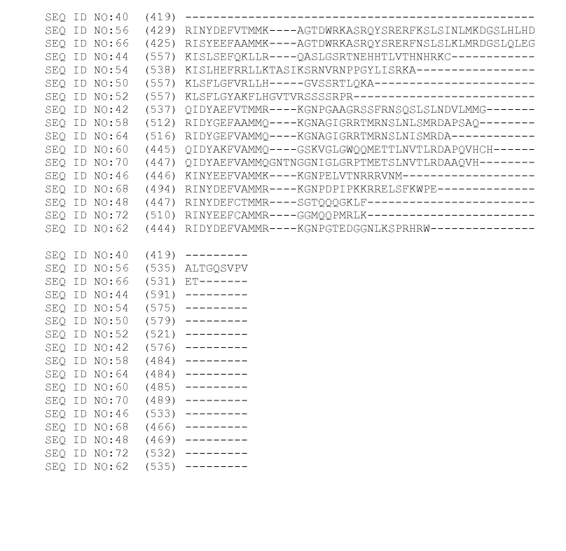

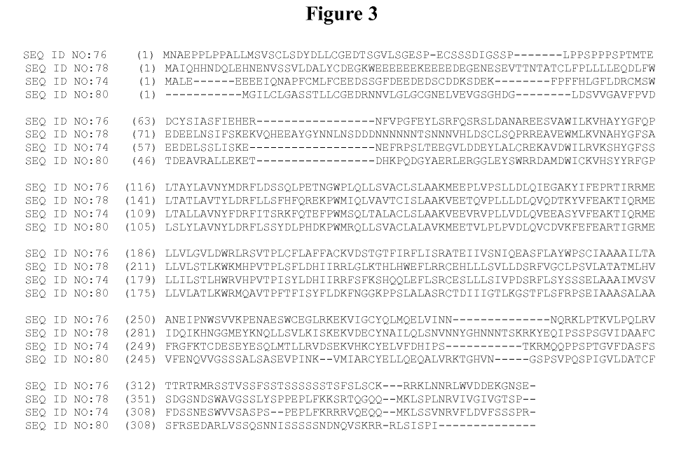

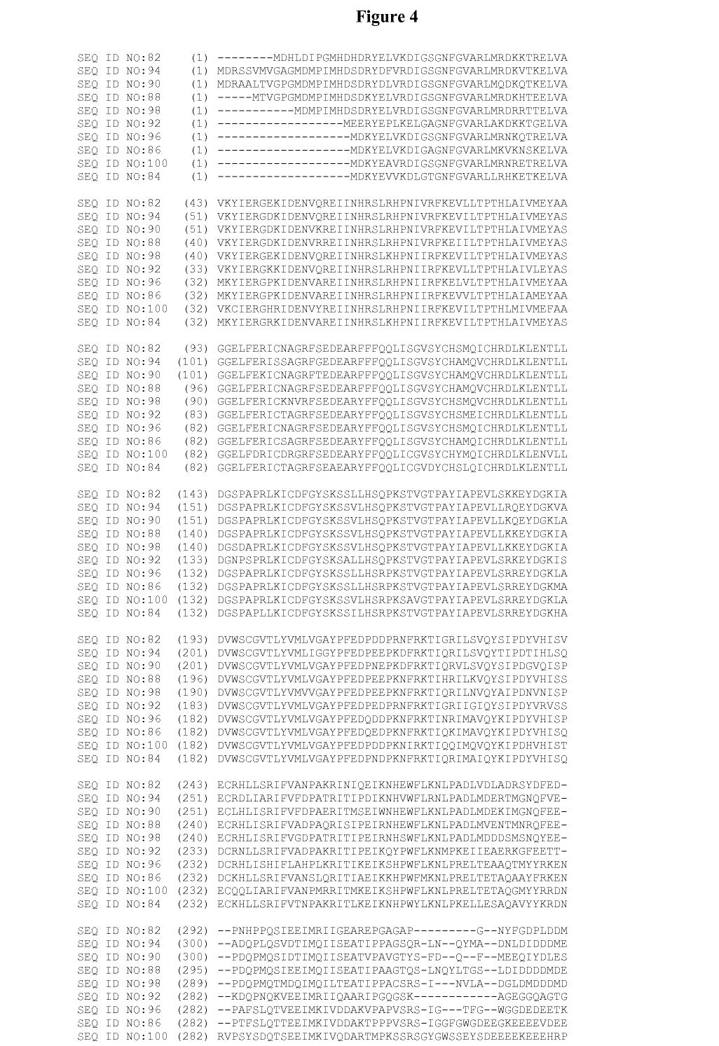

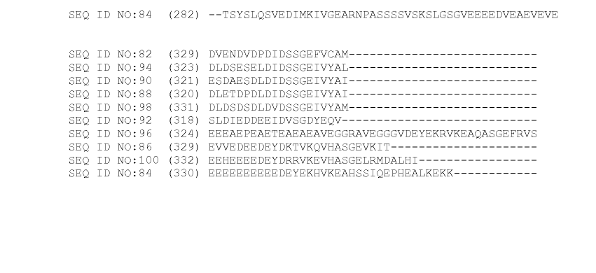

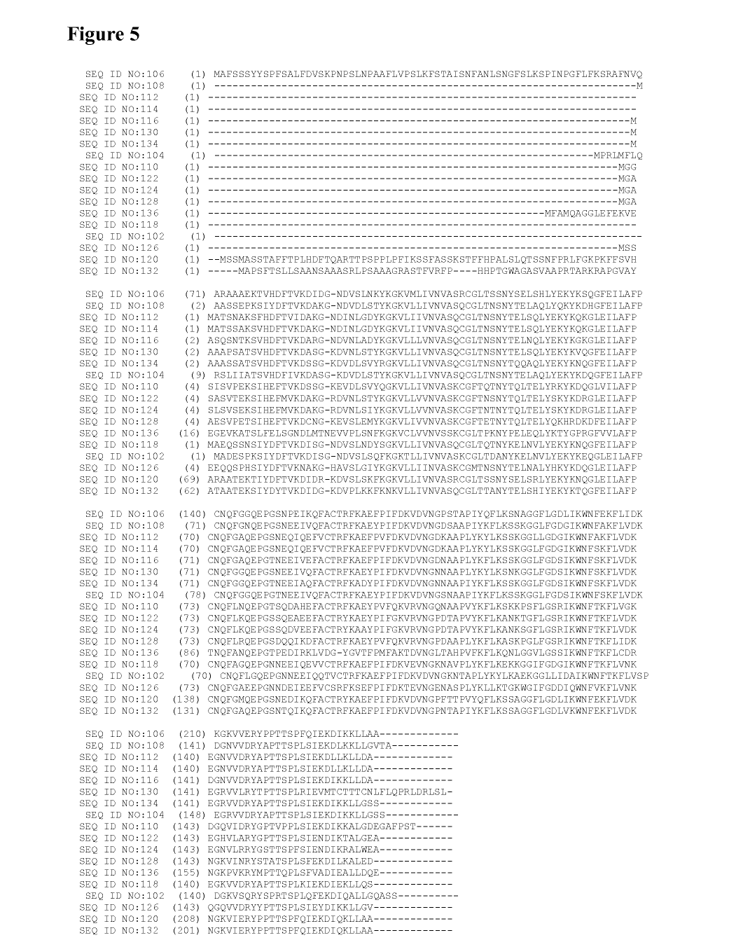

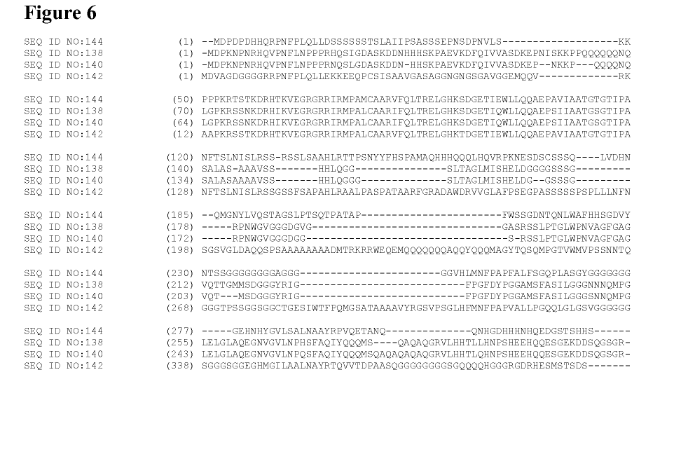

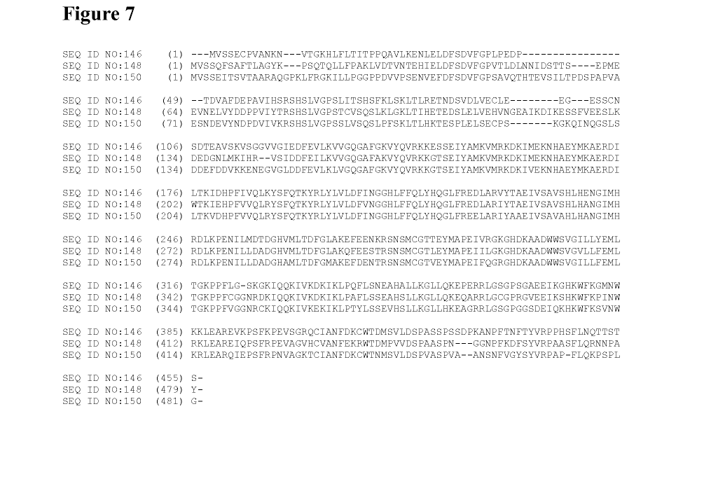

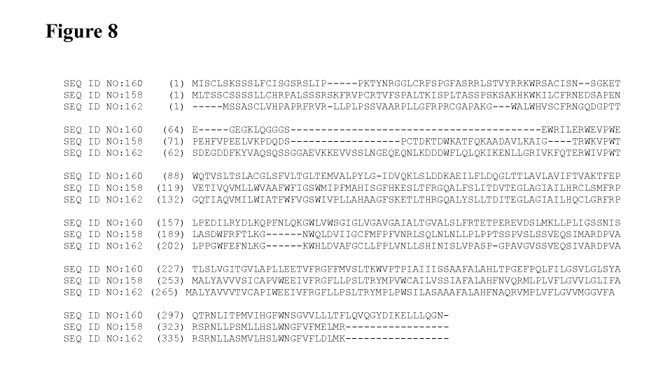

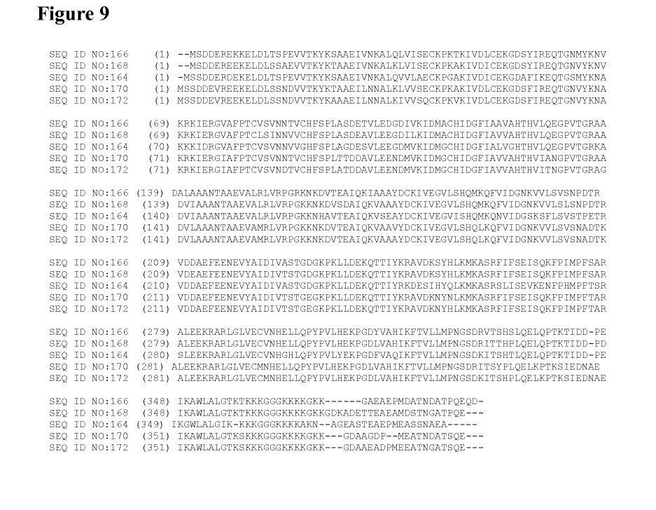

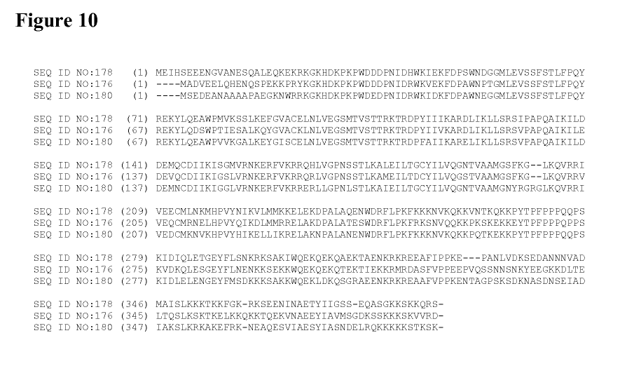

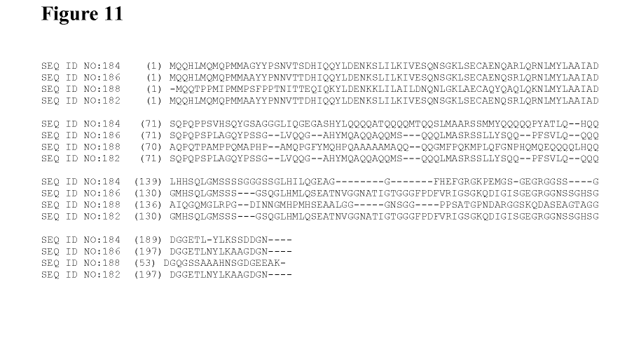

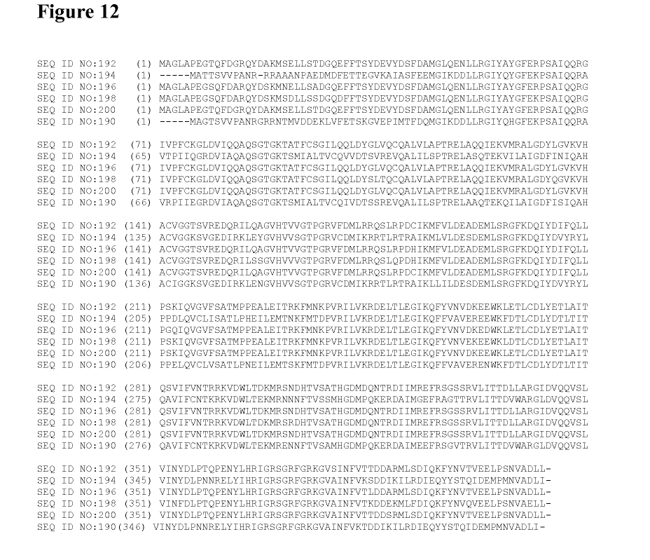

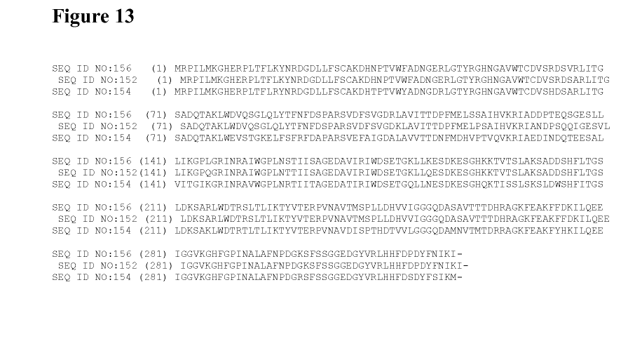

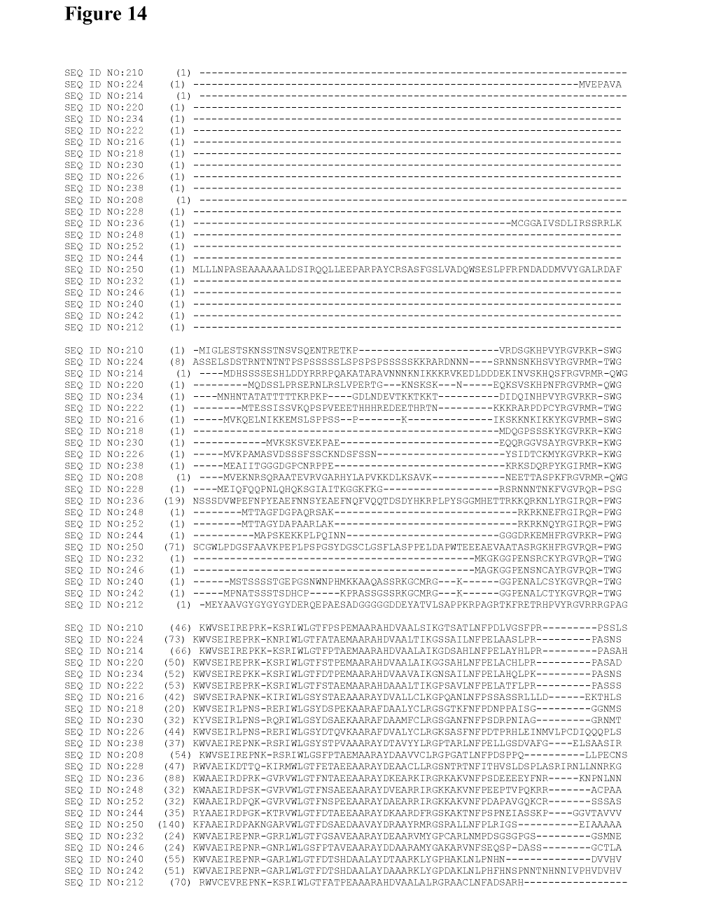

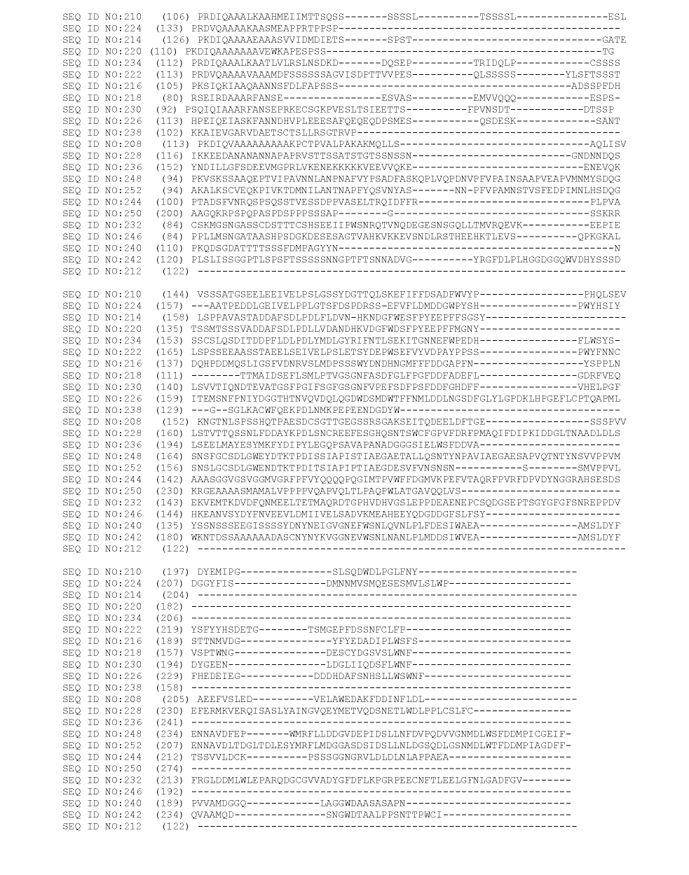

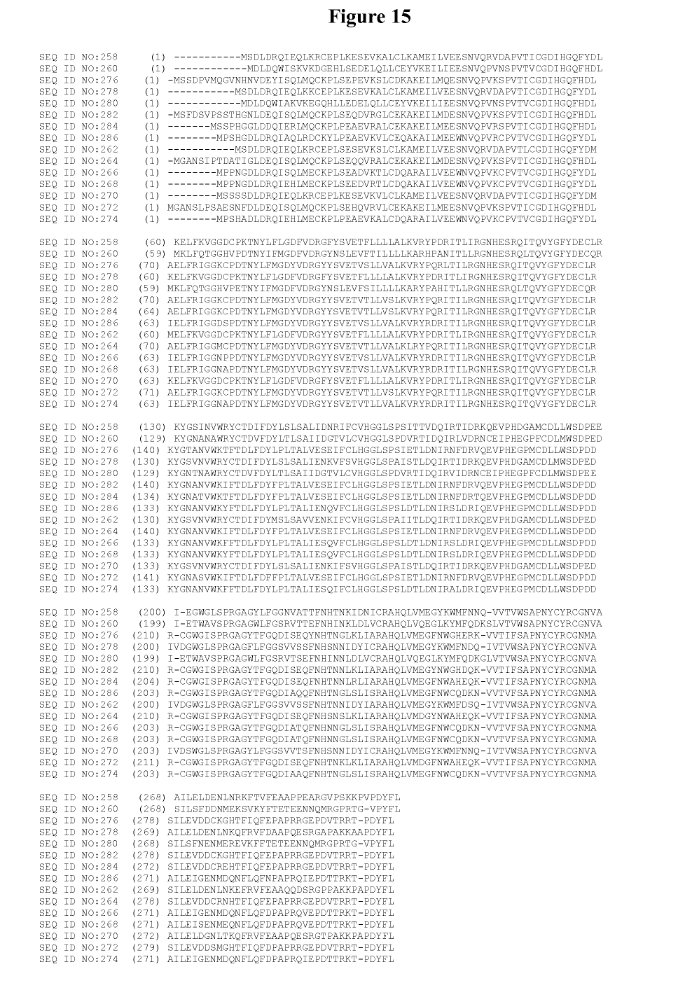

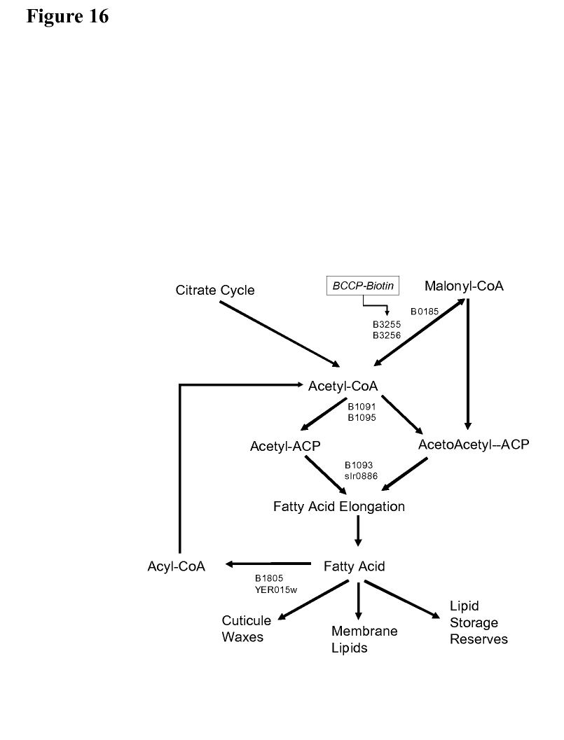

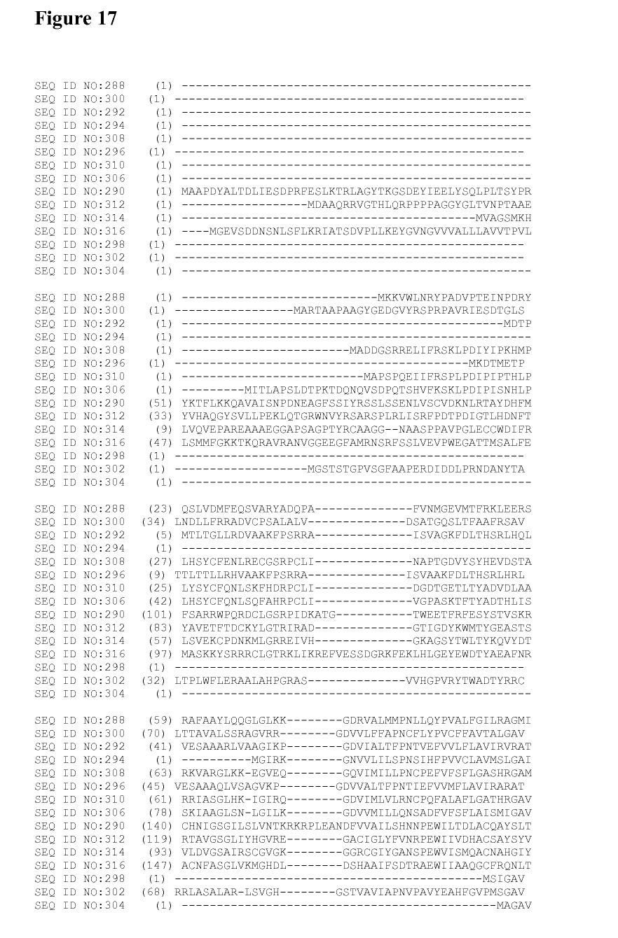

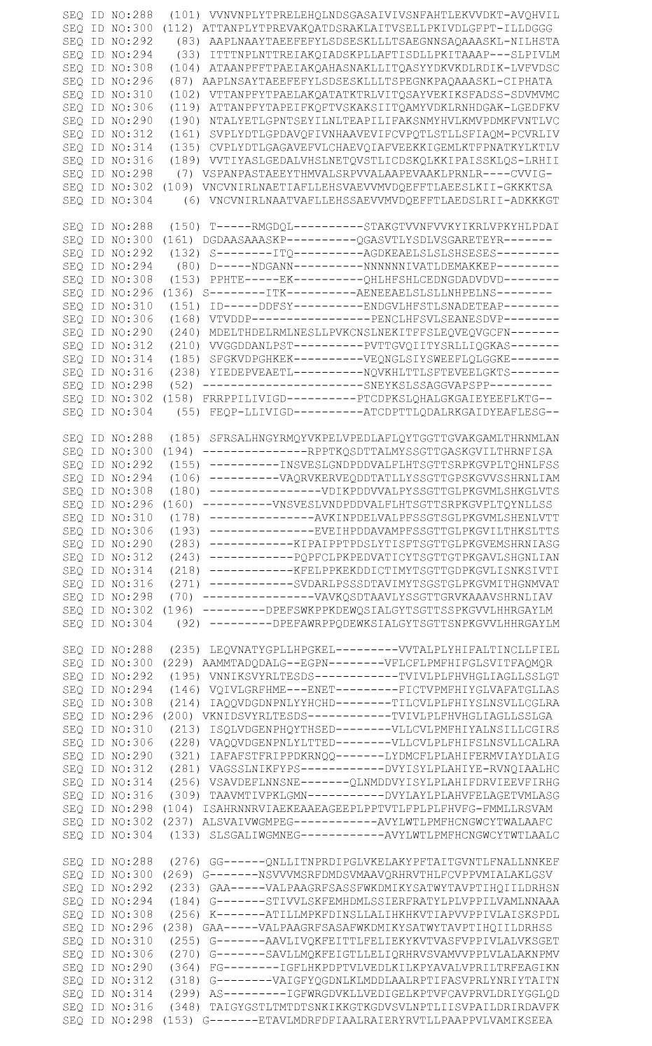

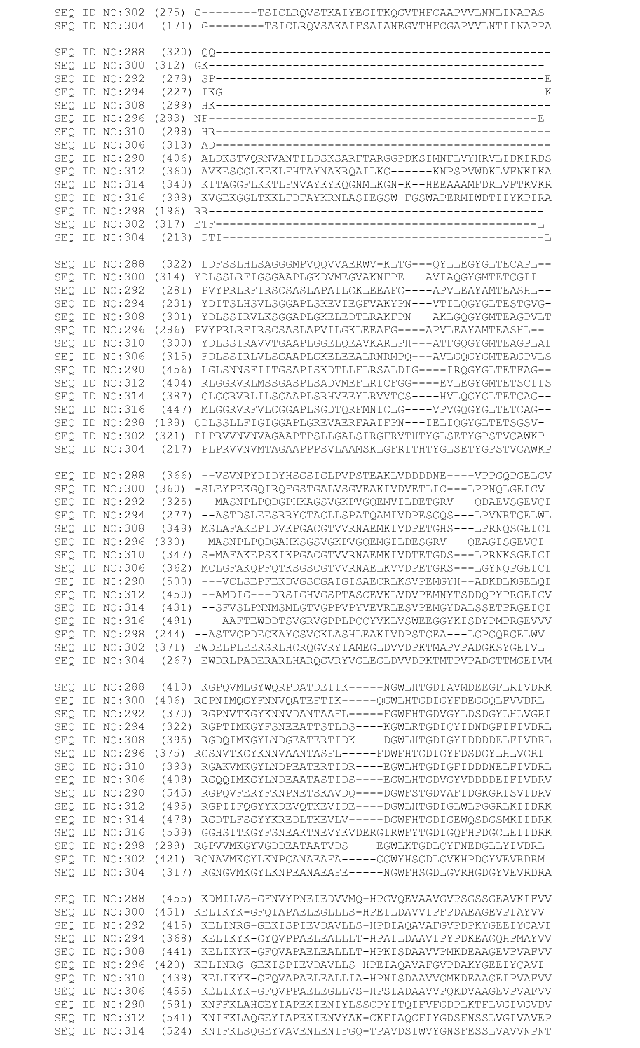

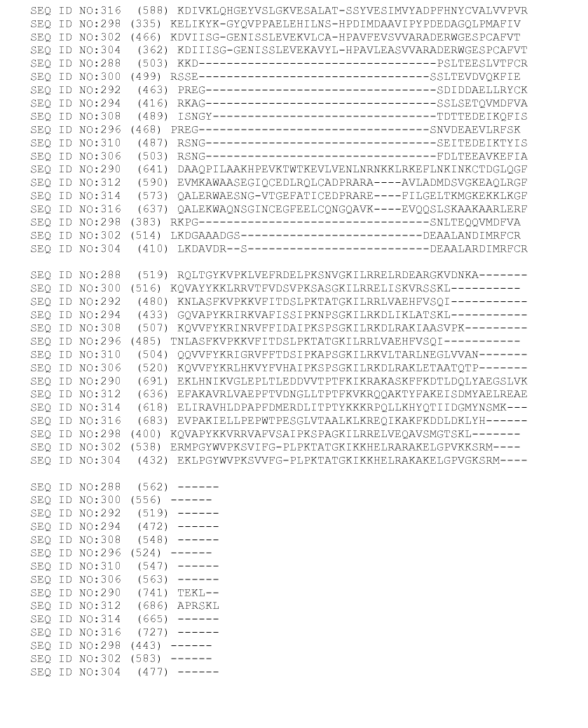

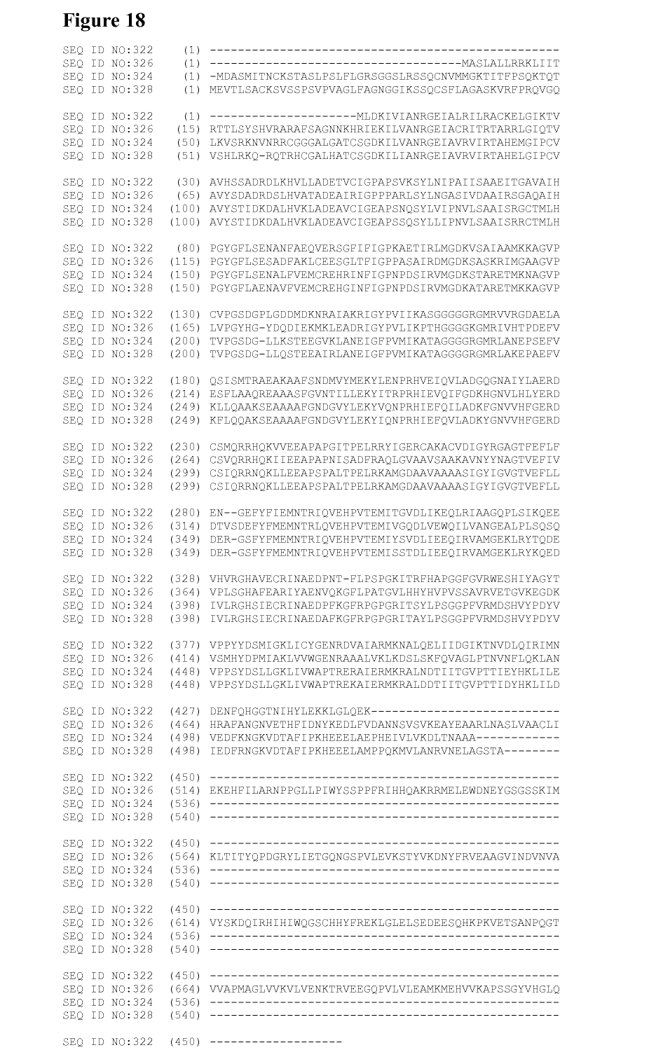

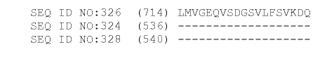

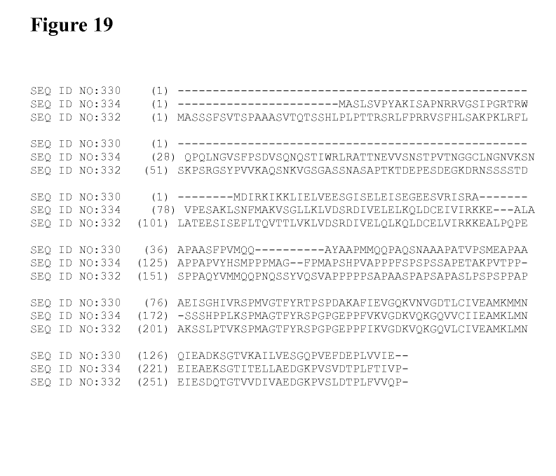

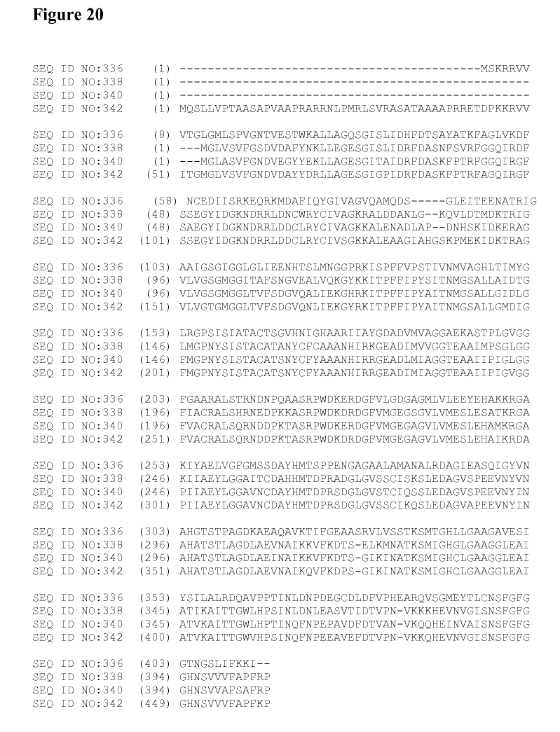

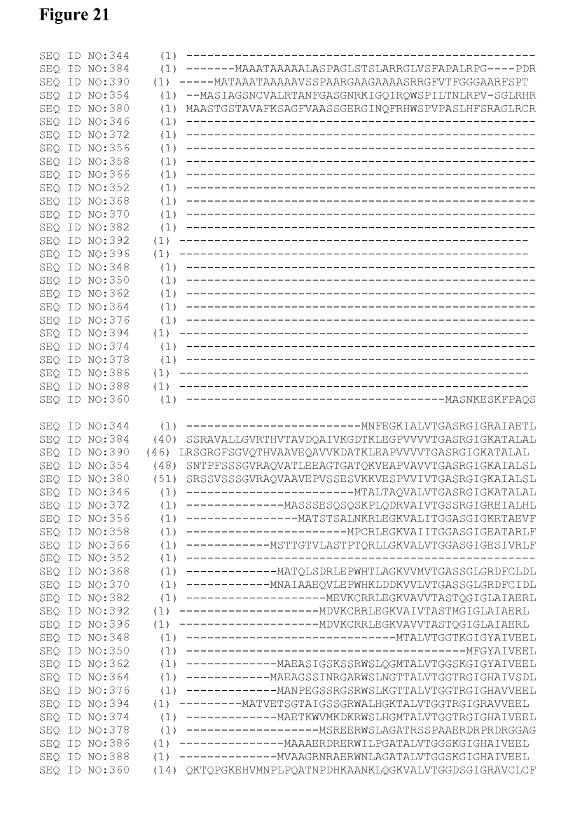

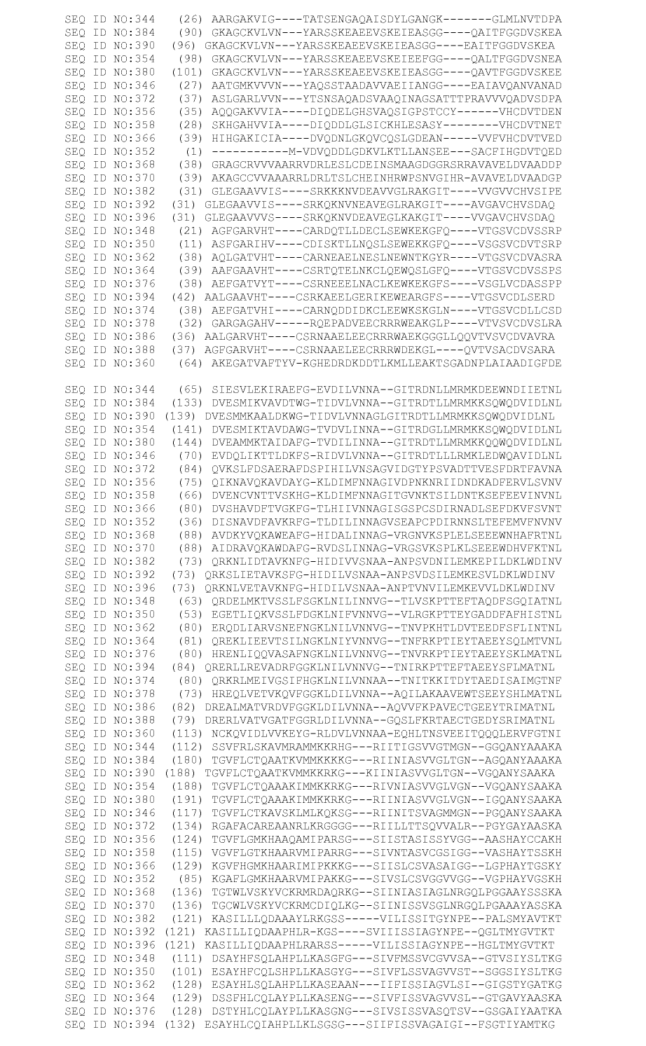

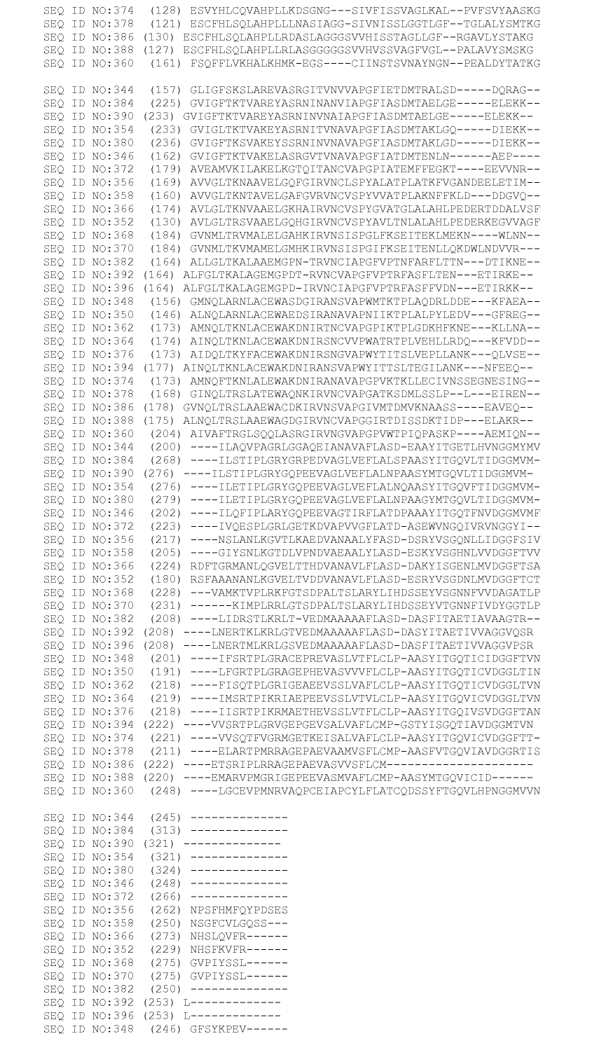

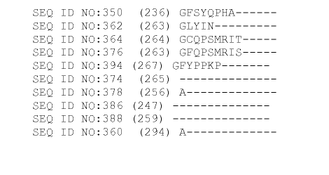

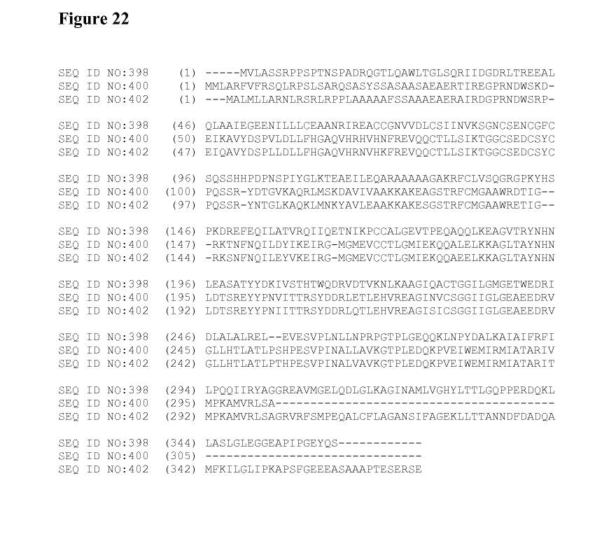

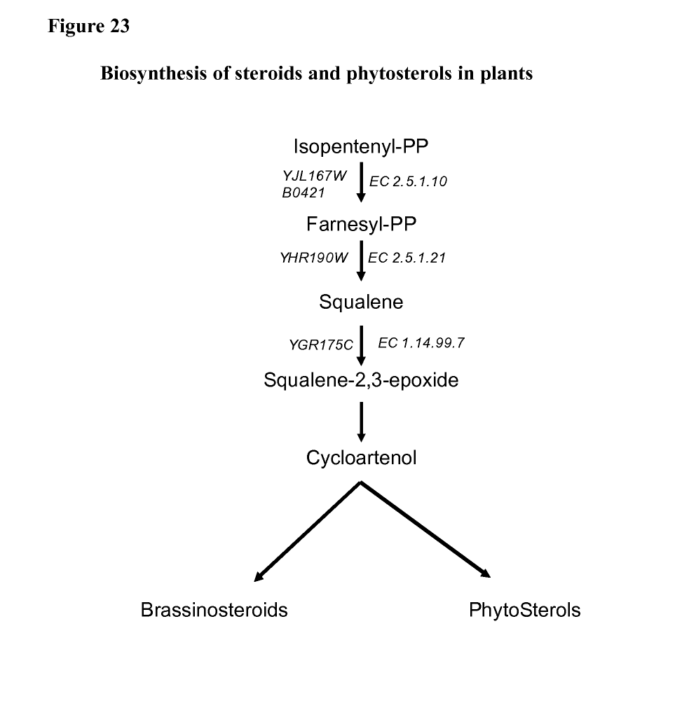

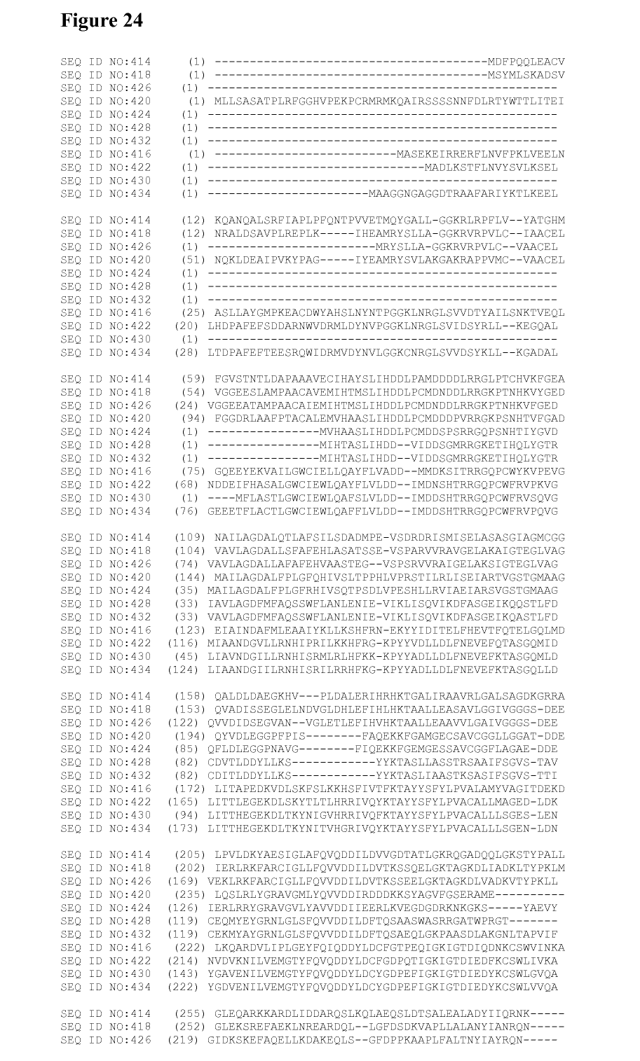

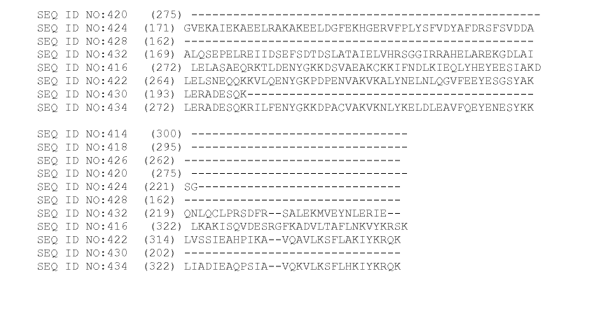

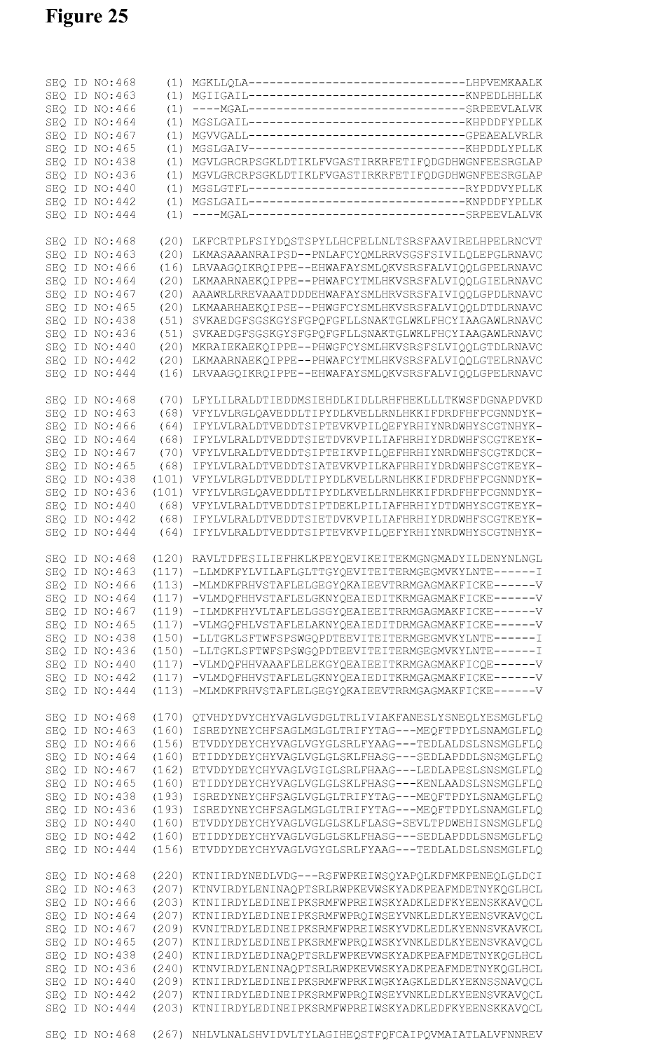

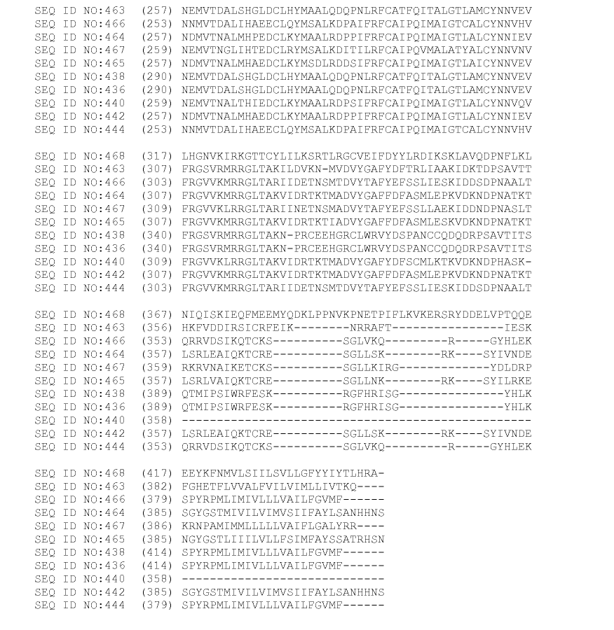

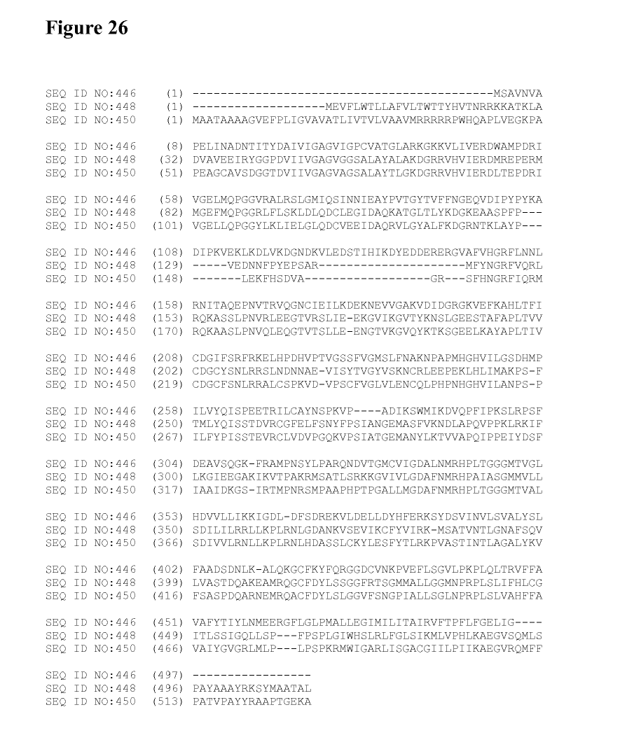

1. A transgenic plant transformed with an expression cassette comprising a polynucleotide encoding a full-length polypeptide
a) having mitogen activated protein kinase activity, wherein the polypeptide comprises a domain having a sequence selected from the group consisting of amino acids 42 to 329 of SEQ ID NO:4; amino acids 32 to 319 of SEQ ID NO:2; amino acids 32 to 319 of SEQ ID NO:6; amino acids 32 to 310 of SEQ ID NO:8; amino acids 32 to 319 of SEQ ID NO:10; amino acids 32 to 319 of SEQ ID NO:12; amino acids 28 to 318 of SEQ ID NO:14; amino acids 32 to 326 of SEQ ID NO:16; amino acids 38 to 325 of SEQ ID NO:18; amino acids 44 to 331 of SEQ ID NO:20; amino acids 40 to 357 of SEQ ID NO:22; amino acids 60 to 346 of SEQ ID NO:24; amino acids 74 to 360 of SEQ ID NO:26; and amino acids 47 to 334 of SEQ ID NO:28 amino acids 47 to 334 of SEQ ID NO:28; amino acids 38 to 325 of SEQ ID NO:30; amino acids 32 to 319 of SEQ ID NO:32; amino acids 41 to 327 of SEQ ID NO:34; amino acids 43 to 329 of SEQ ID NO:36; and amino acids 58 to 344 of SEQ ID NO:38, or
b) having phospholipid hydroperoxide glutathione peroxidase activity, wherein the polypeptide comprises a glutathione peroxidase domain selected from the group consisting of 9 to 117 of SEQ ID NO:102; amino acids 17 to 125 of SEQ ID NO:104; amino acids 79 to 187 of SEQ ID NO:106; amino acids 10 to 118 of SEQ ID NO:108; amino acids 12 to 120 of SEQ ID NO:110; amino acids 9 to 117 of SEQ ID NO:112; amino acids 9 to 117 of SEQ ID NO:114; amino acids 10 to 118 of SEQ ID NO:116; amino acids 9 to 117 of SEQ ID NO:118; amino acids 77 to 185 of SEQ ID NO:120; amino acids 12 to 120 of SEQ ID NO:122; amino acids 12 to 120 of SEQ ID NO:124; amino acids 12 to 120 of SEQ ID NO:126; amino acids 12 to 120 of SEQ ID NO:128; amino acids 10 to 118 of SEQ ID NO:130; amino acids 70 to 178 of SEQ ID NO:132; amino acids 10 to 118 of SEQ ID NO:134; and amino acids 24 to 132 of SEQ ID NO:136, or
c) comprising a TCP family transcription factor domain having a sequence selected from the group consisting of amino acids 57 to 249 of SEQ ID NO:138; amino acids 54 to 237 of SEQ ID NO:140; amino acids 43 to 323 of SEQ ID NO:142; or amino acids 41 to 262 of SEQ ID NO:144, or
d) comprising an AP2 domain having a sequence at least 64% identical to amino acids 44 to 99 of SEQ ID NO:208, or
e) comprising a polynucleotide encoding a full-length brassinosteroid biosynthetic LKB-like polypeptide selected from the group consisting of amino acids 1 to 566 of SEQ ID NO:254, CAN79299, AAK15493, P93472, AAM47602, and AAL91175, or
f) comprising, in operative association i) an isolated polynucleotide encoding a promoter capable of enhancing gene expression in leaves; and ii) an isolated polynucleotide encoding a full-length polypeptide which is a long-chain-fatty-acid-CoA ligase subunit of acyl-CoA synthetase; wherein the transgenic plant demonstrates increased yield as compared to a wild type plant of the same variety which does not comprise the expression cassette, or
g) comprising, in operative association, i) an isolated polynucleotide encoding a promoter capable of enhancing gene expression in leaves; ii) an isolated polynucleotide encoding a mitochondrial transit peptide; and iii) an isolated polynucleotide encoding a full-length farnesyl diphosphate synthase polypeptide; wherein the transgenic plant demonstrates increased yield as compared to a wild type plant of the same variety which does not comprise the expression cassette.

2. The transgenic plant of claim 1, wherein the polypeptide having mitogen activated protein kinase activity comprises amino acids 1 to 376 of SEQ ID NO:4; amino acids 1 to 368 of SEQ ID NO:2; amino acids 1 to 368 of SEQ ID NO:6; amino acids 1 to 369 of SEQ ID NO:8; amino acids 1 to 371 of SEQ ID NO:10; amino acids 1 to 375 of SEQ ID NO:12; amino acids 1 to 523 of SEQ ID NO:14; amino acids 1 to 494 of SEQ ID NO:16; amino acids 1 to 373 of SEQ ID NO:18; amino acids 1 to 377 of SEQ ID NO:20; amino acids 1 to 404 of SEQ ID NO:22; amino acids 1 to 394 of SEQ ID NO:24; amino acids 1 to 415 of SEQ ID NO:26; amino acids 1 to 381 of SEQ ID NO:28 amino acids 1 to 381 of SEQ ID NO:28; amino acids 1 to 376 of SEQ ID NO:30; amino acids 1 to 368 of SEQ ID NO:32; amino acids 1 to 372 of SEQ ID NO:34; amino acids 1 to 374 of SEQ ID NO:36; or amino acids 1 to 372 of SEQ ID NO:38.

3. A transgenic plant transformed with an expression cassette comprising an isolated polynucleotide encoding a full-length polypeptide having calcium dependent protein kinase activity, wherein the polypeptide comprises:
a) a protein kinase domain selected from the group consisting of a domain having a sequence comprising amino acids 59 to 317 of SEQ ID NO:40; amino acids 111 to 369 of SEQ ID NO:42; amino acids 126 to 386 of SEQ ID NO:44; amino acids 79 to 337 of SEQ ID NO:46; amino acids 80 to 338 of SEQ ID NO:48; amino acids 125 to 287 of SEQ ID NO:50; amino acids 129 to 391 of SEQ ID NO:52; amino acids 111 to 371 of SEQ ID NO:54; amino acids 61 to 319 of SEQ ID NO:56; amino acids 86 to 344 of SEQ ID NO:58; amino acids 79 to 337 of SEQ ID NO:60; amino acids 78 to 336 of SEQ ID NO:62; amino acids 90 to 348 of SEQ ID NO:64; amino acids 56 to 314 of SEQ ID NO:66; amino acids 67 to 325 of SEQ ID NO:68; amino acids 81 to 339 of SEQ ID NO:70; and amino acids 83 to 341 of SEQ ID NO:72; and
b) at least one EF hand domain having a sequence selected from the group consisting of amino acids 364 to 392 of SEQ ID NO:40; amino acids 416 to 444 of SEQ ID NO:42; amino acids 433 to 461 of SEQ ID NO:44; amino acids 384 to 412 of SEQ ID NO:46; amino acids 385 to 413 of SEQ ID NO:48; amino acids 433 to 461 of SEQ ID NO:50; amino acids 436 to 463 of SEQ ID NO:52; amino acids 418 to 446 of SEQ ID NO:54; amino acids 366 to 394 of SEQ ID NO:56; amino acids 391 to 419 of SEQ ID NO:58; amino acids 384 to 412 of SEQ ID NO:60; amino acids 418 to 446 of SEQ ID NO:62; amino acids 395 to 423 of SEQ ID NO:64; amino acids 372 to 400 of SEQ ID NO:68; amino acids 388 to 416 of SEQ ID NO:72; amino acids 452 to 480 of SEQ ID NO:42; amino acids 470 to 498 of SEQ ID NO:44; amino acids 420 to 448 of SEQ ID NO:46; amino acids 421 to 449 of SEQ ID NO:48; amino acids 470 to 498 of SEQ ID NO:50; amino acids 472 to 500 of SEQ ID NO:52; amino acids 455 to 483 of SEQ ID NO:54; amino acids 402 to 430 of SEQ ID NO:56; amino acids 427 to 455 of SEQ ID NO:58; amino acids 420 to 448 of SEQ ID NO:60; amino acids 454 to 482 of SEQ ID NO:62; amino acids 444 to 472 of SEQ ID NO:68; amino acids 460 to 488 of SEQ ID NO:72; amino acids 488 to 516 of SEQ ID NO:42; amino acids 512 to 540 of SEQ ID NO:44; amino acids 456 to 484 of SEQ ID NO:46; amino acids 457 to 485 of SEQ ID NO:48; amino acids 510 to 535 of SEQ ID NO:50; amino acids 512 to 537 of SEQ ID NO:52; amino acids 497 to 525 of SEQ ID NO:54; amino acids 438 to 466 of SEQ ID NO:56; amino acids 463 to 491 of SEQ ID NO:58; amino acids 456 to 484 of SEQ ID NO:60; amino acids 522 to 550 of SEQ ID NO:42; amino acids 546 to 570 of SEQ ID NO:44; amino acids 491 to 519 of SEQ ID NO:46; amino acids 492 to 520 of SEQ ID NO:48; amino acids 542 to 570 of SEQ ID NO:50; amino acids 542 to 570 of SEQ ID NO:52; amino acids 531 to 555 of SEQ ID NO:54; amino acids 474 to 502 of SEQ ID NO:56; amino acids 497 to 525 of SEQ ID NO:58; and amino acid 490 to 518 of SEQ ID NO:60; amino acids 489 to 517 of SEQ ID NO:62; amino acids 501 to 529 of SEQ ID NO:64; amino acids 470 to 498 of SEQ ID NO:66; amino acids 479 to 507 of SEQ ID NO:68; amino acids 492 to 520 of SEQ ID NO:70; and amino acids 495 to 523 of SEQ ID NO:72.

4. The transgenic plant of claim 3, wherein the polypeptide has a sequence comprising amino acids 1 to 418 of SEQ ID NO:40; amino acids 1 to 575 of SEQ ID NO:42; amino acids 1 to 590 of SEQ ID NO:44; amino acids 1 to 532 of SEQ ID NO:46; amino acids 1 to 528 of SEQ ID NO:48; amino acids 1 to 578 of SEQ ID NO:50; amino acids 1 to 580 of SEQ ID NO:52; amino acids 1 to 574 of SEQ ID NO:54; amino acids 1 to 543 of SEQ ID NO:56; amino acids 1 to 549 of SEQ ID NO:58; amino acids 1 to 544 of SEQ ID NO:60; amino acids 1 to 534 of SEQ ID NO:62; amino acids 1 to 549 of SEQ ID NO:64; amino acids 1 to 532 of SEQ ID NO:66; amino acids 1 to 525 of SEQ ID NO:68; amino acids 1 to 548 of SEQ ID NO:70; or amino acids 1 to 531 of SEQ ID NO:72.

5. A transgenic plant transformed with an expression cassette comprising an isolated polynucleotide encoding a full-length polypeptide having cyclin dependent protein kinase activity, wherein the polypeptide comprises:
a) a cyclin N terminal domain having a sequence selected from the group consisting of amino acids 59 to 190 of SEQ ID NO:74; amino acids 63 to 197 of SEQ ID NO:76; amino acids 73 to 222 of SEQ ID NO:78; and amino acids 54 to 186 of SEQ ID NO:80, and
b) a cyclin C terminal domain having a sequence selected from the group consisting of amino acids 192 to 252 of SEQ ID NO:74; amino acids 199 to 259 of SEQ ID NO:76; amino acids 224 to 284 of SEQ ID NO:78; and amino acids 188 to 248 of SEQ ID NO:80.

6. The transgenic plant of claim 5, wherein the polypeptide has a sequence comprising amino acids 1 to 355 of SEQ ID NO:74; amino acids 1 to 360 of SEQ ID NO:76; amino acids 1 to 399 of SEQ ID NO:78; or amino acids 1 to 345 of SEQ ID NO:80.

7. A transgenic plant transformed with an expression cassette comprising an isolated polynucleotide encoding a full-length polypeptide having serine/threonine-specific protein kinase activity, wherein the polypeptide comprises a domain selected from the group consisting of a domain having a sequence comprising amino acids 15 to 271 of SEQ ID NO:82; amino acids 4 to 260 of SEQ ID NO:84; amino acids 4 to 260 of SEQ ID NO:86; amino acids 18 to 274 of SEQ ID NO:88; amino acids 23 to 279 of SEQ ID NO:90; amino acids 5 to 261 of SEQ ID NO:92; amino acids 23 to 279 of SEQ ID NO:94; amino acids 4 to 260 of SEQ ID NO:96; amino acids 12 to 268 of SEQ ID NO:98; and amino acids 4 to 260 of SEQ ID NO:100.

8. The transgenic plant of claim 7, wherein the polypeptide has a sequence comprising amino acids 1 to 348 of SEQ ID NO:82; amino acids 1 to 364 of SEQ ID NO:84; amino acids 1 to 354 of SEQ ID NO:86; amino acids 1 to 359 of SEQ ID NO:88; amino acids 1 to 360 of SEQ ID NO:90; amino acids 1 to 336 of SEQ ID NO:92; amino acids 1 to 362 of SEQ ID NO:94; amino acids 1 to 370 of SEQ ID NO:96; amino acids 1 to 350 of SEQ ID NO:98; or amino acids 1 to 361 of SEQ ID NO:100.

9. An isolated polynucleotide having a sequence selected from the group consisting of the polynucleotide sequences set forth in Table 1.

10. An isolated polypeptide having a sequence selected from the group consisting of the polypeptide sequences set forth in Table 1.

11. A method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of:
(a) introducing into a plant cell an expression vector comprising at least one polynucleotide listed in Table 1, and
(b) generating from the plant cell a transgenic plant that expresses the polynucleotide,
wherein expression of the polynucleotide in the transgenic plant results in the plant having increased growth or yield under normal or water-limited conditions or increased tolerance to environmental stress, as compared to a wild type variety of the plant.

12. A method of increasing a plant's growth or yield under normal or water-limited conditions or increasing a plant's tolerance to an environmental stress comprising the steps of
(a) introducing into a plant cell an expression vector comprising at least one polynucleotide listed in Table 1, and
(b) generating from the plant cell a transgenic plant that expresses the polynucleotide,
wherein expression of the polynucleotide in the transgenic plant results in the plant having increased growth or yield under normal or water-limited conditions or increased tolerance to environmental stress, as compared to a wild type variety of the plant.
